(19) United States
(12) Patent Application Publication
(10) Pub. No.: US 2008/0199480 A1
(43) Pub. Date: Aug. 21, 2008

(54) Methods for Identifying Risk of Type II Diabetes and Treatments Thereof

(75) Inventors: Maria L. Langdown, San Diego, CA (US); Matthew R. Nelson, Chapel Hill, NC (US); Rikard H. Reneland, Knivsta (SE); Stefan M. Kammerer, San Diego, CA (US); Andreas Braun, San Diego, CA (US); Mikhail F. Denissenko, Poway, CA (US); Josephine M. Atienza, San Diego, CA (US); Eddine Saiah, Cambridge, MA (US); James W. Zapf, San Diego, CA (US)

Correspondence Address: GRANT ANDERSON LLP;C/O PORTFOLIOIP, PO BOX 52050, MINNEAPOLIS, MN, 55402, US

(73) Assignee: SEQUENOM, INC., San Diego, CA (US)

(21) Appl. No.: 11/572,420

(22) PCT Filed: Jul. 22, 2004
(86) PCT No.: PCT/US2004/023813

§ 371 Date: Feb. 7, 2008

(51) Int. Cl. A61K 39/395; C12Q 1/68; C07H 21/00; C12N 5/06; A61P 3/10; C40B 40/08; C07K 14/00

(52) U.S. Cl. 424/172.1; 435/6; 536/24.31; 536/23.5; 506/17; 530/350; 435/366

(57) ABSTRACT

Provided herein are methods for identifying a risk of type II diabetes in a subject, reagents and kits for carrying out the methods, methods for identifying candidate therapeutics for treating type II diabetes, and therapeutic and preventative methods applicable to type II diabetes. These embodiments are based upon an analysis of polymorphic variations in nucleotide sequences within the human genome.

### FIELD OF THE INVENTION

The invention relates to genetic methods for identifying predisposition to type I diabetes, also known as non-insulin dependent diabetes, and treatments that specifically target the disease.

### BACKGROUND

Diabetes is among the most common of all metabolic disorders, affecting up to 11% of the population by age 70. Type I diabetes (insulin-dependent diabetes) represents about 5 to 10% of this group and is the result of progressive autoimmune destruction of the pancreatic beta-cells with subsequent insulin deficiency.

Type II diabetes (non-insulin dependent diabetes) represents 90-95% of the affected population, more than 100 million people worldwide. Approximately 17 million Americans suffer from type II diabetes, although 6 million do not even know they have the disease. The prevalence of the disease has jumped 33% in the last decade and is expected to rise further as the baby boomer generation gets older and more overweight. The global figure of people with diabetes is set to rise to an estimated 150 to 220 million in 2010, and 300 million in 2025. The widespread problem of diabetes has crept up on an unsuspecting health care community and has already imposed a huge burden on health-care systems (Zimmet et al. (2001) Nature 414: 782-787).

Often, the onset of type II diabetes can be insidious, or even clinically unapparent, making diagnosis difficult. Even when the disease is properly diagnosed, many of those treated do not have adequate control over their diabetes, resulting in elevated sugar levels in the bloodstream that slowly destroys the kidneys, eyes, blood vessels and nerves. This late damage is an important factor contributing to mortality in diabetics.

Type II diabetes is associated with peripheral insulin resistance, elevated hepatic glucose production, and inappropriate insulin secretion (DeFronzo, R. A. (1988) Diabetes 37:667-687), although the primary pathogenic lesion on type II diabetes remains elusive. Many have suggested that primary insulin resistance of the peripheral tissues is the initial event. Genetic epidemiological studies have supported this view. Similarly, insulin secretion abnormalities have been argued as the primary defect in type II diabetes. It is likely that both phenomena are important in the development of type II diabetes, and genetic defects predisposing to both are likely to be important contributors to the disease process (Rimoin, D. L., et al. (1996) Emery and Rimoin's Principles and Practice of Medical Genetics 3rd Ed. 1: 1401-1402).

Evidence from familial aggregation and twins studies point to a genetic component in the etiology of diabetes (Newman et al. (1987) Diabetologia 30:763-768; Kobberling, J. (1971) Diabetologia 7:46-49; Cook, J. T. E. (1994) Diabetologia 37:1231-1240), however, there is little agreement as to the nature of the genetic factors involved. This confusion can largely be attributed to the genetic heterogeneity known to exist in diabetes.

### SUMMARY

It has been discovered that certain polymorphic variations in human genomic DNA are associated with the occurrence of type II diabetes, also known as non-insulin dependent diabetes. In particular, polymorphic variants in a locus containing an EPHA3 gene region in human genomic DNA have been associated with risk of type II diabetes.

Thus, featured herein are methods for identifying a subject at risk of type II diabetes and/or a risk of type II diabetes in a subject, which comprise detecting the presence or absence of one or more polymorphic variations associated with type II diabetes in and around the locus described herein in a human nucleic acid sample. In an embodiment, two or more polymorphic variations are detected and in some embodiments, 3 or more, or 4, 5, 6, 7, 8, 9, 10, 11, 12, 13, 14, 15, 16, 17, 19, 19 or 20 or more polymorphic variants are detected.

Also featured are nucleic acids that include one or more polymorphic variations associated with occurrence of type II diabetes, as well as polypeptides encoded by these nucleic acids. In addition, provided are methods for identifying candidate therapeutic molecules for treating type II diabetes and other insulin-related disorders. In specific embodiments, featured are methods for identifying molecules that inhibit an interaction (e.g., binding) between the EPHA3 gene product (“EPHA3”) and one of its binding, partners, such as the binding partner ephrin-A5 or ephrin-M. In specific embodiments, an antibody is identified that specifically binds an EPHA3 isoform, ephrin-A5 or ephrin-A and decreases or blocks binding with EPHA3 in vitro and/or in vivo. Also provided are methods for treating type II diabetes in a subject by identifying a subject at risk of type II diabetes and treating the subject with a suitable prophylactic, treatment or therapeutic molecule. In specific embodiments, a method for treating type II diabetes is provided which comprises administering a molecule to a subject in need thereof that inhibits EPHA3 function, for example, by disrupting an interaction between EPHA3 and one of its binding partners, such as the binding partner ephrin-A5 or ephrin-A2, in an amount sufficient to reduce the interaction between the two proteins and to treat type II diabetes. Such a molecule may affect levels of C-peptide (e.g., often increasing levels of C-peptide), enhance glucose uptake in cells, increase triacylglycerol levels, and/or decrease resistin levels. In an embodiment, the molecule administered to the subject is an antibody that specifically binds to an EPHA3 isoform, ephrin-A5 or ephrin-A2 and inhibits or blocks binding between the two proteins. In another embodiment, the molecule administered to the subject is an epidermal growth factor (EGF), Src proto-oncogene tyrosine-protein kinase SRC), vascular endothelial growth factor (VEGF), or kinase insert domain receptor (KDR) inhibitor that also inhibits EPHA3. In yet another embodiment, the molecule administered to the subject is an EphA2 or EphB4 inhibitor that also inhibits EPHA3.

Also provided are compositions comprising a cell from a subject having type II diabetes or at risk of type II diabetes and/or an EPHA4 nucleic acid, with a nucleic acid that hybridizes to an EPHA3 nucleic acid under conditions of high stringency, or a RNAi, siRNA, antisense DNA or RNA, or a ribozyme nucleic acid designed from an EPHA3 nucleotide sequence. In an embodiment, the RNAi, siRNA, antisense DNA or RNA, or ribozyme nucleic acid is designed from an EPHA3 nucleotide sequence that includes one or more type II diabetes associated polymorphic variations, and in some instances, specifically interacts with such a nucleotide sequence. Further, provided are arrays of nucleic acids bound to a solid surface, in which one or more nucleic acid molecules of the array have an EPHA3 nucleotide sequence, or a fragment or substantially identical nucleic acid thereof, or a complementary nucleic acid of the foregoing. Featured also are compositions comprising a cell from a subject having type II diabetes or at risk of type II diabetes and/or an EPHA3 polypeptide, with an antibody that specifically binds to the polypeptide. In one an embodiment, the antibody specifically binds to an epitope in the polypeptide that includes a non-synonymous amino acid modification associated with type II diabetes (e.g. results in an amino acid substitution in the encoded polypeptide associated with type II diabetes). In embodiment, the antibody specifically binds to an epitope comprising an arginine at position 924, or a tryptophan at position 924, in an EPHA3 polypeptide (SEQ NO: 4).

### BRIEF DESCRIPTION OF THE DRAWINGS

FIGS. 1A-1C show proximal SNP p-values (based on allelotyping results in the discovery cohort) in an EPHA3 region for females, males, and males and females combined, respectively. FIGS. 1D-1F show proximal SNP p-values based on allelotyping results in a replication cohort in an EPHA3 region for females, males, and males and females combined, respectively. Positions of each SNP on the chromosome are shown on the x-axis and the y-axis provides the negative logarithm of the p-value comparing the estimated allele frequency in the cases to that of the control group. Also shown are exons and introns of genes in approximate chromosomal positions.

FIG. 2 shows meta-analysis results for EPHA3.

### DETAILED DESCRIPTION

It has been discovered that polymorphic variants in an EPHA3 locus in human genomic DNA are associated with occurrence of type II diabetes in subjects. Thus, detecting genetic determinants in and around this locus associated with an increased risk of type II diabetes occurrence can lead to early identification of a risk of type II diabetes and early application of preventative and treatment measures. Associating the polymorphic variants with type II diabetes also has provided new targets for diagnosing type II diabetes, and methods for screening molecules useful in diabetes treatments and diabetes preventatives.

EphA3, also known as Cek4, Mek4, Hek, Tyro4, and Hek4 (Unified nomenclature for Eph family receptors and their ligands, the ephrins. Eph Nomenclature Committee [letter]. Cell 90(3):403-404 (1997)), is a member of the Eph receptor family which binds members of the ephrin ligand family. EPHA3 has two isoforms produced by alternate splicing: transcript variant 1 is a membrane protein, and transcript variant 2 is secreted (see SEQ ID NO: 2 and 3). Both variants have an extracellular region consisting of a globular domain, a cysteine-rich domain, and two fibronectin type III domains, followed by the transmembrane region and cytoplasmic region. The cytoplasmic region contains a juxtamembrane motif with two tyrosine residues, which are the major autophosphorylation sites, a kinase domain, and a conserved sterile alpha motif (SAM) in the carboxy tail which contains one conserved tyrosine residue. Activation of kinase activity occurs after ligand recognition and binding. EphA3 has been shown to bind ephrin-A5, ephrin-A2, ephrin-A3, ephrin-A1, ephrin-A4, and ephrin-B1. (Flanagan, J. G. and P. Vanderhaegen, The ephrins and Eph receptors in neural development, Ann. Rev. Neuro Sci. 21:309-345 (1998); Pasquale, E. B. the Eph family of receptors, curr. Opin. Cell. Bio. 9:5):608-615 (1997)). However, high affinity ligands of EPHA3 include ephrin-A2 (which is expressed highly in the pancreas) and ephrin-A5 (which is highly expressed in heart and kidney). The extracellular domains of mouse and human EphA3 share greater than 96% amino acid identity. Only membrane-bound or Fc-clustered ligands are capable of activating the receptor in vitro. Soluble monomeric ligands bind the receptor but do not induce receptor autophosphorylation and activation. (Flanagan, J. G. and P. Vanderhaegen, The ephrins and Eph receptors in neural development, ann. Rev. neuro sci. 21:309-345 (1998)).

Type II Diabetes and Sample Selection

The term “type II diabetes” as used herein refers to non-insulin-dependent diabetes. Type II diabetes refers to an insulin-related disorder in which there is a relative disparity between endogenous insulin production and insulin requirements, leading to elevated hepatic glucose production, elevated blood glucose levels, inappropriate insulin secretion, and peripheral insulin resistance. Type II diabetes has been regarded as a relatively distinct disease entity, but type II diabetes is often a manifestation of a much broader underlying disorder (Zimmet et al (2001) Nature 414: 782-787), which may include metabolic syndrome (syndrome X), diabetes (e.g., type II diabetes, type II diabetes, gestational diabetes, autoimmune diabetes), hyperinsulinemia, hyperglycemia, impaired glucose tolerance (IGT), hypoglycemia, B-cell failure, insulin resistance, dyslipidemias, atheroma, insulinoma, hypertension, hypercoaguability, microalbuminuria, and obesity and obesity-related disorders such as visceral obesity, central fat, obesity-related type II diabetes, obesity-related atherosclerosis, heart disease, obesity-related insulin resistance, obesity-related hypertension, microangiopathic lesions resulting from obesity-related type II diabetes, ocular lesions caused by microangiopathy in obese individuals with obesity-related type U diabetes, and renal lesions caused by microangiopathy in obese individuals with obesity-related type II diabetes.

Some of the more common adult onset diabetes symptoms include fatigue, excessive thirst, frequent urination, blurred vision, a high rate of infections, wounds that heal slowly, mood changes and sexual problems. Despite these known symptoms, the onset of type II diabetes is often not discovered by health care professionals until the disease is well developed. Once identified, type II diabetes can be recognized in a patient by measuring fasting plasma glucose levels and/or casual plasma glucose levels, measuring fasting plasma insulin levels and/or casual plasma insulin levels, or administering oral glucose tolerance tests or hyperinsulinemic euglycemic clamp tests.

Based in part upon selection criteria set forth above, individuals having type II diabetes can be selected for genetic studies. Also, individuals having no history of metabolic disorders, particularly type II diabetes, often are selected for genetic studies as controls. The individuals selected for each pool of case and controls, were chosen following strict selection criteria in order to make the pools as homogenous as possible. Selection criteria for the study described herein included patient age, ethnicity, BMI, GAD (Glutamic Acid Decarboxylase) antibody concentration, and HbA1c (glycosylated hemoglobin A1c) concentration. GAD antibody is present in association with islet cell destruction, and therefore can be utilized to differentiate insulin dependent diabetes (type I diabetes) from non-insulin dependent diabetes (type II diabetes). HbA1c levels will reveal the average blood glucose over a period of 2-3 months or more specifically, over the life span of a red blood cell, by recording the number of glucose molecules attached to hemoglobin.

Polymorphic Variants Associated with Type II Diabetes

A genetic analysis provided herein linked type II diabetes with polymorphic variant nucleic acid sequences in the human genome. As used herein, the term “polymorphic site” refers to a region in a nucleic acid at which two or more alternative nucleotide sequences are observed in a significant number of nucleic acid samples from a population of individuals. A polymorphic site may be a nucleotide sequence of two or more nucleotides, an inserted nucleotide or nucleotide sequence, a deleted nucleotide or nucleotide sequence, or a microsatellite, for example. A polymorphic site that is two or more nucleotides in length may be 3, 4, 5, 6, 7, 8, 9, 10, 11, 12, 13, 14, 15 or more, 20 or more, 30 or more, 50 or more, 75 or more, 100 or more, 500 or more, or about 1000 nucleotides in length, where all or some of the nucleotide sequences differ within the region. A polymorphic site is often one nucleotide in length, which is referred to herein as a “single nucleotide polymorphism” or a “SNP.”

Where there are two, three, or four alternative nucleotide sequences at a polymorphic site, each nucleotide sequence is referred to as a “polymorphic variant” or “nucleic acid variant.” Where two polymorphic variants exist, for example, the polymorphic variant represented in a minority of samples from a population is sometimes referred to as a “minor allele” and the polymorphic variant that is more prevalently represented is sometimes referred to as a “major allele.” Many organisms possess a copy of each chromosome (e.g., humans), and those individuals who possess two major alleles or two minor alleles are often referred to as being “homozygous” with respect to the polymorphism, and those individuals who possess one major allele and one minor allele are normally referred to as being “heterozygous” with respect to the polymorphism. Individuals who are homozygous with respect to one allele are sometimes predisposed to a different phenotype as compared to individuals who are heterozygous or homozygous with respect to another allele.

In genetic analysis that associate polymorphic variants with type II diabetes, samples from individuals having type II diabetes and individuals not having type II diabetes often are allelotyped and/or genotyped. The term “allelotype” as used herein refers to a process for determining the allele frequency for a polymorphic variant in pooled DNA samples from cases and controls. By pooling DNA from each group, an allele frequency for each SNP in each group is calculated. These allele frequencies are then compared to one another. The term “genotyped” as used herein refers to a process for determining a genotype of one or more individuals, where a “genotype” is a representation of one or more polymorphic variants in a population.

A genotype or polymorphic variant may be expressed in terms of a “haplotype,” which as used herein refers to two or more polymorphic variants occurring within genomic DNA in a group of individuals within a population. For example, two SNPs may exist within a gene where each SNP position includes a cytosine variation and an adenine variation. Certain individuals in a population may carry one allele (heterozygous) or two alleles (homozygous) having the gene with a cytosine at each SNP position. As the two cytosines corresponding to each SNP in the gene travel together on one or both alleles in these individuals, the individuals can be characterized as having a cytosine/cytosine haplotype with respect to the two SNPs in the gene.

As used herein, the term “phenotype” refers to a trait which can be compared between individuals, such as presence or absence of a condition, a visually observable difference in appearance between individuals, metabolic variations, physiological variations, variations in the function of biological molecules, and the like. An example of a phenotype is occurrence of type II diabetes.

Researchers sometimes report a polymorphic variant in a database without determining whether the variant is represented in a significant fraction of a population. Because a subset of these reported polymorphic variants are not represented in a statistically significant portion of the population, some of them are sequencing errors and/or not biologically relevant. Thus, it is often not known whether a reported polymorphic variant is statistically significant or biologically relevant until the presence of the variant is detected in a population of individuals and the frequency of the variant is determined. Methods for detecting a polymorphic variant in a population are described herein, specifically in Example 2. A polymorphic variant is statistically significant and often biologically relevant if it is represented in 5% or more of a population, sometimes 10% or more, 15% or more, or 20% or more of a population, and often 25% or more, 300/a or more, 35% or more, 40% or more, 45% or more, or 50% or more of a population.

A polymorphic variant may be detected on either or both strands of a double-stranded nucleic acid. Also, a polymorphic variant may be located within an intron or exon of a gene or within a portion of a regulatory region such as a promoter, a 5′ untranslated region (UTR), a 3′ UTR, and in DNA (e.g., genomic DNA (gDNA) and complementary DNA (cDNA)), RNA (e.g., mRNA, tRNA, and rRNA), or a polypeptide. Polymorphic variations may or may not result in detectable differences in gene expression, polypeptide structure or polypeptide function.

It was determined that polymorphic variations associated with an increased risk of type H diabetes exist in an EPHA3 locus. An incident polymorphic variant described in Table 1 was associated with type II diabetes.

TABLE 1

| | | | Position in | | | | | | |
|---|---|---|---|---|---|---|---|---|---|
| SNP | | Chromosome | SEQ ID | Contig | Contig | Sequence | | Sequence | Allelic |
| Reference | Chromosome | Position | NO: 1 | Identification | Position | Identification | Locus | Position | Variability |
| rs1512183 | 3 | 89425955 | 50155 | NT_022459 | 23199742 | NM_005233 | EphA3 | intron | T/A |

Public information pertaining to the polymorphism and the genomic sequence that includes the polymorphism are indicated. The genomic sequence identified in Table 1 may be accessed at the http address www.ncbi.nih.gov/entrez/query.fcgi, for example, by using the publicly available SNP reference number (e.g., rs1512183). The chromosome position refers to the position of the SNP within NCBI's Genome build 34, which may be accessed at the following http address: www.ncbi.nlm.nih.gov/mapview/map_search.cgi?chr=hum_chr.inf&query=. The “Contig Position” provided in Table 1 corresponds to a nucleotide position set forth in the contig sequence, and designates the polymorphic site corresponding to the SNP reference number. The sequence containing the polymorphisms also may be referenced by the “Sequence Identification” set forth in Table 1. The “Sequence Identification” corresponds to cDNA sequence that encodes associated polypeptides (e.g., EPHA3) of the invention. The position of the SNP within the cDNA sequence is provided in the “Sequence Position” column of Table 1. Also, the allelic variation at the polymorphic site is specified in Table 1, where the allelic variant identified as associated with type II diabetes is a thymine. All nucleotide sequences referenced and accessed by the parameters set forth in Table 1 are incorporated herein by reference.

The polymorphic variant in Table 1 and others proximal to it were associated with Type II diabetes. In the EPHA3 locus, polymorphic variants corresponding to those selected from the group consisting of rs3792573, rs3792572, rs1398195, rs3828462, rs3805091, rs1512185, rs1028013, rs987748, rs1398197, rs1028011, rs2881488, rs1473598, rs1512183, rs157607, rs1157608, rs192965, rs1912966, rs1982096, AA at position 66765 in SEQ ID NO:1, AB at position 66794 in SEQ ID NO:1, rs1054750, rs211737, rs1499780, rs2117138, rs2346840, rs2048518, rs2048519, rs2048520, rs2048521, rs3762718, rs2196083, rs1512187, rs972030, rs2346837, rs1036286, rs1036285, rs1512188, rs1112189, rs1567657, rs1567658, and rs1028012 were tested for association with occurrence of type II diabetes. Polymorphic variants rs1512183, rs1512185, rs1028013, rs987748, rs2881488, rs1157607, rs1157608, rs1912965, rs1912966, rs1054750, rs1499780, rs1117138, rs2346840, rs2048518, rs2048519, rs2048521, rs3762718, rs2196083, rs972030, rs1036286, rs1036285, rs1512188, rs1512189, rs1567657, rs1567658, rs1028012, AA at position 66765 in SEQ ID NO: 1, AB at position 66794 in SEQ ID NO: 1 were in particular associated with an increased risk of type II diabetes. At these positions in SEQ ID NO: 1 an adenine at position 18716, a cytosine at position 29369, a thymine at position 39131, a guanine at position 45589, a thymine at position 50155, a thymine at position 51465, a guanine at position 51565, a guanine at position 63433, an adenine at position 63565, a cytosine at position 66826, a cytosine at position 71173, a guanine at position 76623, a guanine at position 78368, a cytosine at position 79006, an adenine at position 79079, an adenine at position 79354, a guanine at position 80167; a cytosine at position 81647, a thymine at position 83599, a thymine at position 88778, a guanine at position 89162, a guanine at position 91284, a guanine at position 91433, an adenine at position 93620, an adenine at position 93707, and a thymine at position 94523 was associated with risk of type II diabetes. An arginine at position 924 in an EPHA3 polypeptide (SEQ ID NO: 4) was associated with an increased risk of type II diabetes, which corresponds to position 66794 in SEQ ID NO: 1. Also, a histidine at position 914 in an EPHA3 polypeptide (SEQ ID NO: 4) was associated with an increased risk of type II diabetes, which corresponds to position 66765 in SEQ ID NO: 1. In addition, rs1512183 was associated with an increase in C-peptide levels in males and females.

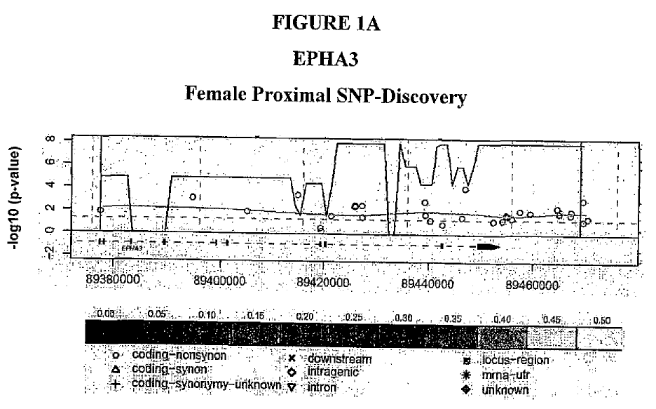

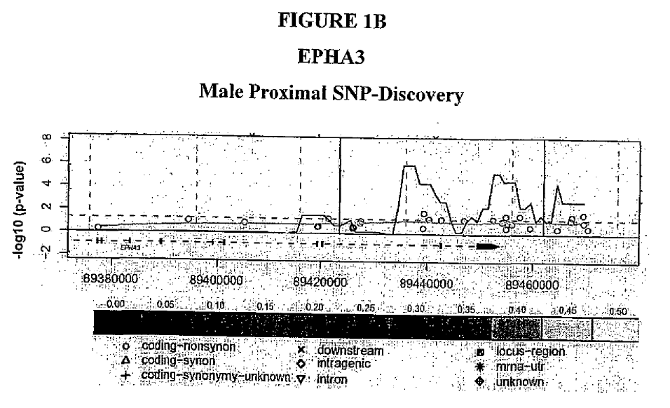

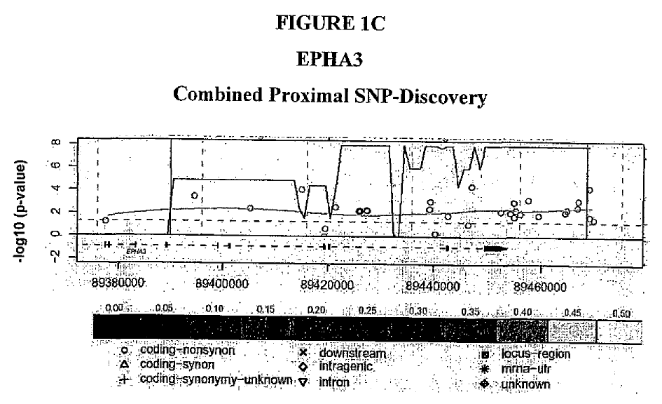

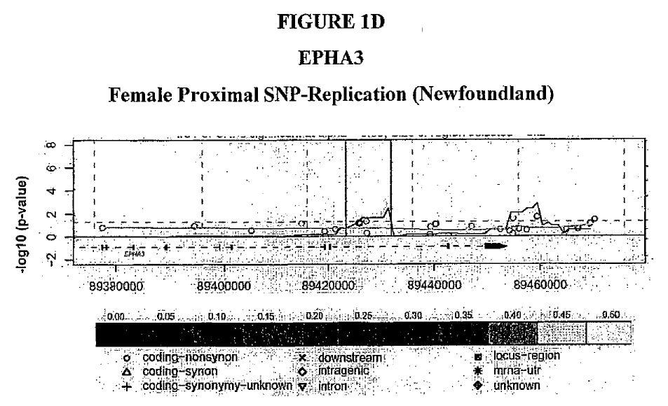

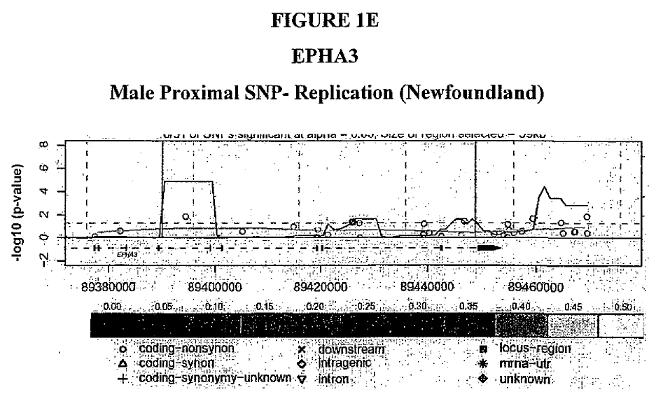

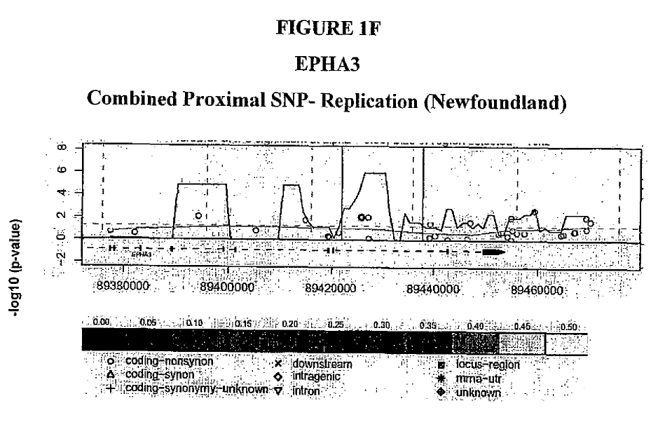

Based in part upon analyses summarized in FIGS. 1A-1F, regions with significant association have been identified in an EPHA3 locus associated with type II diabetes. Any polymorphic variants associated with type II diabetes in a region of significant association can be utilized for embodiments described herein. The following reports such regions, where “begin” and “end” designate the boundaries of the region according to chromosome positions within NCBI's Genome build 34. The chromosome on which the EPHA3 locus resides and an incident polymorphism in the locus also are noted.

| | | | | |
|---|---|---|---|---|
| Incident | chr | begin | end | size |

FEMALES

| | | | | |
|---|---|---|---|---|
| rs1512183 | 3 | 89377456 | 89470323 | 92867 |

MALES

| | | | | |
|---|---|---|---|---|
| rs1512183 | 3 | 89421389 | 89469420 | 48031 |

COMBINED

| | | | | |
|---|---|---|---|---|
| rs1512183 | 3 | 89394516 | 89470323 | 75807 |

For example, polymorphic variants in a region spanning chromosome positions 89336543 to 89428043 in the EPHA3 locus have significant association based upon a combined analysis of genetic information from males and females.

Additional Polymorphic Variants Associated with Type II Diabetes

Also provided is a method for identifying polymorphic variants proximal to an incident, founder polymorphic variant associated with type II diabetes Thus, featured herein are methods for identifying a polymorphic variation associated with type II diabetes that is proximal to an incident polymorphic variation associated with type II diabetes, which comprises identifying a polymorphic variant proximal to the incident polymorphic variant associated with type II diabetes, where the incident polymorphic variant is in an EPHA3 nucleotide sequence. The nucleotide sequence often comprises a polynucleotide sequence selected from the group consisting of (a) a polynucleotide sequence of SEQ ID NO: 1; (b) a polynucleotide sequence that encodes a polypeptide having an amino acid sequence encoded by a polynucleotide sequence of SEQ ID NO: 1; and (c) a polynucleotide sequence that encodes a polypeptide having an amino acid sequence that is 90% or more identical to an amino acid sequence encoded by a nucleotide sequence of SEQ ID NO: 1 or a polynucleotide sequence 90% or more identical to the polynucleotide sequence of SEQ ID NO: 1. The presence or absence of an association of the proximal polymorphic variant with type II diabetes then is determined using a known association method, such as a method described in the Examples hereafter. In an embodiment, the incident polymorphic variant is a polymorphic variant associated with type II diabetes described herein. In another embodiment, the proximal polymorphic variant identified sometimes is a publicly disclosed polymorphic variant, which for example, sometimes is published in a publicly available database. In other embodiments, the polymorphic variant identified is not publicly disclosed and is discovered using a known method, including, but not limited to, sequencing a region surrounding the incident polymorphic variant in a group of nucleic samples. Thus, multiple polymorphic variants proximal to an incident polymorphic variant are associated with type II diabetes using this method.

The proximal polymorphic variant often is identified in a region surrounding the incident polymorphic variant. In certain embodiments, this surrounding region is about 50 kb flanking the first polymorphic variant (e.g. about 50 kb 5′ of the first polymorphic variant and about 50 kb 3′ of the first polymorphic variant), and the region sometimes is composed of shorter flanking sequences, such as flanking sequences of about 40 kb, about 30 kb, about 25 kb, about 20 kb, about 15 kb, about 10 kb, about 7 kb, about 5 kb, or about 2 kb 5′ and 3, of the incident polymorphic variant. In other embodiments, the region is composed of longer flanking sequences, such as flanking sequences of about 55 kb, about 60 kb, about 65 kb, about 70 kb, about 75 kb, about 80 kb, about 85 kb, about 90 kb, about 95 kb, or about 100 kb 5′ and 3, of the incident polymorphic variant.

In certain embodiments, polymorphic variants associated with type II diabetes are identified iteratively. For example) a first proximal polymorphic variant is associated with type II diabetes using the methods described above and then another polymorphic variant proximal to the first proximal polymorphic variant is identified (e.g., publicly disclosed or discovered) and the presence or absence of an association of one or more other polymorphic variants proximal to the first proximal polymorphic variant with type II diabetes is determined.

The methods described herein are useful for identifying or discovering additional polymorphic variants that may be used to further characterize a gene, region or loci associated with a condition, a disease (e.g., type II diabetes), or a disorder. For example, allelotyping or genotyping data from the additional polymorphic variants may be used to identify a functional mutation or a region of linkage disequilibrium. In certain embodiments, polymorphic variants identified or discovered within a region comprising the first polymorphic variant associated with type II diabetes are genotyped using the genetic methods and sample selection techniques described herein, and it can be determined whether those polymorphic variants are in linkage disequilibrium with the first polymorphic variant. The size of the region in linkage disequilibrium with the first polymorphic variant also can be assessed using these genotyping methods. Thus, provided herein are methods for determining whether a polymorphic variant is in linkage disequilibrium with a first polymorphic variant associated with type II diabetes, and such information can be used in prognosis/diagnosis methods described herein.

Isolated Nucleic Acids

Featured herein are isolated EPHA3 nucleic acid variants depicted in SEQ ID NO: 1-3, and substantially identical nucleic acids thereof. A nucleic acid variant may be represented on one or both strands in a double-stranded nucleic acid or on one chromosomal complement (heterozygous) or both chromosomal complements (homozygous)).

As used herein, the term “nucleic acid” includes DNA molecules (e.g., a complementary DNA (cDNA) and genomic DNA (gDNA)) and RNA molecules (e.g., mRNA, rRNA, siRNA and tRNA) and analogs of DNA or RNA, for example, by use of nucleotide analogs. The nucleic acid molecule can be single-stranded and it is often double-stranded. The term “isolated or purified nucleic acid” refers to nucleic acids that are separated from other nucleic acids present in the natural source of the nucleic acid. For example, with regard to genomic DNA, the term “isolated” includes nucleic acids which are separated from the chromosome with which the genomic DNA is naturally associated. An “isolated” nucleic acid is often free of sequences which naturally flank the nucleic acid (i.e., sequences located at the 5′ and/or 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb or 0.1 kb of 5′ and/or 3′ nucleotide sequences which flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “gene” refers to a nucleotide sequence that encodes a polypeptide. In certain embodiments, the nucleic acid comprises an adenine or guanine at position 66765 in SEQ ID NO: 1 (corresponding chromosome position 89442565 from NCBI's build 34), which are associated with an increased risk and decreased risk of type II diabetes, respectively. The nucleic acid also may comprise a cytosine or thymine at position 66794 in SEQ ID NO: 1 (corresponding chromosome position 89442594 from NCBI's build 34), which are associated with an increased risk and decreased risk of type I diabetes, respectively.

The nucleic acid often comprises a part of or all of a nucleotide sequence in SEQ ID NO: 1, 2 and/or 3, or a substantially identical sequence thereof. Such a nucleotide sequence sometimes is a 5′ and/or 3′ sequence flanking a polymorphic variant described above that is 5-10000 nucleotides in length, or in some embodiments 5-5000, 5-1000, 5-500, 5-100, 5-75, 5-50, 5-45, 5-40, 5-35, 5-30, 5-25 or 5-20 nucleotides in length. In certain embodiments, the nucleic acid comprises one or more of the following nucleotides: an adenine or guanine at position 66765 in SEQ ID NO: 1 (corresponding chromosome position 89442565 from NCBI's build 34) or a cytosine or thymine at position 66794 in SEQ ID NO: 1 (corresponding chromosome position 89442594 from NCBI's build 34). Other embodiments are directed to methods of identifying a polymorphic variation at one or more positions in a nucleic acid (e.g., genotyping at one or more positions in the nucleic acid), such as at a position corresponding to position 66765 in SEQ ID NO: 1 or position 66794 in SEQ ID NO: 1.

Also included herein are nucleic acid fragments. These fragments often are a nucleotide sequence identical to a nucleotide sequence of SEQ ID NO: 1-3, a nucleotide sequence substantially identical to a nucleotide sequence of SEQ ID NO: 1-3, or a nucleotide sequence that is complementary to the foregoing. The nucleic acid fragment may be identical, substantially identical or homologous to a nucleotide sequence in an exon or an intron in a nucleotide sequence of SEQ ID NO: 1, and may encode a domain or part of a domain of a polypeptide. Sometimes, the fragment will comprises one or more of the polymorphic variations described herein as being associated with type II diabetes. Examples of EPHA3 nucleic acid fragments include but are not limited to those that encode an Ephrin receptor ligand binding domain (310-831 bp of SEQ ID NO: 2 and 172-696 bp of SEQ ID NO: 3); fibronectin type III domains (1210-1476 bp and 1534-1779 bp of SEQ ID NO: 2 and 1072-1338 bp and 1396-1641 bp of SEQ ID NO: 3>; a tyrosine kinase, catalytic domain (2086-2859 bp of SEQ ID NO: 2), and a sterile alpha motif (SAM) (2947-3150 bp of SEQ ID NO: 2). The nucleic acid fragment is often 50, 100, or 200 or fewer base pairs in length, and is sometimes about 300, 400, 500, 600, 700, 800, 900, 1000, 1100, 1200, 1300, 1400, 1500, 2000, 3000, 4000, 5000, 10000, 15000, or 20000 base pairs in length. A nucleic acid fragment that is complementary to a nucleotide sequence identical or substantially identical to a nucleotide sequence in SEQ ID NO: 1-3 and hybridizes to such a nucleotide sequence under stringent conditions is often referred to as a “probe.” Nucleic acid fragments often include one or more polymorphic sites, or sometimes have an end that is adjacent to a polymorphic site as described hereafter.

An example of a nucleic acid fragment is an oligonucleotide. As used herein, the term “oligonucleotide” refers to a nucleic acid comprising about 8 to about 50 covalently linked nucleotides, often comprising from about 8 to about 35 nucleotides, and more often from about 10 to about 25 nucleotides. The backbone and nucleotides within an oligonucleotide may be the same as those of naturally occurring nucleic acids, or analogs or derivatives of naturally occurring nucleic acids, provided that oligonucleotides having such analogs or derivatives retain the ability to hybridize specifically to a nucleic acid comprising a targeted polymorphism. Oligonucleotides described herein may be used as hybridization probes or as components of prognostic or diagnostic assays, for example, as described herein.

Oligonucleotides are typically synthesized using standard methods and equipment, such as the ABI™3900 High Throughput DNA Synthesizer and the EXPEDITE™ 8909 Nucleic Acid Synthesizer, both of which are available from Applied Biosystems (Foster City, Calif.). Analogs and derivatives are exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; WO 00/56746; WO 01/14398, and related publications. Methods for synthesizing oligonucleotides comprising such analogs or derivatives are disclosed, for example, in the patent publications cited above and in U.S. Pat. Nos. 5,614,622; 5,739,314; 5,955,599; 5,962,674; 6,117,992; in WO 00/75372; and in related publications.

Oligonucleotides may also be linked to a second moiety. The second moiety may be an additional nucleotide sequence such as a tail sequence (e.g., a polyadenosine tail), an adapter sequence (e.g., phage M13 universal tail sequence), and others. Alternatively, the second moiety may be a non-nucleotide moiety such as a moiety which facilitates linkage to a solid support or a label to facilitate detection of the oligonucleotide. Such labels include, without limitation, a radioactive label, a fluorescent label, a chemiluminescent label, a paramagnetic label, and the like. The second moiety may be attached to any position of the oligonucleotide, provided the oligonucleotide can hybridize to the nucleic acid comprising the polymorphism.

Uses for Nucleic Acid Sequence

Nucleic acid coding sequences (e.g., SEQ ID NO: 2-3) may be used for diagnostic purposes for detection and control of polypeptide expression. Also, included herein are oligonucleotide sequences such as antisense RNA, small-interfering RNA (siRNA) and DNA molecules and ribozymes that function to inhibit translation of a polypeptide. Antisense techniques and RNA interference techniques are known in the art and are described herein.

Ribozymes are enzymatic ANA molecules capable of catalyzing the specific cleavage of RNA. The mechanism of ribozyme action involves sequence specific hybridization of the ribozyme molecule to complementary target RNA, followed by endonucleolytic cleavage. For example, hammerhead motif ribozyme molecules may be engineered that specifically and efficiently catalyze endonucleolytic cleavage of RNA sequences corresponding to or complementary to EPHA3 nucleotide sequences. Specific ribozyme cleavage sites within any potential RNA target are initially identified by scanning the target molecule for ribozyme cleavage sites which include the following sequences, GUA, GUU and GUC. Once identified, short RNA sequences of between fifteen (15) and twenty (20) ribonucleotides corresponding to the region of the target gene containing the cleavage site may be evaluated for predicted structural features such as secondary structure that may render the oligonucleotide sequence unsuitable. The suitability of candidate targets may also be evaluated by testing their accessibility to hybridization with complementary oligonucleotides, using ribonuclease protection assays.

Antisense RNA and DNA molecules, siRNA and ribozymes may be prepared by any method known in the art for the synthesis of RNA molecules. These include techniques for chemically synthesizing oligodeoxyribonucleotides well known in the art such as solid phase phosphoramidite chemical synthesis. Alternatively, RNA molecules may be generated by in vitro and ill vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

DNA encoding a polypeptide also may have a number of uses for the diagnosis of diseases, including type II diabetes, resulting from aberrant expression of a target gene described herein. For example, the nucleic acid sequence may be used in hybridization assays of biopsies or autopsies to diagnose abnormalities of expression or function (e.g., Southern or Northern blot analysis, in situ hybridization assays).

In addition, the expression of a polypeptide during embryonic development may also be determined using nucleic acid encoding the polypeptide. As addressed, infra, production of functionally impaired polypeptide is the cause of various disease states, such as Type II diabetes. In situ hybridizations using polypeptide as a probe may be employed to predict problems related to type II diabetes. Further, as indicated, infra, administration of human active polypeptide, recombinantly produced as described herein, may be used to treat disease states related to functionally impaired polypeptide. Alternatively, gene therapy approaches may be employed to remedy deficiencies of functional polypeptide or to replace or compete with dysfunctional polypeptide.

Expression Vectors, Host Cells, and Genetically Engineered Cells

Provided herein are nucleic acid vectors, often expression vectors, which contain an EPHA3 nucleotide sequence or a substantially identical sequence thereof. As used herein, the term “vector” refers to a nucleic acid molecule capable of transporting another nucleic acid to which it has been linked and can include a plasmid, cosmid, or viral vector. The vector can be capable of autonomous replication or it can integrate into a host DNA. Viral vectors may include replication defective retroviruses, adenoviruses and adeno-associated viruses for example.

A vector can include an EPHA3 nucleotide sequence in a form suitable for expression of an encoded target polypeptide or target nucleic acid in a host cell. A “target polypeptide” is a polypeptide encoded by an EPHA3 nucleotide sequence or a substantially identical nucleotide sequence thereof. The recombinant expression vector typically includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of polypeptide desired, and the like. Expression vectors can be introduced into host cells to produce target polypeptides, including fusion polypeptides.

Recombinant expression vectors can be designed for expression of target polypeptides in prokaryotic or eukaryotic cells. For example, target polypeptides can be expressed in E. coli, insect cells (e.g., using baculovirus expression vectors), yeast cells, or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith & Johnson, Gene 67: 31-40 (1988)), pMAL (New England Biolabs, Beverly, Mass.) and pRIT5 (Pharmacia, Piscataway, N.J.) which fuse glutathione S-transferase (GST), maltose E binding polypeptide, or polypeptide A, respectively, to the target recombinant polypeptide.

Purified fusion polypeptides can be used in screening assays and to generate antibodies specific for target polypeptides. In a therapeutic embodiment, fusion polypeptide expressed in a retroviral expression vector is used to infect bone marrow cells that are subsequently transplanted into irradiated recipients. The pathology of the subject recipient is then examined after sufficient time has passed (e.g., six (6) weeks).

Expressing the polypeptide in host bacteria with an impaired capacity to proteolytically cleave the recombinant polypeptide is often used to maximize recombinant polypeptide expression (Gottesman, S., Gene Expression Technology: Methods in Enzymology, Academic Press, San Diego, Calif. 185: 119-128 (1990)). Another strategy is to alter the nucleotide sequence of the nucleic acid to be inserted into an expression vector so that the individual codons for each amino acid are those preferentially utilized in E. coli (Wada et al., Nucleic Acids Res. 20: 2111-2118 (1992)). Such alteration of nucleotide sequences can be carried out by standard DNA synthesis techniques.

When used in mammalian cells, the expression vector's control functions are often provided by viral regulatory elements. For example, commonly used promoters are derived from polyoma, Adenovirus 2, cytomegalovirus and Simian Virus 40. Recombinant mammalian expression vectors are often capable of directing expression of the nucleic acid in a particular cell type (e.g., tissue-specific regulatory elements are used to express the nucleic acid). Non-limiting examples of suitable tissue-specific promoters include an albumin promoter (liver-specific; Pinkert et al., Genes Dev. 1: 268-277 (1987)), lymphoid-specific promoters (Calame & Eaton, Adv. Immunol 43: 235-275 (1988)), promoters of T cell receptors (Winoto & Baltimore, EMBO J. 8: 729-733 (1989)) promoters of immunoglobulins (Banerji et al., Cell 33: 729-740 (1983); Queen & Baltimore, Cell 33: 741-748 (1983) neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985), and mammary gland-specific promoters (e.g., milk whey promoter; U.S. Pat. No. 4,873,316 and European Application Publication No. 264,166). Developmentally-regulated promoters are sometimes utilized, for example, the murine hox promoters Kessel & Gruss, Science 249: 374-379 (1990)) and the alpha-fetopolypeptide promoter (Camper & Tilghman, Genes Dev. 3: 537-546 (1989)).

An EPHA3 nucleic acid may also be cloned into an expression vector in an antisense orientation. Regulatory sequences (e.g., viral promoters and/or enhancers operatively linked to an EPHA3 nucleic acid cloned in the antisense orientation can be chosen for directing constitutive, tissue specific or cell type specific expression of antisense RNA in a variety of cell types. Antisense expression vectors can be in the form of a recombinant plasmid, phagemid or attenuated virus. For a discussion of the regulation of gene expression using antisense genes see, e.g., Weintraub et al., Antisense RNA as a molecular tool for genetic analysis, Reviews—Trends in Genetics, Vol. 1(1) (1986).

Also provided herein are host cells that include an EPHA3 nucleotide sequence within a recombinant expression vector or a fragment of such a nucleotide sequence which facilitate homologous recombination into a specific site of the host cell genome. The terms “host cell” and “recombinant host cell” are used interchangeably herein. Such terms refer not only to the particular subject cell but rather also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a target polypeptide can be expressed in bacterial cells such as E. coli, insect cells, yeast or mammalian cells (such as Chinese hamster ovary cells (CHO) or COS cells). Other suitable host cells are known to those skilled in the art.

Vectors can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction/infection, DEAE-dextran-mediated transfection, lipofection, or electroporation.

A host cell provided herein can be used to produce (i.e., express) a target polypeptide or a substantially identical polypeptide thereof. Accordingly, further provided are methods for producing a target polypeptide using host cells described herein. In one embodiment, the method includes culturing host cells into which a recombinant expression vector encoding a target polypeptide has been introduced in a suitable medium such that a target polypeptide is produced. In another embodiment, the method further includes isolating a target polypeptide from the medium or the host cell.

Also provided are cells or purified preparations of cells which include an EPHA3 transgene, or which otherwise misexpress target polypeptide. Cell preparations can consist of human or non-human cells, e.g., rodent cells, e.g., mouse or rat cells, rabbit cells, or pig cell. In preferred embodiments, the cell or cells include an EPHA3 transgene (e.g., a heterologous form of an EPHA3 gene, such as a human gene expressed in non-human cells). The transgene can be misexpressed, e.g., overexpressed or underexpressed. In other preferred embodiments, the cell or cells include a gene which misexpress an endogenous target polypeptide (e.g., expression of a gene is disrupted, also known as a knockout). Such cells can serve as a model for studying disorders which are related to mutated or mis-expressed alleles or for use in drug screening. Also provided are human cells (e.g., a hematopoietic stem cells) transformed with an EPHA3 nucleic acid.

Also provided are cells or a purified preparation thereof (e.g., human cells) in which an endogenous EPHA3 nucleic acid is under the control of a regulatory sequence that does not normally control the expression of the endogenous gene corresponding to an EPHA3 nucleotide sequence. The expression characteristics of an endogenous gene within a cell (e.g., a cell line or microorganism) can be modified by inserting a heterologous DNA regulatory element into the genome of the cell such that the inserted regulatory element is operably linked to the corresponding endogenous gene. For example, an endogenous corresponding gene (e.g., a gene which is “transcriptionally silent,” not normally expressed, or expressed only at very low levels) may be activated by inserting a regulatory element which is capable of promoting the expression of a normally expressed gene product in that cell. Techniques such as targeted homologous recombinations, can be used to insert the heterologous. DNA as described in, e.g., Chappel, U.S. Pat. No. 5,272,071; WO 91/06667, published on May 16, 1991.

Transgenic Animals

Non-human transgenic animals that express a heterologous target polypeptide (e.g., expressed from an EPHA3 nucleic acid or substantially identical sequence thereof) can be generated. Such animals are useful for studying the function and/or activity of a target polypeptide and for identifying and/or evaluating modulators of the activity of EPHA3 nucleic acids and encoded polypeptides. As used herein, a “transgenic animal” is a non-human animal such as a mammal (e.g., a non-human primate such as chimpanzee, baboon, or macaque; an ungulate such as an equine, bovine, or caprine; or a rodent such as a rat, a mouse, or an Israeli sand rat, a bird (e.g., a chicken or a turkey), an amphibian (e.g., a frog, salamander, or newt), or an insect (e.g., Drosophila melanogaster), in which one or more of the cells of the animal includes a transgene. A transgene is exogenous DNA or a rearrangement (e.g., a deletion of endogenous chromosomal DNA) that is often integrated into or occurs in the genome of cells in a transgenic animal. A transgene can direct expression of an encoded gene product in one or more cell types or tissues of the transgenic animal, and other transgenes can reduce expression (e.g., a knockout). Thus, a transgenic animal can be one in which an endogenous nucleic acid homologous to an EPHA3 nucleic acid has been altered by homologous recombination between the endogenous gene and an exogenous DNA molecule introduced into a cell of the animal (e.g., an embryonic cell of the animal) prior to development of the animal.

Intronic sequences and polyadenylation signals can also be included in the transgene to increase expression efficiency of the transgene. One or more tissue-specific regulatory sequences can be operably linked to an EPHA3 nucleotide sequence to direct expression of an encoded polypeptide to particular cells. A transgenic founder animal can be identified based upon the presence of an EPHA3 nucleotide sequence in its genome and/or expression of encoded mRNA in tissues or cells of the animals. A transgenic founder animal can then be used to breed additional animals carrying the transgene. Moreover, transgenic animals, carrying an EPHA3 nucleotide sequence can further be bred to other transgenic animals carrying other transgenes.

Target polypeptides can be expressed in transgenic animals or plants by introducing, for example, an EPHA3 nucleic acid into the genome of an animal that encodes the target polypeptide. In preferred embodiments the nucleic acid is placed under the control of a tissue specific promoter, e.g., a milk or egg specific promoter, and recovered from the milk or eggs produced by the animal. Also included is a population of cells from a transgenic animal.

Target Polypeptides

Also featured herein are isolated target polypeptides, which are encoded by an EPHA3 nucleotide sequence (e.g., SEQ ID NO: 1-3) or a substantially identical nucleotide sequence thereof such as the polypeptides having amino acid sequences in SEQ ID NO: 4 or 5. The term “polypeptide” as used herein includes proteins and peptides. An “isolated” or “purified” polypeptide or protein is substantially free of cellular material or other contaminating proteins from the cell or tissue source from which the protein is derived, or substantially free from chemical precursors or other chemicals when chemically synthesized. In one embodiment, the language “substantially free” means preparation of a target polypeptide having less than about 30%, 20%, 10% and more preferably 5% (by dry weighty, of non-target polypeptide (also referred to herein as a “contaminating protein”), or of chemical precursors or non-target chemicals. When the target polypeptide or a biologically active portion thereof is recombinantly produced, it is also preferably substantially free of culture medium, specifically, where culture medium represents less than about 20%, sometimes less than about 10%, and often less than about 5% of the volume of the polypeptide preparation. Isolated or purified target polypeptide preparations are sometimes 0.01 milligrams or more or 0.1 milligrams or more, and often 1.0 milligrams or more and 10 milligrams or more in dry weight.

An EPHA3 polypeptide may be an isoform. For example, transcript variant 1 of EPHA3 is a 135 kDa, 983 amino acid type I transmembrane glycoprotein that contains a 20 amino acid signal sequence, a 521 amino acid extracellular region (21-541), a 24 amino acid transmembrane domain (542-564) and a 418 amino acid cytoplasmic segment (565-983 of SEQ ID NO: 4). Transcript variant 2 (SEQ ID NO: 5) uses an alternate splice site in the 3′ coding region, compared to variant 1, that results in a frameshift. It encodes an isoform which has a shorter and distinct C-terminus compared to variant 1. Transcript variant 2 (also known as an isoform b variant) lacks a transmembrane domain, contains a 20 amino acid signal sequence and may be a secreted form of the EPHA3 receptor. The isoform b variant of EPHA3 is capable of binding Ephrin-A2 or Ephrin-A5. Also, the 521 amino acid extracellular domain (21-541 of SEQ ID NO:4) is capable of binding Ephrin-A5. The EPHA3 polypeptide also may include an arginine at position 924 in SEQ ID NO: 4, which is a form associated with risk of type II diabetes, or a tryptophan at position 924 in SEQ ID NO: 4, which is a form associated with less risk of type II diabetes. The EPHA3 polypeptide also may include a histidine at position 914 in SEQ ID NO: 4, which is a form associated with risk of type II diabetes, or an arginine at position 914 in SEQ ID NO: 4, which is a form associated with less risk of type II diabetes. Positions 914 and 924 lie in a SAM domain described hereafter.

Further included herein are target polypeptide fragments. The polypeptide fragment may be a domain or part of a domain of a target polypeptide. For example, EPHA3 domains include but are not limited to an Ephrin receptor ligand binding (Eph_ldb) domain from about amino acids 29-202 of SEQ ID NO: 4 or 5; fibronectin type 3 (FN3) domains from about amino acids 326417 and 437-521 of SEQ ID NO: 4, and amino acids 329-417 and 437-518 of SEQ ID NO: 5; tyrosine kinase, catalytic (TyrKc) domain from about amino acids 621-878 of SEQ ID NO: 4; and a sterile alpha motif (SAM) from about amino acids 908-975 of SEQ ID NO: 4. The polypeptide fragment may have increased, decreased or unexpected biological activity. The polypeptide fragment is often 50 or fewer, 100 or fewer, or 200 or fewer amino acids in length, and is sometimes 300, 400, 500, 600, 700, or 900 or fewer amino acids in length.

Substantially identical target polypeptides may depart from the amino acid sequences of target polypeptides in different manners. For example, conservative amino acid modifications may be introduced at one or more positions in the amino acid sequences of target polypeptides. A “conservative amino acid substitution” is one in which the amino acid is replaced by another amino acid having a similar structure and/or chemical function. Families of amino acid residues having similar structures and functions are well known. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, typtophan), beta-branched side chains (e.g., threonine, valine, isoleucine) and aromatic side chains (e.g., tyrosine, phenylalanine, tryptophan, histidine). Also, essential and non-essential amino acids may be replaced. A “non-essential” amino acid is one that can be altered without abolishing or substantially altering the biological function of a target polypeptide, whereas altering an “essential” amino acid abolishes or substantially alters the biological function of a target polypeptide. Amino acids that are conserved among target polypeptides are typically essential amino acids.

Also, target polypeptides may exist as chimeric or fusion polypeptide. As used herein, a target “chimeric polypeptide” or target “fusion polypeptide” includes a target polypeptide linked to a non-target polypeptide. A “non-target polypeptide” refers to a polypeptide having an amino acid sequence corresponding to a polypeptide which is not substantially identical to the target polypeptide, which includes, for example, a polypeptide that is different from the target polypeptide and derived from the same or a different organism. The target polypeptide in the fusion polypeptide can correspond to an entire or nearly entire target polypeptide or a fragment thereof. The non-target polypeptide can be fused to the N-terminus or C-terminus of the target polypeptide.

Fusion polypeptides can include a moiety having high affinity for a ligand. For example, the fusion polypeptide can be a GST-target fusion polypeptide in which the target sequences are fused to the C-terminus of the EST sequences, or a polyhistidine-target fusion polypeptide in which the target polypeptide is fused at the N- or C-terminus to a string of histidine residues. Such fusion polypeptides can facilitate purification of recombinant target polypeptide. Expression vectors are commercially available that already encode a fusion moiety (e.g., a GST polypeptide), and a nucleotide sequence in SEQ ID NO: 1-3, or a substantially identical nucleotide sequence thereof; can be cloned into an expression vector such that the fusion moiety is linked in-frame to the target polypeptide. Further, the fission polypeptide can be a target polypeptide containing a heterologous signal sequence at its N-terminus. In certain host cells (e.g., mammalian host cells), expression, secretion, cellular internalization, and cellular localization of a target polypeptide can be increased through use of a heterologous signal sequence. Fusion polypeptides can also include all or a part of a serum polypeptide (e.g., an IgG constant region or human serum albumin).

Target polypeptides can be incorporated into pharmaceutical compositions and administered to a subject in vivo. Administration of these target polypeptides can be used to affect the bioavailability of a substrate of the target polypeptide and may effectively increase target polypeptide biological activity in a cell. Target fusion polypeptides may be useful therapeutically for the treatment of disorders caused by, for example, (i) aberrant modification or mutation of a gene encoding a target polypeptide; (ii) mis-regulation of the gene encoding the target polypeptide; and (iii) aberrant post-translational modification of a target polypeptide. Also, target polypeptides can be used as immunogens to produce anti-target antibodies in a subject, to purify target polypeptide ligands or binding partners, and in screening assays to identify molecules which inhibit or enhance the interaction of a target polypeptide with a substrate.

In addition, polypeptides can be chemically synthesized using techniques known in the art (See, e.g., Creighton, 1983 Proteins. New York, N.Y.: W.H. Freeman and Company; and Hunkapiller et al., (1994) Nature July 12-18; 310(5973):105-11). For example, a relative short fragment can be synthesized by use of a peptide synthesizer. Furthermore, if desired, non-classical amino acids or chemical amino acid analogs can be introduced as a substitution or addition into the fragment sequence. Non-classical amino acids include, but are not limited to, to the D-isomers of the common amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoroamino acids, designer amino acids such as b-methyl amino acids, Ca-methyl amino acids, Na-methyl amino acids, and amino acid analogs in general. Furthermore, the amino acid can be D (dextrorotary) or L (levorotary).

Polypeptides and polypeptide fragments sometimes are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH4; acetylation, formylation, oxidation, reduction; metabolic synthesis in the presence of tunicamycin; and the like. Additional post-translational modifications include, for example, N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The polypeptide fragments may also be modified with a detectable label such as an enzymatic, fluorescent, isotopic or affinity label to allow for detection and isolation of the polypeptide.

Also provided are chemically modified derivatives of polypeptides that can provide additional advantages such as increased solubility, stability and circulating time of the polypeptide, or decreased immunogenicity (see e.g., U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog).

The polymers should be attached to the polypeptide with consideration of effects on functional or antigenic domains of the polypeptide. There are a number of attachment methods available to those skilled in the art (e.g., EP 0 401 384 (coupling PEG to G-CSF) and Malik et al. (1992) Exp Hematol. September; 20(8): 1028-35 (pegylation of GM-CSF using tresyl chloride)). For example, polyethylene glycol may be covalently bound through amino acid residues via a reactive group, such as a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues, glutamic acid residues and the C-terminal amino acid residue. Sulfhydryl groups may also be used as a reactive group for attaching the polyethylene glycol molecules. For therapeutic purposes, the attachment sometimes is at an amino group, such as attachment at the N-terminus or lysine group.

Proteins can be chemically modified at the N-terminus. Using polyethylene glycol as an illustration of such a composition, one may select from a variety of polyethylene glycol molecules (by molecular weight, branching, and the like), the proportion of polyethylene glycol molecules to protein (polypeptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus may be accomplished by reductive alkylation, which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

Substantially Identical Nucleic Acids and Polypeptides

Nucleotide sequences and polypeptide sequences that are substantially identical to an EPHA3 nucleotide sequence and the target polypeptide sequences encoded by those nucleotide sequences, respectively, are included herein. The term “substantially identical” as used herein refers to two or more nucleic acids or polypeptides sharing one or more identical nucleotide sequences or polypeptide sequences, respectively. Included are nucleotide sequences or polypeptide sequences that are 55% or more, 60% or more, 65% or more, 70% or more, 75% or more, 80% or more, 85% or more, 90% or more, 95% or more (each often within a 1%, 2%, 3% or 4% variability) identical to an EPHA3 nucleotide sequence or the encoded target polypeptide amino acid sequences. One test for determining whether two nucleic acids are substantially identical is to determine the percent of identical nucleotide sequences or polypeptide sequences shared between the nucleic acids or polypeptides.

Calculations of sequence identity are often performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (19891. Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 71% SDS at 65° C., followed by one or more washes at 0.2×SSC, 1% SDS at 65° C.

An example of a substantially identical nucleotide sequence to a nucleotide sequence in SEQ ID NO: 1-3 is one that has a different nucleotide sequence but still encodes the same polypeptide sequence encoded by the nucleotide sequence in SEQ ID NO: 1-3. Another example is a nucleotide sequence that encodes a polypeptide having a polypeptide sequence that is more than 70% or more identical to, sometimes more than 75% or more, 80% or more, or 85% or more identical to, and often more than 90% or more and 95% or more identical to a polypeptide sequence encoded by a nucleotide sequence in SEQ ID NO: 1-3. As used herein, “SEQ ID NO: 1-3” typically refers to one or more sequences in SEQ ID NO: 1, 2 and/or 3. Many of the embodiments described herein are applicable to (a) a nucleotide sequence of SEQ ID NO; 1, 2 and/or 3; (b) a nucleotide sequence which encodes a polypeptide consisting of an amino acid sequence encoded by a nucleotide sequence of SEQ ID NO: 1, 2 and/or 3; (c) a nucleotide sequence which encodes a polypeptide that is 90% or more identical to an amino acid sequence encoded by a nucleotide sequence of SEQ ID NO: 1, 2 and/or 3, or a nucleotide sequence about 90% or more identical to a nucleotide sequence of SEQ ID NO: 1, 2 and/or 3; (d) a fragment of a nucleotide sequence of (a), (b), or (c); and/or a nucleotide sequence complementary to the nucleotide sequences of (a), (b), (c) and/or (d), where nucleotide sequences of (b) and (c), fragments of (b) and (c) and nucleotide sequences complementary to (b) and (c) are examples of substantially identical nucleotide sequences. Examples of substantially identical nucleotide sequences include nucleotide sequences from subjects that differ by naturally occurring genetic variance, which sometimes is referred to as background genetic variance (e.g., nucleotide sequences differing by natural genetic variance sometimes are 90%, 91%, 92%, 93%, 94%, 95%, 96%, 97%, 98%, or 99% identical to one another).

Nucleotide sequences in SEQ ID NO: 1-3 and amino acid sequences of encoded polypeptides can be used as “query sequences” to perform a search against public databases to identify other family members or related sequences, for example. Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences in SEQ ID NO: 1-3. BLAST polypeptide searches can be performed with the XBLAST program, score=50, wordlength=3 to obtain amino acid sequences homologous to polypeptides encoded by the nucleotide sequences of SEQ ID NO: 1-3. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

A nucleic acid that is substantially identical to a nucleotide sequence in SEQ ID NO: 1-3 may include polymorphic sites at positions equivalent to those described herein when the sequences are aligned. For example, using the alignment procedures described herein, SNPs in a sequence substantially identical to a sequence in SEQ ID NO: 1-3 can be identified at nucleotide positions that match with or correspond to (i.e., align) nucleotides at SNP positions in each nucleotide sequence in SEQ ID NO: 1-3. Also, where a polymorphic variation results in an insertion or deletion, insertion or deletion of a nucleotide sequence from a reference sequence can change the relative positions of other polymorphic sites in the nucleotide sequence.

Substantially identical nucleotide and polypeptide sequences include those that are naturally occurring, such as allelic variants (same locus), splice variants, homologs (different locus), and orthologs (different organism) or can be non-naturally occurring. Non-naturally occurring variants can be generated by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared in the encoded product). Orthologs, homologs, allelic variants, and splice variants can be identified using methods known in the art. These variants normally comprise a nucleotide sequence encoding a polypeptide that is 50% or more, about 55% or more, often about 70-75% or more or about 80-85% or more, and sometimes about 90-95% or more identical to the amino acid sequences of target polypeptides or a fragment thereof. Such nucleic acid molecules can readily be identified as being able to hybridize under stringent conditions to a nucleotide sequence in SEQ ID NO: 1-3 or a fragment of this sequence. Nucleic acid molecules corresponding to orthlogs, homologs, and allelic variants of a nucleotide sequence in SEQ ID NO: 1-3 can further be identified by mapping the sequence to the same chromosome or locus as the nucleotide sequence in SEQ ID NO: 1-3.

Also, substantially identical nucleotide sequences may include codons that are altered with respect to the naturally occurring sequence for enhancing expression of a target polypeptide in a particular expression system. For example, the nucleic acid can be one in which one or more codons are altered, and often 10% or more or 20% or more of the codons are altered for optimized expression in bacteria (e.g., E. coli.), yeast (e.g., S. cervesiae), human (e.g., 293 cells), insect or rodent (e.g., hamster) cells.

Methods for Identifying Subjects at Risk of Diabetes and Risk of Diabetes in a Subject

Methods for prognosing and diagnosing type II diabetes and its related disorders (e.g., metabolic disorders, syndrome X, obesity, hypertension, insulin resistance, hyperglycemia) are included herein. These methods include detecting the presence or absence of one or more polymorphic variations in a nucleotide sequence associated with type II diabetes, such as variants in or around the locus set forth herein, or a substantially identical sequence thereof, in a sample from a subject, where the presence of a polymorphic variant described herein is indicative of a risk of type II diabetes or one or more type II diabetes related disorders (e.g., metabolic disorders, syndrome X, obesity, hypertension, insulin resistance, hyperglycemia). Determining a risk of type U diabetes refers to determining whether an individual is at an increased risk of type II diabetes (e.g., intermediate risk or higher risk).

Thus, featured herein is a method for identifying a subject who is at risk of type II diabetes, which comprises detecting a type II diabetes-associated aberration in a nucleic acid sample from the subject. An embodiment is a method for detecting risk of type II diabetes in a subject, which comprises detecting the presence or absence of a polymorphic variation associated with type II diabetes at a polymorphic site in a nucleotide sequence in a nucleic acid sample from a subject, where the nucleotide sequence comprises a polynucleotide sequence selected from the group consisting of: (a) a nucleotide sequence of SEQ ID NO: 1-3; (b) a nucleotide sequence which encodes a polypeptide consisting of an amino acid sequence encoded by a nucleotide sequence of SEQ ID NO: 1-3; (c) a nucleotide sequence which encodes a polypeptide that is 90% or more identical to an amino acid sequence encoded by a nucleotide sequence of SEQ ID NO: 1-3, or a nucleotide sequence about 90% or more identical to a nucleotide sequence of SEQ ID NO: 1-3; and (d) a fragment of a nucleotide sequence of (a), (b), or (c) comprising the polymorphic site; whereby the presence of the polymorphic variation is indicative of a predisposition to type II diabetes in the subject. In certain embodiments, polymorphic variants at the positions described herein are detected for determining a risk of type II diabetes, and polymorphic variants at positions in linkage disequilibrium with these positions are detected for determining a risk of type II diabetes.

Results from prognostic tests may be combined with other test results to diagnose type II diabetes related disorders, including metabolic disorders, syndrome X, obesity, hypertension, insulin resistance, hyperglycemia. For example, prognostic results may be gathered, a patient sample may be ordered based on a determined predisposition to type II diabetes, the patient sample is analyzed, and the results of the analysis may be utilized to diagnose the type II diabetes related condition (e.g., metabolic disorders, syndrome X, obesity, hypertension, insulin resistance, hyperglycemia). Also type II diabetes diagnostic methods can be developed from studies used to generate prognostic methods in which populations are stratified into subpopulations having different progressions of a type II diabetes related disorder or condition. In another embodiment, prognostic results may be gathered, a patient's risk factors for developing type II diabetes (e.g., age, weight, race, diet) analyzed, and a patient sample may be ordered based on a determined predisposition to type II diabetes.

Risk of type II diabetes sometimes is expressed as a probability, such as an odds ratio, percentage, or risk factor. The risk sometimes is expressed as a relative risk with respect to a population average risk of type F diabetes, and sometimes is expressed as a relative risk with respect to the lowest risk group. Such relative risk assessments often are based upon penetrance values determined by statistical methods and are particularly useful to clinicians and insurance companies for assessing risk of type II diabetes (e.g., a clinician can target appropriate detection, prevention and therapeutic regimens to a patient after determining the patient's risk of type II diabetes, and an insurance company can fine tune actuarial tables based upon population genotype assessments of type II diabetes risk). Risk of type II diabetes sometimes is expressed as an odds ratio, which is the odds of a particular person having a genotype has or will develop type II diabetes with respect to another genotype group (e.g., the most disease protective genotype or population average). The risk often is based upon the presence or absence of one or more polymorphic variants described herein, and also may be based in part upon phenotypic traits of the individual being tested. In an embodiment, two or more polymorphic variations are detected in an EPHA3 locus. In certain embodiments, 3 or more, or 4, 5, 6, 7, 8, 9, 10, 11, 12, 13, 14, 15, 16, 17, 18, 19 or 20 or more polymorphic variants are detected in the sample. Methods for calculating risk based upon patient data are well known (wee, e.g., Agresti, Categorical Data Analysis, 2nd Ed. 2002. Wiley). Allelotyping and genotyping analyses may be carried out in populations other than those exemplified herein to enhance the predictive power of the prognostic method.

The nucleic acid sample typically is isolated from a biological sample obtained from a subject. For example, nucleic acid can be isolated from blood, saliva, sputum, urine, cell scrapings, and biopsy tissue. The nucleic acid sample can be isolated from a biological sample using standard techniques, such as the technique described in Example 2. As used herein, the term “subject” refers primarily to humans but also refers to other mammals such as dogs, cats, and ungulates (e.g., cattle, sheep, and swine). Subjects also include avians (e.g., chickens and turkeys), reptiles, and fish (e.g., salmon), as embodiments described herein can be adapted to nucleic acid samples isolated from any of these organisms. The nucleic acid sample may be isolated from the subject and then directly utilized in a method for determining the presence of a polymorphic variant, or alternatively, the sample may be isolated and then stored (e.g., frozen) for a period of time before being subjected to analysis.

The presence or absence of a polymorphic variant is determined using one or both chromosomal complements represented in the nucleic acid sample. Determining the presence or absence of a polymorphic variant in both chromosomal complements represented in a nucleic acid sample from a subject having a copy of each chromosome is useful for determining the zygosity of an individual for the polymorphic variant (i.e., whether the individual is homozygous or heterozygous for the polymorphic variant). Any oligonucleotide-based diagnostic may be utilized to determine whether a sample includes the presence or absence of a polymorphic variant in a sample. For example, primer extension methods, ligase sequence determination methods (e.g. U.S. Pat. Nos. 5,679,524 and 5,952,174, and WO 01/27326), mismatch sequence determination methods (e.g., U.S. Pat. Nos. 5,851,770; 5,958,692; 6,110,684; and 6,183,958), microarray sequence determination methods, restriction fragment length polymorphism (RFLP), single strand conformation polymorphism detection (SSCP) (e.g., U.S. Pat. Nos. 5,891,625 and 6,013,499), PCR-based assays (e.g., TAQMAN® PCR System (Applied Biosystems)), and nucleotide sequencing methods may be used.

Oligonucleotide extension methods typically involve providing a pair of oligonucleotide primers in a polymerase chain reaction (PCR) or in other nucleic acid amplification methods for the purpose of amplifying a region from the nucleic acid sample that comprises the polymorphic variation. One oligonucleotide primer is complementary to a region 3′ of the polymorphism and the other is complementary to a region 5′ of the polymorphism. A PCR primer pair may be used in methods disclosed in U.S. Pat. Nos. 4,683,195; 4,683,202, 4,965,188; 5,656,493; 5,998,143; 6,140,054; WO 01/27327; and WO 01/27329 for example. PCR primer pairs may also be used in any commercially available machines that perform PCR, such as any of the GENEAMP® Systems available from Applied Biosystems. Also, those of ordinary skill in the art will be able to design oligonucleotide primers based upon an EPHA3 nucleotide sequence using knowledge available in the art.

Also provided is an extension oligonucleotide that hybridizes to the amplified fragment adjacent to the polymorphic variation. As used herein, the term “adjacent” refers to the 3′ end of the extension oligonucleotide being often 1 nucleotide from the 5′ end of the polymorphic site, and sometimes 2, 3, 4, 5, 6, 7, 8, 9, or 10 nucleotides from the 5′ end of the polymorphic site, in the nucleic acid when the extension oligonucleotide is hybridized to the nucleic acid. The extension oligonucleotide then is extended by one or more nucleotides, and the number and/or type of nucleotides that are added to the extension oligonucleotide determine whether the polymorphic variant is present. Oligonucleotide extension methods are disclosed, for example, in U.S. Pat. Nos. 4,656,117; 4,851,331; 5,679,524; 5,834,189; 5,876,934; 5,908,755; 5,912,118; 5,976,802; 5,981,186; 6,004,744; 6,013,431; 6,017,702; 6,046,005; 6,087,095; 6,210,891; and WO 01/20039. Oligonucleotide extension methods using mass spectrometry are described, for example, in U.S. Pat. Nos. 5,547,835; 5,605,798; 5,691,141; 5,849,542; 5,869,242; 5,928,906; 6,043,031; and 6,194,144, and a method often utilized is described herein in Example 2.

A microarray can be utilized for determining whether a polymorphic variant is present or absent in a nucleic acid sample. A microarray may include any oligonucleotides described herein, and methods for making and using oligonucleotide microarrays suitable for diagnostic use are disclosed in U.S. Pat. Nos. 5,492,806; 5,525,464; 5,589,330; 5,695,940; 5,849,483; 6,018,041; 6,045,996; 6,136,541; 6,142,681; 6,156,501; 6,197,506; 6,223,127; 6,225,625; 6,229,911; 6,239,273; WO 00/52625; WO 01/25485; and WO 01/29259. The microarray typically comprises a solid support and the oligonucleotides may be linked to this solid support by covalent bonds or by non-covalent interactions. The oligonucleotides may also be linked to the solid support directly or by a spacer molecule. A microarray may comprise one or more oligonucleotides complementary to a polymorphic site set forth herein.

A kit also may be utilized for determining whether a polymorphic variant is present or absent in a nucleic acid sample. A kit often comprises one or more pairs of oligonucleotide primers useful for amplifying a fragment of a nucleotide sequence of SEQ ID NO: 1-3 or a substantially identical sequence thereof where the fragment includes a polymorphic site. The kit sometimes comprises a polymerizing agent, for example, a thermostable nucleic acid polymerase such as one disclosed in U.S. Pat. No. 4,889,818 or U.S. Pat. No. 6,077,664. Also, the kit often comprises an elongation oligonucleotide that hybridizes to an EPHA3 nucleotide sequence in a nucleic acid sample adjacent to the polymorphic site. Where the kit includes an elongation oligonucleotide, it also often comprises chain elongating nucleotides, such as dATP, dTTP, dGTP, dCTP, and dITP, including analogs of dATP, dTTP, dGTP, dCTP and dITP, provided that such analogs are substrates for a thermostable nucleic acid polymerase and can be incorporated into a nucleic acid chain elongated from the extension oligonucleotide. Along with chain elongating nucleotides would be one or more chain terminating nucleotides such as ddATP, ddTTP, ddGTP, ddCTP, and the like. (In an embodiment, the kit comprises one or more oligonucleotide primer pairs a polymerizing agent, chain elongating nucleotides, at least one elongation oligonucleotide, and one or more chain terminating nucleotides. Kits optionally include buffers, vials, microtiter plates, and instructions for use.

An individual identified as being at risk of type II diabetes may be heterozygous or homozygous with respect to the allele associated with a higher risk of type II diabetes. A subject homozygous for an allele associated with an increased risk of Type II diabetes is at a comparatively high risk of type II diabetes, a subject heterozygous for an allele associated with an increased risk of type II diabetes is at a comparatively intermediate risk of type II diabetes, and a subject homozygous for an allele associated with a decreased risk of type II diabetes is at a comparatively low risk of type II diabetes. A genotype may be assessed for a complementary strand, such that the complementary nucleotide at a particular position is detected.

Also featured are methods for determining risk of type II diabetes and/or identifying a subject at risk of type II diabetes by contacting a polypeptide or protein encoded by an EPHA3 nucleotide sequence from a subject with an antibody that specifically binds to an epitope associated with increased risk of type II diabetes in the polypeptide. In an embodiment, the antibody specifically binds to an epitope comprising an arginine at position 924 in an EPHA3 polypeptide (SEQ ID NO: 4).

Applications of Prognostic and Diagnostic Results to Pharmacogenomic Methods

Pharmacogenomics is a discipline that involves tailoring a treatment for a subject according to the subject's genotype as a particular treatment regimen may exert a differential effect depending upon the subject's genotype. For example, based upon the outcome of a prognostic test described herein, a clinician or physician may target pertinent information and preventative or therapeutic treatments to a subject who would be benefited by the information or treatment and avoid directing such information and treatments to a subject who would not be benefited (e.g., the treatment has no therapeutic effect and/or the subject experiences adverse side effects).

The following is an example of a pharmacogenomic embodiment. A particular treatment regimen can exert a differential effect depending upon the subject's genotype. Where a candidate therapeutic exhibits a significant interaction with a major allele and a comparatively weak interaction with a minor allele (e.g., an order of magnitude or greater difference in the interaction), such a therapeutic typically would not be administered to a subject genotyped as being homozygous for the minor allele, and sometimes not administered to a subject genotyped as being heterozygous for the minor allele. In another example, where a candidate therapeutic is not significantly toxic when administered to subjects who are homozygous for a major allele but is comparatively toxic when administered to subjects heterozygous or homozygous for a minor allele, the candidate therapeutic is not typically administered to subjects who are genotyped as being heterozygous or homozygous with respect to the minor allele.

The methods described herein are applicable to pharmacogenomic methods for preventing, alleviating or treating type II diabetes conditions such as metabolic disorders, syndrome X, obesity, hypertension, insulin resistance, hyperglycemia. For example, a nucleic acid sample from an individual may be subjected to a prognostic test described herein. Where one or more polymorphic variations associated with increased risk of type II diabetes are identified in a subject, information for preventing or treating type II diabetes and/or one or more type II diabetes treatment regimens then may be prescribed to that subject.

In certain embodiments, a treatment or preventative regimen is specifically prescribed and/or administered to individuals who will most benefit from it based upon their risk of developing type II diabetes assessed by the methods described herein. Thus, provided are methods for identifying a subject predisposed to type II diabetes and then prescribing a therapeutic or preventative regimen to individuals identified as having a predisposition. Thus, certain embodiments are directed to a method for reducing type II diabetes in a subject, which comprises: detecting the presence or absence of a polymorphic variant associated with type II diabetes in a nucleotide sequence in a nucleic acid sample from a subject where the nucleotide sequence comprises a polynucleotide sequence selected from the group consisting of: (a) a nucleotide sequence of SEQ ID NO: 1-3; (b) a nucleotide sequence which encodes a polypeptide consisting of an amino acid sequence encoded by a nucleotide sequence of SEQ ID NO: 1-3; (c) a nucleotide sequence which encodes a polypeptide that is 90% or more identical to an amino acid sequence encoded by a nucleotide sequence of SEQ ID NO: 1-3, or a nucleotide sequence about 90% or more identical to a nucleotide sequence of SEQ ID NO: 1-3; and (d) a fragment of a polynucleotide sequence of (a), (b), or (c); and prescribing or administering a treatment regimen to a subject from whom the sample originated where the presence of a polymorphic variation associated with type II diabetes is detected in the nucleotide sequence. In these methods, predisposition results may be utilized in combination with other test results to diagnose type II diabetes associated conditions, such as metabolic disorders, syndrome X, obesity, hypertension, insulin resistance, hyperglycemia.

Certain preventative treatments often are prescribed to subjects having a predisposition to type II diabetes and where the subject is diagnosed with type II diabetes or is diagnosed as having symptoms indicative of early stage type II diabetes, (e.g., impaired glucose tolerance, or IGT). For example, recent studies have highlighted the potential for intervention in IGT subjects to reduce progression to type II diabetes. One such study showed that over three years lifestyle intervention (targeting diet and exercise) reduced the risk of progressing from IGT to diabetes by 58% (The Diabetes Prevention Program. (1999) Diabetes Care 22:623-634). In a similar Finnish study, the cumulative incidence of diabetes after four years was 11% in the intervention group and 23% in the control group. During the trial, the risk of diabetes was reduced by 58% in the intervention group (Tuomilehto et al. (2001) N. Eng. J. Med. 344:1343-1350). Clearly there is great benefit in the early diagnosis and subsequent preventative treatment of type II diabetes.

The treatment sometimes is preventative (e.g., is prescribed or administered to reduce the probability that a type II diabetes associated condition arises or progresses), sometimes is therapeutic, and sometimes delays, alleviates or halts the progression of a type II diabetes associated condition Any known preventative or therapeutic treatment for alleviating or preventing the occurrence of a type II diabetes associated disorder is prescribed and/or administered. For example, the treatment sometimes includes changes in diet, increased exercise, and the administration of therapeutics such as sulphonylureas (and related insulin secretagogues), which increase insulin release from pancreatic islets; metformin (Glucophage™), which acts to reduce hepatic glucose production; peroxisome proliferator-activated receptor-gamma (PPAR) agonists (thiozolidinediones such as Avandia® and Actos®), which enhance insulin action; alpha-glucosidase inhibitors (e.g., Precose®, Voglibose®, and Miglitol®), which interfere with gut glucose absorption; and insulin itself, which suppresses glucose production and augments glucose utilization (Moller Nature 414, 821-827 (2001)).

As therapeutic approaches for type U diabetes continue to evolve and improve, the goal of treatments for type II diabetes related disorders is to intervene even before clinical signs (e.g., impaired glucose tolerance, or IGT) first manifest. Thus, genetic markers associated with susceptibility to type II diabetes prove useful for early diagnosis, prevention and treatment of type II diabetes.

As type II diabetes preventative and treatment information can be specifically targeted to subjects in need thereof (e.g., those at risk of developing type II diabetes or those that have early stages of type II diabetes), provided herein is a method for preventing or reducing the risk of developing type II diabetes in a subject, which comprises: (a) detecting the presence or absence of a polymorphic variation associated with type II diabetes at a polymorphic site in a nucleotide sequence in a nucleic acid sample from a subject; (b) identifying a subject with a predisposition to type II diabetes, whereby the presence of the polymorphic variation is indicative of a predisposition to type II diabetes in the subject; and (c) if such a predisposition is identified, providing the subject with information about methods or products to prevent or reduce type II diabetes or to delay the onset of type II diabetes. Also provided is a method of targeting information or advertising to a subpopulation of a human population based on the subpopulation being genetically predisposed to a disease or condition, which comprises: (a) detecting the presence or absence of a polymorphic variation associated with type II diabetes at a polymorphic site in a nucleotide sequence in a nucleic acid sample from a subject; (b) identifying the subpopulation of subjects in which the polymorphic variation is associated with type II diabetes; and (c) providing information only to the subpopulation of subjects about a particular product which may be obtained and consumed or applied by the subject to help prevent or delay onset of the disease or condition.

Pharmacogenomics methods also may be used to analyze and predict a response to a type II diabetes treatment or a drug. For example, if pharmacogenomics analysis indicates a likelihood that an individual will respond positively to a type II diabetes treatment with a particular drug, the drug may be administered to the individual. Conversely, if the analysis indicates that an individual is likely to respond negatively to treatment with a particular drug, an alternative course of treatment may be prescribed. A negative response may be defined as either the absence of an efficacious response or the presence of toxic side effects. The response to a therapeutic treatment can be predicted in a background study in which subjects in any of the following populations are genotyped: a population that responds favorably to a treatment regimen, a population that does not respond significantly to a treatment regimen, and a population that responds adversely to a treatment regiment (e.g., exhibits one or more side effects). These populations are provided as examples and other populations and subpopulations may be analyzed. Based upon the results of these analyses, a subject is genotyped to predict whether he or she will respond favorably to a treatment regimen, not respond significantly to a treatment regimen, or respond adversely to a treatment regimen.

The tests described herein also are applicable to clinical drug trials. One or more polymorphic variants indicative of response to an agent for treating type II diabetes or to side effects to an agent for treating type II diabetes may be identified using the methods described herein. Thereafter, potential participants in clinical trials of such an agent may be screened to identify those individuals most likely to respond favorably to the drug and exclude those likely to experience side effects. In that way, the effectiveness of drug treatment may be measured in individuals who respond positively to the drug, without lowering the measurement as a result of the inclusion of individuals who are unlikely to respond positively in the study and without risking undesirable safety problems.

Thus, another embodiment is a method of selecting an individual for inclusion in a clinical trial of a treatment or drug comprising the steps of: (a) obtaining a nucleic acid sample from an individual; (b) determining the identity of a polymorphic variation which is associated with a positive response to the treatment or the drug, or at least one polymorphic variation which is associated with a negative response to the treatment or the drug in the nucleic acid sample, and (ca including the individual in the clinical trial if the nucleic acid sample contains said polymorphic variation associated with a positive response to the treatment or the drug or if the nucleic acid sample lacks said polymorphic variation associated with a negative response to the treatment or the drug. In addition, the methods described herein for selecting an individual for inclusion in a clinical trial of a treatment or drug encompass methods with any further limitation described in this disclosure, or those following, specified alone or in any combination. The polymorphic variation may be in a sequence selected individually or in any combination from the group consisting of (i) a nucleotide sequence of SEQ ID NO: 1-3; (ii) a nucleotide sequence which encodes a polypeptide consisting of an amino acid sequence encoded by a nucleotide sequence of SEQ ID NO: 1-3; (iii) a nucleotide sequence which encodes a polypeptide that is 90% or more identical to an amino acid sequence encoded by a nucleotide sequence of SEQ ID NO: 1-3, or a nucleotide sequence about 90% or more identical to a nucleotide sequence of SEQ ID NO: 1-3; and (iv) a fragment of a polynucleotide sequence of (i), (ii), or (iii) comprising the polymorphic site. The including step (c) optionally comprises administering the drug or the treatment to the individual if the nucleic acid sample contains the polymorphic variation associated with a positive response to the treatment or the drug and the nucleic acid sample lacks said biallelic marker associated with a negative response to the treatment or the drug.

Also provided herein is a method of partnering between a diagnostic/prognostic testing provider and a provider of a consumable product, which comprises: (a) the diagnostic/prognostic testing provider detects the presence or absence of a polymorphic variation associated with type U diabetes at a polymorphic site in a nucleotide sequence in a nucleic acid sample from a subject; (b) the diagnostic/prognostic testing provider identifies the subpopulation of subjects in which the polymorphic variation is associated with type B diabetes; (c) the diagnostic/prognostic testing provider forwards information to the subpopulation of subjects about a particular product which may be obtained and consumed or applied by the subject to help prevent or delay onset of the disease or condition; and (d) the provider of a consumable product forwards to the diagnostic test provider a fee every time the diagnostic/prognostic test provider forwards information to the subject as set forth in step (c) above.

Compositions Comprising Diabetes-Directed Molecules

Featured herein is a composition comprising a cell from a subject having type II diabetes or at risk of type II diabetes and one or more molecules specifically directed and targeted to a nucleic acid comprising an EPHA3 nucleotide sequence or amino acid sequence. Such directed molecules include, but are not limited to, a compound that binds to an EPHA3 nucleotide sequence or amino acid sequence referenced herein; a nucleic acid that hybridizes to an EPHA3 nucleic acid under stringent conditions, a RNAi or siRNA molecule having a strand complementary to an EPHA3 nucleotide sequence; an antisense nucleic acid complementary to an RNA encoded by an EPHA3 nucleotide sequence; a ribozyme that hybridizes to an EPHA3 nucleotide sequence; a nucleic acid aptamer that specifically binds a polypeptide encoded by EPHA3 nucleotide sequence; and an antibody that specifically binds to a polypeptide encoded by EPHA3 nucleotide sequence or binds to a nucleic acid having such a nucleotide sequence. In specific embodiments, the diabetes directed molecule interacts with a nucleic acid or polypeptide variant associated with diabetes, such as variants referenced herein. In other embodiments, the diabetes directed molecule interacts with a polypeptide involved in a signal pathway of a polypeptide encoded by an EPHA3 nucleotide sequence, or a nucleic acid comprising such a nucleotide sequence.

In certain embodiments, the diabetes directed molecule is an antibody that specifically binds to an EPHA3 isoform, for example, to an epitope comprising an arginine or tryptophan at position 924 in an EPHA3 polypeptide (SEQ ID NO: 4) or a histidine or arginine at position 914. The antibody sometimes specifically binds to EPHA3 and inhibits an interaction (e.g., binding) between EPHA3 and an EPHA3 binding partner or ligand, such as Ephrin-A5 or Ephrin-A2. In certain embodiments, the antibody specifically binds to an EPHA3 binding partner or ligand (e.g., the antibody specifically binds to Ephrin-A2 or Ephrin-A5) and inhibits binding between EPHA3 and that binding partner or ligand. In another embodiment, the antibody specifically binds to a metalloprotease enzyme (e.g., a disintegrin and metalloproteinase domain 10 (ADAM10)) that catalyzes the aggregation between EPHA3 and its binding partner or ligand (e.g., Ephrin-A2). Hattori et al. shows ephrin-A2 forms a stable complex with the metalloprotease Kuzbanian or ADAM10 (NM_{—}001110) (Science. 2000 Aug. 25; 299(5483):1360-5). Binding inhibition may be partial (e.g., 50% of less binding, 25% of less binding, 20% or less binding, or 5% or less binding) or complete. In some embodiments, a composition described herein includes an EPHA3 binding partner or ligand, such as Ephrin-A2, Ephrin-A5 or the peptide fragments disclosed in U.S. Pat. No. 6,063,903. The diabetes directed molecule sometimes is an EPHA3 polypeptide fragment. In certain embodiments, isoform b of EPHA3 (SEQ ID NO-5), the extracellular domain of isoform a (21-541 of SEQ ID NO:4), or a fragment of the foregoing, specifically binds to an EPHA3 binding partner ligand (e.g., Ephrin-A2 or Ephrin-A5) and inhibits binding between EPHA3 and that binding partner or ligand.

Compositions sometimes include an adjuvant known to stimulate an immune response, and in certain embodiments, an adjuvant that stimulates a T-cell lymphocyte response. Adjuvants are known, including but not limited to an aluminum adjuvant (e.g., aluminum hydroxide); a cytokine adjuvant or adjuvant that stimulates a cytokine response (e.g., interleukin (II)-12 and/or γ-interferon cytokines); a Freund-type mineral oil adjuvant emulsion (e.g., Freund's complete or incomplete adjuvant); a synthetic lipoid compound; a copolymer adjuvant (e.g., TitreMax); a saponin; Quil A; a liposome; an oil-in-water emulsion (e.g., an emulsion stabilized by Tween 80 and pluronic polyoxyethylene/polyoxypropylene block copolymer (Syntex Adjuvant Formulation), TitreMax; detoxified endotoxin (MPL) and mycobacterial cell wall components (TDW, CWS) in 2% squalene (Ribi Adjuvant System)); a muramyl dipeptide; an immune-stimulating complex (ISCOM, e.g., an Ag-modified saponin/cholesterol micelle that forms stable cage-like structure); an aqueous phase adjuvant that does not have a depot effect (e.g., Gerbu adjuvant); a carbohydrate polymer (e.g., AdjuPrime); L-tyrosine; a manide-oleate compound (e.g., Montanide); an ethylene-vinyl acetate copolymer (e.g., Elvax 40W1,2); or lipid A, for example. Such compositions are useful for generating an immune response against a diabetes directed molecule (e.g., an HLA-binding subsequence within a polypeptide encoded by an EPHA3 nucleotide sequence). In such methods, a peptide having an amino acid subsequence of a polypeptide encoded by an EPHA3 nucleotide sequence is delivered to a subject, where the subsequence binds to an HLA molecule and induces a CTL lymphocyte response. The peptide sometimes is delivered to the subject as an isolated peptide or as a minigene in a plasmid that encodes the peptide. Methods for identifying HLA-binding subsequences in such polypeptides are known (see e.g., publication WO02/20616 and PCT application US98/01373 for methods of identifying such sequences).

The cell may be in a group of cells cultured in vitro or in a tissue maintained in vitro or present in an animal in vivo (e.g., a rat, mouse, ape or human). In certain embodiments, a composition comprises a component from a cell such as a nucleic acid molecule (e.g. genomic DNA), a protein mixture or isolated protein, for example. The aforementioned compositions have utility in diagnostic, prognostic and pharmacogenomic methods described previously and in diabetes therapeutics described hereafter. Certain diabetes directed molecules are described in greater detail below.

Compounds

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993>; Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994).

Libraries of compounds may be presented in solution (e.g., Houghten, Biotechniques 13: 412-421 (1992), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992>) or on phage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes alters expression and sometimes alters activity of a polypeptide target and may be a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

In certain embodiments, compounds include, but are not limited to, inhibitors of tyrosine protein kinases that inhibit EPHA3. Tyrosine kinases include epidermal growth factor receptor protein kinase (EGFR), vascular endothelial growth factor receptor protein kinase (VEGFR), or kinase insert domain receptor (KDR). Thus, EPHA3 compounds include inhibitors of EGFR, VEGFR and KDR, for which structures and methods of synthesis are described in PCT international patent publications: WO0132651, WO0047212, WO9813354, WO9813350, WO9732856, WO9730035 and WO9730035. Examples of compound structures are provided hereafter.

In certain embodiments, diabetes directed molecules include compounds of formula I:

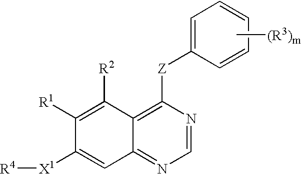

where Z represents —O—, —NH— or —S—; m is an integer from 1 to 5; R¹ represents hydrogen, hydroxy, halogeno, nitro, trifluoromethyl, cyano, C1-3alkyl, C1-3alkoxy, C1-3alkylthio, or —NR⁵R⁶ (wherein R⁵ and R⁶, which may be the same or different, each represents hydrogen or C1-3alkyl); R² represents hydrogen, hydroxy, halogeno, methoxy, amino or nitro; R³ represents hydroxy, halogeno, C1-3alkyl, C1-3alkoxy, C1-3alkanoyloxy, trifluoromethyl, cyano, amino or nitro; X¹ represents —O—, —CH₂—, —S—, —SO—, —SO₂—, —NR⁷—, —NR⁸CO—, —CONR⁹—, —SO₂NR³⁰— or —NR¹¹SO₂—, (where R⁷, R⁸, R⁹, R¹⁰ and R¹¹ each represents hydrogen, C1-3alkyl or C1-3alkoxyC2-3alkyl); and R⁴ represents a group which is alkenyl, alkynyl or optionally substituted alkyl, which alkyl group may contain a heteroatom linking group, which alkenyl, alkynyl or alkyl group may carry a terminal optionally substituted 5 or 6 membered saturated carbocyclic or heterocyclic group and salts thereof. Processes for their preparation, pharmaceutical compositions containing a compound of the formula, and pharmaceutically acceptable salts are described in further detail in WO 9730035.

In some embodiments, diabetes directed molecules include compounds of formula II:

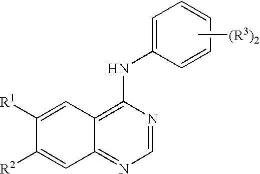

where R¹ represents hydrogen or methoxy; R¹ represents methoxy, ethoxy, 2-methoxyethoxy, 3-methoxypropoxy, 2-ethoxyethoxy, trifluoromethoxy, 2,2,2-trifluoroethoxy, 2-hydroxyethoxy, 3-hydroxypropoxy, 2-(N,N-dimethylamino)ethoxy, 3-(N,N-dimethylamino)propoxy, 2-morpholinoethoxy, 3-morpholinopropoxy, 4-morpholinobutoxy, 2-piperidinoethoxy, 3-piperidinopropoxy, 4-piperidinobutoxy, 2-(piperazin-1-yl)ethoxy, 3-(piperazin-1-yl)propoxy, 4-(piperazin-1-yl)butoxy, 2-(4-methylpiperazin-1-yl)ethoxy, 3-(4-methylpiperazin-1-yl)propoxy or 4-(4-methylpiperazin-1-yl)butoxy; and the phenyl group bearing (R³)₂ is selected from: 2-fluoro-5-hydroxyphenyl, 4-bromo-2-fluorophenyl, 2,4-difluorophenyl, 4-chloro-2-fluorophenyl, 2-fluoro-4-methylphenyl, 2-fluoro-4-methoxyphenyl, 4-bromo-3-hydroxyphenyl, 4-fluoro-3-hydroxyphenyl, 4-chloro-3-hydroxyphenyl, 3-hydroxy-4-methylphenyl, 3-hydroxy-4-methoxyphenyl and 4-cyano-2-fluorophenyl); and salts thereof. Processes for their preparation, pharmaceutical compositions containing a compound of the formula and pharmaceutically acceptable salts are described in further detail in WO 9732856.

In certain embodiments, diabetes directed molecules include compounds of formula III:

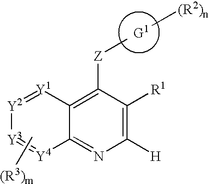

where R² represents hydroxy, halogeno, C1-3alkyl, C1-3alkoxy, C1-3alkanoyloxy, trifluoromethyl, cyano, amino or nitro; n is an integer from 0 to 5; Z represents —O—, —NH—, —S— or —CH2-; G¹ represents phenyl or a 5-10 membered heteroaromatic cyclic or bicyclic group; Y¹, Y², Y³ and Y⁴ each independently represents carbon or nitrogen; R¹ represents fluoro or hydrogen; m is an integer from 1 to 3; R³ represents hydrogen, hydroxy, halogeno, cyano, nitro, trifluoromethyl; C1-3alkyl, —NR⁴R⁵ (wherein R⁴ and R⁵ can each be hydrogen or C1-3alkyl), or a group R⁶—X¹— wherein X¹ represents —CH2- or a heteroatom linker group and R⁶ is an alkyl, alkenyl or alkynyl chain optionally substituted by for example hydroxy, amino, nitro, alkyl, cycloalkyl, alkoxyalkyl, or an optionally substituted group selected from pyridone, phenyl and a heterocyclic ring, which alkyl, alkenyl or alkynyl chain may have a heteroatom linker group, or R⁶ is an optionally substituted group selected from pyridone, phenyl and a heterocyclic ring and salts thereof. Processes for their preparation, pharmaceutical compositions containing a compound of the formula and pharmaceutically acceptable salts are described in further detail in WO 9813350.

In some embodiments, diabetes directed molecules may include compounds of formula IV:

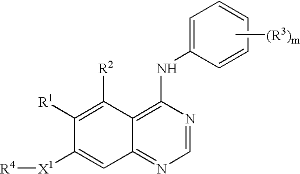

where m is an integer from 1 to 2; R¹ represents hydrogen, hydroxy, halogeno, nitro, trifluoromethyl, cyano, C1-3alkyl, C1-3alkoxy, C1-3alkylthio, or —NR⁵R⁶ (wherein R⁵ and R⁶, which may be the same or different, each represents hydrogen or C1-3alkyl); R² represents hydrogen, hydroxy, halogeno, methoxy, amino or nitro; R³ represents hydroxy, halogeno, C1-3alkyl, C1-3 alkoxy, C1-3alkanoyloxy, trifluoromethyl, cyano, amino or nitro; X¹ represents —O—, —CH2-, —S—, —SO—, —SO2-, —NR⁷CO—, —CONR⁸—, —SO2NR⁹—, —NR¹⁰SO2- or —NR¹¹— (wherein R⁷R⁸, R⁹, R¹⁰ and R¹¹ each independently represents hydrogen, C1-3alkyl or C1-3alkoxyC2-3alkyl); R⁴ represents an optionally substituted 5 or 6 membered saturated carbocyclic or heterocyclic group or a group which is alkenyl, alkynyl or optionally substituted alkyl, which alkyl group may contain a heteroatom linking group, which alkenyl, alkynyl or alkyl group may carry a terminal optionally substituted group selected from alkyl and a 5 or 6 membered saturated carbocyclic or heterocyclic group, and salts thereof. Processes for their preparation, pharmaceutical compositions containing a compound of the formula and pharmaceutically acceptable salts are described in WO 9813354.

In certain embodiments, diabetes directed molecules include compounds of formula V:

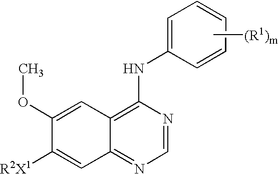

where m is an integer from 1 to 3; R¹ represents halogeno or C1-3alkyl; X¹ represents —O—; R² is selected from one of the following three groups: 1) C1-5alkylR³ (wherein R³ is piperidin-4-yl which may bear one or two substituents selected from hydroxy, halogeno, C1-4alkyl, C1-4-hydroxyalkyl and C-1-4alkoxy; 2) C2-5alkenylR³ (wherein R³ is as defined hereinbefore); 3) C2-5alkynylR³ (where R³ is as defined hereinbefore); and where any alkyl, alkenyl or alkynyl group may bear one or more substituents selected from hydroxy, halogeno and amino; and salts thereof. Processes for their preparation, pharmaceutical compositions containing a compound of the formula and pharmaceutically acceptable salts are described in further detail in WO 0132651.

In some embodiments, diabetes directed molecules include compounds of formula VI:

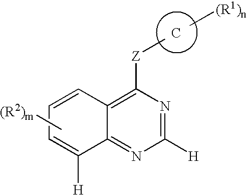

where ring C is an 8, 9, 10, 12 or 13-membered bicyclic or tricyclic moiety which optionally may contain 1-3 heteroatoms selected independently from O, N and S; Z is —O—, —NH—, —S—, —CH2- or a direct bond; n (which characterizes R¹) is 0-5; m (which characterizes R²) is 0-3; R² represents hydrogen, hydroxy, halogeno, cyano, nitro, trifluoromethyl, C1-3alkyl, C1-3alkoxy, C1-3alkylsulphanyl, —NR³R⁴ (wherein R³ and R⁴, which may be the same or different, each represents hydrogen or C1-3alkyl), or R⁵X¹— (wherein X¹ and R⁵ are as defined herein; R¹ represents hydrogen, oxo, halogeno, hydroxy, C1-4alkoxy, C1-4alkyl, C1-4-alkoxymethyl, C1-4alkanoyl, C1-4haloalkyl, cyano, amino, C2-5alkenyl, C2-5alkynyl, C1-3alkanoyloxy, nitro, C1-4alkanoylamino, C1-4-alkoxycarbonyl, C1-4alkylsulphanyl, C1-4alkylsulphinyl, C1-4alkylsulphonyl, carbamoyl, N—C1-4-alkylcarbamoyl, N,N-di(C1-4alkyl)carbamoyl, aminosulphonyl, N—C1-4alkylaminosulphonyl, N,N-di(C1-4alkyl)aminosulphonyl, N—(C1-4alkylsulphonyl)amino, N—(C1-4alkylsulphonyl)-N—(C1-4alkyl)amino, N,N-di(C1-4alkylsulphonyl)amino, a C3-7alklylene chain joined to two ring C carbon atoms, C1-4alkanoylaminoC1-4alkyl, carboxy or a group R⁵⁶X¹⁰ (wherein X¹⁰ and R⁵⁶ are as defined herein); and salts thereof. Processes for their preparation, pharmaceutical compositions containing a compound of the formula and pharmaceutically acceptable salts are described in further detail in WO 0047212.

In certain embodiments, diabetes directed molecules include compounds of formula VII:

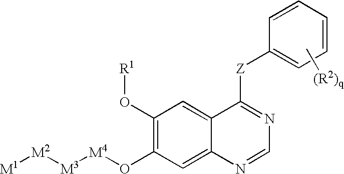

where R¹ is C₁-C₃ alkyl optionally substituted with between one and three R⁵⁰ substituents; R² is selected from —H, halogen, tribalomethyl, —CN, —NH₂, —NO₂, —R³, —N(R³)R⁴, —S(O)₀₋₂R⁴, —SO₂N(R³)R⁴, —CO₂R³, —C(═ON(R³)R⁴, —N(R³)SO₂R⁴, —N(3)C(═O)R³, —N(R³)CO₂R⁴, —C(═O)R, optionally substituted lower alkyl, optionally substituted lower alkenyl, and optionally substituted lower alkynyl; R³ is —H or R⁴; R⁴ is selected from optionally substituted lower alkyl, optionally substituted aryl, optionally substituted lower arylakyl, optionally substituted heterocyclyl, and optionally substituted lower heterocyclylalkyl; or R³ and R⁴, when taken together with a common nitrogen to which they are attached, form an optionally substituted five- to seven-membered heterocyclyl, said optionally substituted five- to seven-membered heterocyclyl optionally containing at least one additional heteroatom selected from N, O, S, and P; q is zero to five; Z is selected from —OCH₂—, —O—, —S(O)₀₋₂, —N(R⁵)CH₂—, and —NR⁵—; R⁵ is —H or optionally substituted lower alkyl; M¹ is —H, C₁-C₈ alkyl-L²-L¹- optionally substituted by R⁵⁰, G(CH₂)₀₋₃—, or R⁵³(R⁵⁴)N(CH₂)₀₋₃—; wherein G is a saturated five- to seven-membered heterocyclyl containing one or two annular heteroatoms and optionally substituted with between one and three R⁵⁰ substituents; L′ is —C═O— or —SO₂—; L² is a direct bond, —O—, or NH—; and R⁵³ and R⁵⁴ are independently C₁-C₃ alkyl optionally substituted with between one and three R⁵⁰ substituents; M² is a saturated or mono- or poly-unsaturated C₃-C₁₄ mono- or fused-polycyclic hydrocarbyl optionally containing one, two, or three annular heteroatoms per ring and optionally substituted with between zero and four R⁵⁰ substituents; and M³ is —NR⁹—, —O—, or absent; M⁴ is CH₂—, —CH₂CH₂,—, —CH₂CH₂CH₂—, or absent; R⁹ is —H or optionally substituted lower alkyl; (50 is —H, halo, trihalomethyl, —OR³, —N(R³)R⁴, —S(O)₀₋₂R⁴, —SO₂N(R³)R⁴, —CO₂R³, —C(═O)N(R³)R⁴, —C(═NR²⁵)(R³)R⁴, —C(═NR²⁵)R⁴, —N(R³)S(O)₂R⁴—N(R³)C(O)R³, —NCO₂R³, —C(═O)R³, optionally substituted alkoxy, optionally substituted lower alkyl, optionally substituted aryl, optionally substituted lower arylalkyl, optionally substituted heterocyclyl, and optionally substituted lower heterocyclylalkyl; or two of R⁵⁰, when taken together on the same carbon are oxo; or two of R⁵⁰, when taken together with a common carbon to which they are attached, form an optionally substituted three- to seven-membered spirocyclyl, said optionally substituted three- to seven-membered spirocyclyl optionally containing at least one additional heteroatom selected from N, O, S, and P; and R²⁵ is selected from —H, —CN, —NO₂, —OR³, —S(O)₀₋₂R⁴, —CO₂R³, optionally substituted lower alkyl, optionally substituted lower alkenyl, and optionally substituted lower alkynyl, and salts thereof. Processes for their preparation, pharmaceutical compositions containing a compound of the formula and pharmaceutically acceptable salts are described in further detail in WO 2004006846.

Certain embodiments pertain to the following compounds, pharmaceutically acceptable salts thereof, and compositions comprising the foregoing.

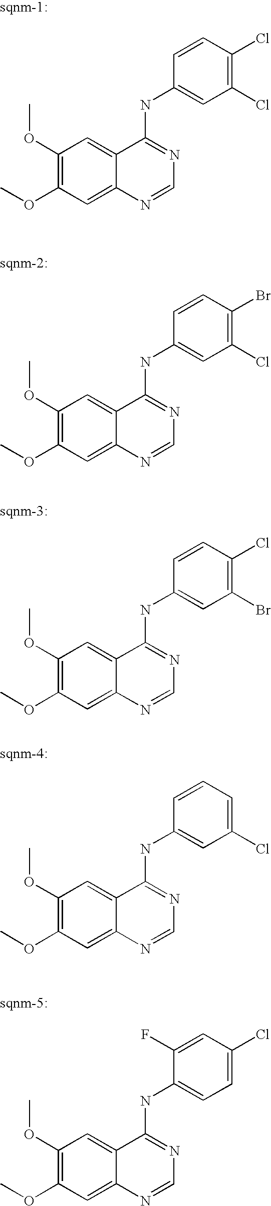

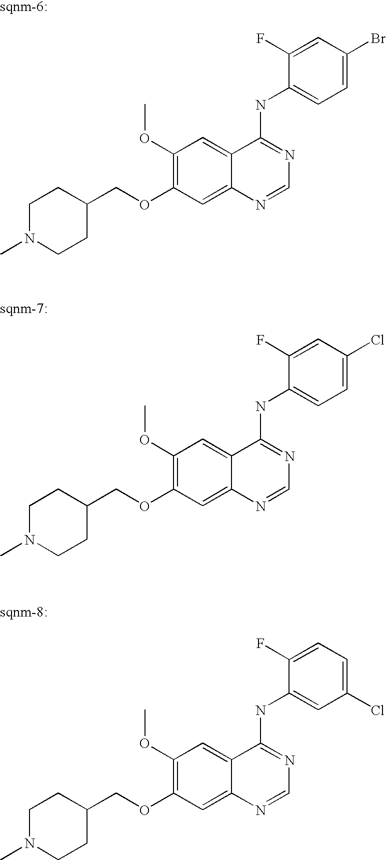

In other embodiments, examples of compounds include, but are not limited to, EphA2 and EphB4 inhibitors. Examples of EphA2 and EphB4 inhibitors are described in PCT international patent publication WO2004006846. Examples of compound structures are shown below,

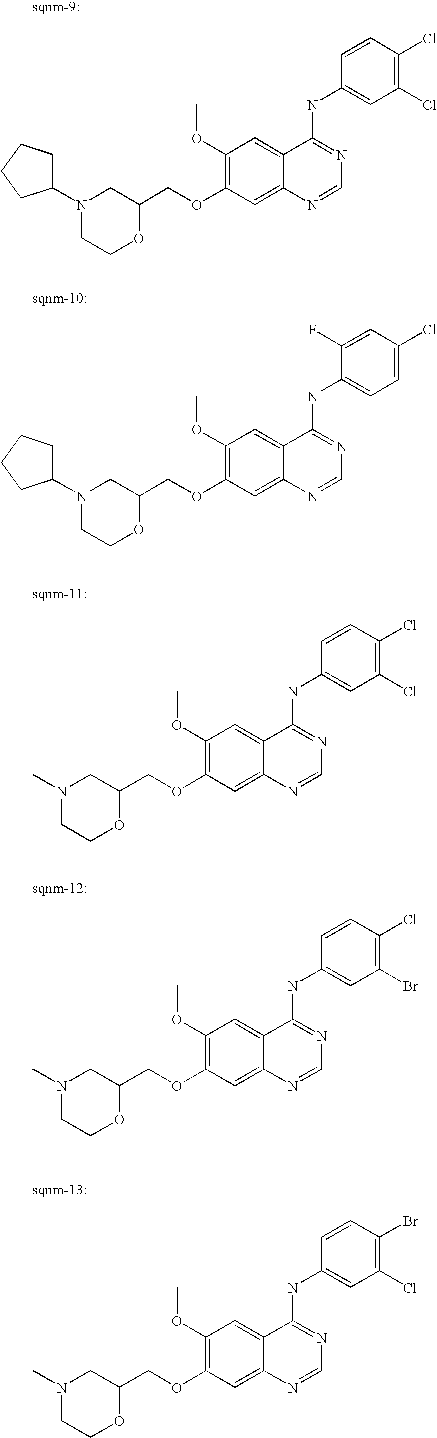

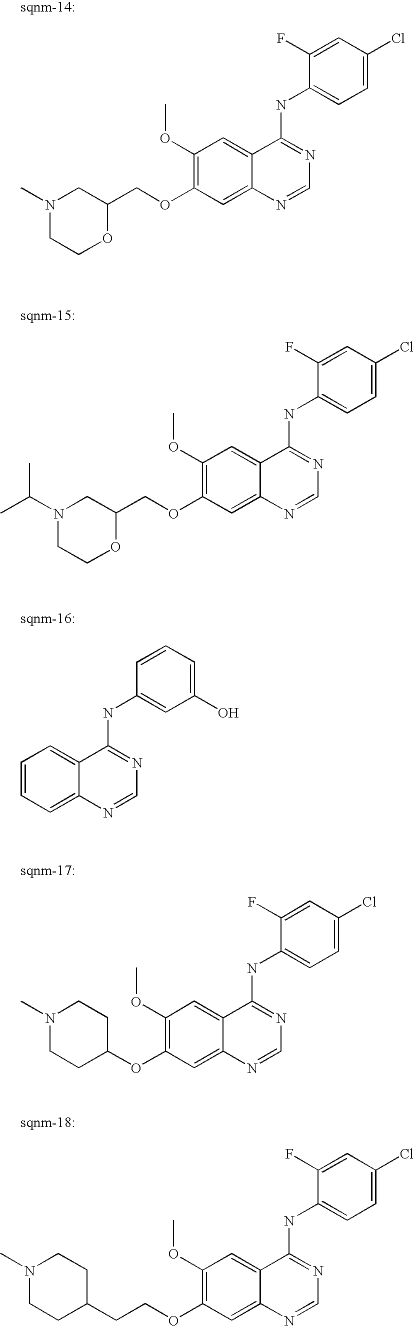

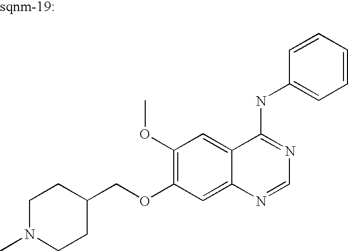

and some embodiments are directed to pharmaceutically acceptable salts and formulations of the foregoing.

In certain embodiments, a compound specifically binds to EPHA3 and inhibits an interaction (e.g., binding) between EPHA3 and an EPHA3 binding partner or ligand, such as Ephrin-A5 or Ephrin-A2. In some embodiments, the compound specifically binds to an EPHA3 binding partner or ligand (e.g., the antibody specifically binds to Ephrin-A2 or Ephrin-A5) and inhibits binding between EPHA3 and that binding partner or ligand. In an embodiment, the compound specifically binds to a metalloprotease enzyme (e.g., a disintegrin and metalloproteinase domain 10 (ADAM10)) that catalyzes the aggregation between EPHA3 and its binding partner or ligand (e.g., Ephrin-A2).

Antisense Nucleic Acid Molecules, Ribozymes, RNAi siRNA and Modified Nucleic Acid Molecules

An “antisense” nucleic acid refers to a nucleotide sequence complementary to a “sense” nucleic acid encoding a polypeptide, e.g., complementary to the coding strand of a double-stranded cDNA molecule or complementary to an mRNA sequence. The antisense nucleic acid can be complementary to an entire coding strand (e.g., SEQ ID NO: 2-3), or to a portion thereof or a substantially identical sequence thereof. In another embodiment, the antisense nucleic acid molecule is antisense to a “noncoding region” of the coding strand of a nucleotide sequence (e.g., 5′ and 3′ untranslated regions in SEQ ID NO: 1).

An antisense nucleic acid can be designed such that it is complementary to the entire coding region of an mRNA encoded by a nucleotide sequence (e.g., SEQ ID NO: 1-3), and often the antisense nucleic acid is an oligonucleotide antisense to only a portion of a coding or noncoding region of the mRNA. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length. The antisense nucleic acids, which include the ribozymes described hereafter, can be designed to target an EPHA3 nucleotide sequence, often a variant associated with diabetes, or a substantially identical sequence thereof. Among the variants, minor alleles and major alleles can be targeted, and those associated with a higher risk of diabetes are often designed, tested, and administered to subjects.

An antisense nucleic acid can be constructed using chemical synthesis and enzymatic ligation reactions using standard procedures. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized as therapeutics, antisense nucleic acids typically are administered to a subject (e.g., by direct injection at a tissue site) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a pol II or pol III promoter, in the vector construct.

Antisense nucleic acid molecules sometimes are alpha-anomeric nucleic acid molecules. An alpha-anomeric nucleic acid molecule forms specific double-stranded hybrids with complementary RNA in which, contrary to the usual beta-units, the strands run parallel to each other (Gaultier et al., Nucleic Acids. Res. 15: 6625-6641 (1987)). Antisense nucleic acid molecules can also comprise a 2'-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

In another embodiment, an antisense nucleic acid is a ribozyme. A ribozyme having specificity for an EPHA3 nucleotide sequence can include one or more sequences complementary to such a nucleotide sequence, and a sequence having a known catalytic region responsible for mRNA cleavage (see e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (see e.g., Cech et al. U.S. Pat. No. 4,987,071; and Cech et al. U.S. Pat. No. 5,116,742). Also, target mRNA sequences can be used to select a catalytic RNA having a specific ribonuclease activity from a pool of RNA molecules (see e.g., Bartel & Szostak, Science 261: 1411-1418 (1993)).

Diabetes directed molecules include in certain embodiments nucleic acids that can form triple helix structures with an EPHA3 nucleotide sequence or a substantially identical sequence thereof, especially one that includes a regulatory region that controls expression of a polypeptide. Gene expression can be inhibited by targeting nucleotide sequences complementary to the regulatory region of a nucleotide sequence referenced herein or a substantially identical sequence (e.g., promoter and/or enhancers) to form triple helical structures that prevent transcription of a gene in target cells (see e.g., Helene, Anticancer Drug Des. 6(6): 569-84 (1991); Helene et al. Ann. N.Y. Acad. Sci-660: 27-36 (1992); and Maher, Bioassays 14(12): 807-15 (1992). Potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

Diabetes directed molecules include RNAi and siRNA nucleic acids. Gene expression may be inhibited by the introduction of double-stranded RNA (dsRNA), which induces potent and specific gene silencing, a phenomenon called RNA interference or RNAi. See, e.g., Fire et al. U.S. Pat. No. 6,506,559; Tuschl et al. PCT International Publication No. WO 01/75164; Kay et al. PCT International Publication No. WO 03/01018 OA 1; or Bosher J M, Labouesse, Nat Cell Biol 2000 February; 2(2):E31-6. This process has been improved by decreasing the size of the double-stranded RNA to 20-24 base pairs (to create small-interfering RNAs or siRNAs) that “switched off” genes in mammalian cells without initiating an acute phase response, i.e., a host defense mechanism that often results in cell death (see, e.g., Caplen et al. Proc Natl Acad Sci USA. 2001 Aug. 14; 98(17):9742-7 and Elbashir et al. Methods 2002 February; 26(2):199-213). There is increasing evidence of post-transcriptional gene silencing by RNA interference (RNAi) for inhibiting targeted expression in mammalian cells at the mRNA level, in human cells. There is additional evidence of effective methods for inhibiting the proliferation and migration of tumor cells in human patients, and for inhibiting metastatic cancer development (see, e.g., U.S. Patent Application No. US2001000993183; Caplen et al. Proc Natl Acad Sci USA; and Abderrahmani et al. Mol Cell Biol 2001 Nov. 21(21):7256-67).

An “siRNA” or “RNAi” refers to a nucleic acid that forms a double stranded RNA and has the ability to reduce or inhibit expression of a gene or target gene when the siRNA is delivered to or expressed in the same cell as the gene or target gene. “siRNA” refers to short double-stranded RNA formed by the complementary strands. Complementary portions of the siRNA that hybridize to form the double stranded molecule often have substantial or complete identity to the target molecule sequence. In one embodiment, an siRNA refers to a nucleic acid that has substantial or complete identity to a target gene and forms a double stranded siRNA.

When designing the siRNA molecules, the targeted region often is selected from a given DNA sequence beginning 50 to 100 nucleotides downstream of the start codon. See, e.g., Elbashir et al., Methods 26:199-213 (2002). Initially, 5′ or 3′ UTRs and regions nearby the start codon were avoided assuming that UTR-binding proteins and/or translation initiation complexes may interfere with binding of the siRNP or RISC endonuclease complex. Sometimes regions of the target 23 nucleotides in length conforming to the sequence motif AA(N19)TT (N, an nucleotide, and regions with approximately 30% to 70% G/C-content (often about 50% G/C-content) often are selected. If no suitable sequences are found, the search often is extended using the motif NA(N21). The sequence of the sense siRNA sometimes corresponds to (N19) TT or N21 (position 3 to 23 of the 23-nt motif), respectively. In the latter case, the 3′ end of the sense siRNA often is converted to TT. The rationale for this sequence conversion is to generate a symmetric duplex with respect to the sequence composition of the sense and antisense 3′ overhangs. The antisense siRNA is synthesized as the complement to position 1 to 21 of the 23-nt motif. Because position 1 of the 23-nt motif is not recognized sequence-specifically by the antisense siRNA, the 3′-most nucleotide residue of the antisense siRNA can be chosen deliberately. However, the penultimate nucleotide of the antisense siRNA (complementary to position 2 of the 23-nt motif) often is complementary to the targeted sequence. For simplifying chemical synthesis, TT often is utilized. siRNAs corresponding to the target motif NAR(N17)YNN, where R is purine (A,G) and Y is pyimidine (C,U), often are selected. Respective 21 nucleotide sense and antisense siRNAs often begin with a purine nucleotide and can also be expressed from pol III expression vectors without a change in targeting site. Expression of RNAs from pol III promoters often is efficient when the first transcribed nucleotide is a purine.

The sequence of the siRNA can correspond to the full length target gene, or a subsequence thereof. Often, the siRNA is about 15 to about 50 nucleotides in length (e.g., each complementary sequence of the double stranded siRNA is 15-50 nucleotides in length, and the double stranded siRNA is about 15-50 base pairs in length, sometimes about 20-30 nucleotides in length or about 20-25 nucleotides in length, e.g., 20, 21, 22, 23, 24, 25, 26, 27, 28, 29, or 30 nucleotides in length. The siRNA sometimes is about 21 nucleotides in length. Methods of using siRNA are well known in the art, and specific siRNA molecules may be purchased from a number of companies including Dharmacon Research, Inc. An siRNA molecule sometimes is of a different chemical composition as compared to native RNA that imparts increased stability in cells (e.g., decreased susceptibility to degradation), and sometimes includes one or more modifications in siSTABLE RNA described at the http address www.dharmacon.com.

Antisense, ribozyme, RNAi and siRNA nucleic acids can be altered to form modified nucleic acid molecules. The nucleic acids can be altered at base moieties, sugar moieties or phosphate backbone moieties to improve stability, hybridization, or solubility of the molecule. For example, the deoxyribose phosphate backbone of nucleic acid molecules can be modified to generate peptide nucleic acids (see Hyrup et al., Bioorganic & Medicinal Chemistry 4 (1): 5-23 ((1996)). As used herein, the terms “peptide nucleic acid” or “PNA” refers to a nucleic acid mimic such as a DNA mimic, in which the deoxyribose phosphate backbone is replaced by a pseudopeptide backbone and only the four natural nucleobases are retained. The neutral backbone of a PNA can allow for specific hybridization to DNA and RNA under conditions of low ionic strength. Synthesis of PNA oligomers can be performed using standard solid phase peptide synthesis protocols as described, for example, in Hyrup et al., (1996) supra and Perry-O'Keefe et al., Proc. Natl. Acad. Sci. 93: 14670-675 (1996).

PNA nucleic acids can be used in prognostic, diagnostic, and therapeutic applications. For example, PNAs can be used as antisense or antigene agents for sequence-specific modulation of gene expression by, for example, inducing transcription or translation arrest or inhibiting replication. PNA nucleic acid molecules can also be used in the analysis of single base pair mutations in a gene, (e.g., by PNA-directed PCR clamping); as “artificial restriction enzymes” when used in combination with other enzymes, (e.g., S1 nucleases (Hyrup (1996) supra)); or as probes or primers for DNA sequencing or hybridization Syrup et al., (1996) supra; Perry-O'Keefe supra).

In other embodiments, oligonucleotides may include other appended groups such as peptides (e.g., for targeting host cell receptors in vivo), or agents facilitating transport across cell membranes (see e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO88/09810) or the blood-brain barrier (see, erg, PCT Publication No. WO89/10134). In addition, oligonucleotides can be modified with hybridization-triggered cleavage agents (See, e.g., Krol et al., Bio-Techniques 6: 958-976 (1988)) or intercalating agents. (See, e.g., Zon, Pharm. Res. 5: 539-549 (1988)). To this end, the oligonucleotide may be conjugated to another molecule, (e.g., a peptide, hybridization triggered cross-linking agent, transport agent, or hybridization-triggered cleavage agent).

Also included herein are molecular beacon oligonucleotide primer and probe molecules having one or more regions complementary to an EPHA3 nucleotide sequence or a substantially identical sequence thereof two complementary regions one having a fluorophore and one a quencher such that the molecular beacon is useful for quantifying the presence of the nucleic acid in a sample. Molecular beacon nucleic acids are described, for example, in Lizardi et al., U.S. Pat. No. 5,854,033; Nazarenko et al., U.S. Pat. No. 5,866,336, and Livak et al., U.S. Pat. No. 5,876,930.

Antibodies

The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as, pepsin. An antibody sometimes is a polyclonal, monoclonal, recombinant (e.g., a chimeric or humanized), fully human, non-human (e.g., murine), or a single chain antibody. An antibody may have effector function and can fix complement, and is sometimes coupled to a toxin or imaging agent.

A full-length polypeptide or antigenic peptide fragment encoded by a nucleotide sequence referenced herein can be used as an immunogen or can be used to identify antibodies made with other immunogens, e.g., cells, membrane preparations, and the like. An antigenic peptide often includes at least 8 amino acid residues of the amino acid sequences encoded by a nucleotide sequence referenced herein, or substantially identical sequence thereof, and encompasses an epitope. Antigenic peptides sometimes include 10 or more amino acids, 15 or more amino acids, 20 or more amino acids, or 30 or more amino acids. Hydrophilic and hydrophobic fragments of polypeptides sometimes are used as immunogens.

Epitopes encompassed by the antigenic peptide are regions located on the surface of the polypeptide (e.g., hydrophilic regions) as well as regions with high antigenicity. For example, an Emini surface probability analysis of the human polypeptide sequence can be used to indicate the regions that have a particularly high probability of being localized to the surface of the polypeptide and are thus likely to constitute surface residues useful for targeting antibody production. The antibody may bind an epitope on any domain or region on polypeptides described herein.

Also, chimeric, humanized, and completely human antibodies are useful for applications which include repeated administration to subjects. Chimeric and humanized monoclonal antibodies, comprising both human and non-human portions, can be made using standard recombinant DNA techniques. Such chimeric and humanized monoclonal antibodies can be produced by recombinant DNA techniques known in the art, for example using methods described in Robinson et al International Application No. PCT/US86/02269; Akira, et al., European Patent Application 184,187; Taniguchi, M., European Patent Application 171,496; Morrison et al., European Patent Application 173,494; Neuberger et al., PCT International Publication No. WO 86/01533; Cabilly et al., U.S. Pat. No. 4,816,567; Cabilly et al., European Patent Application 125,023; Better et al., Science 240: 1041-1043 (1988); Liu et al., Proc. Natl. Acad. Sci. USA 84: 3439-3443 (1987); Liu et al., J. Immunol. 139: 3521-3526 (1987); Sun et al., Proc. Natl. Acad. Sci. USA 84: 214-218 (1987); Nishimura et al, Canc. Res. 47: 999-1005 (1987); Wood et al., Nature 314: 446-449 (1985); and Shaw et al., J. Natl. Cancer Inst. 80: 1553-1559 (1988); Morrison, S. L., Science 229: 1202-1207 (1985); Oi et al., BioTechniques 4: 214 (1986); Winter U.S. Pat. No. 5,225,539; Jones et al., Nature 321: 552-525 (1986); Verhoeyan et al., Science 239: 1534; and Beidler et al., J. Immunol. 141: 4053-4060 (1988).

Completely human antibodies are particularly desirable for therapeutic treatment of human patients. Such antibodies can be produced using transgenic mice that are incapable of expressing endogenous immunoglobulin heavy and light chains genes, but which can express human heavy and light chain genes. See, for example, Lonberg and Huszar, Int. Rev. Immunol. 13: 65-93 (1995); and U.S. Pat. Nos. 5,625,126; 5,633,425; 5,569,825; 5,661,016; and 5,545,806. In addition, companies such as Abgenix, Inc. (Fremont, Calif.) and Medarex, Inc. (Princeton, N.J.), can be engaged to provide human antibodies directed against a selected antigen using technology similar to that described above. Completely human antibodies that recognize a selected epitope also can be generated using a technique referred to as “guided selection.” In this approach a selected non-human monoclonal antibody (e.g., a murine antibody) is used to guide the selection of a completely human antibody recognizing the same epitope. This technology is described for example by Jespers et al., Bio/Technology 12: 899-903 (1994).

An antibody can be a single chain antibody. A single chain antibody (scFV) can be engineered (see, e.g., Colcher et al., Ann. N Y Acad. Sci. 880: 263-80 (1999); and Reiter, Clin. Cancer Res. 2: 245-52 (1996)). Single chain antibodies can be dimerized or multimerized to generate multivalent antibodies having specificities for different epitopes of the same target polypeptide.

Antibodies also may be selected or modified so that they exhibit reduced or no ability to bind an Fc receptor. For example, an antibody may be an isotype or subtype, fragment or other mutant, which does not support binding to an Fc receptor (e.g., it has a mutagenized or deleted Fc receptor binding region).

Also, an antibody (or fragment thereof) may be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1 dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thiotepa chlorambucil, melphalan, carmustine (BCNU) and lomustine (CCNU), cyclophosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Antibody conjugates can be used for modifying a given biological response. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, γ-interferon, α-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Also, an antibody can be conjugated to a second antibody to form an antibody heteroconjugate as described by Segal in U.S. Pat. No. 4,676,980, for example.

An antibody (e.g., monoclonal antibody) can be used to isolate target polypeptides by standard techniques, such as affinity chromatography or immunoprecipitation. Moreover, an antibody can be used to detect a target polypeptide (e.g., in a cellular lysate or cell supernatant) in order to evaluate the abundance and pattern of expression of the polypeptide. Antibodies can be used diagnostically to monitor polypeptide levels in tissue as part of a clinical testing procedure, e.g., to determine the efficacy of a given treatment regimen. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Also, an antibody can be utilized as a test molecule for determining whether it can treat diabetes, and as a therapeutic for administration to a subject for treating diabetes.

An antibody can be made by immunizing with a purified antigen, or a fragment thereof, e.g., a fragment described herein, a membrane associated antigen, tissues, e.g., crude tissue preparations, whole cells, preferably living cells, lysed cells, or cell fractions.

Included herein are antibodies which bind only a native polypeptide, only denatured or otherwise non-native polypeptide, or which bind both, as well as those having linear or conformational epitopes. Conformational epitopes sometimes can be identified by selecting antibodies that bind to native but not denatured polypeptide. Also featured are antibodies that specifically bind to a polypeptide variant associated with diabetes. In other embodiments, antibodies may be directed to EPHA43 ligands, namely Ephrin-A2 or Ephrin-A5. Antibodies directed to Ephrin-A5 are disclosed in U.S. Pat. No. 6,169,167.

Methods for Identifying Candidate Therapeutics for Treating Type II Diabetes

Current therapies for the treatment of type II diabetes have limited efficacy, limited tolerability and significant mechanism-based side effects, including weight gain and hypoglycemia. Few of the available therapies adequately address underlying defects such as obesity and insulin resistance (Moller D. Nature. 414:821-827 (2001)). Current therapeutic approaches were largely developed in the absence of defined molecular targets or even a solid understanding of disease pathogenesis. Therefore, provided are methods of identifying candidate therapeutics that target biochemical pathways related to the development of diabetes.

Thus, featured herein are methods for identifying a candidate therapeutic for treating type II diabetes. The methods comprise contacting a test molecule with a target molecule in a system. A “target molecule” as used herein refers to an EPHA3 nucleic acid, a substantially identical nucleic acid thereof, or a fragment thereof, an encoded polypeptide of the foregoing or a binding partner. The methods also comprise determining the presence or absence of an interaction between the test molecule and the target molecule, where the presence of an interaction between the test molecule and the nucleic acid or polypeptide identifies the test molecule as a candidate type 1 diabetes therapeutic. The interaction between the test molecule and the target molecule may be quantified.

In certain embodiments, the target molecule is an EPHA3 polymorphic variant, such as a polypeptide comprising an arginine at position 924 in SEQ ID NO: 4. In other embodiments, the target molecule is a binding partner or ligand of EPHA3, such as Ephrin-A2, Ephrin-A5, or a peptide fragments disclosed in U.S. Pat. No. 6,063,903. In certain screening assay embodiments, an interaction, such as binding, between EPHA3 and a binding partner or ligand is monitored and test molecules are assessed for their effect on the interaction. For example, see the assays disclosed in U.S. Pat. Nos. 5,674,691 and 6,599,709. Some assay embodiments monitor the effect of a test molecule on certain cell functions, such as glucose uptake by cells; glucose transport molecule activity or levels in cells (e.g., GLUT4 levels or activities in cells); triacylglycerol content in cells; resistin levels or activities in cells; levels or activities of molecules involved in resistin levels in cells such as PPAR gamma, PI3 kinase, Akt and C/EBP alpha; levels or activities of EPHA3 binding partners or ligands such as Ephrin-A2 and Ephrin-A5; and levels or activities of EPHA3-related enzymes such as ADAM10. ADAM10 cDNA and amino acid sequences are publicly accessible and are provided in SEQ ID Nos: 8 and 9, respectively. Hattori et al. describes such assays in Science. 2000 Aug. 25; 289(5483):1360-5.

In assay embodiments in which EPHA3 binding partners, ligands and signal pathway members are monitored, the modulatory effect on the following specific interactions sometimes is assessed: EPHA3 and its natural ligand ephrin-A5 and/or EPHA3 and its natural ligand ephrin-A2 and/or two or more EPHA3 moieties and/or domains of EPHA3 and/or within one or more domain(s) of an EPHA3 moiety and/or EPHA3 and downstream moieties with which EPHA3 interacts. In specific embodiments, the test molecule sometimes is an antibody or protein that may specifically bind to EPHA3 or an EPHA3 binding partner, ligand or signal pathway member. Such antibodies and proteins are disclosed in U.S. Pat. Nos. 6,169,167; 6,063,903; 6,057,124; 5,798,448; and Ahsan M, et al. Biochem Biophys Res Commun. 2002 Jul. 12; 295(2):348-53 A soluble form of EPHA3 (e.g., isoform b of EPHA3) which binds to ephrin-A5 and/or ephrin-2, preventing or diminishing the binding of ephrin-A5 to membrane bound EPHA3, may be used. Variant 2 of EPHA3 (SEQ ID NO:3) uses an alternate splice site in the 3′ coding region, compared to variant 1, that results in a frameshift. It encodes isoform b (SEQ ID NO:5) which has a shorter and distinct C-terminus compared to isoform a. This isoform lacks a transmembrane domain and may be a secreted form of the Epha3 receptor. Inter-EPHA3 interactions may also be inhibited by use of the foregoing moieties. Ehprin-A5 cDNA and amino acid sequences are publicly accessible and are provided in SEQ ID Nos: 6 and 7; respectively.

Specific assay embodiments include but are not limited to monitoring the modulatory effect of a test molecule on (a) circulating (e.g., blood, serum or plasma) levels (e.g., concentration) of glucose, where test molecules that lower the glucose levels often are selected; (b) cell or tissue sensitivity to insulin, particularly in muscle, adipose, liver or brain, where molecules that increase sensitivity often are selected; (c) progression from impaired glucose tolerance to insulin resistance, where molecules that inhibit progression often are selected; (d) glucose uptake in skeletal muscle cells, where molecules, that increase glucose uptake often are selected; (e) glucose uptake in adipose cells, where molecules that increase uptake often are selected; (f) glucose uptake in neuronal cells, where molecules that increase uptake often are selected; (g) glucose uptake in red blood cells, where molecules that increase uptake often are selected; (h) glucose uptake in the brain, where molecules that increase uptake often are selected; and (i) postprandial increase in plasma glucose following a meal, particularly a high carbohydrate meal, where molecules that reduce significantly the postprandial increase often are selected.

Test molecules and candidate therapeutics include, but are not limited to, compounds, antisense nucleic acids, siRNA molecules, ribozymes; polypeptides or proteins encoded by an EPHA3 nucleotide sequence, or a substantially identical sequence or fragment thereof, and immunotherapeutics (e.g., antibodies and HLA-presented polypeptide fragments). Antibodies directed to Ephrin-A5, an EPHA3 ligand, are disclosed in U.S. Pat. No. 6,169,167. A test molecule or candidate therapeutic may act as a modulator of target molecule concentration or target molecule function in a system. A “modulator” may agonize (i.e., up-regulates) or antagonize (i.e., down-regulates) a target molecule concentration partially or completely in a system by affecting such cellular functions as DNA replication and/or DNA processing (e.g., DNA methylation or DNA repair), RNA transcription and/or RNA processing (e.g., removal of intronic sequences and/or translocation of spliced mRNA from the nucleus), polypeptide production (e.g., translation of the polypeptide from mRNA), and/or polypeptide post-translational modification (e.g., glycosylation, phosphorylation, and proteolysis of pro-polypeptides). A modulator may also agonize or antagonize a biological function of a target molecule partially or completely, where the function may include adopting a certain structural conformation, interacting with one or more binding partners, ligand binding, catalysis (e.g., phosphorylation, dephosphorylation, hydrolysis, methylation, and isomerization), and an effect upon a cellular event (e.g., effecting progression of type II diabetes). In certain embodiments, a candidate therapeutic increases glucose uptake in cells of a subject (e.g., in certain cells of the pancreas).

As used herein, the term “system” refers to a cell free in vitro environment and a cell-based environment such as a collection of cells, a tissue, an organ, or an organism. A system if “contacted” with a test molecule in a variety of manners, including adding molecules in solution and allowing them to interact with one another by diffusion, cell injection, and any administration routes in an animal. As used herein, the term “interaction” refers to an effect of a test molecule on test molecule, where the effect sometimes is binding between the test molecule and the target molecule, and sometimes is an observable change in cells, tissue, or organism.

There are many standard methods for detecting the presence or absence of interaction between a test molecule and a target molecule. For example, titrametric, acidimetric, radiometric, NMR, monolayer, polarographic, spectrophotometric, fluorescent, and ESR assays probative of a target molecule interaction may be utilized.

Test molecule/target molecule interactions can be detected and/or quantified using assays known in the art. For example, an interaction can be determined by labeling the test molecule and/or the target molecule, where the label is covalently or non-covalently attached to the test molecule or target molecule. The label is sometimes a radioactive molecule such as ¹²⁵I, ¹³¹I, ³⁵S or ³H, which can be detected by direct counting of radioemission or by scintillation counting. Also, enzymatic labels such as horseradish peroxidase, alkaline phosphatase, or luciferase may be utilized where the enzymatic label can be detected by determining conversion of an appropriate substrate to product. In addition, presence or absence of an interaction can be determined without labeling. For example, a microphysiometer (e.g., Cytosensor) is an analytical instrument that measures the rate at which a cell acidifies its environment using a light-addressable potentiometric sensor (LAPS). Changes in this acidification rate can be used as an indication of an interaction between a test molecule and target molecule McConnell, H. M. et al., Science 257: 1906-1912 (1992)).

In cell-based systems, cells typically include an EPHA3 nucleic acid, an encoded polypeptide, or substantially identical nucleic acid or polypeptide thereof, and are often of mammalian origin, although the cell can be of any origin. Whole cells, cell homogenates, and cell fractions (e.g., cell membrane fractions) can be subjected to analysis. Where interactions between a test molecule with a target polypeptide are monitored, soluble and/or membrane bound forms of the polypeptide may be utilized. Where membrane-bound forms of the polypeptide are used, it may be desirable to utilize a solubilizing agent. Examples of such solubilizing agents include non-ionic detergents such as n-octyglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)-ₙ, 3-[(3-cholamidopropyl)dimethylaminio]-1-propane sulfonate (CHAPS), 3-[-(3-cholamidopropyl)dimethylamminio]-2-hydroxy-1-propane sulfonate (CHAPSO), or N-dodecyl-N,N-dimethyl-3-ammonio-1-propane sulfonate.

An interaction between a test molecule and target molecule also can be detected by monitoring fluorescence energy transfer (FET) (see, e.g., Lakowicz et al., U.S. Pat. No. 5,631,169; Stavrianopoulos et al. U.S. Pat. No. 4,868,103). A fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. An FET binding event can be conveniently measured through standard fluorometric detection means well known in the art (e.g., using a fluorimeter).

In another embodiment, determining the presence or absence of an interaction between a test molecule and a target molecule can be effected by monitoring surface plasmon resonance (see, e.g., Sjolander & Urbaniczk, Anal. Chem. 63: 2338-2345 (1991) and Szabo et al., Curr. Opin. Struct. Biol 5: 699-705 (1995)). “Surface plasmon resonance” or “biomolecular interaction analysis (BIA)” can be utilized to detect biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

In another embodiment the target molecule or test molecules are anchored to a solid phase, facilitating the detection of target molecule/test molecule complexes and separation of the complexes from free, uncomplexed molecules. The target molecule or test molecule is immobilized to the solid support. In an embodiment, the target molecule is anchored to a solid surface, and the test molecule, which is not anchored, can be labeled, either directly or indirectly, with detectable labels discussed herein. In certain embodiments, EPHA3, EPHA3-related test peptides, or a compound according to the invention is non-diffusably bound to an insoluble support having isolated sample-receiving areas (for example, a microtiter plate, an array, or the like.). The insoluble support may be made of any composition to which the compositions can be bound, is readily separated from soluble material, and is otherwise compatible with the overall method of screening. The surface of such supports may be solid or porous and of any convenient shape. Examples of suitable insoluble supports include microliter plates, arrays, membranes and beads. These are typically made of glass, plastic (for example, polystyrene), polysaccharides, nylon or nitrocellulose, Teflon™, and the like. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Exemplary methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein or other innocuous protein or other moiety.

One measure of inhibition is Kᵢ. For compounds with IC₅₀'s less than 1 μM, the Kᵢ or K_{d} is defined as the dissociation rate constant for the interaction of the agent with EPHA3. Exemplary compositions have Kᵢ's of, for example, less than about 100 μM, less than about 10 μM, less than about 1 μM, and further for example having Kᵢ's of less than about 100 nM, and still further, for example, less than about 10 nM. The Kᵢ for a compound is determined from the IC₅₀ based on three assumptions. First, only one compound molecule binds to the enzyme and there is no cooperativity. Second, the concentrations of active enzyme and the compound tested are known (i.e., there are no significant amounts of impurities or inactive forms in the preparations). Third, the enzymatic rate of the enzyme-inhibitor complex is zero. The rate (i.e., compound concentration) data are fitted to the equation:

$$V = {V}_{max} {E}_{0} [ I - \frac{( {E}_{0} + {I}_{0} + {K}_{d} ) - \sqrt{{{E}_{0} + {I}_{0} + {K}_{d} )}^{2} - 4 {E}_{0} {I}_{0}}}{2 {E}_{0}} ]$$

Where V is the observed rate, Vₘₐₓ, is the rate of the free enzyme, I₀ is the inhibitor concentration, E₀ is the enzyme concentration, and K_{d} is the dissociation constant of the enzyme-inhibitor complex.

Another measure of inhibition is GI₅₀, defined as the concentration of the compound that results in a decrease in the rate of cell growth by fifty percent. Exemplary compounds have GI₅₀'s of, for example, less than about 1 μM, less than about 10 μM, less than about 1 μM, and further, for example, having GI₅₀'s of less than about 100 nM, still further having GI₅₀'s of less than about 10 nM. Measurement of GI₅₀ is done using a cell proliferation assay.

It may be desirable to immobilize a target molecule, an anti-target molecule antibody, and/or test molecules to facilitate separation of target molecule/test molecule complexes from uncomplexed forms, as well as to accommodate automation of the assay. The attachment between a test molecule and/or target molecule and the solid support may be covalent or non-covalent (see, e.g., U.S. Pat. No. 6,022,688 for non-covalent attachments). The solid support may be one or more surfaces of the system, such as one or more surfaces in each well of a microtiter plate, a surface of a silicon wafer, a surface of a bead (see, e.g., Lam, Nature 354: 82-84 (1991)) that is optionally linked to another solid support, or a channel in a microfluidic device, for example. Types of solid supports, linker molecules for covalent and non-covalent attachments to solid supports, and methods for immobilizing nucleic acids and other molecules to solid supports are well known (see, e.g., U.S. Pat. Nos. 6,261,776; 5,900,481; 6,133,436; and 6,022,688; and WIPO publication WO 01/18234).

In an embodiment, target molecule may be immobilized to surfaces via biotin and streptavidin. For example, biotinylated target polypeptide can be prepared from biotin-NHS (N-hydroxy-succinimide) using techniques known in the art (e.g., biotinylation kit, Pierce Chemicals, Rockford, Ill.), and immobilized in the wells of streptavidin-coated 96 well plates (Pierce Chemical). In another embodiment, a target polypeptide can be prepared as a fusion polypeptide. For example, glutathione-S-transferase/target polypeptide fusion can be adsorbed onto glutathione sepharose beads (Sigma Chemical, St. Louis, Mo.) or glutathione derivatized microtiter plates, which are then combined with a test molecule under conditions conducive to complex formation (e.g., at physiological conditions for salt and pH). Following incubation, the beads or microtiter plate wells are washed to remove any unbound components, or the matrix is immobilized in the case of beads, and complex formation is determined directly or indirectly as described above. Alternatively, the complexes can be dissociated from the matrix, and the level of target molecule binding or activity is determined using standard techniques.

In an embodiment, the non-immobilized component is added to the coated surface containing the anchored component. After the reaction is complete, unreacted components are removed (e.g., by washing) under conditions such that a significant percentage of complexes formed will remain immobilized to the solid surface. The detection of complexes anchored on the solid surface can be accomplished in a number of manners. Where the previously non-immobilized component is pre-labeled, the detection of label immobilized on the surface indicates that complexes were formed. Where the previously non-immobilized component is not pre-labeled, an indirect label can be used to detect complexes anchored on the surface, e.g., by adding a labeled antibody specific for the immobilized component, where the antibody, in turn, can be directly labeled or indirectly labeled with, e.g., a labeled anti-Ig antibody.

In another embodiment an assay is performed utilizing antibodies that specifically bind target molecule or test molecule but do not interfere with binding of the target molecule to the test molecule. Such antibodies can be derivatized to a solid support, and unbound target molecule may be immobilized by antibody conjugation. Methods for detecting such complexes, in addition to those described above for the GST-immobilized complexes, include immunodetection of complexes using antibodies reactive with the target molecule, as well as enzyme-linked assays which rely on detecting an enzymatic activity associated with the target molecule.

Cell free assays also can be conducted in a liquid phase. In such an assay, reaction products are separated from unreacted components, by any of a number of standard techniques, including but not limited to: differential centrifugation (see, e.g., Rivas, G., and Minton, Trends Biochem Sci August; 18(8): 284-7 (1993)); chromatography (gel filtration chromatography, ion-exchange chromatography); electrophoresis (see, e.g., Ausubel et al., eds. Current Protocols in Molecular Biology, J Wiley: New York (1999)); and immunoprecipitation (see, e.g., Ausubel et al. eds., supra). Media and chromatographic techniques are known to one skilled in the art (see, e.g., Heegaard, J Mol. Recognit. Winter; 11(1-6): 141-8 (1998); Hage & Tweed, J. Chromatogr. B Biomed. Sci. Appl. October 10; 699 (1-2): 499-525 (1997)). Further, fluorescence energy transfer may also be conveniently utilized, as described herein, to detect binding without further purification of the complex from solution.

In another embodiment, modulators of target molecule expression are identified. For example, a cell or cell free mixture is contacted with a candidate compound and the expression of target mRNA or target polypeptide is evaluated relative to the level of expression of target mRNA or target polypeptide in the absence of the candidate compound. When expression of target mRNA or target polypeptide is greater in the presence of the candidate compound than in its absence, the candidate compound is identified as an agonist of target mRNA or target polypeptide expression. Alternatively, when expression of target mRNA or target polypeptide is less (e.g., less with statistical significance) in the presence of the candidate compound than in its absence, the candidate compound is identified as an antagonist or inhibitor of target mRNA or target polypeptide expression. The level of target mRNA or target polypeptide expression can be determined by methods described herein.

In another embodiment, binding partners that interact with a target molecule are detected. The target molecules can interact with one or more cellular or extracellular macromolecules, such as polypeptides in vivo, and these interacting molecules are referred to herein as “binding partners” Binding partners can agonize or antagonize target molecule biological activity. Also, test molecules that agonize or antagonize interactions between target molecules and binding partners can be useful as therapeutic molecules as they can up-regulate or down-regulated target molecule activity in Vivo and thereby treat type II diabetes.

Binding partners of target molecules can be identified by methods known in the art. For example, binding partners may be identified by lysing cells and analyzing cell lysates by electrophoretic techniques. Alternatively, a two-hybrid assay or three-hybrid assay can be utilized (see, e.g., U.S. Pat. No. 5,283,317; Zervos et al., Cell 72:223-232 (1993); Madura et al., J. Biol. Chem. 268: 12046-12054 (1993>; Bartel et al., Biotechniques 14: 920-924 (1993>; Iwabuchi et al., Oncogene 8: 1693-1696 (1993); and Brent WO94/10300). A two-hybrid system is based on the modular nature of most transcription factors, which consist of separable DNA-binding and activation domains. The assay often utilizes two different DNA constructs. In one construct, an EPHA3 nucleic acid (sometimes referred to as the “bait”) is fused to a gene encoding the DNA binding domain of a known transcription factor (e.g., GAL-4). In another construct, a DNA sequence from a library of DNA sequences that encodes a potential binding partner (sometimes referred to as the “prey”) is fused to a gene that encodes an activation domain of the known transcription factor. Sometimes, an EPHA3 nucleic acid can be fused to the activation domain. If the ‘bait’ and the “prey” molecules interact in vivo, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to identify the potential binding partner.

In an embodiment for identifying test molecules that antagonize or agonize complex formation between target molecules and binding partners, a reaction mixture containing the target molecule and the binding partner is prepared, under conditions and for a time sufficient to allow complex formation. The reaction mixture often is provided in the presence or absence of the test molecule. The test molecule can be included initially in the reaction mixture, or can be added at a time subsequent to the addition of the target molecule and its binding partner. Control reaction mixtures are incubated without the test molecule or with a placebo. Formation of any complexes between the target molecule and the binding partner then is detected. Decreased formation of a complex in the reaction mixture containing test molecule as compared to in a control reaction mixture indicates that the molecule antagonizes target molecule/binding partner complex formation. Alternatively, increased formation of a complex in the reaction mixture containing test molecule as compared to in a control reaction mixture indicates that the molecule agonizes target molecule/binding partner complex formation. In another embodiment, complex formation of target molecule/binding partner can be compared to complex formation of mutant target molecule/binding partner (e.g., amino acid modifications in a target polypeptide). Such a comparison can be important in those cases where it is desirable to identify test molecules that modulate interactions of mutant but not non-mutated target gene products.

The assays can be conducted in a heterogeneous or homogeneous, format. In heterogeneous assays, target molecule and/or the binding partner are immobilized to a solid phase, and complexes are detected on the solid phase at the end of the reaction. In homogeneous assays, the entire reaction is carried out in a liquid phase. In either approach, the order of addition of reactants can be varied to obtain different information about the molecules being tested. For example, test compounds that agonize target molecule/binding partner interactions can be identified by conducting the reaction in the presence of the test molecule in a competition format. Alternatively, test molecules that agonize preformed complexes, e.g. molecules with higher binding constants that displace one of the components from the complex, can be tested by adding the test compound to the reaction mixture after complexes have been formed.

In a heterogeneous assay embodiment, the target molecule or the binding partner is anchored onto a solid surface (e.g., a microtiter plate), while the non-anchored species is labeled, either directly or indirectly. The anchored molecule can be immobilized by non-covalent or covalent attachments. Alternatively, an immobilized antibody specific for the molecule to be anchored can be used to anchor the molecule to the solid surface. The partner of the immobilized species is exposed to the coated surface with or without the test molecule. After the reaction is complete, unreacted components are removed (e.g., by washing) such that a significant portion of any complexes formed will remain immobilized on the solid surface. Where the non-immobilized species is pre-labeled, the detection of label immobilized on the surface is indicative of complex. Where the non-immobilized species is not pre-labeled, an indirect label can be used to detect complexes anchored to the surface; e.g., by using a labeled antibody specific for the initially non-immobilized species. Depending upon the order of addition of reaction components, test compounds that inhibit complex formation or that disrupt preformed complexes can be detected.

In another embodiment, the reaction can be conducted in a liquid phase in the presence or absence of test molecule, where the reaction products are separated from unreacted components, and the complexes are detected (e.g., using an immobilized antibody specific for one of the binding components to anchor any complexes formed in solution, and a labeled antibody specific for the other partner to detect anchored complexes). Again, depending upon the order of addition of reactants to the liquid phase, test compounds that inhibit complex or that disrupt preformed complexes can be identified.

In an alternate embodiment, a homogeneous assay can be utilized. For example, a preformed complex of the target gene product and the interactive cellular or extracellular binding partner product is prepared. One or both of the target molecule or binding partner is labeled, and the signal generated by the label(s) is quenched upon complex formation (e.g., U.S. Pat. No. 4,109,496 that utilizes this approach for immunoassays). Addition of a test molecule that competes with and displaces one of the species from the preformed complex will result in the generation of a signal above background. In this way, test substances that disrupt target molecule/binding partner complexes can be identified.

Candidate therapeutics for treating type II diabetes are identified from a group of test molecules that interact with a target molecule. Test molecules are normally ranked according to the degree with which they modulate (e.g., agonize or antagonize) a function associated with the target molecule (e.g., DNA replication and/or processing, RNA transcription and/or processing, polypeptide production and/or processing, and/or biological function/activity, and then top ranking modulators are selected. Also, pharmacogenomic information described herein can determine the rank of a modulator. The top 10% of ranked test molecules often are selected for further testing as candidate therapeutics, and sometimes the top 15%, 20%, or 25% of ranked test molecules are selected for further testing as candidate therapeutics. Candidate therapeutics typically are formulated for administration to a subject.

Therapeutic Formulations

Formulations and pharmaceutical compositions typically include in combination with a pharmaceutically acceptable carrier one or more target molecule modulators. The modulator often is a test molecule identified as having an interaction with a target molecule by a screening method described above. The modulator may be a compound, an antisense nucleic acid, a ribozyme, an antibody, or a binding partner. Also, formulations may comprise a target polypeptide or fragment thereof in combination with a pharmaceutically acceptable carrier.

As used herein, the term “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. Pharmaceutical compositions can be included in a container, pack, or dispenser together with instructions for administration.

A pharmaceutical composition typically is formulated to be compatible with its intended route of administration. Examples of routes of administration include parenteral, e.g. intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphate and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

For administration by inhalation, the compounds are delivered in the form of an aerosol spray from pressured container or dispenser which contains a suitable propellants e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Molecules can also be prepared in the form of suppositories (e.g., with conventional suppository bases such as cocoa butter and other glycerides) or retention enemas for rectal delivery.

In one embodiment, active molecules are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Molecules which exhibit high therapeutic indices are preferred. While molecules that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such molecules lies preferably within a range of circulating concentrations that include the ED₅₀ with little or no toxicity. The dosage may vary within this range depending upon the dosage form employed and the route of administration utilized. For any molecules used in the methods described herein, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of protein or polypeptide (i.e., an effective dosage) ranges from about 0.001 to 30 mg/kg body weight, sometimes about 0.01 to 25 mg/kg body weight, often about 0.1 to 20 mg/kg body weight, and more often about 1 to 10 mg/kg, 2 to 9 mg/kg, 3 to 8 mg/kg, 4 to 7 mg/kg, or 5 to 6 mg/kg body weight. The protein or polypeptide can be administered one time per week for between about 1 to 10 weeks, sometimes between 2 to 8 weeks, often between about 3 to 7 weeks, and more often for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

With regard to polypeptide formulations, featured herein is a method for treating type II diabetes in a subject, which comprises contacting one or more cells in the subject with a first polypeptide, where the subject comprises a second polypeptide having one or more polymorphic variations associated with cancer, and where the first polypeptide comprises fewer polymorphic variations associated with cancer than the second polypeptide. The first and second polypeptides are encoded by a nucleic acid which comprises a nucleotide sequence in SEQ ID NO: 1-3; a nucleotide sequence which encodes a polypeptide consisting of an amino acid sequence encoded by a nucleotide sequence referenced in SEQ ID NO: 1-3; a nucleotide sequence which encodes a polypeptide that is 90% or more identical to an amino acid sequence encoded by a nucleotide sequence of SEQ ID NO: 1-3 and a nucleotide sequence 90% or more identical to a nucleotide sequence in SEQ ID NO: 1-3. The subject often is a human.

For antibodies, a dosage of 0.1 mg/kg of body weight (generally 10 mg/kg to 20 mg/kg is often utilized. If the antibody is to act in the brain, a dosage of 5 mg/kg to 100 mg/kg is often appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology 14:193 (1997).

Antibody conjugates can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a polypeptide such as tumor necrosis factor, .alpha.-interferon, .beta.-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors. Alternatively, an antibody can be conjugated to a second antibody to form an antibody heteroconjugate as described by Segal in U.S. Pat. No. 4,676,980.

For compounds, exemplary doses include milligram or microgram amounts of the compound per kilogram of subject or sample weight, for example, about 1 microgram per kilogram to about 500 milligrams per kilogram, about 160 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

With regard to nucleic acid formulations, gene therapy vectors can be delivered to a subject by, for example, intravenous injection, local administration (see, e.g., U.S. Pat. No. 5,328,470) or by stereotactic injection (see e.g., Chen et al, (1994) Proc. Natl. Acad. Sci. USA 91:3054-3057). Pharmaceutical preparations of gene therapy vectors can include a gene therapy vector in an acceptable diluent, or can comprise a slow release matrix in which the gene delivery vehicle is imbedded. Alternatively, where the complete gene delivery vector can be produced intact from recombinant cells (e.g., retroviral vectors) the pharmaceutical preparation can include one or more cells which produce the gene delivery system. Examples of gene delivery vectors are described herein.

Therapeutic Methods

A therapeutic formulation described above can be administered to a subject in need of a therapeutic for inducing a desired biological response. Therapeutic formulations can be administered by any of the paths described herein. With regard to both prophylactic and therapeutic methods of treatment such treatments may be specifically tailored or modified, based on knowledge obtained from pharmacogenomic analyses described herein.

As used herein, the term “treatment” is defined as the application or administration of a therapeutic formulation to a subject, or application or administration of a therapeutic agent to an isolated tissue or cell line from a subject with the purpose to cure, heal, alleviate, relieve, alter, remedy, ameliorate, improve or affect type II diabetes, symptoms of type II diabetes or a predisposition towards type II diabetes. A therapeutic formulation includes, but is not limited to, small molecules, peptides, antibodies, ribozymes and antisense oligonucleotides. In certain embodiments, a peptide therapeutic formulation comprises isoform b of EPHA3 (SEQ ID NO:5) or the extracellular domain of isoform a (21-541 of SEQ ID NO:4) that specifically binds to an EPHA3 binding partner ligand (e.g., Ephrin-A2 or Ephrin-A5) and inhibits binding between EPHA3 and that binding partner or ligand. Thus, provided herein is a method which comprises administering a peptide therapeutic formulation comprising isoform b of EPHA3 (SEQ ID NO:5) or the extracellular domain of isoform a (21-541 of SEQ ID NO:4) for the improvement of glucose control in type II diabetes patients. Administration of a therapeutic formulation can occur prior to the manifestation of symptoms characteristic of type II diabetes, such that Type II diabetes is prevented or delayed in its progression. The appropriate therapeutic composition can be determined based on screening assays described herein.

In related aspects, embodiments include methods of causing or inducing a desired biological response in an individual comprising the steps of: providing or administering to an individual a composition comprising a polypeptide described herein, or a fragment thereof, or a therapeutic formulation described herein, wherein said biological response is selected from the group consisting of: (a) modulating circulating (either blood, serum or plasma) levels (concentration) of glucose, wherein said modulating is preferably lowering; (b) increasing cell or tissue sensitivity to insulin, particularly muscle, adipose, liver or brain; (c) inhibiting the progression from impaired glucose tolerance to insulin resistance; (d) increasing glucose uptake in skeletal muscle cells; (e) increasing glucose uptake in adipose cells; (f) increasing glucose uptake in neuronal cells; (g) increasing glucose uptake in red blood cells; (h) increasing glucose uptake in the brain; and (i) significantly reducing the postprandial increase in plasma glucose following a meal, particularly a high carbohydrate meal.

In other embodiments, a pharmaceutical or physiologically acceptable composition can be utilized as an insulin sensitizer, or can be used in: a method to improve insulin sensitivity in some persons with type II diabetes in combination with insulin therapy; a method to improve insulin sensitivity in some persons with type II diabetes without insulin therapy; or a method of treating individuals with gestational diabetes. Gestational diabetes refers to the development of diabetes in an individual during pregnancy, usually during the second or third trimester of pregnancy. In further embodiments, the pharmaceutical or physiologically acceptable composition can be used in a method of treating individuals with impaired fasting glucose (IFG). Impaired fasting glucose (IFG) is a condition in which fasting plasma glucose levels in an individual are elevated but not diagnostic of overt diabetes (i.e. plasma glucose levels of less than 126 mg/dl and greater than or equal to 10 mg/dl).

In other embodiments, the pharmaceutical or physiologically acceptable composition can be used in a method of treating and preventing impaired glucose tolerance (IGT) in an individual. By providing therapeutics and methods for reducing or preventing IGT (i.e., for normalizing insulin resistance) the progression to type II diabetes can be delayed or prevented. Furthermore, by providing therapeutics and methods for reducing or preventing insulin resistance, provided are methods for reducing and/or preventing the appearance of Insulin-Resistance Syndrome (IRS). In further embodiments, the pharmaceutical or physiologically acceptable composition can be used in a method of treating a subject having polycystic ovary syndrome (PCOS). PCOS is among the most common disorders of premenopausal women, affecting 5-10% of this population. Insulin-sensitizing agents (e.g., troglitazone) have been shown to be effective in PCOS and that, in particular, the defects in insulin action, insulin secretion, ovarian steroidogenosis and fibrinolysis are improved (Ehrman et al. (1997) J Clin Invest 100:1230, such as in insulin-resistant humans Accordingly, provided are methods for reducing insulin resistance, normalizing blood glucose thus treating and/or preventing PCOS.

In certain embodiments, the pharmaceutical or physiologically acceptable composition can be used in a method of treating a subject having insulin resistance, where a subject having insulin resistance is treated to reduce or cure the insulin resistance. As insulin resistance is also often associated with infections and cancer, preventing or reducing insulin resistance may prevent or reduce infections and cancer.

In other embodiments, the pharmaceutical compositions and methods described herein are useful for: preventing the development of insulin resistance in a subject, e.g., those known to have an increased risk of developing insulin resistance; controlling blood glucose in some persons with type II diabetes in combination with insulin therapy; increasing cell or tissue sensitivity to insulin, particularly muscle, adipose, liver or brain; inhibiting or preventing the progression from impaired glucose tolerance to insulin resistance; improving glucose control of type II diabetes patients alone, without an insulin secretagogue or an insulin sensitizing agent; and administering a complementary therapy to type II diabetes patients to improve their glucose control in combination with an insulin secretagogue (preferably oral form) or an insulin sensitizing (preferably oral form) agent. In the latter embodiment, the oral insulin secretagogue sometimes is 1,1-dimethyl-2-(2-morpholino phenyl)guanidine fumarate (BTS67582) or a sulphonylurea selected from tolbutamide, tolazamide, chlorpropamide, glibenclamide, glimepiride, glipizide and glidazide. The insulin sensitizing agent sometimes is selected from metformin, ciglitazone, troglitazone and pioglitazone.

Further embodiments include methods of administering a pharmaceutical or physiologically acceptable composition concomitantly or concurrently, with an insulin secretagogue or insulin sensitizing agent, for example, in the form of separate dosage units to be used simultaneously, separately or sequentially (e.g., before or after the secretagogue or before or after the sensitizing agent). Accordingly, provided is a pharmaceutical or physiologically acceptable composition and an insulin secretagogue or insulin sensitizing agent as a combined preparation for simultaneous, separate or sequential use for the improvement of glucose control in type II diabetes patients.

Thus, any test known in the art or a method described herein can be used to determine that a subject is insulin resistant, and an insulin resistant patient can then be treated according to the methods described herein to reduce or cure the insulin resistance. Alternatively, the methods described herein also can be used to prevent the development of insulin resistance in a subject, e.g., those known to have an increased risk of developing insulin-resistance.

In certain embodiments the therapeutic molecule administered to a subject to treat type II diabetes specifically interacts with (e.g., binds to) an EPHA3 polymorphic variant, such as a polypeptide comprising an arginine at position 924 in SEQ ID NO: 4, or sometimes a tryptophan at position 924. In other embodiments, the therapeutic molecule specifically interacts with a binding partner, ligand or signal partner of EPHA3, such as Ephrin-A2 and/or Ephrin-A5. In other embodiments, the therapeutic molecule specifically interacts with a EPHA3-related enzyme such as ADAM10. In other embodiments, the therapeutic molecule also modulates other tyrosine kinases, such as EGF (NM_{—}001963), Src (NM_{—}005417), VEGF (NM₀₃₃₇₆) or KDR (NM_{—}002253). In yet another embodiment, the therapeutic molecule also modulates proteins that shares homology with EPHA3, such as EphA2 (NM_{—}004431) or EphB4 (N_{—}004444). The therapeutic molecule sometimes modulates certain cell functions and/or activities or levels of certain cellular molecules, such as glucose uptake by cells; glucose transport molecule activity or levels in cells (e.g., GLUT4 levels or activities in cells); triacylglycerol content in cells; resistin levels or activities in cells; levels or activities of molecules involved in resistin levels in cells such as PPAR gamma, PI3 kinase, Akt and C/EBP alpha; and levels or activities of EPHA3 binding partners or ligands such as Ephrin-A2 and Ephrin-A5. In certain embodiments, the type II diabetes therapeutic molecule modulates interactions between the following cellular molecules: EPHA3 and its natural ligand ephrin-A5 and/or EPHA3 and its natural ligand ephrin-A2 and/or two or more EPHA3 moieties and/or domains of EPHA3 and/or within one or more domain(s) of an EPHA3 moiety and/or EPHA3 and downstream moieties with which EPHA3 interacts. The therapeutic molecule sometimes modulates one or more of the following: (a) circulating (e.g., blood, serum or plasma) levels (e.g., concentration) of glucose, where the therapeutic molecule often lowers glucose levels; (b) cell or tissue sensitivity to insulin, particularly in muscle, adipose, liver or brain, where the therapeutic molecule often increases sensitivity; (c) progression from impaired glucose tolerance to insulin resistance, where the therapeutic molecule often inhibits the progression; (d) glucose uptake in skeletal muscle cells, where the therapeutic molecule often increases glucose uptake; (e) glucose uptake in adipose cells, where the therapeutic molecule often increases uptake; (f) glucose uptake in neuronal cells, where the therapeutic molecule often increases uptake; (g) glucose uptake in red blood cells, where the therapeutic molecule often increases uptake; (h) glucose uptake in the brain, where the therapeutic molecule often increases uptakes; and (i) postprandial increase in plasma glucose following a meal, particularly a high carbohydrate meal, where the therapeutic molecule often reduces significantly the postprandial increase.

In specific embodiments, the test molecule is an antibody or protein that specifically binds to EPHA3 or an EPHA3 binding partner, ligand or signal pathway member. Such antibodies and proteins are disclosed in U.S. Pat. Nos. 6,169,167; 6,063,903; 6,057,124; 5,798,448; and Ahsan M, et al. Biochem Biophys Res. Commun. 2002 Jul. 12; 295(2):348-53. A soluble form of EPHA3 which binds to ephrin-A5 and/or ephrin-A, preventing or diminishing the binding of ephrin-A5 to membrane bound EPHA3, may be used. Inter-EPHA3 interactions may also be inhibited by use of the foregoing moieties.

As discussed, successful treatment of type II diabetes can be brought about by techniques that serve to agonize target molecule expression or function, or alternatively, antagonize target molecule expression or function. These techniques include administration of modulators that include, but are not limited to, small organic or inorganic molecules; antibodies (including, for example, polyclonal, monoclonal, humanized, anti-idiotypic, chimeric or single chain antibodies, and Fab, F(ab′)₂ and Fab expression library fragments, scFV molecules, and epitope-binding fragments thereof); and peptides, phosphopeptides, or polypeptides.

Further, antisense and ribozyme molecules that inhibit expression of the target gene can also be used to reduce the level of target gene expression, thus effectively reducing the level of target gene activity. Still further, triple helix molecules can be utilized in reducing the level of target gene activity. Antisense, ribozyme and triple helix molecules are discussed above. It is possible that the use of antisense, ribozyme, and/or triple helix molecules to reduce or inhibit mutant gene expression can also reduce or inhibit the transcription (triple helix) and/or translation (antisense, ribozyme) of mRNA produced by normal target gene alleles, such that the concentration of normal target gene product present can be lower than is necessary for a normal phenotype. In such cases, nucleic acid molecules that encode and express target gene polypeptides exhibiting normal target gene activity can be introduced into cells via gene therapy method. Alternatively, in instances in that the target gene encodes an extracellular polypeptide, it can be preferable to co-administer normal target gene polypeptide into the cell or tissue in order to maintain the requisite level of cellular or tissue target gene activity.

Another method by which nucleic acid molecules may be utilized in treating or preventing type II diabetes is use of aptamer molecules specific for target molecules. Aptamers are nucleic acid molecules having a tertiary structure which permits them to specifically bind to ligands (see, e.g., Osborne, et al, Curr. Opin. Chem. Biol. 1(1): 5-9 (1997); and Patel, D. J., Curr. Opin. Chem. Biol June; 1 (1): 3246 (1997)).

Yet another method of utilizing nucleic acid molecules for type II diabetes treatment is gene therapy, which can also be referred to as allele therapy. Provided herein is a gene therapy method for treating type II diabetes in a subject, which comprises contacting one or more cells in the subject or from the subject with a nucleic acid having a first nucleotide sequence. Genomic DNA in the subject comprises a second nucleotide sequence having one or more polymorphic variations associated with type II diabetes (e.g., the second nucleic acid has a nucleotide sequence in SEQ ID NO: 1-3). The first and second nucleotide sequences typically are substantially identical to one another, and the first nucleotide sequence comprises fewer polymorphic variations associated with type II diabetes than the second nucleotide sequence. The first nucleotide sequence may comprise a gene sequence that encodes a full-length polypeptide or a fragment thereof. The subject is often a human. Allele therapy methods often are utilized in conjunction with a method of first determining whether a subject has genomic DNA that includes polymorphic variants associated with type II diabetes.

In another allele therapy embodiment, provided herein is a method which comprises contacting one or more cells in the subject or from the subject with a polypeptide encoded by a nucleic acid having a first nucleotide sequence. Genomic DNA in the subject comprises a second nucleotide sequence having one or more polymorphic variations associated with type II diabetes (e.g., the second nucleic acid has a nucleotide sequence in SEQ ID NO: 1-3). The first and second nucleotide sequences typically are substantially identical to one another, and the first nucleotide sequence comprises fewer polymorphic variations associated with type II diabetes than the second nucleotide sequence. The first nucleotide sequence may comprise a gene sequence that encodes a full-length polypeptide or a fragment thereof. The subject is often a human.

For antibody-based therapies, antibodies can be generated that are both specific for target molecules and that reduce target molecule activity. Such antibodies may be administered in instances where antagonizing a target molecule function is appropriate for the treatment of type II diabetes.

In circumstances where stimulating antibody production in an animal or a human subject by injection with a target molecule is harmful to the subject, it is possible to generate an immune response against the target molecule by use of anti-idiotypic antibodies (see, e.g., Herlyn, Ann. Med.; 31(1): 66-78 (1999); and Bhattacharya-Chatterjee & Foon, Cancer Treat. Res.; 94: 51-68 (1998)). Introducing an anti-idiotypic antibody to a mammal or human subject often stimulates production of anti-anti-idiotypic antibodies, which typically are specific to the target molecule. Vaccines directed to type II diabetes also may be generated in this fashion.

In instances where the target molecule is intracellular and whole antibodies are used, internalizing antibodies may be preferred. Lipofectin or liposomes can be used to deliver the antibody or a fragment of the Fab region that binds to the target antigen into cells. Where fragments of the antibody are used, the smallest inhibitory fragment that binds to the target antigen is preferred. For example, peptides having an amino acid sequence corresponding to the Fv region of the antibody can be used. Alternatively, single chain neutralizing antibodies that bind to intracellular target antigens can also be administered. Such single chain antibodies can be administered, for example, by expressing nucleotide sequences encoding single-chain antibodies within the target cell population (see, e.g., Marasco et al., Proc. Natl. Acad. Sci. USA 90: 7889-7893 (1993)).

Modulators can be administered to a patient at therapeutically effective doses to treat type II diabetes. A therapeutically effective dose refers to an amount of the modulator sufficient to result in amelioration of symptoms of type II diabetes. Toxicity and therapeutic efficacy of modulators can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Modulators that exhibit large therapeutic indices are preferred. While modulators that exhibit toxic side effects can be used, care should be taken to design a delivery system that targets such molecules to the site of affected tissue in order to minimize potential damage to uninfected cells, thereby reducing side effects.

Data obtained from cell culture assays and animal studies can be used in formulating a range of dosages for use in humans. The dosage of such compounds lies preferably within a range of circulating concentrations that include the ED₅₀ with little or no toxicity. The dosage can vary within this range depending upon the dosage form employed and the route of administration utilized. For any compound used in the methods described herein, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans, Levels in plasma can be measured, for example, by high performance liquid chromatography.

Another example of effective dose determination for an individual is the ability to directly assay levels of “free” and “bound” compound in the serum of the test subject. Such assays may utilize antibody mimics and/or “biosensors” that have been created through molecular imprinting techniques. Molecules that modulate target molecule activity are used as a template, or “imprinting molecule”, to spatially organize polymerizable monomers prior to their polymerization with catalytic reagents. The subsequent removal of the imprinted molecule leaves a polymer matrix which contains a repeated “negative image” of the compound and is able to selectively rebind the molecule under biological assay conditions. A detailed review of this technique can be seen in Ansell et al., Current Opinion in Biotechnology 7: 89-94 (1996) and in Shea, Trends in Polymer Science 2: 166-173 (1994). Such “imprinted” affinity matrixes are amenable to ligand-binding, assays, whereby the immobilized monoclonal antibody component is replaced by an appropriately imprinted matrix. An example of the use of such matrixes in this way can be seen in Vlatakis, et al., Nature 361: 645-647 (1993). Through the use of isotope-labeling, the “free” concentration of compound which modulates target molecule expression or activity readily can be monitored and used in calculations of IC₅₀. Such “imprinted” affinity matrixes can also be designed to include fluorescent groups whose photon-emitting properties measurably change upon local and selective binding of target compound. These changes readily can be assayed in real time using appropriate fiberoptic devices, in turn allowing the dose in a test subject to be quickly optimized based on its individual IC₅₆. An example of such a “biosensor” is discussed in Kriz et al., Analytical Chemistry 67: 2142-2144 (1995).

The examples set forth below are intended to illustrate but not limit the invention.

### EXAMPLES

In the following studies a group of subjects was selected according to specific parameters pertaining to type II diabetes. Nucleic acid samples obtained from individuals in the study group were subjected to genetic analyses that identified associations between type II diabetes and certain polymorphic variants in human genomic DNA. This procedure was repeated in a second group and third group of subjects that served as replication cohorts. See Examples 3-4. Polymorphic variants proximal to the incident SNP were identified and analyzed in cases and controls. See Example 5. Methods are described for producing EPHA3 polypeptides encoded by the nucleic acids of SEQ ID NO: 1-3 in vitro or in vivo, which can be utilized in methods that screen test molecules for those that interact with EPHA3 polypeptides. Test molecules identified as being interactors with EPHA3 polypeptides can be screened further as type II diabetes therapeutics.

### Example 1

### Samples and Pooling Strategies

Sample Selection

Blood samples were collected from individuals diagnosed with type U diabetes, which were referred to as case samples. Also, blood samples were collected from individuals not diagnosed with type II diabetes or a history of type II diabetes; these samples served as gender and age-matched controls. A database was created that listed all phenotypic trait information gathered from individuals for each case and control sample. Genomic DNA was extracted from each of the blood samples for genetic analyses.

DNA Extraction from Blood Samples

Six to ten milliliters of whole blood was transferred to a 50 ml tube containing 27 ml of red cell lysis solution (RCL). The tube was inverted until the contents were mixed. Each tube was incubated for 10 minutes at room temperature and inverted once during the incubation. The tubes were then centrifuged for 20 minutes at 3000×g and the supernatant was carefully poured off. 100-200 μl of residual liquid was left in the tube and was pipetted repeatedly to resuspend the pellet in the residual supernatant. White cell lysis solution (WCL) was added to the tube and pipetted repeatedly until completely mixed. While no incubation was normally required, the solution was incubated at 37° C. or room temperature if cell clumps were visible after mixing until the solution was homogeneous. 2 ml of protein precipitation was added to the cell lysate. The mixtures were vortexed vigorously at high speed for 20 see to mix the protein precipitation solution uniformly with the cell lysate, and then centrifuged for 10 minutes at 3000×g. The supernatant containing the DNA was then poured into a clean 15 ml tube, which contained 7 ml of 100% isopropanol. The samples were mixed by inverting the tubes gently until white threads of DNA were visible. Samples were centrifuged for 3 minutes, at 2000×g and the DNA was visible as a small white pellet. The supernatant was decanted and 5 ml of 70% ethanol was added to each tube. Each tube was inverted several times to wash the DNA pellet, and then centrifuged for 1 minute at 2000×g. The ethanol was decanted and each tube was drained on clean absorbent paper. The DNA was dried in the tube by inversion for 10 minutes, and then 1000 μl of 1×TE was added. The size of each sample was estimated, and less TE buffer was added during the following DNA hydration step if the sample was smaller. The DNA was allowed to rehydrate overnight at room temperature, and DNA samples were stored at 2-8° C.

DNA was quantified by placing samples on a hematology mixer for at least 1 hour. DNA was serially diluted (typically 1:80, 1:160, 1:320, and 1:640 dilutions) so that it would be within the measurable range of standards. 125 μl of diluted DNA was transferred to a clear U-bottom microtitre plate, and 125 μl of 1×TE buffer was transferred into each well using a multichannel pipette. The DNA and 1×TE were mixed by repeated pipetting at least 15 times, and then the plates were sealed. 50 μl of diluted DNA was added to wells A5-H12 of a black flat bottom microtitre plate. Standards were inverted six times to mix them, and then 50 μl of 1×TE buffer was pipetted into well A1, 1000 ng/ml of standard was pipetted into well A2,500 ng/ml of standard was pipetted into well A3, and 250 ng/ml of standard was pipetted into well A4. PicoGreen (Molecular Probes, Eugene, Oreg.) was thawed and freshly diluted 1:200 according to the number of plates that were being measured. PicoGreen was vortexed and then 50411 was pipetted into all wells of the black plate with the diluted DNA. DNA and PicoGreen were mixed by pipetting repeatedly at least 10 times with the multichannel pipette. The plate was placed into a Fluoroskan Ascent Machine (microplate fluorometer produced by Labsystems) and the samples were allowed to incubate for 3 minutes before the machine was run using filter pairs 485 nm excitation and 538 nm emission wavelengths. Samples having measured DNA concentrations of greater than 450 ng/4 μl were re-measured for conformation. Samples having measured DNA concentrations of 20 ng/μl or less were re-measured for confirmation.

Pooling Strategies

Samples were placed into one of four groups based on disease status. The four groups were female case samples, female control samples, male case samples and male control samples. A select set of samples from each group were utilized to generate pools, and one pool was created for each group. Each individual sample in a pool was represented by an equal amount of genomic DNA. For example, where 25 ng of genomic DNA was utilized in each PCR reaction and there were 200 individuals in each pool, each individual would provide 125 pg of genomic DNA. Inclusion or exclusion of samples for a pool was based upon the following criteria and detailed in the tables below: patient ethnicity, diagnosis with type II diabetes, GAD antibody concentration, HbA1c concentration, body, mass (BMI), patient age, date of primary diagnosis, and age of individual as of primary diagnosis. (See Table 2 below). Cases with elevated GAD antibody titers and low age of diagnosis were excluded to increase the homogeneity of the diabetes sample in terms of underlying pathogenesis. Controls with elevated HbA1c were excluded to remove any potentially undiagnosed diabetics. Control samples were derived from non-diabetic individuals with no family history of type II diabetes. Secondary phenotypes were also measured in the diabetic cases, including HDL levels, LDL levels, triglyceride levels, insulin levels, C-peptide levels, nephropathy status, and neuropathy status, to name a few. The phenotype data collected may be used to perform secondary analysis of the cases in order to elucidate the potential pathway of a disease gene.

TABLE 2

| | No. of | | |
|---|---|---|---|
| | individuals | Actual no. of | |
| | fulfilling | samples | No. of |
| | exclusion | excluded after | samples |
| Exclusion Criteria | criteria | each stage | remaining |
| ALL SAMPLES Lack of | 34 | 34 | 1591 |
| availability of sample | | | |
| ALL SAMPLES Non-German | 261 | 239 | 1352 |
| ethnicity | | | |
| CASES GAD Ab > 0.9 | 102 | 84 | 1268 |
| CONTROLS HbA1c ≧ 6 or | 21 | 20 | 1248 |
| BMI > 40 | | | |
| CASES age < 90 | 17 | 6 | 1242 |
| CASES Age of | 150 | 203 | 1039 |
| Diagnosis < 35, | | | |
| CONTROLS Family History | 170 | | |
| of Diabetes | | | |
| CONTROLS Age-matching | 43 | 43 | 996 |
| to case pool | | | |

The selection process yielded the pools described in Table 3, which were used in the studies described herein.

TABLE 3

| | Female | Female | Male | Male |
|---|---|---|---|---|
| | case | control | cases | control |
| Pool size | 244 | 244 | 254 | 254 |
| (Number) | | | | |
| Pool Criteria | case | control | case | control |
| (ex: case/control) | | | | |
| Mean Age | 52.49 | 49.02 | 49.78 | 50.57 |
| (ex: years) | | | | |

### Example 2

### Association of Polymorphic Variants with Type II Diabetes

A whole-genome screen was performed to identify particular SNPs associated with occurrence of type II diabetes. As described in Example 1, two sets of samples were utilized female individuals having type II diabetes (female cases) and samples from female individuals not having type II diabetes or any history of type II diabetes (female controls), and male individuals having type B: diabetes (male cases) and samples from male individuals not having type I diabetes or any history of type II diabetes (male controls). The initial screen of each pool was performed in an allelotyping study, in which certain samples in each group were pooled. By pooling DNA from each group, an allele frequency for each SNP in each group was calculated. These allele frequencies were then compared to one another. Particular SNPs were considered as being associated with type II diabetes when allele frequency differences calculated between case and control pools were statistically significant. SNP disease association results obtained from the allelotyping study were then validated by genotyping each associated SNP across all samples from each pool. The results of the genotyping were then analyzed, allele frequencies for each group were calculated from the individual genotyping results, and a p-value was calculated to determine whether the case and control groups had statistically significantly differences in allele frequencies for a particular SNP. When the genotyping results agreed with the original allelotyping results, the SNP disease association was considered validated at the genetic level.

SNP Panel Used for Genetic Analyses

A whole-genome SNP screen began with an initial screen of approximately 25,000 SNPs over each set of disease and control samples using a pooling, approach. The pools studied in the screen are described in Example 1. The SNPs analyzed in this study were part of a set of 25,488 SNPs confined as being statistically polymorphic as each is characterized as having a minor allele frequency of greater than 10%. The SNPs in the set reside in genes or in close proximity to genes, and many reside in gene exons. Specifically, SNPs in the set are located in exons, introns, and within 5,000 base-pairs upstream of a transcription start site of a gene. In addition, SNPs were selected according, to the following criteria: they are located in ESTs; they are located in Locuslink or Ensemble genes; and they are located in Genomatix promoter predictions. An additional 3088 SNPs were included with these 25,488 SNPs and these additional SNPs had been chosen on the basis of gene location, with preference to non-synonymous coding SNPs located in disease candidate genes. SNPs in the set were also selected on the basis of even spacing across the genome, as depicted in Table 4.

TABLE 4

| General Statistics | | Spacing Statistics | |
|---|---|---|---|
| Total # of SNPs | 25,488 | Median | 37,058 bp |
| # of Exonic SNPs | >4,335 (17%) | Minimum* | 1,000 bp |
| # SNPs with refSNP | 20,776 (81%) | Maximum* | 3,000,000 bp |
| ID | | | |
| Gene Coverage | >10,000 | Mean | 122,412 bp |
| Chromosome | All | Std Deviation | 373,325 bp |
| Coverage | | | |

*Excludes outliers

### Allelotyping and Genotyping Results

The genetic studies summarized above and described in more detail below identified allelic variants associated with type II diabetes. The allelic variants identified from the SNP panel described in Table 4 are summarized below in Table 5.

TABLE 5

| | | | Position | | | | | | |
|---|---|---|---|---|---|---|---|---|---|
| SNP | | Chromosome | in SEQ ID | Contig | Contig | Sequence | | Sequence | Allelic |
| Reference | Chromosome | Position | NO: 1 | Identification | Position | Identification | Locus | Position | Variability |
| rs1512183 | 3 | 89425955 | 50155 | NT_022459 | 23199742 | NM_005233 | EphA3 | intron | T/A |

Table 5 includes information pertaining to the incident polymorphic variant associated with type II diabetes identified herein. Public information pertaining to the polymorphism and the genomic sequence that includes the polymorphism are indicated. The genomic sequences identified in Table 5 may be accessed at the http address www.ncbi.nih.gov/entrez/query.fcgi, for example, by using the publicly available SNP reference number (e.g., rs1512183). The chromosome position refers to the position of the SNP within NCBI's Genome Build 34, which may be accessed at the following http address: www.ncbi.nlm.nih.gov/mapview/mapsearch.cgi?chr=hum_chr.inf&query=. The “Contig Position” provided in Table 5 corresponds to a nucleotide position set forth in the contig sequence; and designates the polymorphic site corresponding to the SNP reference number. The sequence containing the polymorphisms also may be referenced by, the “Sequence Identification” set forth in Table 5. The “Sequence Identification” corresponds to cDNA sequence that encodes associated polypeptides (e.g., EPHA3) of the invention. The position of the SNP within the cDNA sequence is provided in the “Sequence Position” column of Table 5. Also, the allelic variation at the polymorphic site and the allelic variant identified as associated with type II diabetes is specified in Table 5. All nucleotide sequences referenced and accessed by the parameters set forth in Table 5 are incorporated herein by reference.

Assay for Verifying, Allelotyping and Genotyping SNPs

A MassARRAY™ system (Sequenom, Inc.) was utilized to perform SNP genotyping in a high-throughput fashion. This genotyping platform was complemented by a homogeneous single-tube assay method (hME™ or homogeneous MassEXTEND® (Sequenom, Inc.)) in which two genotyping primers anneal to and amplify a genomic target surrounding a polymorphic site of interest. A third primer (the MassEXTEND® primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassEXTEND® primer which where used to genotype the polymorphism. Other primer design software could be used or one of ordinary skill in the art could manually design primers based on his or her knowledge of the relevant factors and considerations in designing such primers. Table 6 shows PCR primers and Table 7 shows an extension probe used for analyzing the polymorphism set forth in Table 5. The initial PCR amplification reaction was performed in a 5 μl total volume containing 1×PCR buffer with 1.5 mM MgCl₂ (Qiagen), 200 μM each of dATP, dGTP, dCTP, dCTP (Gibco-BRL) 2.5 ng of genomic DNA, 0.1 units of HotStar DNA polymerase (Qiagen), and 200 nM each of forward and reverse PCR primers specific for the polymorphic region of interest.

TABLE 6
PCR Primers

| SNP | | |
|---|---|---|
| Reference | Forward PCR primer | Reverse PCR primer |
| rs1512183 | CAGGGCTTAG | CTCGTTCTTCA |
| | AGTTTATTGAG | TCACTATTT |

Samples were incubated at 95° C. for 15 minutes, followed by 4 cycles of 9500 for 20 seconds, 56° C. for 30 seconds, and 72° C. for 1 minute, finishing with a 3 minute final extension at 72° C. Following amplification, shrimp alkaline phosphatase (SAP) (0.3 units in a 2× volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific MassEXTEND® primer cocktail to each sample. Each MassEXTEND® cocktail included a specific combination of dideoxynucleotides (ddNTPs) and deoxynucleotides (dNTPs) used to distinguish polymorphic alleles from one another. Methods for verifying, allelotyping and genotyping SNPs are disclosed, for example, in U.S. Pat. No. 6,258,538, the content of which is hereby incorporated by reference. In Table 7, ddNTPs are shown and the fourth nucleotide not shown is the dNTP.

TABLE 7
Extension Primers

| | | |
|---|---|---|
| SNP | | |
| Reference | Extend Probe | Termination Mix |

| | | |
|---|---|---|
| rs1512183 | ACTATTTTAATTCTTTTTCTGTG | CGT |

The MassEXTEND® reaction was performed in a total volume of 9 μl, with the addition of 1× ThermoSequenase buffer, 0.576 units of ThermoSequenase (Amersham Pharmacia), 600 nM MassEXTEND® primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectoCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCHIP® (Sequenom Inc.)). Subsequently, MALDI-TOF mass spectrometry (Biflex and Autoflex MALDI-TOF mass spectrometers (Bruker Daltonics) can be used) and SpectroTYWER RT™ software (Sequenom, Inc.) were used to analyze and interpret the SNP genotype for each sample.

Genetic Analysis

The minor allelic frequency for the polymorphism set forth in Table 5 was verified as being 10% or greater using the extension assay described above in a group of samples isolated from 92 individuals originating from the state of Utah in the United States, Venezuela and France (Coriell cell repositories).

Genotyping results for the allelic variant set forth in Table 5 are shown for female pools in Table 8 and for male pools in Table 9. In Table 8, “F case” and “F control” refer to female case and female control groups, and in Table 9, “M case” and “M control” refer to male case and male control groups.

TABLE 8
Female Genotyping Results

| SNP | | | | Odds | Disease Associated |
|---|---|---|---|---|---|
| Reference | F case | F control | p-value | Ratio | Allele |
| rs1512183 | A = 0.706 | A = 0.801 | 0.0007 | 0.59 | T |
| | T = 0.294 | T = 0.199 | | | |

TABLE 9
Male Genotyping Results

| SNP | | | | Odds | Disease Associated |
|---|---|---|---|---|---|
| Reference | M case | M control | p-value | Ratio | Allele |
| rs1512183 | A = 0.736 | A = 0.790 | 0.0478 | 0.74 | T |
| | T = 0.264 | T = 0.210 | | | |

The single marker alleles set forth in Table 5 were considered validated, since the genotyping data for the females and males were significantly associated with type II diabetes, and because the genotyping results agreed with the original allelotyping results. Particularly significant associations with type II diabetes are indicated by a calculated p-value of less than 0.05 for genotype results, which are set forth in bold text.

Odds ratio results are shown in Tables 8 and 9. An odds ratio is an unbiased estimate of relative risk which can be obtained from most case-control studies. Relative risk (RR) is an estimate of the likelihood of disease in the exposed group (susceptibility allele or genotype carriers) compared to the unexposed group (pot carriers). It can be calculated by the following equation:

RR=IA/Ia

IA is the incidence of disease in the A carriers and Ia is the incidence of disease in the non-carriers.

RR>1 indicates the A allele increases disease susceptibility.

RR<1 indicates the a allele increases disease susceptibility.

For example, RR=1.5 indicates that carriers of the A allele have 1.5 times the risk of disease than non-carriers, i.e., 50% more likely to get the disease.

Case-control studies, do not allow the direct estimation of IA and Ia, therefore relative risk cannot be directly estimated. However, the odds ratio (OR) can be calculated using the following equation:

OR=(nDAnda)/(ndAnDa)=pDA(1−pdA)/pdA(1−pDA), or

OR=((case f)/(1−case f)/((control f)/(1−control f),

where f=susceptibility allele frequency.

An odds ratio can be interpreted in the same way a relative risk is interpreted and can be directly estimated using the data from case-control studies, i.e., case and control allele frequencies. The higher the odds ratio value, the larger the effect that particular allele has, on the development of breast cancer. Possessing an allele associated with a relatively high odds ratio translates to having a higher risk of developing or having type II diabetes.

### Example 3

### Samples and Pooling Strategies for the Replication Cohort

The single marker polymorphism set forth in Table 5 was genotyped again in two replication cohorts to further validate its association with type II diabetes. Like the original study population described in Examples 1 and 2, the replication cohorts consisted of type II diabetics (cases) and non-diabetics (controls). The case and control samples were selected and genotyped as described below.

Sample Selection and Pooling Strategies Newfoundland

Blood samples were collected from individuals diagnosed with type II diabetes, which were referred to as case samples. Also, blood samples were collected from individuals not diagnosed with type II diabetes or a history of type II diabetes; these samples served as gender and age-matched controls. All of the samples were collected from individuals residing in Newfoundland, Canada. Residents of Newfoundland represent a preferred population for genetic studies because of their relatively small founder population and resulting homogeneity.

Genetic linkage studies from Newfoundland have proved particularly useful for mapping disease genes for both monogenic and complex diseases as evidenced in studies of autosomal dominant polycystic kidney disease, von Hippel-Lindau disease, ankylosing spondylitis, major depression, Grave's eye disease, retinitis pignentosa, hereditary nonopolyposis colorectal cancer, Kallman syndrome, ocular albinism type I, late infantile type 2 neuronatceroid lipofuscinosis, Bardet-Biedl syndrome, adenine phosphoriboysl-transferase deficiency, and arthropathy-camptodactyly syndrome, Familial multiple endocrine neoplasia type 1 (MEN1). Thus Newfoundland's genetically enriched population provides a unique setting to rapidly identify disease-related genes in selected complex diseases.

Phenotypic trait information was gathered from individuals for each case and control sample, and genomic DNA was extracted from each of the blood samples for genetic analyses.

Samples were placed into one of four groups based on disease status. The four groups were female case samples, female control samples, mate case samples, and male control samples. A select set of samples from each group were utilized to generate pools, and one pool was created for each group.

Patients were included in the case pools if a) they were diagnosed with type II diabetes as documented in their medical record, b) they were treated with either insulin or oral hypoglycemic agents, and c) they were of Caucasian ethnicity. Patients were excluded in the case pools if a) they were diabetic or had a history of diabetes, b) they suffered from diet controlled glucose intolerance, or c) they (or any their relatives) were diagnosed with MODY or gestational diabetes.

Phenotype information included, among others, patient ethnicity, country or origin of mother and father, diagnosis with type II diabetes (date of primary diagnosis, age of individual as of primary diagnosis), body weight, onset of obesity, retinopathy, glaucoma, cataracts, nephropathy, heart disease, hypertension, myocardial infarction, ulcers, required treatment (onset of insulin treatment, oral hypoglycemic agent), blood glucose levels, and MODY.

In total, the final selection consisted of 199 female cases, 241 Female Controls, 140 Male Case, and 62 Male Controls as set forth in Table 10.

TABLE 10

| | Female | Female | Male | Male |
|---|---|---|---|---|
| | case | control | cases | control |
| Pool size | 199 | 241 | 140 | 62 |
| (Number) | | | | |
| Pool Criteria | case | control | case | control |
| (ex: case/control) | | | | |
| Mean Age | 58 | 49 | 59 | 51 |
| (ex: years) | | | | |

Sample Selection and Pooling Strategies Denmark

The polymorphism described in Table 5 was genotyped again in a second replication cohort, consisting of individuals of Danish ancestry, to further validate its association with type II diabetes. Blood samples were collected from individuals diagnosed with type II diabetes, which were referred to case samples. Also, blood samples were collected from individuals not diagnosed with Type II diabetes or a history of type II diabetes; these samples served as gender and age-matched controls.

Phenotypic trait information was gathered from individuals for each case and control sample, and genomic DNA was extracted from each of the blood samples for genetic analyses.

Samples were placed into one of four groups based on disease status. The four groups were female case samples, female control samples, male case samples and male control samples. A select set of samples from each group were utilized to generate pools, and one pool was created for each group.

In total, the final selection consisted of 197 female cases (average age 63) and 277 male cases (average age 60) as set forth in Table 11. All cases had been diagnosed with Type II diabetes in their mid 50's, and were of Danish ancestry. Members selected for the cohort were recruited through the outpatient clinic at Steno Diabetes Center, Copenhagen. Diabetes was diagnosed according to the 1985 World Health Organization criteria. For the controls, 152 females (average age 50), and 136 males (average age 55) were selected. All control subjects underwent a 2-hour oral glucose tolerance test (OGTT) and were deemed to be glucose tolerant, and all were of Danish ancestry. In addition, all control subjects were living in the same area of Copenhagen as the type II diabetic patients.

Additional phenotype were measured in both the case and control group. Phenotype information included, among others, e.g. body mass index, waist/hip ratio, blood pressure, serum insulin, glucose, C-peptide, cholesterol, hdl, triglyceride, Hb_{AlC}, urine, creatinine, free fatty acids (mmol/l), GAD antibodies.

TABLE 11

| | Female | Female | Male | Male |
|---|---|---|---|---|
| | case | control | cases | control |
| Pool size | 197 | 152 | 277 | 136 |
| (Number) | | | | |
| Pool Criteria | case | control | case | control |
| (ex: case/control) | | | | |
| Mean Age | 63 | 50 | 59 | 55 |
| (ex: years) | | | | |

DNA Extraction from Blood Samples

Blood samples for DNA preparation were taken in 5 EDTA tubes. If it was not possible to get a blood sample from a patient, a sample from the cheek mucosa was taken. Red blood cells were lysed to facilitate their separation from the white blood cells. The white cells were pelleted and lysed to release the DNA. Lysis was done in the presence of a DNA preservative using an anionic detergent to solubilize the cellular components. Contaminating RNA was removed by treatment with an RNA digesting enzyme. Cytoplasmic and nuclear proteins were removed by salt precipitation.

Genomic DNA was then isolated by precipitation with alcohol (2-propanol and then ethanol) and rehydrated in water. The DNA was transferred to 2-ml tubes and stored at 4° C. for short-term storage and at −70° C. for long-term storage.

### Example 4

### Association of Polymorphic Variants with Type II Diabetes in the Replication Cohorts

The associated SNP from the initial scan was re-validated by, genotyping the associated SNP across the replication cohorts described in Example 3. The results of the genotyping were then analyzed, allele frequencies for each group were calculated from the individual genotyping results, and a p-value was calculated to determine whether the case and control groups had statistically significant differences in allele frequencies for a particular SNP. The replication genotyping results with a calculated p-value of less than 0.05 were considered particularly significant, which are set forth in bold text. See. Tables 12 and 13 herein.

Assay for Verifying, Allelotyping, and Genotyping SNPs

Genotyping of the replication cohort was performed using the same methods used for the original genotyping, as described herein. A MassARRAY™ system (Sequenom, Inc.) was utilized to perform SNP genotyping in a high-throughput fashion. This genotyping platform was complemented by a homogeneous, single-tube assay method (hME™ or homogeneous MassEXTEND® (Sequenom, Inc.)) in which two genotyping primers anneal to and amplify a genomic target surrounding a polymorphic site of interest. A third primer (the MassEXTEND® primer), which is complementary to the amplified target up to but not including the polymorphism, was then enzymatically extended one or a few bases through the polymorphic site and then terminated.

For each polymorphism, SpectroDESIGNER™ software (Sequenom, Inc.) was used to generate a set of PCR primers and a MassXTEND® primer which where used to genotype the polymorphism. Other primer design software could be used or one of ordinary skill in the art could manually design primers based on his or her knowledge of the relevant factors and considerations in designing such primers. Table 6 shows PCR primers and Table 7 shows extension probes used for analyzing (e.g., genotyping) polymorphisms in the replication cohorts. The initial PCR amplification reaction was performed in a 5 μl total volume containing 1×PCR buffer with 1.5 mM MgCl₂ (Qiagen), 200 μM each of dATP, dGTP, dCTP, dTTP (Gibco-BRL), 2.5 ng of genomic DNA, 0.1 units of HotStar DNA polymerase (Qiagen), and 200 nM each of forward and reverse PCR primers specific for the polymorphic region of interest.

Samples were incubated at 95° C. for 15 minutes, followed by 45 cycles of 95° C. for 20 seconds, 56° C. for 30 seconds, and 72° C. for 1 minute, finishing with a 3 minute final extension at 72° C. Following amplification, shrimp alkaline phosphatase (SAP) (0.3 units in a 2 μl volume) (Amersham Pharmacia) was added to each reaction (total reaction volume was 7 μl) to remove any residual dNTPs that were not consumed in the PCR step. Samples were incubated for 20 minutes at 37° C., followed by 5 minutes at 85° C. to denature the SAP.

Once the SAP reaction was complete, a primer extension reaction was initiated by adding a polymorphism-specific. MassEXTEND® primer cocktail to each sample. Each MassEXTEND® cocktail included a specific combination of dideoxynucleotides (ddNTPs) and deoxynucleotides (dNTPs) used to distinguish polymorphic alleles from one another. Methods for verifying, allelotyping and genotyping SNPs are disclosed, for example, in U.S. Pat. No. 6,258,538, the content of which is hereby incorporated by reference. In Table 7, ddNTPs are shown and the fourth nucleotide not shown is the dNTP.

The MassEXTEND® reaction was performed in a total volume of 9 μl, with the addition of 1× ThermoSequenase buffer, 0.576 units of ThermoSequenase (Amersham Pharmacia), 600 nM MassEXTEND® primer, 2 mM of ddATP and/or ddCTP and/or ddGTP and/or ddTTP, and 2 mM of dATP or dCTP or dGTP or dTTP. The deoxy nucleotide (dNTP) used in the assay normally was complementary to the nucleotide at the polymorphic site in the amplicon. Samples were incubated at 94° C. for 2 minutes, followed by 55 cycles of 5 seconds at 94° C., 5 seconds at 52° C., and 5 seconds at 72° C.

Following incubation, samples were desalted by adding 16 μl of water (total reaction volume was 25 μl), 3 mg of SpectroCLEAN™ sample cleaning beads (Sequenom, Inc.) and allowed to incubate for 3 minutes with rotation. Samples were then robotically dispensed using a piezoelectric dispensing device (SpectroJET™ (Sequenom, Inc.)) onto either 96-spot or 384-spot silicon chips containing a matrix that crystallized each sample (SpectroCUW® (Sequenom, Inc.)). Subsequently, MALDI-TOF mass spectrometry (Biflex and Autoflex MALDI-TOF mass spectrometers (Bruker Daltonics) can be used) and SpectroTYPER RT™ software (Sequenom, Inc.) were used to analyze and interpret the SNP genotype for each sample.

Genetic Analysis

The minor allelic frequency for the polymorphism set forth in Table 5 was verified as being 10% or greater using the extension assay described above in a group of samples isolated from 92 individuals originating from the state of Utah in the United States, Venezuela and France (Coriell cell repositories).

Replication genotyping results in both cohorts are shown for female pools in Table 12 and for male pools in Table 13.

TABLE 12
Female Replication Genotyping Results

| SNP | Replication | | | | Odds |
|---|---|---|---|---|---|
| Reference | Population | F case | F control | p-value | Ratio |
| rs1512183 | Newfoundland | T = 0.320 | T = 0.248 | 0.021 | 0.72 |
| | | A = 0.680 | A = 0.752 | | |
| rs1512183 | Denmark | T = 0.249 | T = 0.212 | 0.273 | 0.84 |
| | | A = 0.751 | A = 0.788 | | |

TABLE 13
Male Replication Genotyping Results

| | Replication | | | | Odds |
|---|---|---|---|---|---|
| SNP Reference | Population | M case | M | p-value | Ratio |
| rs1512183 | Newfoundland | T = 0.289 | T = 0.242 | 0.324 | 0.68 |
| | | A = 0.711 | A = 0.758 | | |
| rs1512183 | Denmark | T = 0.203 | T = 0.250 | 0.144 | 1.3 |
| | | A = 0.797 | A = 0.750 | | |

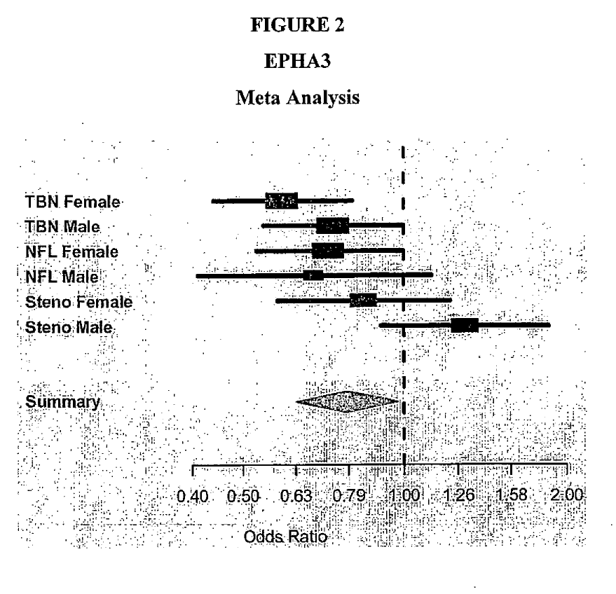

Meta-analysis was performed on rs1512183 based on genotype results provided in Tables 8, 9, 12 and 13. FIG. 2 depicts the combined meta analysis odds ratio for rs1512183 in males, females and combined genders (see Examples 1-4). In FIG. 2, “TBN” is the abbreviation for the discovery cohort, “NFL” is the abbreviation for the Newfoundland replication cohort, and “Steno” is the abbreviation for the Denmark replication cohort. The boxes are centered over the odds ratio for each sample, with the size of the box correlated to the contribution of each sample to the combined meta analysis odds ratio. The lines extending from each box are the 95% confidence interval values. The diamond is centered over the combined meta analysis odds ratio with the ends of the diamond depicting the 95% confidence interval values. The meta-analysis further illustrates the strong association each of the incident SNPs has with Type II diabetes across multiple case and control samples.

The subjects available for discovery from Germany included 498 cases and 498 controls. The subjects available for replication from Newfoundland included 350 type 2 diabetes cases and 300 controls. The subjects available for replication from Denmark included 474 type 2 diabetes cases and 287 controls. Meta analyses, combining the results of the German discovery sample and both the Canadian and Danish replication sample, were carried out using a random effects (DerSimonian-Laird) procedure.

The absence of a statistically significant association in the replication cohort for males should not be interpreted as minimizing the value of the original finding. There are many reasons why a biologically derived association identified in a sample from one population would not replicate in a sample from another population. The most important reason is differences in population history. Due to bottlenecks and founder effects, there may be, common disease predisposing, alleles present in one population that are relatively rare in another, leading to a lack of association in the candidate region. Also, because common diseases such as diabetes are the result of susceptibilities in many genes and many environmental risk factors, differences in population-specific genetic and environmental backgrounds could mask the effects of a biologically relevant allele. For these and other reasons, statistically strong results in the original, discovery sample that did not replicate in the replication Newfoundland sample may be further evaluated in additional replication cohorts and experimental systems.

### Example 5

### EPHA3 Proximal SNPs

The SNP rs1512183 associated with type II diabetes in the examples above fits within the EPHA3 gene. EPHA3 is an ephrin-like tyrosine kinase that has two isoforms produced by alternate splicing: transcript variant 1 is a membrane protein, and transcript variant 2 is secreted (see SEQ ID NO: 2 and 3). High affinity ligands of EPHA3 include ephrin-A2 (which is expressed highly in the pancreas) and ephrin-A5 (which is highly expressed in heart and kidney).

Forty additional allelic variants proximal to rs1512183 were identified and subsequently allelotyped in diabetes case and control sample sets as described in Examples 1 and 2. The polymorphic variants are set forth in Table 14. The chromosome position provided in column three of Table 14 is based on Genome “Build 34” of NCBI's GenBank. The “genome letter” corresponds to the particular allele that appears in NCBI's build 34 genomic sequence of the region (chromosome 3: positions 89375801-89470550), and the “deduced iupac” corresponds to the single letter IUPAC code for the EPHA3 polymorphic variants as they appear in SEQ ID NO:1. The “genome letter” may differ from the alleles (A1/A2) provided in Table 14 if the genome letter is on one strand and the alleles are on the complementary strand, thus they have different strand orientations (i.e., reverse vs forward).

TABLE 14

| | Position | | | | | |
|---|---|---|---|---|---|---|
| | in | | | | | |
| | SEQ ID | Chro- | Chromosome | Alleles | genome | deduced |
| dbSNP | NO: 1 | mosome | Position | (A1/A2) | letter | iupac |
| 3792573 | 225 | 3 | 89376025 | A/C | t | K |
| 3792572 | 1656 | 3 | 89377456 | A/T | t | W |
| 1398195 | 5432 | 3 | 89381232 | C/T | g | R |
| 3828462 | 5652 | 3 | 89381452 | G/T | a | M |
| 3805091 | 6343 | 3 | 89382143 | G/T | a | M |
| 1512185 | 18716 | 3 | 89394516 | A/G | g | R |
| 1028013 | 29369 | 3 | 89405169 | T/C | t | Y |
| 987748 | 39131 | 3 | 89414931 | T/G | c | M |
| 1398197 | 43529 | 3 | 89419329 | T/C | g | R |
| 1028011 | 43720 | 3 | 89419520 | T/C | a | R |
| 2881488 | 45589 | 3 | 89421389 | C/G | c | S |
| 1473598 | 48922 | 3 | 89424722 | A/C | a | M |
| 1512183 | 50155 | 3 | 89425955 | T/A | a | W |
| 1157607 | 51465 | 3 | 89427265 | C/T | c | Y |
| 1157608 | 51565 | 3 | 89427365 | T/G | t | K |
| 1912965 | 63433 | 3 | 89439233 | G/A | a | R |
| 1912966 | 63565 | 3 | 89439365 | A/T | t | W |
| 1982096 | 64496 | 3 | 89440296 | C/T | t | Y |
| AA | 66765 | 3 | 89442565 | G/A | g | R |
| AB | 66794 | 3 | 89442594 | T/C | t | Y |
| 1054750 | 66826 | 3 | 89442626 | C/T | t | Y |
| 2117137 | 70606 | 3 | 89446406 | C/T | a | R |
| 1499780 | 71173 | 3 | 89446973 | C/G | g | S |
| 2117138 | 76623 | 3 | 89452423 | G/A | a | R |
| 2346840 | 78368 | 3 | 89454168 | T/G | t | K |
| 2048518 | 79006 | 3 | 89454806 | C/T | t | Y |
| 2048519 | 79079 | 3 | 89454879 | G/A | g | R |
| 2048520 | 79349 | 3 | 89455149 | T/C | t | Y |
| 2048521 | 79354 | 3 | 89455154 | G/A | g | R |
| 3762718 | 80167 | 3 | 89455967 | T/G | t | K |
| 2196083 | 81647 | 3 | 89457447 | T/C | t | Y |
| 1512187 | 82179 | 3 | 89457979 | T/C | c | Y |
| 972030 | 83599 | 3 | 89459399 | G/T | g | K |
| 2346837 | 87700 | 3 | 89463500 | T/C | c | Y |
| 1036286 | 88778 | 3 | 89464578 | T/G | c | M |
| 1036285 | 89162 | 3 | 89464962 | A/G | t | Y |
| 1512188 | 91284 | 3 | 89467084 | A/G | a | R |
| 1512189 | 91433 | 3 | 89467233 | A/G | a | R |
| 1567657 | 93620 | 3 | 89469420 | A/G | g | R |
| 1567658 | 93707 | 3 | 89469507 | T/A | t | W |
| 1028012 | 94523 | 3 | 89470323 | C/T | c | Y |

Assay for Verifying and Allelotyping SNPs

The methods used to verify and allelotype the forty proximal SNPs of Table 14 are the same methods described in Examples 1 and 2 herein. The primers and probes used in these assays are provided in Table 15 and Table 16, respectively.

TABLE 15

| | | |
|---|---|---|
| dbSNP rs# | Forward PCR primer | Reverse PCR primer |

| | | |
|---|---|---|
| 3792573 | CTTTTACACGCTCTGCTATG | TGACCATGTCATTCCTATGC |
| 3792572 | ATTGGGAGGTGAGTTCACAG | AGCAATCAGTGGCAGCAAAG |
| 1398195 | TCTTGGCAAAAAAGACAAGC | CAACAGTTTCTCATGCTAGG |
| 3828462 | GGATAGCAGCTGCTCTTTTG | AGGGCAAATAGGGAATGCAG |
| 3805091 | TACCATAGGAGTTACATTGC | CCCTGAGTTTVAAAAAAGTG |
| 1512185 | AACAAACCGACAAATGTCAC | AGGTTTAAGATTTCCTGTAC |
| 1028013 | ATCAGCATTCCTGGAGTAAG | TATGAGGTATGGTTATGAAG |
| 987748 | CCATTATCTGCTAATAGGTAG | GATAAGAGTCUGAATCTGTG |
| 1393197 | CATCACCAGAAGCTATAGCC | TCACCTCCCAGAGAACAATC |
| 1028011 | ATAATGTAACTGGGTGGCTC | CTTGCAAGACTTGGAAACAG |
| 2881488 | CAGTGGAAAAAAGAAGGAAG | TAATTACTCTGAGAGGCACC |
| 1473598 | GTCAGCAGGGCTTTTTAAAC | GATATAGTTTCAGTCACCTG |
| 1157607 | GGGMCACTACAGAAAAGGGC | CCTATAGTCTAGCAAAACCG |
| 1157608 | ATAGATCAGGGATCCTCAGC | TTTGCCTTCTGCTTTGCCAC |
| 1912965 | CTGTCCACTAGACTAAACTC | TCTCTGAGAG1TAGTAAAAC |
| 1912966 | ATTGCAACATTGGTTCTGAC | CCTCCACTACACAAATGACC |
| 1982096 | ATAATGGCTACTTTTCAGGG | GCAATGATCTTGTTAACCACC |
| 1054750 | CAGCACACTGCAAGGAAATC | GTCATCTGTGGAAATCTTGG |
| 2117137 | TGAACTGAAGCACTCATCCC | GGTGCAGCTATGAAGAAGTG |
| 1499780 | ACTATTATTTTAGGAAGGGGAAAG | CTTCTTCCAATACCATATCC |
| 2117138 | TCCCCAGATAATTCTGTCAC | CAAAGTTAATAAACACAACTG |
| 2346840 | GCTAGCTACATAGTTAAGTTC | ATTGCAAAGAAGGCCACCTG |
| 2048518 | GCTCTACCTTGGTTAATGCC | GGGATTTACCACTTGTGAAG |
| 2048519 | GATCAGACTGTAGGGTATGC | CTTCACAAGTGGTAAATCCC |
| 2048520 | CTAAGGTGGCATTGTTAGGG | TCTCAAGTTGACTCACTTGG |
| 2048521 | CTAAGGTGGCATTGTTAGGG | TCTCAAGTTGACTCACTTGG |
| 3762718 | GTAGTTCTCTGAGTAUCCAC | GGACCTGAAGMTGATTAGAG |
| 2196083 | GAACATGTTTGCTTGAAGGG | GGAACAGGCAGTTATCCTTG |
| 1512187 | TTGAGTATTAAGGGCTCTCC | TTGGACACAATCCTTCAGAG |
| 972030 | TTGTCTCTGACCCAGAAACC | AAGCCAACAATTTGGCCTGC |
| 2346837 | AAGAGCACGAAGAACACAAG | ACTATTCAGGACCCTCTTGC |
| 1036286 | AAGTCCTGCTGTGCATTTTG | GTACTCTGAACCAAATGAGC |
| 1036285 | GAAGTCGGGAACATTTTGTG | AGTGTCATAAATGTCCAGGC |
| 1512188 | GAGGCAGATGTAGAAACAAG | AAGCAAGTCACTGTACAGAC |
| 1512189 | GCATACCATGAAATTCACCC | ACTGTGTTGATGCTAGCAAG |
| 1567657 | GGCTAAGGAAAAACTGAAACC | ATGCTTTCTCTGATGTTGGG |
| 1567658 | TCAGAACTCCAAAGAGCAAG | TCCTAAGATGGTTTGGTGAC |
| 1028012 | TTCCCTACTCACCACATAGC | TGCTCAGGTAACAGTTCTGG |

TABLE 16

| dbSNP | Extend | Term |
|---|---|---|
| rs# | Primer | Mix |
| 3792573 | GAAAGTTATAAAGAGCCAAAAT | ACT |
| 3792572 | CTGTTTCACTATTCACTTTC | CGT |
| 1398195 | AAAAAAGACAAGCTAGGGCT | ACG |
| 3528462 | GCTCTTTTGTTGTAGCTGT | CGT |
| 3805091 | GTTACATTGCAATTTCCTTT | CGT |
| 1512185 | AAAGCATTTCTTTTCGAGA | ACT |
| 1028013 | ACTTAGAATGCTGCCTC | ACT |
| 987748 | TGCTAATAGGTAGCTTATTAGAC | ACT |
| 1398197 | GAAGCTATAGCCTACCGCAA | ACT |
| 1028011 | TGGGTGGCTCCTGGTTT | ACT |
| 2881488 | AGATGAATTTTGTCAGGGG | ACT |
| 1473598 | ACAACCAAAAGGAGATTTTTTTA | ACT |
| 1157607 | AGGATACTTCCAAATCACAT | ACG |
| 1157608 | CCCCCGTGCCATGGACTGCT | ACT |
| 1912965 | AACTCAATTACCTCAATCCTTT | ACG |
| 1912966 | CATTGGTTCTGACTCTATTT | CGT |
| 1982096 | CTACTTTTCAGGGTTGTTA | ACG |
| 1054750 | AAGGAAATCTTCACGGG | ACG |
| 2117137 | TTGCAATCAAAAGAGAACTG | ACG |
| 1499780 | GAAGGGGAAGGGAAGA | ACT |
| 2117138 | TTGCAATCAAAAGAGAACTG | ACG |
| 2346840 | AATATGITTTTTAACACAGAATGA | ACT |
| 2048518 | TGAGTATGGTGGAAGTACTAG | ACG |
| 2048519 | AGGGTATGCCTGAGCAAG | ACG |
| 2048520 | ACTGAAGAAAAGGCAGAG | ACT |
| 2048521 | TTAGACACTGAAGAAAAGG | ACG |
| 3762718 | ATGCATCATAGCTTTTGACTTTTT | ACT |
| 2196083 | CTTGAAOGGTTCACTGT | ACT |
| 1512187 | AGGGCTCTCCAGACCAA | ACT |
| 972030 | GAAACCTCATGACAACTG | CGT |
| 2346837 | GTGCTTAAAACTCCTCTTAT | ACT |
| 1036286 | TATGATTTTGACTTTTCAAAGTT | ACT |
| 1036285 | CCTAATAACTTATGTCTTACACA | ACT |
| 1512188 | AAGGTGTAAGTTTCATAAATGG | ACT |
| 1512189 | AATTCACCCATTTAAGTGTATA | ACT |
| 1567657 | AAAAACTGAAACCTGAAACT | ACT |
| 1567658 | CCAAAGAGCAAGAAAGCTTTAA | CGT |
| 1028012 | CACATAGCAAATATATGACACAA | ACG |

Genetic Analysis

Allelotyping results are shown for female (F), male (M), and combined cases and controls in Table 17, 18 and 19 respectively. The allele frequency for the A2 allele is noted in the fifth and sixth columns for diabetes case pools and control pools, respectively, where “AF” is allele frequency. The allele frequency for the A1 allele can be easily calculated by subtracting the A2 allele frequency from 1 (A1 AF=1-A2 AF). For example, the SNP rs3792572 has the following case and control allele frequencies: case A1 (A)=0.568; case A2 (T)=0.432; control A1 (A)=0.655; and control A2 (T)=0.345, where the nucleotide is provided in parenthesis. Some SNPs may be labeled “untyped” because of failed assays.

TABLE 17
Female Allelotyping Results

| | Position | | | | | | | Diabetes |
|---|---|---|---|---|---|---|---|---|
| | in SEQ | Chromosome | A1/A2 | Female A2 | Female A2 | Female | Female | Associated |
| dbSNP | ID NO: 1 | Position | Allele | Case AF | Control AF | p-Value | OR | Allele |
| 3792573 | 225 | 89376025 | [A/C] | 0.002 | 0.002 | 0.97610 | 0.89 | A |
| 3792572 | 1,656 | 89377456 | [A/T] | 0.432 | 0.345 | 0.01355 | 1.45 | T |
| 1398195 | 5,432 | 89381232 | [C/T] | 0.001 | 0.001 | 0.94899 | 0.57 | C |
| 3828462 | 5,652 | 89381452 | [G/T] | 0.998 | 0.998 | 0.99029 | 1.05 | T |
| 3805091 | 6,343 | 89382143 | [G/T] | 0.991 | 0.980 | 0.30023 | 2.36 | T |
| 1512185 | 18,716 | 89394516 | [A/G] | 0.576 | 0.689 | 0.00080 | 0.61 | A |
| 1028013 | 29,369 | 89405169 | [T/C] | 0.257 | 0.183 | 0.01145 | 1.54 | C |
| 987748 | 39,131 | 89414931 | [T/G] | 0.364 | 0.489 | 0.00039 | 0.60 | T |
| 1398197 | 43,529 | 89419329 | [T/C] | 0.879 | 0.902 | 0.29098 | 0.79 | T |
| 1028011 | 43,720 | 89419520 | [T/C] | 0.841 | 0.823 | 0.54141 | 1.13 | C |
| 2881488 | 45,589 | 89421389 | [C/G] | 0.273 | 0.207 | 0.02527 | 1.43 | G |
| 1473598 | 48,922 | 89424722 | [A/C] | 0.001 | 0.000 | 0.96969 | 1.66 | C |
| 1157607 | 51,465 | 89427265 | [C/T] | 0.286 | 0.196 | 0.00276 | 1.64 | T |
| 1157608 | 51,565 | 89427365 | [T/G] | 0.227 | 0.164 | 0.03123 | 1.49 | G |
| 1912965 | 63,433 | 89439233 | [G/A] | 0.614 | 0.716 | 0.00128 | 0.63 | G |
| 1912966 | 63,565 | 89439365 | [A/T] | 0.650 | 0.726 | 0.01868 | 0.70 | A |
| 1982096 | 64,496 | 89440296 | [C/T] | 0.915 | 0.951 | 0.05630 | 0.56 | C |
| 1054750 | 66,826 | 89442626 | [C/T] | 0.707 | 0.756 | 0.12386 | 0.78 | C |
| 2117137 | 70,606 | 89446406 | [C/T] | 0.478 | 0.556 | 0.02971 | 0.73 | C |
| 1499780 | 71,173 | 89446973 | [C/G] | 0.514 | 0.651 | 0.00008 | 0.57 | C |
| 2117138 | 76,623 | 89452423 | [G/A] | 0.643 | 0.703 | 0.05989 | 0.76 | G |
| 2346840 | 78,368 | 89454168 | [T/G] | 0.223 | 0.169 | 0.05000 | 1.41 | G |
| 2048518 | 79,006 | 89454806 | [C/T] | 0.655 | 0.730 | 0.01811 | 0.70 | C |
| 2048519 | 79,079 | 89454879 | [G/A] | 0.292 | 0.216 | 0.02150 | 1.49 | A |
| 2048520 | 79,349 | 89455149 | [T/C] | 0.018 | 0.006 | 0.24350 | 3.06 | C |
| 2048521 | 79,354 | 89455154 | [G/A] | 0.322 | 0.252 | 0.02290 | 1.41 | A |
| 3762718 | 80,167 | 89455967 | [T/G] | 0.217 | 0.159 | 0.03599 | 1.46 | G |
| 2196083 | 81,647 | 89457447 | [T/C] | 0.241 | 0.166 | 0.00807 | 1.59 | C |
| 1512187 | 82,179 | 89457979 | [T/C] | 0.991 | 0.994 | 0.71104 | 0.62 | T |
| 972030 | 83,599 | 89459399 | [G/T] | 0.351 | 0.266 | 0.01187 | 1.50 | T |
| 2346837 | 87,700 | 89463500 | [T/C] | 0.997 | 0.999 | 0.82719 | 0.39 | T |
| 1036286 | 88,778 | 89464578 | [T/G] | 0.854 | 0.919 | 0.00429 | 0.51 | T |
| 1036285 | 89,162 | 89464962 | [A/G] | 0.267 | 0.194 | 0.01271 | 1.51 | G |
| 1512188 | 91,284 | 89467084 | [A/G] | 0.256 | 0.184 | 0.01715 | 1.53 | G |
| 1512189 | 91,433 | 89467233 | [A/G] | 0.311 | 0.228 | 0.00834 | 1.53 | G |
| 1567657 | 93,620 | 89469420 | [A/G] | 0.541 | 0.655 | 0.00085 | 0.62 | A |
| 1567658 | 93,707 | 89469507 | [T/A] | 0.254 | 0.200 | 0.06059 | 1.36 | A |
| 1028012 | 94,523 | 89470323 | [C/T] | 0.237 | 0.178 | 0.03523 | 1.44 | T |

TABLE 18
Male Allelotyping Results

| | Position | | | | Male A2 | | | Diabetes |
|---|---|---|---|---|---|---|---|---|
| | in SEQ | Chromosome | A1/A2 | Male A2 | Control | Male | Male | Associated |
| dbSNP | ID NO: 1 | Position | Allele | Case AF | AF | p-Value | OR | Allele |
| 3792573 | 225 | 89376025 | [A/C] | 0.002 | 0.000 | 0.80788 | 4.89 | C |
| 3792572 | 1,656 | 89377456 | [A/T] | 0.410 | 0.388 | 0.52606 | 1.09 | T |
| 1398195 | 5,432 | 89381232 | [C/T] | 0.001 | 0.002 | 0.89083 | 0.30 | C |
| 3828462 | 5,652 | 89381452 | [G/T] | 0.999 | 1.000 | 0.93099 | 0.02 | G |
| 3805091 | 6,343 | 89382143 | [G/T] | 0.996 | 0.996 | 0.97331 | 0.90 | G |
| 1512185 | 18,716 | 89394516 | [A/G] | 0.665 | 0.723 | 0.08628 | 0.76 | A |
| 1028013 | 29,369 | 89405169 | [T/C] | 0.213 | 0.170 | 0.12147 | 1.33 | C |
| 987748 | 39,131 | 89414931 | [T/G] | untyped | 0.480 | | | |
| 1398197 | 43,529 | 89419329 | [T/C] | 0.919 | 0.896 | 0.24720 | 1.33 | C |
| 1028011 | 43,720 | 89419520 | [T/C] | 0.880 | 0.849 | 0.23952 | 1.30 | C |
| 2881488 | 45,589 | 89421389 | [C/G] | 0.248 | 0.192 | 0.04777 | 1.39 | G |
| 1473598 | 48,922 | 89424722 | [A/C] | 0.000 | 0.001 | 0.94560 | 0.00 | A |
| 1157607 | 51,465 | 89427265 | [C/T] | 0.242 | 0.195 | 0.11191 | 1.32 | T |
| 1157608 | 51,565 | 89427365 | [T/G] | 0.247 | 0.197 | 0.07481 | 1.34 | G |
| 1912965 | 63,433 | 89439233 | [G/A] | 0.629 | 0.666 | 0.28523 | 0.85 | G |
| 1912966 | 63,565 | 89439365 | [A/T] | 0.661 | 0.742 | 0.01409 | 0.68 | A |
| 1982096 | 64,496 | 89440296 | [C/T] | 0.957 | 0.911 | 0.04603 | 2.19 | T |
| 1054750 | 66,826 | 89442626 | [C/T] | 0.686 | 0.749 | 0.05024 | 0.73 | C |
| 2117137 | 70,606 | 89446406 | [C/T] | 0.510 | 0.517 | 0.85039 | 0.97 | C |
| 1499780 | 71,173 | 89446973 | [C/G] | 0.571 | 0.639 | 0.05268 | 0.75 | C |
| 2117138 | 76,623 | 89452423 | [G/A] | 0.635 | 0.701 | 0.04460 | 0.74 | G |
| 2346840 | 78,368 | 89454168 | [T/G] | 0.222 | 0.172 | 0.07047 | 1.37 | G |
| 2048518 | 79,006 | 89454806 | [C/T] | 0.638 | 0.714 | 0.02368 | 0.71 | C |
| 2048519 | 79,079 | 89454879 | [G/A] | 0.259 | 0.219 | 0.25605 | 1.25 | A |
| 2048520 | 79,349 | 89455149 | [T/C] | 0.004 | 0.003 | 0.87086 | 1.54 | C |
| 2048521 | 79,354 | 89455154 | [G/A] | 0.307 | 0.250 | 0.08642 | 1.33 | A |
| 3762718 | 80,167 | 89455967 | [T/G] | 0.226 | 0.182 | 0.11149 | 1.31 | G |
| 2196083 | 81,647 | 89457447 | [T/C] | 0.223 | 0.163 | 0.02500 | 1.47 | C |
| 1512187 | 82,179 | 89457979 | [T/C] | 0.995 | 0.995 | 0.97683 | 1.08 | C |
| 972030 | 83,599 | 89459399 | [G/T] | 0.327 | 0.288 | 0.24448 | 1.20 | T |
| 2346837 | 87,700 | 89463500 | [T/C] | 0.993 | 0.995 | 0.86322 | 0.76 | T |
| 1036286 | 88,778 | 89464578 | [T/G] | 0.909 | 0.933 | 0.32768 | 0.72 | T |
| 1036285 | 89,162 | 89464962 | [A/G] | untyped | 0.194 | | | |
| 1512188 | 91,284 | 89467084 | [A/G] | 0.247 | 0.194 | 0.05761 | 1.36 | G |
| 1512189 | 91,433 | 89467233 | [A/G] | 0.292 | 0.225 | 0.02826 | 1.42 | G |
| 1567657 | 93,620 | 89469420 | [A/G] | 0.598 | 0.674 | 0.01723 | 0.72 | A |
| 1567658 | 93,707 | 89469507 | [T/A] | 0.266 | 0.214 | 0.09116 | 1.33 | A |
| 1028012 | 94,523 | 89470323 | [C/T] | 0.221 | 0.190 | 0.28643 | 1.21 | T |

TABLE 19
Combined Allelotyping Results

| | Position | | | | A2 | | | Diabetes |
|---|---|---|---|---|---|---|---|---|
| | in SEQ | Chromosome | A1/A2 | A2 Case | Control | | | Associated |
| dbSNP | ID NO: 1 | Position | Allele | AF | AF | p-Value | OR | Allele |
| 3792573 | 225 | 89376025 | [A/C] | 0.002 | 0.001 | 0.87897 | 1.65 | C |
| 3792572 | 1,656 | 89377456 | [A/T] | 0.413 | 0.367 | 0.05913 | 1.21 | T |
| 1398195 | 5,432 | 89381232 | [C/T] | 0.000 | 0.002 | 0.85746 | 0.26 | C |
| 3828462 | 5,652 | 89381452 | [G/T] | 0.999 | 0.999 | 0.97553 | 0.82 | G |
| 3805091 | 6,343 | 89382143 | [G/T] | 0.993 | 0.988 | 0.57801 | 1.72 | T |
| 1512185 | 18,716 | 89394516 | [A/G] | 0.621 | 0.706 | 0.00035 | 0.68 | A |
| 1028013 | 29,369 | 89405169 | [T/C] | 0.235 | 0.176 | 0.00387 | 1.43 | C |
| 987748 | 39,131 | 89414931 | [T/G] | 0.364 | 0.484 | 0.00008 | 0.61 | T |
| 1398197 | 43,529 | 89419329 | [T/C] | 0.900 | 0.899 | 0.95556 | 1.01 | C |
| 1028011 | 43,720 | 89419520 | [T/C] | 0.861 | 0.837 | 0.21158 | 1.20 | C |
| 2881488 | 45,589 | 89421389 | [C/G] | 0.260 | 0.199 | 0.00270 | 1.41 | G |
| 1473598 | 48,922 | 89424722 | [A/C] | 0.000 | 0.000 | 0.98139 | 0.74 | A |
| 1157607 | 51,465 | 89427265 | [C/T] | 0.255 | 0.196 | 0.00483 | 1.40 | T |
| 1157608 | 51,565 | 89427365 | [T/G] | 0.237 | 0.181 | 0.00553 | 1.41 | G |
| 1912965 | 63,433 | 89439233 | [G/A] | 0.622 | 0.691 | 0.00357 | 0.74 | G |
| 1912966 | 63,565 | 89439365 | [A/T] | 0.658 | 0.734 | 0.00079 | 0.70 | A |
| 1982096 | 64,496 | 89440296 | [C/T] | 0.940 | 0.930 | 0.50606 | 1.17 | T |
| 1054750 | 66,826 | 89442626 | [C/T] | 0.697 | 0.752 | 0.01365 | 0.76 | C |
| 2117137 | 70,606 | 89446406 | [C/T] | 0.492 | 0.536 | 0.07688 | 0.84 | C |
| 1499780 | 71,173 | 89446973 | [C/G] | 0.543 | 0.645 | 0.00004 | 0.65 | C |
| 2117138 | 76,623 | 89452423 | [G/A] | 0.639 | 0.702 | 0.00587 | 0.75 | G |
| 2346840 | 78,368 | 89454168 | [T/G] | 0.223 | 0.171 | 0.00773 | 1.39 | G |
| 2048518 | 79,006 | 89454806 | [C/T] | 0.646 | 0.722 | 0.00091 | 0.70 | C |
| 2048519 | 79,079 | 89454879 | [G/A] | 0.275 | 0.218 | 0.01709 | 1.36 | A |
| 2048520 | 79,349 | 89455149 | [T/C] | 0.011 | 0.004 | 0.31706 | 2.59 | C |
| 2048521 | 79,354 | 89455154 | [G/A] | 0.315 | 0.251 | 0.00473 | 1.38 | A |
| 3762718 | 80,167 | 89455967 | [T/G] | 0.222 | 0.171 | 0.00925 | 1.38 | G |
| 2196083 | 81,647 | 89457447 | [T/C] | 0.231 | 0.165 | 0.00054 | 1.53 | C |
| 1512187 | 82,179 | 89457979 | [T/C] | 0.993 | 0.995 | 0.84511 | 0.78 | T |
| 972030 | 83,599 | 89459399 | [G/T] | 0.336 | 0.277 | 0.01286 | 1.32 | T |
| 2346837 | 87,700 | 89463500 | [T/C] | 0.995 | 0.997 | 0.78608 | 0.65 | T |
| 1036286 | 88,778 | 89464578 | [T/G] | 0.882 | 0.926 | 0.00687 | 0.60 | T |
| 1036285 | 89,162 | 89464962 | [A/G] | 0.267 | 0.194 | 0.00396 | 1.51 | G |
| 1512188 | 91,284 | 89467084 | [A/G] | 0.252 | 0.189 | 0.00235 | 1.44 | G |
| 1512189 | 91,433 | 89467233 | [A/G] | 0.301 | 0.227 | 0.00064 | 1.47 | G |
| 1567657 | 93,620 | 89469420 | [A/G] | 0.570 | 0.665 | 0.00005 | 0.67 | A |
| 1567658 | 93,707 | 89469507 | [T/A] | 0.258 | 0.207 | 0.01627 | 1.33 | A |
| 1028012 | 94,523 | 89470323 | [C/T] | 0.229 | 0.184 | 0.02803 | 1.31 | T |

Allelotyping results were considered particularly significant with a calculated p-value of less than or equal to 0.05 for allelotype results. These values are indicated in bold. The allelotyping p-values were plotted in FIGS. 1A-C for females, males and combined, respectively. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIGS. 1A-C can be determined by consulting Tables 17, 18 and 19. For example, the left-most X on the left graph is at position 89376025. By proceeding down the Table from top to bottom and across the graphs from left to right the allele frequency associated with each symbol shown can be determined.

To aid the interpretation, multiple lines have been added to the graph. The broken horizontal lines are drawn at two common significance levels, 0.05 and 0.01. The vertical broken lines are drawn every 20 kb to assist in the interpretation of distances between SNPs. Two other lines are drawn to expose linear, trends in the association of SNPs to the disease. The light gray, line (or generally bottom-most curve) is a nonlinear smoother through the data points on the graph using a local polynomial regression method (W. S. Cleveland, E. Grosse and W. M. Shyu (1992) Local regression models. Chapter 8 of Statistical Models in S eds J. M. Chambers and T. J. Hastie, Wadsworth & Brooks/Cole.). The black line provides a local test for excess statistical significance to identify regions of association. This was created by use of a 10 kb sliding window with 1 kb step sizes. Within each window, a chi-square goodness of fit test was applied to compare the proportion of SNPs that were significant at a test wise level of 0.01, to the proportion that would be expected by chance alone (0.05 for the methods used here). Resulting p-values that were less than 10⁻⁸ were truncated at that value.

Finally, the exons and introns of the genes in the covered region are plotted, below each graph at the appropriate chromosomal positions. The gene boundary is indicated by the broken horizontal line. The exon positions are shown as thick, unbroken bars. An arrow is place at the 3′ end of each gene to show the direction of transcription.

Proximal SNP Replication

The proximal SNPs disclosed above were also allelotyped in the Newfoundland replication cohort described in Examples 3 and 4. Allelotyping results are shown for female (F), male (M), and combined cases and controls in Table 20, 21 and 22 respectively. The allele frequency for the A2 allele is noted in the fifth and sixth columns for diabetes case pools and control pools, respectively, where “AF” is allele frequency. The allele frequency for the A1 allele can be easily calculated by subtracting the A2 allele frequency from 1 (A1 AF=1-A2 AF). Some SNPs may be labeled “untyped” because of failed assays.

TABLE 20
Female Replication Allelotyping Results

| | | | | | Female | | | |
|---|---|---|---|---|---|---|---|---|
| | Position | | | Female | A2 | | | Diabetes |
| | in SEQ | Chromosome | A1/A2 | A2 Case | Control | Female | Female | Associated |
| dbSNP | ID NO: 1 | Position | Allele | AF | AF | p-Value | OR | Allele |
| 3792573 | 225 | 89376025 | [A/C] | 0.001 | 0.001 | 0.97557 | 1.24 | C |
| 3792572 | 1,656 | 89377456 | [A/T] | 0.403 | 0.358 | 0.16071 | 1.21 | T |
| 1398195 | 5,432 | 89381232 | [C/T] | 0.000 | 0.001 | 0.94608 | 0.04 | C |
| 3828462 | 5,652 | 89381452 | [G/T] | 0.994 | 1.000 | 0.50046 | 0.00 | G |
| 3805091 | 6,343 | 89382143 | [G/T] | 0.991 | 0.990 | 0.90766 | 1.13 | T |
| 1512185 | 18,716 | 89394516 | [A/G] | 0.572 | 0.623 | 0.11500 | 0.81 | A |
| 1028013 | 29,369 | 89405169 | [T/C] | 0.258 | 0.227 | 0.27338 | 1.18 | C |
| 987748 | 39,131 | 89414931 | [T/G] | 0.407 | 0.466 | 0.07313 | 0.79 | T |
| 1398197 | 43,529 | 89419329 | [T/C] | 0.887 | 0.907 | 0.35510 | 0.81 | T |
| 1028011 | 43,720 | 89419520 | [T/C] | 0.838 | 0.835 | 0.89168 | 1.02 | C |
| 2881488 | 45,589 | 89421389 | [C/G] | 0.282 | 0.248 | 0.23045 | 1.19 | G |
| 1473598 | 48,922 | 89424722 | [A/C] | 0.000 | 0.001 | 0.97462 | 0.66 | A |
| 1157607 | 51,465 | 89427265 | [C/T] | 0.282 | 0.225 | 0.04679 | 1.35 | T |
| 1157608 | 51,565 | 89427365 | [T/G] | 0.245 | 0.227 | 0.51251 | 1.10 | G |
| 1912965 | 63,433 | 89439233 | [G/A] | 0.590 | 0.608 | 0.66254 | 0.93 | G |
| 1912966 | 63,565 | 89439365 | [A/T] | 0.641 | 0.687 | 0.14751 | 0.81 | A |
| 1982096 | 64,496 | 89440296 | [C/T] | 0.941 | 0.970 | 0.09041 | 0.50 | C |
| 1054750 | 66,826 | 89442626 | [C/T] | 0.674 | 0.679 | 0.87433 | 0.97 | C |
| 2117137 | 70,606 | 89446406 | [C/T] | 0.476 | 0.510 | 0.33319 | 0.87 | C |
| 1499780 | 71,173 | 89446973 | [C/G] | 0.541 | 0.589 | 0.14521 | 0.82 | C |
| 2117138 | 76,623 | 89452423 | [G/A] | 0.604 | 0.642 | 0.24560 | 0.85 | G |
| 2346840 | 78,368 | 89454168 | [T/G] | 0.247 | 0.222 | 0.35703 | 1.15 | G |
| 2048518 | 79,006 | 89454806 | [C/T] | 0.625 | 0.642 | 0.61569 | 0.93 | C |
| 2048519 | 79,079 | 89454879 | [G/A] | 0.324 | 0.258 | 0.02843 | 1.38 | A |
| 2048520 | 79,349 | 89455149 | [T/C] | 0.008 | 0.006 | 0.80839 | 1.39 | C |
| 2048521 | 79,354 | 89455154 | [G/A] | 0.328 | 0.289 | 0.20537 | 1.20 | A |
| 3762718 | 80,167 | 89455967 | [T/G] | 0.249 | 0.214 | 0.23888 | 1.22 | G |
| 2196083 | 81,647 | 89457447 | [T/C] | 0.263 | 0.234 | 0.29363 | 1.17 | C |
| 1512187 | 82,179 | 89457979 | [T/C] | 0.996 | 0.997 | 0.92664 | 0.81 | T |
| 972030 | 83,599 | 89459399 | [G/T] | 0.401 | 0.329 | 0.02073 | 1.36 | T |
| 2346837 | 87,700 | 89463500 | [T/C] | 0.998 | 0.995 | 0.72396 | 2.99 | C |
| 1036286 | 88,778 | 89464578 | [T/G] | 0.885 | 0.883 | 0.88961 | 1.03 | G |
| 1036285 | 89,162 | 89464962 | [A/G] | 0.293 | 0.262 | 0.28131 | 1.17 | G |
| 1512188 | 91,284 | 89467084 | [A/G] | 0.282 | 0.247 | 0.21329 | 1.20 | G |
| 1512189 | 91,433 | 89467233 | [A/G] | 0.328 | 0.292 | 0.22606 | 1.18 | G |
| 1567657 | 93,620 | 89469420 | [A/G] | 0.528 | 0.581 | 0.09039 | 0.81 | A |
| 1567658 | 93,707 | 89469507 | [T/A] | 0.297 | 0.247 | 0.07396 | 1.29 | A |
| 1028012 | 94,523 | 89470323 | [C/T] | 0.250 | 0.184 | 0.03574 | 1.48 | T |

TABLE 21
Male Replication Allelotyping Results

| | Position | | | | Male A2 | | | Diabetes |
|---|---|---|---|---|---|---|---|---|
| | in SEQ | Chromosome | A1/A2 | Male A2 | Control | Male | Male | Associated |
| dbSNP | ID NO: 1 | Position | Allele | Case AF | AF | p-Value | OR | Allele |
| 3792573 | 225 | 89376025 | [A/C] | 0.001 | 0.005 | 0.64968 | 0.20 | A |
| 3792572 | 1,656 | 89377456 | [A/T] | 0.384 | 0.375 | 0.79686 | 1.04 | T |
| 1398195 | 5,432 | 89381232 | [C/T] | 0.000 | 0.000 | 0.99247 | 0.81 | C |
| 3828462 | 5,652 | 89381452 | [G/T] | 1.000 | 0.997 | 0.77722 | 5.61 | T |
| 3805091 | 6,343 | 89382143 | [G/T] | 0.960 | 0.977 | 0.25633 | 0.57 | G |
| 1512185 | 18,716 | 89394516 | [A/G] | 0.582 | 0.671 | 0.01394 | 0.68 | A |
| 1028013 | 29,369 | 89405169 | [T/C] | 0.245 | 0.211 | 0.28863 | 1.21 | C |
| 987748 | 39,131 | 89414931 | [T/G] | 0.405 | 0.471 | 0.10715 | 0.76 | T |
| 1398197 | 43,529 | 89419329 | [T/C] | 0.902 | 0.905 | 0.90047 | 0.96 | T |
| 1028011 | 43,720 | 89419520 | [T/C] | 0.842 | 0.881 | 0.18933 | 0.72 | T |
| 2881488 | 45,589 | 89421389 | [C/G] | 0.262 | 0.243 | 0.55724 | 1.11 | G |
| 1473598 | 48,922 | 89424722 | [A/C] | 0.000 | 0.000 | 0.99974 | 0.94 | A |
| 1157607 | 51,465 | 89427265 | [C/T] | 0.263 | 0.197 | 0.05327 | 1.45 | T |
| 1157608 | 51,565 | 89427365 | [T/G] | 0.213 | 0.213 | 0.98899 | 1.00 | T |
| 1912965 | 63,433 | 89439233 | [G/A] | 0.613 | 0.635 | 0.56821 | 0.91 | G |
| 1912966 | 63,565 | 89439365 | [A/T] | 0.658 | 0.725 | 0.06349 | 0.73 | A |
| 1982096 | 64,496 | 89440296 | [C/T] | 0.965 | 0.951 | 0.40260 | 1.44 | T |
| 1054750 | 66,826 | 89442626 | [C/T] | 0.702 | 0.712 | 0.78304 | 0.96 | C |
| 2117137 | 70,606 | 89446406 | [C/T] | 0.520 | 0.500 | 0.61596 | 1.08 | T |
| 1499780 | 71,173 | 89446973 | [C/G] | 0.555 | 0.637 | 0.03618 | 0.71 | C |
| 2117138 | 76,623 | 89452423 | [G/A] | 0.639 | 0.665 | 0.48442 | 0.89 | G |
| 2346840 | 78,368 | 89454168 | [T/G] | 0.220 | 0.207 | 0.67619 | 1.08 | G |
| 2048518 | 79,006 | 89454806 | [C/T] | 0.671 | 0.675 | 0.92537 | 0.98 | C |
| 2048519 | 79,079 | 89454879 | [G/A] | 0.290 | 0.225 | 0.07283 | 1.41 | A |
| 2048520 | 79,349 | 89455149 | [T/C] | 0.015 | 0.003 | 0.22937 | 5.64 | C |
| 2048521 | 79,354 | 89455154 | [G/A] | 0.302 | 0.258 | 0.22624 | 1.24 | A |
| 3762718 | 80,167 | 89455967 | [T/G] | 0.212 | 0.184 | 0.38850 | 1.19 | G |
| 2196083 | 81,647 | 89457447 | [T/C] | 0.231 | 0.198 | 0.28951 | 1.22 | C |
| 1512187 | 82,179 | 89457979 | [T/C] | 0.994 | 0.998 | 0.63697 | 0.28 | T |
| 972030 | 83,599 | 89459399 | [G/T] | 0.375 | 0.295 | 0.02255 | 1.43 | T |
| 2346837 | 87,700 | 89463500 | [T/C] | 0.995 | 0.999 | 0.62573 | 0.19 | T |
| 1036286 | 88,778 | 89464578 | [T/G] | 0.866 | 0.919 | 0.04633 | 0.56 | T |
| 1036285 | 89,162 | 89464962 | [A/G] | 0.257 | 0.229 | 0.38929 | 1.16 | G |
| 1512188 | 91,284 | 89467084 | [A/G] | 0.250 | 0.212 | 0.27185 | 1.24 | G |
| 1512189 | 91,433 | 89467233 | [A/G] | 0.306 | 0.269 | 0.28293 | 1.20 | G |
| 1567657 | 93,620 | 89469420 | [A/G] | 0.546 | 0.642 | 0.01337 | 0.67 | A |
| 1567658 | 93,707 | 89469507 | [T/A] | 0.245 | 0.217 | 0.37933 | 1.17 | A |
| 1028012 | 94,523 | 89470323 | [C/T] | 0.233 | untyped | | | |

TABLE 22
Combined Replication Allelotyping Results

| | Position | | | | A2 | | | Diabetes |
|---|---|---|---|---|---|---|---|---|
| | in SEQ | Chromosome | A1/A2 | A2 Case | Control | | | Associated |
| dbSNP | ID NO: 1 | Position | Allele | AF | AF | p-Value | OR | Allele |
| 3792573 | 225 | 89376025 | [A/C] | 0.001 | 0.002 | 0.83108 | 0.48 | A |
| 3792572 | 1,656 | 89377456 | [A/T] | 0.394 | 0.364 | 0.19668 | 1.14 | T |
| 1398195 | 5,432 | 89381232 | [C/T] | 0.000 | 0.000 | 0.95138 | 0.32 | C |
| 3828462 | 5,652 | 89381452 | [G/T] | 0.997 | 0.999 | 0.68794 | 0.29 | G |
| 3805091 | 6,343 | 89382143 | [G/T] | 0.976 | 0.985 | 0.28217 | 0.61 | G |
| 1512185 | 18,716 | 89394516 | [A/G] | 0.577 | 0.639 | 0.00842 | 0.77 | A |
| 1028013 | 29,369 | 89405169 | [T/C] | 0.252 | 0.222 | 0.14710 | 1.18 | C |
| 987748 | 39,131 | 89414931 | [T/G] | 0.406 | 0.468 | 0.01677 | 0.78 | T |
| 1398197 | 43,529 | 89419329 | [T/C] | 0.894 | 0.906 | 0.45175 | 0.87 | T |
| 1028011 | 43,720 | 89419520 | [T/C] | 0.840 | 0.851 | 0.58005 | 0.92 | T |
| 2881488 | 45,589 | 89421389 | [C/G] | 0.273 | 0.247 | 0.21430 | 1.15 | G |
| 1473598 | 48,922 | 89424722 | [A/C] | 0.000 | 0.000 | 0.97548 | 0.56 | A |
| 1157607 | 51,465 | 89427265 | [C/T] | 0.273 | 0.216 | 0.00877 | 1.37 | T |
| 1157608 | 51,565 | 89427365 | [T/G] | 0.230 | 0.222 | 0.70870 | 1.04 | G |
| 1912965 | 63,433 | 89439233 | [G/A] | 0.601 | 0.617 | 0.55456 | 0.93 | G |
| 1912966 | 63,565 | 89439365 | [A/T] | 0.649 | 0.700 | 0.03038 | 0.79 | A |
| 1982096 | 64,496 | 89440296 | [C/T] | 0.952 | 0.963 | 0.36677 | 0.77 | C |
| 1054750 | 66,826 | 89442626 | [C/T] | 0.687 | 0.690 | 0.89251 | 0.98 | C |
| 2117137 | 70,606 | 89446406 | [C/T] | 0.497 | 0.507 | 0.69741 | 0.96 | C |
| 1499780 | 71,173 | 89446973 | [C/G] | 0.547 | 0.605 | 0.02146 | 0.79 | C |
| 2117138 | 76,623 | 89452423 | [G/A] | 0.620 | 0.650 | 0.22149 | 0.88 | G |
| 2346840 | 78,368 | 89454168 | [T/G] | 0.234 | 0.217 | 0.39179 | 1.11 | G |
| 2048518 | 79,006 | 89454806 | [C/T] | 0.647 | 0.653 | 0.79171 | 0.97 | C |
| 2048519 | 79,079 | 89454879 | [G/A] | 0.308 | 0.247 | 0.00827 | 1.36 | A |
| 2048520 | 79,349 | 89455149 | [T/C] | 0.011 | 0.005 | 0.32737 | 2.43 | C |
| 2048521 | 79,354 | 89455154 | [G/A] | 0.316 | 0.278 | 0.10828 | 1.20 | A |
| 3762718 | 80,167 | 89455967 | [T/G] | 0.232 | 0.204 | 0.19864 | 1.18 | G |
| 2196083 | 81,647 | 89457447 | [T/C] | 0.248 | 0.222 | 0.19882 | 1.16 | C |
| 1512187 | 82,179 | 89457979 | [T/C] | 0.995 | 0.997 | 0.72710 | 0.57 | T |
| 972030 | 83,599 | 89459399 | [G/T] | 0.389 | 0.318 | 0.00195 | 1.37 | T |
| 2346837 | 87,700 | 89463500 | [T/C] | 0.997 | 0.997 | 0.97694 | 1.05 | C |
| 1036286 | 88,778 | 89464578 | [T/G] | 0.876 | 0.895 | 0.26522 | 0.83 | T |
| 1036285 | 89,162 | 89464962 | [A/G] | 0.276 | 0.251 | 0.23640 | 1.14 | G |
| 1512188 | 91,284 | 89467084 | [A/G] | 0.267 | 0.235 | 0.14196 | 1.18 | G |
| 1512189 | 91,433 | 89467233 | [A/G] | 0.318 | 0.284 | 0.13534 | 1.17 | G |
| 1567657 | 93,620 | 89469420 | [A/G] | 0.537 | 0.602 | 0.00739 | 0.77 | A |
| 1567658 | 93,707 | 89469507 | [T/A] | 0.273 | 0.237 | 0.08628 | 1.21 | A |
| 1028012 | 94,523 | 89470323 | [C/T] | 0.242 | 0.184 | 0.01783 | 1.42 | T |

Allelotyping results were considered particularly significant with a calculated p-value of less than or equal to 0.05 for allelotype results. These values are indicated in bold. The allelotyping p-values were plotted in FIGS. 1D-F for females, males and combined, respectively. The position of each SNP on the chromosome is presented on the x-axis. The y-axis gives the negative logarithm (base 10) of the p-value comparing the estimated allele in the case group to that of the control group. The minor allele frequency of the control group for each SNP designated by an X or other symbol on the graphs in FIGS. 1D-F can be determined by consulting Tables 20, 21 and 22. For example, the left-most X on the left graph is at position 89376025. By proceeding down the Table from top to bottom and across the graphs from left to right the allele frequency associated with each symbol shown can be determined.

Secondary Phenotype Association

A secondary phenotype analysis was performed to look for associations between EPHA3 SNPs and additional diabetes-related phenotypes. This analysis revealed an association between rs1512183 and C peptide levels both in fasting (by 27%, P<0.08) and post-prandial states (15%, P<0.009). This association exists both within the male and female diabetic cases. C peptide (in nmol/L) is a measure of endogenous insulin production. C-peptide blood levels can indicate whether or not a person is producing insulin and roughly how much. This is because insulin is initially synthesized in the form of proinsulin. In this form, the alpha and beta chains of active insulin are linked by a third polypeptide chain called the connecting peptide, or c-peptide, for short. Because both insulin and c-peptide molecules are secreted, for every molecule of insulin in the blood, there is one of c-peptide. Therefore, levels of c-peptide in the blood can be measured and used as an indicator of insulin production in those cases where exogenous insulin (from injection) is present and mixed with endogenous insulin (that produced by the body) a situation that would make meaningless a measurement of insulin itself. The c-peptide test can also be used to help assess if high blood glucose is due to reduced insulin production or to reduced glucose intake by the cells.

A significant increase in C peptide levels exist in the TT homozygotes, where T is the allele associated with type II diabetes. Therefore, this polymorphism results in an insulin resistant state, and compensatory hyperinsulinemia is observed.

Identification of a Coding, Non-Synonymous SNP at Amino Acid Position 924 in EPHA3

A SNP was identified by fragmentation at chromosome position 89442594, which codes for a non-synonymous SNP at amino acid position 924 in the EPHA3 protein (see SEQ ID NO: 4). Fragmentation is described by Hartmer et al. (Nucleic Acids Res. 2003 May 1; 31(9):e47), Bocker (Bioinformatics. 2003 July; 19 Suppl 1:I44-I53), in U.S. patent application 60/466,006 filed 25 Apr. 2003 and in U.S. patent application 60/429,895 filed 27 Nov. 2002. The following primers were used for fragmentation analysis of this particular SNP: AGTTCCTGCCGATGTTAGT and CTGTGGAAATCTGGCTATT. From fragmentation, the following genotypes were determined from the 12 individuals (6 cases and 6 controls):

TABLE 23

| Sample_Type | Sample_Gender | Sample_Source | Allele 1 | Allele 2 |
|---|---|---|---|---|
| cas | F | TBN | C | C |
| cas | F | TBN | C | C |
| cas | F | TBN | C | C |
| cas | M | TBN | C | C |
| cas | M | TBN | C | C |
| cas | M | TBN | C | C |
| con | F | TBN | T | T |
| con | F | TBN | T | T |
| con | F | TBN | Unk | Unk |
| con | M | TBN | T | T |
| con | M | TBN | T | T |
| con | M | TBN | T | T |

More specifically, the thymine/cytosine polymorphic variation at position 201 of exon 16 in EPHA3 codes for a tryptophan (W) to arginine (R) amino acid change at position 924 of the polypeptide sequence (see SEQ ID NO: 4) The W924R change occurs in the SAM domain, and represents a dramatic change as typtophan is highly hydrophobic and arginine is hydrophilic and positively charged under physiological conditions.

The SNP at chromosome position 89442594 is polymorphic and was genotyped in the German diabetic population samples described herein using the primers provided in Tables 24 and 25.

TABLE 24

| | | |
|---|---|---|
| Ref | Forward | Reverse |
| SNP ID | PCR primer | PCR primer |

| | | |
|---|---|---|
| AB | CCGTGAAGATTTCCTTGCAG | GTGGATATCACTACCTTCCG |

TABLE 25

| | | |
|---|---|---|
| Reference | Extend | Term |
| SNP ID | Probe | Mix |

| | | |
|---|---|---|
| AB | CTTGCAGTGTGCTGTCC | ACT |

Tables 26, 27 and 28 show the genotyping results for the SNP at position 89442594 in the Discovery and Newfoundland cohorts for females, males and combined.

TABLE 26
Female Genotyping Results

| SNP | | | | | Odds |
|---|---|---|---|---|---|
| Reference | Population | F case | F control | p-value | Ratio |
| AB | Discovery | T = 0.529 | T = 0.639 | 0.000563 | 1.58 |
| | | C = 0.471 | C = 0.361 | | |
| AB | Newfoundland | T = 0.569 | T = 0.605 | 0.459 | 1.16 |
| | | C = 0.431 | C = 0.395 | | |

TABLE 27
Male Genotyping Results

| SNP | | | | | Odds |
|---|---|---|---|---|---|
| Reference | Population | M case | M control | p-value | Ratio |
| AB | Discovery | T = 0.573 | T = 0.593 | 0.525 | 1.09 |
| | | C = 0.427 | C = 0.407 | | |
| AB | Newfoundland | T = 0.602 | T = 0.549 | 0.284 | 0.8 |
| | | C = 0.398 | C = 0.451 | | |

TABLE 28
Combined Genotyping Results

| SNP | | | | | Odds |
|---|---|---|---|---|---|
| Reference | Population | A case | A control | p-value | Ratio |
| AB | Discovery | T = 0.551 | T = 0.616 | 0.00409 | 1.3 |
| | | C = 0.449 | C = 0.384 | | |
| AB | Newfoundland | T = 0.585 | T = 0.577 | 0.813 | 0.97 |
| | | C = 0.415 | C = 0.423 | | |

The C allele is more frequent in case samples and codes for an arginine at position 924 of EPHA3, therefore arginine is associated with an increased risk of diabetes, while tryptophan is associated with a decreased risk. The tryptophan allele is not conserved amongst species, as the mouse version of the gene possess an arginine at this locus.

Deep Sequencing Reveals Non-synonymous SNP at Amino Acid Position 914 in EPHA3

Deep sequencing was performed on EPHA3 to identify novel SNPs located in the gene. Methods of deep sequencing (or high-throughput comparative sequence analysis) are described by Hartmer et al. (Nucleic Acids Res. 2003 May 1; 31(9):e47) and by Bocker. (Bioinformatics. 2003 July; 19 Suppl 1:I44-I53). Deep sequencing of EPHA3 revealed an allelic variant in exon 16 that codes for an arginine to histidine change at amino acid position 914 of transcript variant 1 of EPHA3 (chromosome position 89442565 of Build 34). See Table 23, below, which shows the allele frequencies for male and female cases. The forward primer used is AGTTCCTGCCGATGTTAGT and the reverse primer used is CTGTGGAAATCTTGGCTATT. Amino acid 914 is located in the SAM domain and is not conserved amongst species. The mouse and rat versions of the gene possess a histidine at this locus and the chicken version of the gene possesses an arginine at the position. Both amino acids are hydrophilic, although arginine normally is fully charged under physiological conditions while histidine normally is partially charged.

Tables 29, 30 and 31 show the genotyping results for the SNP at position 89442565 in the Discovery and Newfoundland cohorts for females, males and combined.

TABLE 29
Female Genotyping Results

| SNP | | | | | Odds |
|---|---|---|---|---|---|
| Reference | Population | F case | F control | p-value | Ratio |
| AA | Discovery | G = 0.895 | G = 0.898 | 0.848 | 1.04 |
| | | A = 0.105 | A = 0.102 | | |
| AA | Newfoundland | G = 0.919 | G = 0.917 | 0.939 | 0.98 |
| | | A = 0.081 | A = 0.083 | | |

TABLE 30
Male Genotyping Results

| SNP | | | | | Odds |
|---|---|---|---|---|---|
| Reference | Population | F case | F control | p-value | Ratio |
| AA | Discovery | G = 0.900 | G = 0.883 | 0.393 | 0.84 |
| | | A = 0.100 | A = 0.117 | | |
| AA | Newfoundland | G = 0.902 | G = 0.893 | 0.757 | 0.91 |
| | | A = 0.098 | A = 0.107 | | |

TABLE 31
Combined Genotyping Results

| SNP | | | | | Odds |
|---|---|---|---|---|---|
| Reference | Population | F case | F control | p-value | Ratio |
| AA | Discovery | G = 0.897 | G = 0.890 | 0.627 | 0.93 |
| | | A = 0.103 | A = 0.110 | | |
| AA | Newfoundland | G = 0.911 | G = 0.909 | 0.929 | 0.98 |
| | | A = 0.089 | A = 0.091 | | |

### Example 6

### EPHA3 Expression Profile

Expression of EPHA3 isoforms and its ligands ephrin-A2 and ephrin-A5 was determined in a panel of cDNA generated from tumorigenic cell lines and normal tissues. The transmembrane isoform of EPHA3, isoform 1, was expressed at higher levels than the soluble isoforms, isoform 2. Specifically, EPHA3, isoform 1, expression was initially detected in normal brain, adipose prostate, liver, cardiac muscle tissues, and several tumorigenic cell lines of neuronal, hematopoietic, mammary and prostate origins. Ephrin-A5 was expressed at higher levels than ephrin-A2 in the same panel of cDNA, and expression in normal tissue was detected for ephrin-A5 in adipose, brain and liver tissues. To analyze these expressions in greater detail, additional cDNA was generated from new samples of skeletal muscle, liver and pancreas. Full length EPHA3 was detected in adipose, two liver tissues, pancreas, skeletal muscle and prostate. Ephrin-A5 expression was detected in adipose, skeletal muscle and prostate, while ephrin-A2 was only detected in liver tissue.

Immunohistochemistry.

Blood glucose level is tightly regulated by the interplay of several tissues including the brain, liver, pancreas, small intestine, skeletal muscle and adipose tissues. Changes in blood glucose level is sensed by the pancreas, which results in the secretion of hormones that reinstate normal blood glucose levels through the stimulation of glucose production in the liver or absorption from the intestine, and uptake and metabolism in peripheral tissues, particularly adipose and skeletal muscles Several of these tissues are composed of a small percentage of specialized cells that are responsible for these specific functions. As a result, detection of expression of candidate genes that may be involved in the pathology of diabetes can be overlooked when looking at whole tissue. To determine specific cellular expression within a tissue, gene expression was detected using immunohistochemistry.

Methods

Mice were perfused with 4% paraformaldehyde/PBS solution. After perfusion, pancreas, and white adipocyte tissue from the peritoneal cavity, was dissected out, and additionally fixed for 3 hours in 4% paraformaldehyde/PBS solution. Pancreatic tissues were then washed with PBS, and sucrose treated overnight in sucrose/PBS solution. After rinsing in PBS, tissues were embedded in OCT, and frozen overnight at −80 deg. 7u tissue sections were generated using a cryosection, and stored at −0 deg. For white adipocyte tissues, tissues were washed with PBS after additional fixing, and dehydrated in a series of ethanol and xylene treatments. Adipocytes were then embedded in paraffin blocks.

Prior to staining, cryosections were thawed at room temperature and sections washed three times in PBS. For paraffin sections, sections were deparaffinized with xylene and ethanol treatments, and subsequently hydrated with PBS . . . . Sections were blocked in 4% donkey serum in PBS (blocking solution) for one hour. Blocking solution was aspirated, and slide incubated with primary antibodies, anti-EPHA3, -ephrin-M, and -ephrin-A5 at 1:50 and anti-insulin at 1:100 in blocking solution for 24 hours. Samples were washed three times in PBS. After washes, slides stained with anti-EPHA3, -ephrin-A2, and -ephrin-A5 were incubated with secondary antibodies, anti-TRITC and sections stained with anti-insulin were incubated with an anti-FITC secondary antibody for one hour. Slides were washed three times. Excess fluid was removed from sections, and mounted using a non-fading mounting media.

Results

Using primary antibodies specific to EPHA3, ephrin-A5 and ephrin-M, expression was detected in mouse adipocytes. Sections of mouse pancreas probed with primary antibodies against EPHA3 and ephrin-A5 showed specific fluorescent signal in the islet regions of the pancreas. However, sections of mouse pancreas stained with anti-ephrin-A2 antibodies did not show any expression. Pancreatic islets are cellular structures within the pancreas that contain insulin-secreting cells, and therefore stain positive for insulin. To verify specificity of staining in islets, double staining with antibodies against insulin and EPHA3, or with ephrin-A5, was performed.

Results showed specific staining and colocalization of insulin with ephrin-A5, and with EPHA3 in mouse pancreatic islets indicating expression in this area of the pancreas. It was determined EPHA3 and ephrin-A5, but not ephrin-A2, were expressed in islets of mouse pancreas as demonstrated by single staining with EPHA3 and ephrin-A5, and co-staining with insulin EPHA3 ephrin-A5, and ephrin-A2 expression also were detected in mouse white adipose tissue. The absence of fluorescent signal from sections stained with secondary antibodies alone underscore the specificity of these results. The specific expression of EPHA3 and its ligands, ephrin-A5 and ephrin-A2, in both the islets of pancreas and white adipose tissue—tissues centrally involved in the control of glucose and energy homeostasis—further indicates a role for EPHA3 and its ligands in type II diabetes.

### Example 7

### Glucose Uptake Assay

One of the many responses of adipocytes and muscle cells after exposure, to insulin is the transport of glucose intracellularly. This transport is mediated by GLUT4, an insulin-regulatable glucose transporter. Insulin binding to insulin receptors on the cell surface results in autophosphorylation and activation of the intrinsic tyrosine kinase activity of the insulin receptor. Phosphorylated tyrosine residues on the insulin receptor and its endogenous targets activate several intracellular signaling pathways that eventually lead to the translocation of GLUT4 from intracellular stores to the extracellular membrane.

Methods

Cells were plated in 6-well dishes, and grown to confluency. Cells were then differentiated with DMEM plus 10% fetal calf serum (FCS), 10 ug/mL insulin, 390 ng/mL dexamethasone and 112 ug/mL isobutylmethylxanthine for 2 days. After 2 days of differentiation, media was changed to maintenance media DMEM plus 10% FCS and 5 ug/mL insulin. Media was changed every 2 days thereafter. Cells were assayed for insulin-mediated glucose uptake 10 days after differentiation. On the day of the assay, cells were washed once with PBS, and serum starved by adding 2 mL of DMEM plus 2 mg/mL BSA for 3 hours. During serum starvation, recombinant rat ephrin-A5/Fc chimeric ligand was preclustered. In a solution of PBS plus 2 mg/mL BSA, recombinant rat ephrin-A5/Fc chimera was added to a concentration of 1.75 ug/mL, and anti-human IgG, Fc γ fragment specific antibody to a final concentration of 17.5 ug/mL. After 3 hours of serum starvation, media was replaced with 2 mL of preclustered ephrin-A5, and incubated for 10, 40 and 90 min at 37 deg. After 10 min, porcine insulin was added to a final concentration of 100 nM for 10 min at 37 deg. For every 2 mL of media, 100 uL of PBS-2-DOG label was added to, give a final concentration of 2 uCi. Cells were immediately placed on ice, washed three times with ice cold PBS, and lysed with 0.7 mL of 0.2 N NaOH. Lysates were read in a Wallac 1450 Microbeta Liquid Scintillation and Luminescence Counter.

Results

Differentiated 3T3-L1, when treated with 100 nM insulin for 10 minutes, resulted in a 22-fold increase in uptake of radioactive glucose. When cells were pretreated with pre-clustered ephrin-A5 for 10 min prior to insulin treatment, a 20% decrease in uptake of radioactive glucose was observed. However, when pretreated for 40 minutes, no change in glucose uptake compared to cells treated with insulin alone was observed. The inhibition was reinstated after 90 min of preincubation with pre-clustered ephrin-A5, where a 15% decrease in glucose uptake was observed. These results fall within a range of inhibition seen in similar metabolic-related experiments performed by others. For example, a range of inhibition of 18%-35% was reported for the inhibition of AKT using siRNA (Katome et al. JBC, July 2003; 278:28312-28323). AKT is downstream of PI3-Kinase which is one of the substrates for the insulin receptor. In addition, Cho et al. (Han Cho et al. Science 2001 Jun. 1; 292:1728-1731) report that target disruption of AKT2 causes insulin resistance and type II diabetes phenotype.

Ephrin-A5 binds with high affinity, to EPHA3. This binding has been shown to activate the intrinsic receptor tyrosine kinase activity of EPHA3. This activation results in inhibition of one of the steps leading to the translocation of GLUT4 to the membrane, or of the insulin mediated increase in the intrinsic transport activity of GLUT4. The cumulative and overall decrease in glucose transport as a result of EPHA3 activation can lead to chronic hyperglycemia and eventual onset of diabetes.

### Example 8

### Triacylglycerol (TG) Assay

A direct metabolic consequence of glucose transport intracellularly is its incorporation into the fatty acid and glycerol moieties of triacylglycerol (TG). TGs are highly concentrated stores of metabolic energy, and are the major energy reservoir of cells. In mammals, the major site of accumulation of triacylglycerols is the cytoplasm of adipose cells. Adipocytes are specialized for the synthesis, and storage of TG, and for their mobilization into fuel molecules that are transported to other tissues through the bloodstream. It is likely that changes in the transport of glucose intracellularly can affect cytoplasmic stores of triacylglycerols.

Methods

Cells were plated in 6-well dishes, and grown to confluency. When cells reached confluency, cells were differentiated with DMEM plus 10% fetal calf serum (FCS), 10 ug/mL insulin, 390 ng/mL dexamethasone and 112 ug/mL isobutylmethylxanthine for 2 days. After 2 days of differentiation, media was changed to maintenance media DMEM plus 10%. FCS and 5 ug/1 nm insulin. On the day of the assay (day 9 post-differentiation), cells were washed once with PBS, and serum starved by adding 2 mL of DMEM plus 2 mg/ml BSA for 3 hours. During serum starvation, recombinant rat ephrin-A5/Fc chimeric ligand was preclustered. In a solution of PBS plus 2 mg/ml BSA recombinant rat ephrin-A5/Fc chimeria was added to a concentration of 1.75 ug/mL, and anti-human IgG, Fc γ fragment specific antibody to a final concentration of 17.5 ug/mL. After 3 hours of serum starvation, media was replaced with pre-clustered ephrin-A5 solution, and incubated for 10 minutes at 37 degrees. Cells were then treated with 100 nM porcine insulin for 2 hours at 37 degrees. Cells were immediately placed on ice, and washed twice with ice cold PBS. Cells were lysed with 1% SDS, 1.2 mM Tris, pH 7.0 and heat treated at 95 degrees for 5 minutes. Samples were assayed using INFINITY Tryglyceride reagent. In a 96-well, flat bottom, transparent microtiter plate, 3 uL of sample were added to 300 uL of INFINITY Triglyceride Reagent. Samples were incubated at room temperature for 10 minutes. The assay was read at 500-550 nm.

Results

Differentiated 3T3-L1 treated with 100 nM insulin showed an increase in TG stores by about 23%. When cells were pretreated with ephrin-A5 for 10 minutes prior to insulin treatment a 10% decrease in insulin-mediated TG stores was observed. Adipocytes transport glucose intracellularly when exposed to insulin. Transported glucose are primarily converted to triglycerides, the primary source of cellular energy for adipocytes. Because ephrin-A5 binds with high affinity to EPHA3, pretreatment with ephrin-A5 is thought to activate EPHA3, which then inhibits glucose transport intracellularly. The decrease in glucose imported contributes to the observed decrease in measured intracellular triglycerides. Alternatively, activation of EPHA3 by ephrin-A5 binding results in the transcriptional inhibition of genes necessary for the conversion of glucose to triglyceride. This downregulation of genes necessary for lipogenesis contributes to the observed decrease in measured TG.

### Example 9

### Quantitative Assessment of mResistin Levels

Resistin is a secreted factor specifically expressed in white adipocyte. It was initially discovered in a screen for genes downregulated in adipocytes by PPAR gamma, and expression was found to be attenuated by insulin. Elevated levels of resistin have been measured in genetically obese, and high fat fed obese mice. It is therefore thought that resistin contributes to peripheral tissue insulin unresponsiveness, one of the pathological hallmarks of diabetes.

Methods

3T3-L1 cells were differentiated for 3 days as previously described and maintained for three days prior to splitting. At day 5 post-differentiation, differentiated cells were plated in 10 cm dish at a cell density of 3×10⁶ cells. Cells were then serum starved on day 7 after initiation of differentiation. On day 8, cells were treated with pre-clustered recombinant rat ephrin-A5/Fc chimera as described above for 10 min and treated with 10 nM insulin for 2 hours. Cells were harvested, mRNA extracted using magnetic DYNAL beads and reverse transcribed to cDNA using. Superscript First-Strand Synthesis as described by the manufacturer. The following primers: forward primer; 5′ GTC GCT TCC TGA TGT CGG TCA 3′, and reverse primer, 5′ GGC CAG CCT GGA CTA TAT GAG 3′, were used in 15 uL PCR reaction using 55 deg annealing temperature and 30 cycles of amplification.

Results

Differentiated 3T3-L1 cells treated with insulin showed a decrease in resistin mRNA levels. When cells were pretreated with ephrin-A5 prior to insulin treatment, the observed inhibition in resistin levels as a result of insulin treatment was relieved. C/EBP alpha, a transcription factor upregulated in the early steps of adipocyte differentiation, has been found to positively regulate resistin mRNA expression. In addition, overexpression of PPAR gamma, and PI3-kinase and Akt, signaling intermediates downstream of the insulin receptor, down-regulates resistin levels. It is formally possible that EPHA3 activation as a result of ephrin-A5 binding results in the inactivation of the activity of PPAR gamma, or the inhibition of the insulin-PI3-K-Akt pathway, or may conversely activate positive regulators such as C/EBP alpha. The additional effect of an increase in secreted resistin levels as a result of ephrin-A5 treatment can result in the loss or decrease in sensitivity of peripheral tissues, such as adipocyte, to insulin. This loss or decrease in insulin sensitivity can affect eventual transport and metabolism of glucose and result in a diabetic phenotype.

### Example 10

### In Vitro Tests of Metabolic-Related Activity

In vitro assays described hereafter are useful for identifying therapeutics for treating human diabetes. As used in Examples hereafter directed to in vitro assays, rodent models and studies in humans, the term “test molecule” refers to a molecule that is added to a system, where an agonist effect, antagonist effect, or lack of an effect of the molecule on EPHA3 function or a related physiological function in the system is assessed. An example of a test molecule is a test compound, such as a test compound described in the section “Compositions Comprising Diabetes-Directed Molecules” above. Another example of a test molecule is a test peptide, which includes, for example, an EPHA3-related test peptide such as a soluble, extracellular form of EPHA3 (e.g., isoform b of EPHA3 and the extracellular domain of isoform a of EPHA3), an EPHA3 binding partner or ligand (e.g., Ephrin-A2 or Ephrin-A5), or a functional fragment of the foregoing. A concentration range or amount of test molecule utilized in the assays and models is selected from a variety of available ranges and amounts. For example, a test molecule sometimes is introduced to an assay system in a concentration range between 1 nanomolar and 100 micromolar or a concentration range between 1 nanograms/mL and 100 micrograms/mL. An effect of a test molecule on EPHA3 function or a related physiological function often is determined by comparing an effect in a system administered the test molecule against an effect in system not administered the test molecule. Described directly hereafter are examples of in vitro assays.

Effect on Muscle Differentiation

C2C12 cells (murine skeletal muscle cell line; ATCC CRL 1772, Rockville, Md.) are seeded sparsely (about 15-20%) in complete DMEM (w/glutamine, pen/strep, etc)+10% FCS. Two days later they become 80-90% confluent. At this time, the media is changed to DMEM+2% horse serum to allow differentiation. The media is changed daily. Abundant myotube formation occurs after 34 days of being in 2% horse serum, although the exact time course of C2C12 differentiation depends on how long they have been passaged and how they have been maintained, among other factors.

To test the effect of the presence of test molecules on muscle differentiation, test molecules (e.g., test peptides added in a range of 1 to 2.5 μg/mL) are added the day after seeding when the cells are still in DMEM with 10% FCS. Two days after plating the cells (one day after the test molecule was first added), at about 80-90% confluency, the media is changed to DMEM+2% horse serum plus the test molecule.

Effect on Muscle Cell Fatty Acid Oxidation

C2C12 cells are differentiated in the presence or absence of 2 μg/mL test molecules for 4 days. On day 4, oleate oxidation rates are determined by measuring conversion of 1-¹⁴C-oleate (0.2 mM) to ¹⁴CO₂ for 90 min. This experiment can be used to screen for active polypeptides and peptides as well as agonists and antagonists or activators and inhibitors of EPHA3 polypeptides or binding partners.

The effect of test molecules on the rate of oleate oxidation can be compared in differentiated C2C12 cells (murine skeletal muscle cells; ATCC, Manassas, Va. CRL-1772) and in a hepatocyte cell line (Hepal-6; ATCC, Manassas, Va. CRL-1830). Cultured cells are maintained according to manufacturer's instructions. The oleate oxidation assay is performed as previously described (Muoio et al (1999) Biochem J 338; 783-791). Briefly, nearly confluent monocytes are kept in low serum differentiation media (DMEM, 2.5% Horse serum) for 4 days, at which time formation of myotubes becomes maximal. Hepatocytes are kept in the same DMEM medium supplemented with 10% FCS for 2 days. One hour prior to the experiment the media is removed and 1 mL of preincubation media (MEM, 2.5% Horse serum, 3 mM glucose, 4 mM Glutamine, 25 mM Hepes, 1% FFA free BSA, 0.25 mM Oleate, 5 μg/mL gentamycin) is added. At the start of the oxidation experiment ¹⁴C-Oleic acid (1 μCi/mL, American Radiolabelled Chemical Inc., St. Louis, Mo.) is added and cells are incubated for 90 min at 37° C. in the absence/presence of test molecule (e.g., 2.5 μg/mL of EPHA3-related test peptide). After the incubation period 0.75 mL of the media is removed and assayed for ¹⁴C-oxidation products as described below for the muscle EFA oxidation experiment.

Triglyceride and Protein Analysis Following Oleate Oxidation in Cultured Cells

Following-transfer of media for oleate oxidation assay, cells are placed on ice. To determine triglyceride and protein content, cells are washed with 1 mL of 1×PBS to remove residual media. To each well 300 μL of cell dissociation solution (Sigma) is added and incubated at 37° C. for 10 min. Plates are tapped to loosen cells, and 0.5 mL of 1×PBS was added. The cell suspension is transferred to an Eppendorf tube, each well is rinsed with an additional 0.5 mL of 1×PBS, and is transferred to the appropriate Eppendorf tube. Samples are centrifuged at 1000 rpm for 10 minutes at room temperature. Each supernatant is discarded and 750 μL of 1×PBS/2% CHAPS is added to cell pellet. The cell suspension is vortexed and placed on ice for 1 hour. Samples are then centrifuged at 13000 rpm for 20 min at 4° C. Each supernatant is transferred to a new tube and frozen at −20° C. until analyzed. Quantitative measure of triglyceride level in each sample is determined using Sigma Diagnostics GPO-TRINDER enzymatic kit. The procedure outlined in the manual is followed, with the following exceptions: the assay is performed in 48 well plate, 350 μL of sample volume is assayed, a control blank consists of 350 μL PBS/2% CHAPS, and a standard contains 10 μL standard provide in the kit with 690 μL PBS/2% CRAPS. Analysis of samples is carried out on a Packard Spectra Count at a wavelength of 550 nm. Protein analysis is carried out on 25 μL of each supernatant sample using the BCA protein assay (Pierce) following manufacturer's instructions. Analysis of samples is carried out on a Packard Spectra Count at a wavelength of 550 nm.

Stimulation of Insulin Secretion in HIT-T15 Cells

HIT-T15 (ATCC CRL#1777) is an immortalized hamster insulin-producing cell line. It is known that stimulation of cAAP in HIT-T15 cells causes an increase in insulin secretion when the glucose concentration in the culture media is changed from 3 mM to 15 mM. Thus, test molecules also are tested for their ability to stimulate glucose-dependent insulin secretion (GSIS) in HIT-T15 cells. In this assay, 30,000 cells/well in a 12-well plate are incubated in culture media containing 3 mM glucose and no serum for 2 hours. The media is then changed, wells receive media containing either 3 mM or 15 mM glucose, and in both cases the media contains either vehicle (DMSO) or test molecule at a concentration of interest. Some wells receive media containing 1 micromolar forskolin as a positive control. All conditions are tested in triplicate. Cells are incubated for 30 minutes, and the amount of insulin secreted into the media is determined by ELISA, using a kit from either Peninsula Laboratories (Cat # ELIS-7536) or Crystal Chem. Inc. (Cat # 90060).

Stimulation of Insulin Secretion in Isolated Rat Islets

As with HT-TI 5 cells, it is known that stimulation of cAMP in isolated rat islets causes an increase in insulin secretion when the glucose concentration in the culture media is changed from 60 mg/dl to 300 mg/dl. Ligands are tested for their ability to stimulate GSIS in rat islet cultures. This assay is performed as follows:
1. Select 75-150 islet equivalents (IEQ) for each assay condition using a dissecting microscope. Incubate overnight in low-glucose culture medium. (Optional.)
2. Divide the islets evenly into triplicate samples of 25-40 islet equivalents per sample. Transfer to 40 μm mesh sterile cell strainers in wells of a 6-well plate with 5 ml of low (60 mg/dl) glucose Krebs-Ringers Buffer MRB) assay medium.
3. Incubate 30 minutes (1 hour if overnight step skipped) at 37° C. and 5% CO₂. Save the supernatants if a positive control for the RIA is desired.
4. Move strainers with islets to new wells with 5 ml/well low glucose KRB. This is the second pre-incubation and serves to remove residual or carryover insulin from the culture medium. Incubate 30 minutes.
5. Move strainers to next wells (Low 1) with 4 or 5 ml low glucose KRB. Incubate at 37° C. for 30 minutes. Collect supernatants into low-binding polypropylene tubes pre-labelled for identification and keep cold.
6. Move strainers to high glucose wets (300 mg/dl, which is equivalent to 16.7 mM). Incubate and collect supernatants as before. Rinse islets in their strainers in low-glucose to remove residual insulin. If the rinse if to be collected for analysis, use one rinse well for each condition (i.e. set of triplicates.)
7. Move strainers to final wells with low-glucose assay medium (Low 2). Incubate and collect supernatants as before.
8. Maintaining a cold temperature, centrifuge supernatants at 1800 rpm for 5 minutes at 4-8° C. to remove small islets/islet pieces that escape the 40 mm mesh. Remove all but lower 0.5-1 ml and distribute in duplicate to pre-labelled low-binding tubes. Freeze and store at <−20° C. until insulin concentrations can be determined.
9. Insulin determinations are performed as above, or by Linco Labs as a custom service, using a rat insulin RIA (Cat. #RI-13K).

### Example 11

### Effect of EPHA3-Related Test Peptides on Mice Fed a High-Fat Diet

Following is a representative rodent model for identifying therapeutics for treating human diabetes. Experiments are performed using approximately 6 week old C57Bl/6 mice (8 per group). All mice are housed individually. The mice are maintained on a high fat diet throughout each experiment. The high fat diet (cafeteria diet; D12331 from Research Diets, Inc.) has the following composition: protein kcal % 16, sucrose kcal % 26, and fat kcal % 58. The fat is primarily composed of coconut oil, hydrogenated.

After the mice are fed a high fat diet for 6 days, micro-osmotic pumps are inserted using isoflurane anesthesia, and are used to provide test molecule, saline, and a control molecule (e.g., an irrelevant peptide) to the mice subcutaneously (s.c.) for 18 days. For example, EPHA3-related test peptides are provided at doses of 100, 50, 25, and 2.5 μg/day and an irrelevant peptide is provided at 10 μg/day. Body weight is measured on the first, third and fifth day of the high fat diet, and then daily after the start of treatment. Final blood samples are taken by cardiac puncture and are used to determine triglyceride (TG), total cholesterol (TC), glucose, leptin, and insulin levels. The amount of food consumed per day is also determined for each group.

### Example 12

### In Vivo Effects of Test Molecules on Glucose Homeostasis in Mice

Following are representative rodent models for identifying therapeutics for treating human diabetes.

Oral Glucose Tolerance Test (oGTT)

Male C57bl/6N mice at age of 8 weeks are fasted for 18 hours and randomly grouped (n=11) to receive an EPHA3-related test peptide, a test molecule at indicated doses, or with control extendin-4 (ex-4, 1 mg/kg), a GLP-1 peptide analog known to stimulate glucose-dependent insulin secretion. Thirty minutes after administration of EPHA3-related test peptides, test compound and control ex-4, mice are administered orally with dextrose at 5 g/kg dose. Test molecule is delivered orally via a gavage needle (p.o. volume at 100 ml). Control Ex-4 is delivered intraperitoneally. Levels of blood glucose are determined at regular time points using Glucometer Elite XL (Bayer).

Acute Response of db Mice to Test Molecule

Male db mice (C57BL/KsOlahsd-Leprdb, diabetic, Harlan) at age of 10 weeks are randomly grouped (n=6) to receive vehicle (oral gavage), EPHA3-related test peptides (at concentration of interest), test molecule (e.g., 60 mg/kg, or another concentration of interest, oral savage), or Ex-4 (1 mg/kg, intraperitoneally). After peptide and/or compound administration, food is removed and blood glucose levels are determined at regular time intervals. Reduction in blood glucose at each time point may be expressed as percentage of original glucose levels, averaged from the number of animals for each group. Results show the effect EPHA3-related test peptides and test molecules for improving glucose homeostasis in diabetic animals.

### Example 13

### Effect of Test Molecules on Plasma Free Fatty Acid in C57 BL/6 Mice

Following is a representative rodent model for identifying therapeutics for treating human diabetes. The effect of test molecules on postprandial lipemia (PPL) in normal C57BL6/J mice is tested.

The mice used in this experiment are fasted for 2 hours prior to the experiment after which a baseline blood sample is taken. All blood samples are taken from the tail using EDTA coated capillary tubes (50 μL each time point). At time 0 (8:30 AM), a standard high fat meal (6 g butter, 6 g sunflower oil, 10 g nonfat dry milk, 10 g sucrose, 12 mL distilled water prepared fresh following Nb#6, JF, pg. 1) is given by gavage (vol.=1% of body weight) to all animals.

Immediately following the high fat meal, a test molecule is injected i.p. in 100 μL saline (e.g., 25 μg of test peptide). The same dose (25 μg/mL in 100 μL) is again injected at 45 min and at 1 hr 45 min. Control animals are injected with saline (3×100 μL). Untreated and treated animals are handled in an alternating mode.

Blood samples are taken in hourly intervals, and are immediately put on ice. Plasma is prepared by centrifugation following each time point. Plasma is kept at −20° C. and free fatty acids (FFA), triglycerides (TG) and glucose are determined within 24 hours using standard test kits (Sigma and Wako). Due to the limited amount of plasma available, glucose is determined in duplicate using pooled samples. For each time point, equal volumes of plasma from all 8 animals per treatment group are pooled.

### Example 14

### Effect of Test Molecules an Plasma FFA, TG and Glucose in C57 BL/6 Mice

Following is a representative rodent model for identifying therapeutics for treating human diabetes. The experimental procedure is similar to that described in Example 13. Briefly, 14 mice are fasted for 2 hours prior to the experiment after which a baseline blood sample is taken. All blood samples are taken from the tail using EDTA coated capillary tubes (50 μL each time point). At time 0 (9:00 AM), a standard high fat meal (see Example 4) is given by gavage (vol.=1% of body weight) to all animals. Immediately following the high fat meal, 4 mice are injected with a test molecule i.p. in 100 μL saline (e.g., 25 μg of test peptide). The same dose is again injected at 45 min and at 1 hr 45 min. A second treatment group receives 3 times a higher amount of the test molecule (e.g., 50 μg of test peptide at the same intervals. Control animals are injected with saline (e.g., 3×100 μL). Untreated and treated animals are handled in an alternating mode.

Blood samples are immediately put on ice. Plasma is prepared by centrifugation following each time point. Plasma is kept at −20° C. and free fatty acids FFA), triglycerides, (TG) and glucose are determined within 24 hours using standard test kits (Sigma and Wako).

### Example 15

### Effect of Test Molecules on EFA following Epinephrine Injection

Following is a representative rodent model for identifying therapeutics for treating human diabetes. In mice, plasma free fatty acids increase after intragastric administration of a high fat/sucrose test meal. These free fatty acids are mostly produced by the activity of lipolytic enzymes i.e. lipoprotein lipase (LPL) and hepatic lipase (HL). In this species, these enzymes are found in significant amounts both bound to endothelium and freely circulating in plasma. Another source of plasma free fatty acids is hormone sensitive lipase (HSL) that releases free fatty acids from adipose tissue after β-adrenergic stimulation. To test whether test molecules also regulate the metabolism of free fatty acid released by HSL, mice are injected with epinephrine.

Two groups of mice are given epinephrine (5 μg) by intraperitoneal injection. A treated group is injected with a test molecule (e.g., 25 μg of test peptide) one hour before and again together with epinephrine, while control animals receive saline. Plasma is isolated and free fatty acids and glucose are measured as described above.

### Example 16

### Effect of Test Molecules on Muscle FFA Oxidation

Following is a representative rodent model for identifying therapeutics for treating human diabetes. To investigate the effect of test molecules on muscle free fatty acid oxidation, intact hind limb muscles from C57BL/6J mice are isolated and EPA oxidation is measured using oleate as substrate (Clee, S. M. et al. Plasma and vessel wall lipoprotein lipase have different roles in atherosclerosis. J Lipid Res 41, 521-531 (2000); Muoio, D. M., Dohm, G. L., Tapscott, E. B. & Coleman, R. A. Leptin opposes insulin's effects on fatty acid partitioning in muscles isolated from obese ob/ob mice. Am J Physiol 276, E913-921 (1999)) Oleate oxidation in isolated muscle is measured as previously described (Cuendet et al (1976) J Clin Invest 58:1078-1088; Le Marchand-Brustel, Y., Jeanrenaud, B. & Freychet, P. Insulin binding and effects in isolated soleus muscle of lean and obese mice. Am J Physiol 234, E348-E358 (1978). Briefly, mice are sacrificed by cervical dislocation and soleus and EDL muscles are rapidly isolated from the hind limbs. The distal tendon of each muscle is tied to a piece of suture to facilitate transfer among different media. All incubations are carried out at 30° C. in 1.5 mL of Krebs-Henseleit bicarbonate buffer (118.6 mM NaCl, 4.76 mM KCl, 1.19 mM KH₂PO₄, 1.19, mM MgSO₄, 2.54 mM CaCl₂, 25 mM NaHCO₃, 10 mM Hepes, pH 7.4) supplemented with 4% FFA free bovine serum albumin (fraction V, RIA grade, Sigma) and 5 mM glucose (Sigma). The total concentration of oleate (Sigma) throughout the experiment is 0.25 mM. All media are oxygenated (95% O₂; 5% CO₂) prior to incubation. The gas mixture is hydrated throughout the experiment by bubbling through a gas washer (Kontes Inc., Vineland, N.J.).

Muscles are rinsed for 30 min in incubation media with oxygenation. The muscles are then transferred to fresh media (1.5 mL) and incubated at 30° C. in the presence of 1 μCi/mL [1-¹⁴C] oleic acid (American Radiolabelled Chemicals). The incubation vials containing this media are sealed with a rubber septum from which a center well carrying a piece of Whatman paper (1.5 cm×11.5 cm) is suspended.

After an initial incubation period of 10 min with constant oxygenation, gas circulation is removed to close the system to the outside environment and the muscles are incubated for 90 min at 30° C. At the end of this period, 0.45 mL of Solvable (Packard Instruments, Meriden, Conn.) is injected onto the Whatman paper in the center well and oleate oxidation by the muscle is stopped by transferring the vial onto ice.

After 5 min, the muscle is removed from the medium, and an aliquot of 0.5 mL medium is also removed. The vials are closed again and 1 mL of 35% perchloric acid is injected with a syringe into the media by piercing through the rubber septum. The CO₂ released from the acidified media is collected by a Solvable in the center well. After a 90 min collection period at 30° C., the Whatman paper is removed from the center well and placed in scintillation vials containing 15 mL of scintillation fluid (HionicFlour, Packard Instruments, Meriden, Conn.). The amount of ¹⁴C radioactivity is quantitated by liquid scintillation counting. The rate of oleate oxidation is expressed as nmol oleate produced in 90 min/g muscle.

To test the effect of test molecules on oleate oxidation, the each test molecule is added to the media (e.g., a final concentration of 25 μg/mL of test peptide) and maintained in the media throughout the procedure.

### Example 17

### Effect of Test Molecules on FFA following Intralipid Injection

Following is a representative rodent model for identifying therapeutics for treating human diabetes. Two groups of mice are intravenously (tail vein) injected with 30 μL bolus of Intralipid-20% (Clintec) to generate a sudden rise in plasma FFAs, thus by-passing intestinal absorption. (Intralipid is an intravenous fat emulsion used in nutritional therapy). A treated group (treated with test molecule) is injected with a test molecule (e.g., 25 μg of a test peptide) at 30 and 60 minutes before Intralipid is given, while control animals receive saline. Plasma is isolated and FFAs are measured as described previously. The effect of a test molecule on the decay in plasma FFAs following the peak induced by Intralipid injection is then monitored.

### Example 18

### In Vivo Tests for Metabolic-related Activity in Rodent Diabetes Models

Following are representative rodent models for identifying therapeutics for treating human diabetes. As metabolic profiles differ among various animal models of obesity and diabetes, analysis of multiple models is undertaken to separate the effects of test molecules on hyperglycemia, hyperinsulinemia, hyperlipidemia and obesity. Mutations within colonies of laboratory animals and different sensitivities to dietary regimens have made the development of animal models with non-insulin dependent diabetes associated with obesity and insulin resistance possible. Genetic models such as db/db and ob/ob (See Diabetes, (1982) 31(1): 1-6) in mice and fa/fa in zucker rats have been developed by the various laboratories for understanding the pathophysiology of disease and testing the efficacy of new antidiabetic compounds (Diabetes, (1983) 32: 830-838; Annu Rep Sanlyo Res Lab (1994) 46: 1-57). The homozygous animals, C57 BL/KsJ-db/db mice developed by Jackson Laboratory, US, are obese, hyperglycemic, hyperinsulinemic and insulin resistant (J Clin Invest, (1990) 85: 962-967), whereas heterozygous animals are lean and normoglycemic. The db/db mice progressively develop insulinopenia with age, a feature commonly observed in late stages of human type II diabetes when blood sugar levels are insufficiently controlled. The state of the pancreas and its course vary according to the models. Since this is a model of type II diabetes mellitus, test molecules are tested for blood sugar and triglycerides lowering activities. Zucker (fa/fa) rats are severely obese, hyperinsulinemic, and insulin resistant (Coleman, Diabetes 31:1, 1982; E. Shafrir, in Diabetes Mellitus; U. Rifkin and D. Porte, Jr. Eds. (Elsevier Science Publishing Co., Inc., New York, ed. 4, 1990), pp 299-340), and the fa/fa mutation may be the rat equivalent of the murine db mutation (Friedman et al., Cell 69:217-220, 1992; Truet et al., Proc. Nat. Acad. Sci. USA 88:7806, 1991). Tubby (tub/tub) mice are characterized by obesity, moderate insulin resistance and hyperinsulinemia without significant hyperglycemia (Coleman et al., J. Heredity 81:424, 1990).

Previously, leptin was reported to reverse insulin resistance and diabetes mellitus in mice with congenital lipodystrophy (Shimomura et al. Nature 401: 73-76 (1999). Leptin is found to be less effective in a different lipodystrophic rodent model of lipoatrophic diabetes (Gavrilova et al Nature 403: 850 (2000); hereby incorporated herein in its entirety including any drawings, figures, or tables).

The streptozotocin (STZ) model for chemically-induced diabetes is tested to examine the effects of hyperglycemia in the absence of obesity. STZ-treated animals are deficient in insulin and severely hyperglycemic (Coleman, Diabetes 31:1, 1982; E. Shafrir, in Diabetes Mellitus; H. Rifkin and D. Porte, Jr. Eds. (Elsevier Science Publishing Co., Inc., New York, ed. 4, 1990), pp. 299-340). The monosodium glutamate (MSG) model for chemically-induced obesity (Olney, Science 164:719, 1969; Cameron et al., Clin Exp Pharmacol Physiol 5:41, 1978), in which obesity is less severe than in the genetic models and develops without hyperphagia, hyperinsulinemia and insulin resistance, is also examined. Also, a non-chemical, non-genetic model for induction of obesity includes feeding rodents a high fat/high carbohydrate (cafeteria diet)-diet ad libitum.

Test molecules are tested for reducing hyperglycemia in any or all of the above rodent diabetes models or in humans with type II diabetes or other metabolic diseases described previously or models based on other mammals. In some assays, the test molecule sometimes is combined with another compatible pharmacologically active antidiabetic agent such as insulin, leptin (U.S. provisional application No. 60/155,506), or troglitazone, either alone or in combination. Tests described in Gavrilova et al. ((2000) Diabetes 49:1910-6; (2000) Nature 403:850) using A-ZIP/F-1 mice sometimes are utilized, test molecules are administered intraperitoneally, subcutaneously, intramuscularly or intravenously. Glucose and insulin levels of the mice are tested, food intake and liver weight monitored, and other factors, such as leptin, FFA, and TG levels, often are measured in these tests.

In Vivo Assay for Anti-hyperglycemic Activity of Test Molecules

Genetically altered obese diabetic mice (db/db) (male, 7-9 weeks old) are housed (7-9 mice/cage) under standard laboratory conditions at 22° C. and 50% relative humidity, and maintained on a diet of Purina rodent chow and water ad libitum. Prior to treatment blood is collected from the tail vein of each animal and blood glucose concentrations are determined using One Touch Basic Glucose Monitor System (Lifescan). Mice that have plasma glucose levels between 250 to 500 mg/dl are used. Each treatment group consists of seven mice that are distributed so that the mean glucose levels are equivalent in each group at the start of the study. db/db mice are dosed by micro-osmotic pumps, inserted using isoflurane anesthesia, to provide test molecules, saline, and an irrelevant peptide to the mice subcutaneously (s.c.). Blood is sampled from the tail vein hourly for 4 hours and at 24, 30 h post-dosing and analyzed for blood glucose concentrations. Food is withdrawn from 0-4 b post dosing and reintroduced thereafter. Individual body weights and mean food consumption (each cagey are also measured after 24 h. Significant differences between groups (comparing test molecule treated to saline-treated) are evaluated using a Student t-test.

### Example 19

### Tests of Metabolic-Related Activity in Humans

Tests of the efficacy of test molecules in humans are performed in accordance with a physician's recommendations and with established guidelines. The parameters tested in mice are also tested in humans (e.g. food intake, weight, TGO TC, glucose, insulin, leptin, FFA). It is expected that the physiological factors are modified over the short term. Changes in weight gain sometimes require a longer period of time. In addition, diet often is carefully monitored. Test molecules often are administered in daily doses (e.g., about 6 mg test peptide per 70 kg person or about 10 mg per day). Other doses are tested, for instance 1 mg or 5 mg per day up to 20 mg, 50 mg, or 100 mg per day.

### Example 20

### Kinase Activity Assays

Tyrosine kinase activity is determined by 1) measurement of kinase-dependent ATP consumption in the presence of a generic substrate such as polyglutamine, tyrosine (pEY), by luciferase/luciferin-mediated chemiluminescence or; 2) incorporation of radioactive phosphate derived from ³³P-ATP into a generic substrate which has been adsorbed onto the well surface of polystyrene microtiter plates. Phosphorylated substrate products are quantified by scintillation spectrometry.

### Materials and Methods

Kinase activity and compound inhibition are investigated using one or more of the four assay formats described below. A brief summary of exemplary assay conditions is listed in Table 32, where [E] is the enzyme concentration and [ATP] is the ATP concentration.

An EPHA3 enzyme construct comprised the human EPHA3 intracellular domain (amino acids 571-986) containing juxtamembrane, kinase and SAM regions. It was expressed in E. coli as a recombinant protein. 6×His and NusA expression tags were used in pET28a and pET44a vectors (Novagen), respectively. Expression was carried out in Rosetta DE cells with IPTG induction followed by recombinant protein purification on a Ni-column using imidazole elution buffer.

TABLE 32
Assay Conditions

| | | | | | | Incubation | | | |
|---|---|---|---|---|---|---|---|---|---|
| | Assay | | | | | Time | | Enzyme | Enzyme |
| Enzyme | Format | [E] | [ATP] | Substrate | [Substrate] | (min) | Assay Buffer | Construct/Preparation | Source |
| EPHA3 | Delfia | 30 nM | 30 μM | Src-biotin | 1 μM | 180 | 10 mM HEPES | Human EphA3 pET28a-EphA3 | Produced as |
| | | | | | | | pH 7.4, 2 mM | | described |
| | | | | | | | MgCl₂, 10 mM | | above |
| | | | | | | | MnCl₂, 1 mM | | |
| | | | | | | | DTT, 0.01% | | |
| | | | | | | | Pluronic F-127 | | |
| EphB4 | Radio- | 5 μM | 5 μM | poly-AEKY | 2 μg/well | 150 | 20 mM TrisHCl, | Kinase domain E605-e890 with | PanVera |
| | metric | | | | | | Ph 7.5, 10 mM | N-terminal His6 tag is | |
| | | | | | | | MgCl₂, 0.1 mM | expressed in baculovirus and | |
| | | | | | | | NaVO₃, 0.01% | puriied by IMAC | |
| | | | | | | | Triton | chromatography | |
| EphA2 | LCCA | 20 μM | 3 μM | poly-EY | 1.6 μM | 180 | 20 mM TrisHCl, | N598-R890 with N-terminal | PanVera |
| | | | | | | | pH 7.5, 10 mM | His6 tag is expressed in | |
| | | | | | | | MgCl₂, 3 mM | baculovirus and purified by IMA | |
| | | | | | | | MnCL₂, 0.01% | Chromatography | |
| | | | | | | | Triton | | |
| EGFR | LCCA | 7 μM | 3 μM | poly-EY | 1.6 μM | 210 | 20 mM TrisHCl, | Amino acids H672-A1210, N- | ProQuinase |
| | | 11 μM | | | | | pH 7.5, 10 mM | terminally fused to GST-HIS6- | |
| | | | | | | | MgCl₂, 3 mM | Thrombin cleavage site, | |
| | | | | | | | MnCl₂, 1 mM | expressed in baculovirus, one- | |
| | | | | | | | DTT, 0.01% Triton | step affinity purification using | |
| | | | | | | | | GSH-agarose | |
| KDR | LCCA | 5 μM | 3 μM | poly = EY | 1.6 μM | 240 | 20 mM TrisHCl, | Human KDR c-DNA, Amino | ProQuinase |
| | | 6 μM | | | | | pH 7.5, 10 mM | Acids D807-V1356, N- | |
| | | | | | | | MgCl₂, 3 mM | terminally fused to GST. One- | |
| | | | | | | | MnCl₂, 1 mM | step affinity purification using | |
| | | | | | | | DTT, 0.01% Triton | GSH-agarose | |

The ATP concentrations are selected near the Michaelis-Menten constant (KM) for each individual kinase. Dose-response experiments are performed at ten different inhibitor concentrations in a 384-well plate format. The data are fitted to a standard four-parameter equation listed below:

Y=Min+(Max−Min)/(1+XIC50)̂H

where Y is the observed signal, X is the inhibitor concentration, Min is the background signal in the absence of enzyme (0% enzyme activity), Max is the signal in the absence of inhibitor (100% enzyme activity), IC₅₀ is the inhibitor concentration at 50% enzyme inhibition and H represents the empirical Hill's slope to measure the cooperatively. Typically H is close to unity. These parameters are obtained by nonlinear regression algorithm built into ActivityBase software (available from ID Business Solutions Ltd., of Guildford, Surrey, UK).

### ³³P Phosphoryl Transfer Assay (Radiometric)

Greiner 384-well white clear bottom high binding plates (available from Greiner Bio-One, Inc., of Longwood, Fla.) are coated with 2 μg/well of protein or peptide substrate in a 50 μL volume overnight at ambient temperature. The coating buffer contains 40 μg/mL substrate, 22.5 mM Na₂CO₃, 27.5 mM NaHCO₃, 150 μM NaCl and 3 mM NaN₃. The coating solution is aspirated and the plates are washed once with 50 μL of assay buffer and padded dry. Subsequently compounds and enzymes are mixed with γ³³ P-ATP (3.3 μCi/nmol) in a total volume of 20 μL in suitable assay buffers (see Table 33). For example the final reaction solution contains 20 mM Tris HCl, pH 7.5, 10 mM MgCl₂, 0.01% Triton X-100, 0.1 mM NaV₃, 5 nM enzyme and 5 μM ATP.

The mixture is incubated at ambient temperature for 1.5-2.5 hrs; as indicated in Table 32 and stopped by aspirating using an EMBLA 96-head washer. The plates are subsequently washed 6-12 times with PBST or TBS buffer. Scintillation fluid (50 μl/well) is then added, the plates are sealed and activity assessed by liquid scintillation spectrometry on a Perkin Elmer MicroBeta TriLux (available from PerkinElmer Life and Analytical Sciences, Inc., of Boston Mass.).

### Luciferase-Coupled Chemiluminescent Assay (LCCA)

In the LCCA assays, kinase activity is measured by the ATP consumption that is accurately measured by luciferase-coupled chemiluminescence. Greiner 384-well white clear bottom medium binding plates are used for LCCA. Briefly the kinase reaction is initiated by mixing compounds, ATP and kinases in a 20 μL volume. The mixture is incubated at ambient temperature for 24 hrs as indicated in Table 32. At the end of the kinase reaction, a 20 μl luciferase-luciferin mix is added and the chemiluminescent signal is read on a Wallac Victor reader. The luciferase-luciferin mix consists of 50 mM HEPES, pH 7.8, 8.5 μg/mL oxalic acid (pH 7.8), 5 (or 50) mM DTT, 0.4% Triton X-100, 0.25 mg/mL coenzyme A, 63 μM AMP, 28 μg/mL luciferin and 40,000 units of light/mL luciferase. For the LCCA assays, the ATP consumption has been kept at 25-45%, where the decrease in substrate concentration has less than 35% effect on IC₅₀ values compared to the “theoretical” values with no substrate turnover. The IC₅₀ values correlates well with those of radiometric assays.

### AlphaScreen

In AlphaScreen, when the donor and acceptor beads are close in proximity, a series of photochemical events will give rise to a fluorescent signal upon light activation. Here we use biotinylated poly-(Glu, Tyr) 4.1 as the kinase substrate, streptavidin-coated donor beads and anti-phosphortyrosine antibody PY100-coated acceptor beads. Upon phosphorylation, the peptide substrate can bind to both donor and acceptor beads, thus gives rise to fluorescence. Compounds, ATP, biotinylated poly-(Glu, Tyr) and kinases are mixed in a volume of 20 μL for 1 hr at ambient temperature using Greiner 384-well white clear bottom medium binding plates. Then 10 μL solution containing 15-30 mg/mL AlphaScreen beads, 75 mM Hepes, pH 7.4, 300 mM NaCl, 120 mM EDTA, 0.3% BSA and 0.03% Tween-20 is added to each well. After 2-16 hr incubation of the beads, plates are read in a Perkin Elmer AlphaQuest reader (available from PerkinElmer Life and Analytical Sciences, Inc., of Boston Mass.). The IC₅₀ values correlate well with those of radiometric assays.

Enzymes may be purchased from Proqinase (of Freiburg, Germany) and Panvera (of Madison, Wis.).

### Delfia Screen

The DELFIA method is a solid-phase, non-homogeneous system that measures enzymatic activity by quantitating the phosphorylation of an immobilized substrate. The DELFIA method described herein yielded the results shown in Table 33. The compound names are provided in the “Compositions Comprising Diabetes-Directed Molecules” section.

In this experiment, EPHA3 (30 nM) was incubated with biotinylated substrate, biotin-Src-peptide (1 μM)+ATP (30 μM) in an assay medium (10 mM HEPES pH 7.4, 2 mM MgCl₂, 10 μM MnCl₂, 1.0 mM DTT, 0.01% Pluronic F-127) in the presence of test compounds. After 3 hr incubation at 37° C., the reaction was stopped (5 mM EDTA) and the substrate phosphorylation was quantified in DELFIA assay using Eu-labeled anti-phosphotyrosine antibody.

TABLE 33
EPHA3 Potency

| Compound | |
|---|---|
| Name | IC₅₀ (μM) |
| sqnm-12 | 0.2 |
| sqnm-9 | 0.2 |
| sqnm-14 | 0.2 |
| sqnm-10 | 0.3 |
| sqnm-11 | 0.4 |
| sqnm-5 | 0.5 |
| sqnm-15 | 0.8 |
| sqnm-7 | 0.9 |
| sqnm-6 | 0.4 to 2.0 |
| sqnm-1 | 2 |
| sqnm-8 | 2 |
| sqnm-4 | 4.5 |
| sqnm-16 | 11 |

All of the compounds provided in Table 33 represent EPHA3 inhibitors that may be used in methods for treating type II diabetes as described herein. Particularly potent EHPA3 inhibitors have a potency of less than 1.0 mM (e.g., sqnm-12, sqnm-9, sqnm-14, sqnm-10, sqnm-11, sqnm-5, sqnm-15, sqnm-7 and sqnm-6).

### Example 21

### mRNA and Protein Expression Analysis

MCF-7 cells were plated on 6-well dish and transfected with 40 nM siRNA designed against EPHA3. The EPHA3 siRNA molecules are provided in Table 34 below, where siGL2 and Lipofectamine serve as negative controls:

TABLE 34

| | | | |
|---|---|---|---|
| | | | SEQ |
| | | | ID |
| siRNA | siRNA Target | Sequence Specificity | NO: |

| | | |
|---|---|---|
| siEphA3_125 | EPHA3 | GCGGAGCATGGTAACTTCT |
| siEphA3_519 | EPHA3 | GCTCAAGTTCACTCTACGA |
| siEphA3_530 | EPHA3 | CTCTACGAGACTGCAATAG |
| siEphA3_626 | EPHA3 | AATTTCGAGAGCATCAGTT |
| siGL2 | GL2 | CGUACGAGGAAUACUUCGA |

48 hours after transfection RNA samples were harvested using a RNeasy Mini Kit and mRNA was converted to cDNA using random hexamers and oligo-dT primers with SuperScript. Amount of mRNA was quantitated by qGE using the following primers forward, 5′ ACGTTGGATGGGTGTGGAGTACAGTTCTTG3′, and reverse, 5′ ACGTTOGATGCGGTGACACCAACCTTTTTC3′, extend primer, 5, TTTTTCATGTCATCTGTG3′, and competitive primer, 5′ CGGTGACACCAACCTTTTTCATGTCATCTGTG[C]AAATCTTGGCTATTGTGTCACAAGA ACTGTACTCCACACC3′. To measure protein expression, cells were harvested on Day 3 post-transfection. Cells were collected and stained with 5 ug/mL mouse anti-EPHA3 antibody and stained with biotin conjugated goat anti-mouse streptavidin and PE-conjugated streptavidin.

Results

EPHA3 mRNA was quantitated by qGE to verify that siRNA treatment resulted in a decrease in EPHA3 mRNA. Also, EPHA3 protein was quantitated by flow cytometry using antibody specific to EPHA3. Cells transfected with active siRNA to EPHA3 (see Table 34) showed a decrease in mRNA compared to control as measured by qGE. The decrease in mRNA resulted in a corresponding decrease in EPHA3 protein as detected by flow cytometry measurement. These results show that each siRNA molecule in Table 34 decreases EPHA3 mRNA and protein expression.

### Example 22

### In Vitro Production of EPHA3 Polypeptides

EPHA3 polypeptides encoded by the polynucleotides in SEQ ID NO: 1-3, or a substantially identical nucleotide sequence thereof, may be produced by the methods described herein. cDNA is cloned into a pIVEX 2.3-MCS vector (Roche Biochem) using a directional cloning method. A cDNA insert is prepared using PCR with forward and reverse primers having 5, restriction site tags (in frame) and 5-6 additional nucleotides in addition to 3′ gene-specific portions, the latter of which is typically about twenty to about twenty-five base pairs in length. A Sal I restriction site is introduced by the forward primer and a Sma I restriction site is introduced by the reverse primer. The ends of PCR products are cut with the corresponding restriction enzymes (i.e., Sal I and Sma I) and the products are gel-purified. The pIVEX 2.3-MCS vector is linearized using the same restriction enzymes, and the fragment with the correct sized fragment is isolated by gel-purification. Purified PCR product is ligated into the linearized pIVEX 2.3-MCS vector and E. coli cells transformed for plasmid amplification. The newly constructed expression vector is verified by restriction mapping and used for protein production.

E. coli lysate is reconstituted with 0.25 ml of Reconstitution Buffer, the Reaction Mix is reconstituted with 0.8 ml of Reconstitution Buffer; the Feeding Mix is reconstituted with 10.5 ml of Reconstitution Buffer; and the Energy Mix is reconstituted with 0.6 ml of Reconstitution Buffer. 0.5 ml of the Energy Mix was added to the Feeding Mix to obtain the Feeding Solution. 0.75 ml of Reaction Mix, 50 μl of Energy Mix, and 10 μg of the template DNA is added to the E. coli lysate.

Using the reaction device (Roche Biochem), 1 ml of the Reaction Solution is loaded into the reaction compartment. The reaction device is turned upside-down and 10 ml of the Feeding Solution is loaded into the feeding compartment. All lids are closed and the reaction device is loaded into the RTS500 instrument. The instrument is run at 30° C. for 24 hours with a stir bar speed of 150 rpm. The pIVEX 2.3 MCS vector includes a nucleotide sequence that encodes six consecutive histidine amino acids on the C-terminal end of the EPHA3 polypeptide for the purpose of protein purification. EPHA3 polypeptide is purified by contacting the contents of reaction device with resin modified with Ni²⁺ ions. EPHA3 polypeptide is eluted from the resin with a solution containing free Ni²⁺ ions.

### Example 23

### Cellular Production of EPHA3 Polypeptides

Nucleic acids are cloned into DNA plasmids having phage recombination cites and EPHA3 polypeptides are expressed therefrom in a variety of host cells. Alpha phase genomic DNA contains short sequences known as attP sites, and E. coli genomic DNA contains unique, short sequences known as attB sites. These regions share homology, allowing for integration of phage DNA into E. coli via directional, site-specific recombination using the phage protein Int and the E. coli protein IHF. Integration produces two new att sites, L and R, which flank the inserted prophage DNA. Phage excision from E. coli genomic DNA can also be accomplished using these two proteins with the addition of a second phage protein, X is. DNA vectors have been produced where the integration/excision process is modified to allow for the directional integration or excision of a target DNA fragment into a backbone vector in a rapid in vitro react ion (Gateway™ Technology (Invitrogen, Inc.)).

A first step is to transfer the nucleic acid insert into a shuttle vector that contains attL sites surrounding the negative selection gene, ccdB (e.g. pENTER vector, Invitrogen, Inc.). This transfer process is accomplished by digesting the nucleic acid from a DNA vector used for sequencing, and to ligate it into the multicloning site of the shuttle vector, which will place it between the two attL sites while removing the negative selection gene ccdB. A second method is to amplify the nucleic acid by the polymerase chain reaction (PCR) with primers containing attB sites. The amplified fragment then is integrated into the shuttle vector using Int and IHF. A third method is to utilize a topoisomerase-mediated process, in which the nucleic acid is amplified via PCR using gene-specific primers with the 5′ upstream primer containing an additional CACC sequence (e.g., TOPO® expression kit (Invitrogen, Inc.)). In conjunction with Topoisomerase I, the PCR amplified fragment can be cloned into the shuttle vector via the attL sites in the correct orientation.

Once the nucleic acid is transferred into the shuttle vector, it can be cloned into an expression vector having attR sites. Several vectors containing attR sites for expression of EPHA3 polypeptide as a native polypeptide, N-fusion polypeptide, and C-fusion polypeptides are commercially available (e.g., pDEST (Invitrogen, Inc.)), and any vector can be converted into an expression vector for receiving a nucleic acid from the shuttle vector by introducing an insert having an attR site flanked by an antibiotic resistant gene for selection using the standard methods described above. Transfer of the nucleic acid from the shuttle vector is accomplished by directional recombination using Int, IHF, and X is (LR clonase). Then the desired sequence can be transferred to an expression vector by carrying out a one hour incubation at room temperature with Int, IHF, and X is, a ten minute incubation at 37° C. with proteinase K, transforming bacteria and allowing expression for one hour, and then plating on selective media. Generally, 90% cloning efficiency is achieved by this method. Examples of expression vectors are pDEST 14 bacterial expression vector with att7 promoter, pDEST 15 bacterial expression vector with a T7 promoter and a N-terminal GST tag, pDEST 17 bacterial vector with a T7 promoter and a N-terminal polyhistidine affinity tag, and pDEST 12.2 mammalian expression vector with a CMV promoter and neo resistance gene. These expression vectors or others like them are transformed or transfected into cells for expression of the EPHA3 polypeptide or polypeptide variants. These expression vectors are often transfected, for example, into murine-transformed cell lines (e.g., adipocyte cell line 3T3-L1, (ATCC), human embryonic kidney cell line 293, and rat cardiomyocyte cell line H9C2).

Provided hereafter is an EPHA3 genomic nucleotide sequence (SEQ ID NO: 1). Polymorphic variants are designated in IUPAC format. The following nucleotide representations are used throughout the specification and figures: “A” or “a” is adenosine, adenine, or adenylic acid; “C” or “c” is cytidine, cytosine, or cytidylic acid; “G” or “g” is guanosine, guanine, or guanylic acid; “T” or “t” is thymidine, thymine, or thymidylic acid; and “I” or “i” is inosine, hypoxanthine, or inosinic acid. SNPs are designated by the following convention: “R” represents A or G, “M” represents A or C; “W” represents A or T; “Y” represents C or T; “S” represents C or G; “K” represents G or T; “V” represents A, C or G; “H” represents A, C, or T; “D” represents A, G, or T; “B” represents C, C, or T; and “N” represents A, G, C, or T.

| EPHA3 Region GENOMIC | |
|---|---|
| >3:89375801-89470550 | |
| 1 | tgtacttaat cagcatttac cgcaagaaca acatatgcaa ataagagcat aaaagaggct |
| 61 | gttccaggaa ttgtgtgtat tttaaaatat ctagagatta ggatgcttat ggagtttgta |
| 121 | ggagatgtgg ctggggattt aaacatgaaa cagattctaa atggctttta cacgctctgc |
| 181 | tatgaaattt gcatcttatc ctgaaagtta taaagagcca aaatKttcct atgcaaaact |
| 241 | tttatgaata ggaatgacat ggtcaaattc atggtttaga aggaatatta tgaaagataa |
| 301 | aatgtgtgga aagcatacaa atagagacca aattaggaat ttctaattat aattcagaag |
| 361 | ataaatgaag acttaaacta ggatatttaa gagtgtttgg tgattaactg gaggcagaat |
| 421 | gattaggaga tggagaggag aaggttattt tatatttatg gtctagattc ttgctcctca |
| 481 | aaatgttgtg gtctggaatc agcagaatca gcactgcctg tgtgttcata agaaatatac |
| 541 | aatctcattc cccactccgg aatgactgaa tctgaaactg cttttccaga gattcttgag |
| 601 | tggtttttgt gaacattaaa gtttgagaag cattggtctg ggtcaagagt cagtaaactt |
| 661 | ttttctgtaa taggccagat aataaatatt ttaggctttg tgcacatcat tctctattgc |
| 721 | tgctactcaa cttgcccatt ctagcataaa atcagtcata gacaatatac aaattaatga |
| 781 | atgattgtgg ctatattcca ataaaattgt atttatagac agcaaaattt gaatttccta |
| 841 | gatttagaat tttcacatgt catgaaatct cattcttttg actttttaca actgttaaaa |
| 901 | gaaatgtgaa accattctaa gctcgtgagc tgtacaaaaa caggggacgg gcaagatttg |
| 961 | gctcctgggc ttttttgcca accatttgtc taggtgaata aatgggccat ttataaaggt |
| 1021 | aggaaataga gggacagttt gttggtggta tgtgttgagg tccatgtatt atatcacttt |
| 1081 | tcaaatgaat atggatattg gagttaaaac aagcttaatt agaaagcttt ttataattgt |
| 1141 | atgctgagaa atcttcctct cttctctaag atcctcccat aacttctggt tcattcaacg |
| 1201 | agttttttgc cattgaggac agagaaaccc agtacagcat attttggaca ggagacattt |
| 1261 | aaataatgtt tgattgatgt cattgaaaca aagtatgtac ccatattaat taggtagaaa |
| 1321 | tttcaagcaa aattatggga aatgtctatt tttttaaaca ttaagtaaag aatactttca |
| 1381 | acattgtatc agacattaag ccatacttca ggtaaaagta gaacacatat taattaatta |
| 1441 | atgaatcaat caactgttcc atgtagatgt tagtcatgat tataatacct gttcttattt |
| 1501 | tttctcttca acctcacagc tttctccatc tctggtgaaa gtagccaagt ggtcatgatc |
| 1561 | gccatttcag cggcagtagc aattattctc ctcactgttg tcatctatgt tttgattggg |
| 1621 | aggtgagttc acagtctgtt tcactattca ctttcWttgt tgctttgttt gctgccactg |
| 1681 | attgctgtta aaatgtgaag agtgtgctca ataaaatatt ttaacaaata agaatgtctc |
| 1741 | cacttgtagt ttaggtcctc tctttctcct ccttttcttt gtttctccat ccttagtttt |
| 1801 | gattttctgc tctacattaa attcctgtcc ccactctttt aacatacacc cctcctcttc |
| 1861 | tctcttctga cactactcct agtttttact ttctttctct aattatctcc tcactcttca |
| 1921 | aataacccca gatttccttg acactttctc acccagaaca ggggtaacac tgcacagcac |
| 1981 | taatcattac ccatagagac tttacccttc attccttcta acagaagttg ttaaaaaata |
| 2041 | ctataaacac attctaaagc cattataaaa actgaactgg agggtctgca cagtgggaag |
| 2101 | caactctgtg tatctcctca ttaggcagct ttccagggtt cccagagaga ggggctttct |
| 2161 | tcagaggatt ttgaaaatat gaagttatta aactttcact aaagcattta attgtttgag |
| 2221 | gaaaatgttt tatttttatg ctttgcacat tctgagcata ggtaactatt ttaaatatat |
| 2281 | tcctcttatg tgttcgcttt ccttgattta cctccaggtt ctgtggctat aagtcaaaac |
| 2341 | atggggcaga tgaaaaaaga cttcattttg gcaatgggca ttgtaagttt ctaaacttgg |
| 2401 | ctttttgttt tgcttcaccg ttttagcttt agcagttatt gatttacaat tggttaactt |
| 2461 | cctcctgaca aagagacttc aaaagtagtt ttatggaaaa actgagtaca ttaaatttat |
| 2521 | tttagaaaaa gagggaaatt catggttctg cattaaataa tttttacatg tgtacatttc |
| 2581 | agcatactta agctaaaata aagggtaatc tgtggtttac attcaaataa cgcacatgct |
| 2641 | aggtagtaga aactagtctg ctctgcatgg ctgaaatgca aagcgtatct tcaatttcct |
| 2701 | tattactagt cctttgctaa actctaaata ggagactttt ctctttccta actgccaagt |
| 2761 | aaatataaac aacctctgtt tatttgaagt gccttatccc attttatcaa ccaacctaga |
| 2821 | aagagccttt aaaataaact gtttattttg cagttacatg atatccaact agtagtaaag |
| 2881 | ccagaacttt ctctccacat tttaatttac ccttttaatt ttttttaaat acacatttca |
| 2941 | gaaacaaagt gtttacaact aacaaaaaga ctttttttct ttttctggtg aaacgaatca |
| 3001 | ttccaggtgt tttgcgttcc aaactatggc tcagcttagt gtggcaatta ctaatcagta |
| 3061 | gcttaaatag catgttctca tggaaattag tacaaagact gtactcatgc ttatgtgctc |
| 3121 | caaaataatc tcaatataac taatgtagtc ttgtaagcat taattttttt agtattcact |
| 3181 | attgccttct aaaattattt gcaaattgac atgatacttt gtatcagccg ttggattatc |
| 3241 | atctaaaaat gatagcttta gttgtaactc aagtaaaaaa cttgactacc tgttgctaaa |
| 3301 | ctgcaggaag catacagtag cttgaaaata atgcctttca aaatataaat tgatatgaga |
| 3361 | agaaagcagt ataggagttt gaagaagcat tattttgttc atccaattgc agcgtctata |
| 3421 | aaataattca cacaaaaaag ccaaagtact acattcctaa aatcaacatc acatcagtcc |
| 3481 | agattaatta ggtgaccaac tcaaaagtct aaaaagaatg cctgaacaag ttacaggatc |
| 3541 | cttgtctttc tttttctcaa tgggcccaca aaacactagt gttacaattt ttatgcataa |
| 3601 | attagagcag tggcattctt gtggattaag tcacactcaa tttgacttgg acaattgtaa |
| 3661 | atatcatctt tagagcaaga ccatgctgat ttgatttttt ttttatggga agactacata |
| 3721 | ttacgaattt tgagctttgg taaatttctg tgtgccaatt attaaacagg ttcctctatc |
| 3781 | gtcccagtct gttttgctat tgtttttatg gtggattttt gttgaaactt taattttttt |
| 3841 | caaatgcaca atatccttat gacaaacaaa agagaaatgc atttcatcct ggctgcaaag |
| 3901 | gcataaagta acttatatgc atgtcataaa ctttctgcat cttaatacta cagctggttt |
| 3961 | atttcagacc ttttatttgg tattacttta aaaagctcta gctacgaact gttccctatt |
| 4021 | gccttgttac aaatcctata atattgcttc aaaaggaaag aaatgtgttt cacctcctaa |
| 4081 | gatcctgtcc ccttgccctt tctggctggg cctttttact tctttgctat ccttagggca |
| 4141 | actcatgctg gtaaaatgga aatgtaaatt attgtgtttg cacagaatga gctgtaaagt |
| 4201 | agtgcagctt aacacgtgtg catgtgttta ttacctggtt ttcatgacag agcccagtta |
| 4261 | ctctgtatgt tgcccttaat tattggtgtg aagggattat gtgcctcgca gcatgaatca |
| 4321 | cctgattcct ggaatctctg gggtcagcag ttaaagatca gcagccagtg cttctacagc |
| 4381 | gaacagacca ttttacttgg gggtgtatat gtttatgttt gtttttttcc cccaaatgtc |
| 4441 | ttatggatgg aaaagaaatt caccttattt taattaatag aaattaatag gattcatttt |
| 4501 | atttgggaat ggttatttaa ttttcaaatt atttagtaat agtagtattg catataggta |
| 4561 | tccatcatac atatattctc tacaagctga ctggtactta agggtaaatc taaatcacac |
| 4621 | aatttcaagt ctgtttaagg ccaatgctta tttaaaatat gtgagacatt gttcctaaca |
| 4681 | actcatttta taatctttgt aaattctgat ggcaggaaat ctcccccacg ttaatttcct |
| 4741 | taacttcagc aaagtaaagg agttataatt atgtacaaaa cagttccatt ttaccttatg |
| 4801 | cttaaagtat cattttattt ccttgaaaaa gattgctaga tttctttacc ttactgttac |
| 4861 | taagtaaagg caaataaaaa ctggaagatc acacattata gggttgatct gatatgagtt |
| 4921 | ttcatcctta aaatgtaact aaattgacta aatgaaaaga ctccaacaag attttatttc |
| 4981 | ctaacatatt ttagtaatgg ttacttactt ttacaatttt tcttactcca catctagaaa |
| 5041 | agtatgatag cttttcctta atcttaaaga agtttcttct tttaaaggta aagggtttta |
| 5101 | gtactggtaa tgttagaagt tattttaatg taacaataaa gcacaacaga ttactgtatt |
| 5161 | aaagggtgac tctcccatct gtatttatag gaaggattca tgcttatatg gaagaagcat |
| 5221 | ataagctgat ctgtgagctc aaaagtaatg gacatttcta agtctaaatt ctaccattag |
| 5281 | gaaacatcaa ttatattaat aattgtaaat aaatgttctt aacaataaaa acgctcatta |
| 5341 | gggcaatgag ctctatcaat acattgagtg agaactatgc aacagtttct catgctaggc |
| 5401 | atttgaatag tatagctttt tttttttctc aRagccctag cttgtctttt ttgccaagat |
| 5461 | attcaagatg aaaaactcta aaagtgaagt tctcacttca acagctacta cactcagagt |
| 5521 | ttgtccaaag caagggacag aacttgtatt tcccaatttc tgtggatatt gacttctaga |
| 5581 | tagtggtgtc ctttcaaatt atccacacct cgcctcaagc tggatagcag ctgctctttt |
| 5641 | gttgtagctg tMtcagcacc aggcagaagt aggagtcgtg ttagcttttc ttctgcattc |
| 5701 | cctatatgcc ctctcaccac catccctgct ttctattatg ctgtgtcaaa gtcatgcaaa |
| 5761 | taatagctga ggaaaataga gtttttaccc aacctctctc tcagccacca tttcttttac |
| 5821 | aacagtttat tctcacactt acgttttttt actcaaaatc tctatgaaag cttcacaaga |
| 5881 | acataaatcg taatgcatta taaccatggg tttcatgaag ttgtcttgta gttcaaagaa |
| 5941 | ttgatcattt gttaacacat actgtgaaat cctagacttg ttggcctaat gttcattgta |
| 6001 | ctgaggcttc ccagaaaaac tgcgggcatc tgcagtctcc acaactattt aatacaaact |
| 6061 | acaaaactaa ctcaaagaaa cgattggttc acgcagggtg ttcaattgca actttcccat |
| 6121 | aattctccca ttatgaagta atattggaaa cagtaatatc tgatttcatt tgttataatt |
| 6181 | taatgtaatg aatgaaaata tatttcacta ttaagtatcc atattctgtg ctaaaacaca |
| 6241 | ctattaagta tacatattat gtgctatgaa tgttatcaaa attaaaagca aaatcagtag |
| 6301 | tcttagcaga tataccatag gagttacatt gcaatttcct ttMattttag atcatattta |
| 6361 | tatagtttag tagatcttag aatcactttt ttaaaactca gggtttttgt ttagagaaag |
| 6421 | ttaatttatt gaaaattgca aattgtctcc actgttattt ttccaaatta taggaatctt |
| 6481 | attaatatta aatcaaatta aaatgattat cctgtatcag ttgtctgtaa tattttaact |
| 6541 | acaggatatg aacattttta ccttcagaat aatttaatga ataaatataa tatgaaggag |
| 6601 | atttttattt aataaattac atatatgact tacgtttgga acttagctcc ttagcgccat |
| 6661 | tattcaactc atagacaata tttattttac ttctaattat gttataaagc tatacaatat |
| 6721 | gattatattt aactgatact atattggtgt gtatacatgt atgtattata tatacacacc |
| 6781 | aatataatat ataatacata catgtataca catactaaca gcttaaagta gatttgtaat |
| 6841 | tacagtggga cattttgatt gtgaatgtta atataagcag tacagtgtca gaaggtatgc |
| 6901 | cattatatat catttaattt atctataatt gcagccatta tctcacctta taattgtatt |
| 6961 | gaattatttt ccaatcttgc attgtaatac atatctggtt attataacat ttgtgtaatc |
| 7021 | ataaatatgc aatagcataa tggtgttcac tcactgttgc tcctggtagt aatgactagg |
| 7081 | tctaatcagt tctgaattat ctttcttctt aagatcccct tttttgagta tgcattttcc |
| 7141 | ttctgatgtt tttgcaaaga aagctcctca ttgtggttcc tttatataat tcatcataga |
| 7201 | gaacatgttt tcttaaattt ttcaacacag tccaatgtca ctgccagttt ctgtgatact |
| 7261 | acagaataaa ctgaaggttg tgcaccttat ggggtaggga tttttttaaa ccaataatgc |
| 7321 | cataaaattt gatctataat tgtttgtaca aatctagcta caattgcgcc tttctttctt |
| 7381 | tcctcaaaca gtaaaacttc caggtctcag gacttatgtt gacccacata catatgaaga |
| 7441 | ccctacccaa gctgttcatg agtttgccaa ggaattggat gccaccaaca tatccattga |
| 7501 | taaagttgtt ggagcaggta accacaatga ccctactgcc aacttagtac tgtatgtgaa |
| 7561 | tcacgattgc tcagtctctg aaacctaagt atattgctaa agaaatggga attttctgat |
| 7621 | ttcatgatca aaggcaagta ataaataagt gcaatattta atggaatgta atgagttcaa |
| 7681 | aggtgccaga cttacaggct cacagaccat ggctgctttt gatttattaa atttctgatg |
| 7741 | tgacagcagc aacaactcaa tattaccata gctgtgctga tcgtgtgtca caaaacctat |
| 7801 | ttggctaaat aaggataact ggataaaagt ctatttcaat ttagacaacc caggtaattt |
| 7861 | gaagttttca ttttcaattt tagaatcatg tttggaatta aaagaacaaa caacacaaag |
| 7921 | taatatcatg actcatctta cataaatttt atcacaacta tcttgggtgg atattttaaa |
| 7981 | taatataagg tgctccattt taatgcttgc ttgactttga atagtcttat gtgttaaagc |
| 8041 | tttttttaat aaatatacct ttgttctaat tattaattta aacaatgata aatttgttaa |
| 8101 | caatcaccta ttaagtggtt aataacagtt gcatatttat aagaaaaatt tcatatattt |
| 8161 | gtaaaataac agtattttat atatttacac ataaacacat gatatgtgca gtatgcatgg |
| 8221 | aagtaaaact actattcaaa agtaaaaatg aaccatgctc aacaataaaa aattttgtac |
| 8281 | catcatctaa tccttcaatg tcactcccca aagcaaacat aaactagtta tgttgataca |
| 8341 | ggaattctct gtgtaacatc agatcacatt ttctgcaagt accgtattta catatttatg |
| 8401 | agtgatgtgt ctttacaaaa atgtacatgt aatgctttgt ttctttttct atacaccaaa |
| 8461 | gtagtactga aaaatcaagc agtagttagt atgcgaacag gacaatcttg taaatataat |
| 8521 | gttaatttac tagagcttct cttctaatta tcttaatttc ttatcctaaa atttacatgt |
| 8581 | gcaggtaata ttaaaatatg aaacttgtgt tcatagaaag aaaatggagt agtaacttct |
| 8641 | tttttacaat ctaagacttg agagtaaaca tattcgctgt acatctgtgc tgatccagtg |
| 8701 | ccaaaacaga tatatattgt ttactctgtt tcatttcttc agtcctgact tctgaacagc |
| 8761 | tccatgatgc agaagggttt agtctggctg gttttaatgt ttcctcacaa gagtgcactt |
| 8821 | ttctccagca cggagcctgc ggtagcaaat gcagcacgag gactttgaac actcaactgg |
| 8881 | agaatatgtt gagaggagga aaaggttact tatgctaagc cactgtccaa aagccaggca |
| 8941 | gacattatta tgatgttgaa atttaggtac ctctccactg ataaaatatc tttatatagt |
| 9001 | tattattgac agactcagat atacagaaca tagtgatttg ggaaaaagca caccactgaa |
| 9061 | aatagcagct gataaatgag ataaggggag gtctatttat actgaatcac ttaatccctg |
| 9121 | atgctcatta aagattagaa tagagactac ttcctatttc tgtcttcaag tatatatgtt |
| 9181 | aatgattaga tatatgccca tgttcagggt tttgcagcca atattcacat ttgccatgtt |
| 9241 | aagaacaaca aaactgtact tatttgcaaa taggtatacc ttcaaggtta gttatacttt |
| 9301 | tcctgaaata attcaataca atatacacac ccaattctag actgccttga tgacctttca |
| 9361 | gagggtttgt atttaccaag ttagaaaatg aaattattat tcctaaaagc taacaatttg |
| 9421 | ttaaacattt tgttttgtaa aatgtaaaat atgtacacaa ataaattaaa tagtgtaata |
| 9481 | ataaataaac ctgtatgtta ctcatttcta aaatagcttt tataacaatg aacaaatggc |
| 9541 | aaccttggtt ggtatataca tttacctact tcctacattc ttaatcttaa aaaaaatatg |
| 9601 | ttatttataa aaatttaata attattctaa attgattacc aactggcttc cattgttaga |
| 9661 | aaggcaaacc tttcagccaa ctacataact ttcagagtca attaaattta cagaaagaat |
| 9721 | atatatatat atatatatat ataagctaat tctcttaaat ttctgtcaga aattttacat |
| 9781 | aaatgaattg atgttatgga aactctcttg tatacattgg actatagaaa gaattatact |
| 9841 | tatactataa atgataaaat taagacatat gaattagctt cccacagtag tagattacta |
| 9901 | gaagtgagga attcatgact acaattccta attttactcc tcaatccaac cattctttca |
| 9961 | acgattgaat actaaaaatt tctgaagaaa gtaaatactt agtaagtcct taatttatcc |
| 10021 | tgtgcattcc catgatattt aattttatta tcctttttag ataggtatta ttatctccat |
| 10081 | ttcagcacgt gaataaacaa aagctgagaa tttgaataga cttgtttgga tcaaatgaaa |
| 10141 | aaccaaaatt tgaatcttat actttccaaa tttaaagccc atgctggaca ctggagatca |
| 10201 | tagtagtgtc ttcttcatag tatttttttt tgaggattaa acaagataag ttgtgcaggg |
| 10261 | gacttagctg acacagaagc attgttcaat atgtgttttt attatgctta ctttatgaaa |
| 10321 | acagtagaag gagggtaggc tttaaagcta gtgttggagg gagtcattct atttaaatgt |
| 10381 | cggctttttt taattgttta aatgaaatta gcataagaaa ttttattttg agcagctttt |
| 10441 | acattgtgca ttttaatcag aatacagtcc ccattcttat aaatatacct atttcaaaga |
| 10501 | gcaaataaaa ttgccgcact tattagaatt aaaaaatcaa gatctggcag ccatgctttt |
| 10561 | atctagaaat gacaagatat gaatttttgt ttcatatggg tgcaaagtta attgaataag |
| 10621 | acaatataaa acttggccag atgaaactat tgcaaattgc atttgcccag tctttgttaa |
| 10681 | cttttacaat acagttcttt tattttttat ttttaatctt tttctatttt tagcagcttt |
| 10741 | attgagatac aatttaccaa ccataaagtt cagctattta agtatacaat tcagtgattt |
| 10801 | ttagtatatt ttcagagttg taataaccat aattgctatc ccaatgcagt tattttaaat |
| 10861 | tattaaaaat ataattgaat taatattaag catttctatg tcttttataa tatattggcg |
| 10921 | gctatttcta gtgtttaact gatgaaataa tcacattact aaatgtgggg aattttaaaa |
| 10981 | actagcattg ctgaaacaca attttcatga atttcgaagg taaatatata catgtacatg |
| 11041 | tttatgtgtc caggtgtccc acttacaaca aagaacactc ttaattggct catttaaaat |
| 11101 | ttgaactcct acttttcata tctctagtat gctatccttt acaaatatat gtccagtaag |
| 11161 | acatgagcct tgttatcgcc atcattacca taaccatctc ttacattaac atagaatttg |
| 11221 | gcattttata aagcactttc atattcattt gtcagtttga tttaaacaga aactcttaaa |
| 11281 | gggtagttag gcaatttatc ctttaggtgg gtctgaactc agagagataa atagtgacca |
| 11341 | acacttagat gtcacctact gtgtgtctgg cactgtttca agtacttaac atacattagt |
| 11401 | tcctttaatc ccacaacagc tttatgaggt agatacaata agtgtccccc atagagatag |
| 11461 | gaaaagatag gtacagagag gttaagttac ttaaaatcac acagctaata tgtggcagag |
| 11521 | tcagaattgg acccgaagca attcggctgc aggatccatg ctcttaacca gagttctgta |
| 11581 | ctatcactcc tgtaaactga caagtgacat gtcacttaga atatgtgatg atgacatcta |
| 11641 | catttaaaac atgctctatt ataccatgat gatgtatgat aatggcagaa ctttctggtt |
| 11701 | ttcccaagct tagtgtttac acaaagtgaa acaaagtaaa tgggagcgct ctgataatgt |
| 11761 | tcggataacc ctatatgagg aaagaaactc ttactcttgt ttacaattct gagaataatt |
| 11821 | ttatcacacc ataacttctc tttctcctaa tattctcata tctaaatggc atattcttcc |
| 11881 | ttcgaattag caagagcaat gaactttctc aagttctact ccctgattct ttctgtatcc |
| 11941 | taaggaacac tatagctgta gatatcatta tcttctctac acttcctcaa agggaataaa |
| 12001 | ctagtcctgt cttgtttacc attatatata tctctgtgtg ttttatactg tatgcaatac |
| 12061 | agttgctatt atgaatattt attaaataaa tgcatgaata tctgcttttg gattttaagt |
| 12121 | gactttgcca aattaaagca taagtcattg ctttgaaaac agggcaagag tggatttctg |
| 12181 | gggatgagtc aagcctgggg aaggttaaag cttcccaggg agagagacac catttgtagt |
| 12241 | aatgtagctg gcaaatcaat aggacaacct gctacttttg tggttcaaac cacttgcttg |
| 12301 | aaaacaaaaa tagagaagaa tcatatcact tcaatctgac tgacacgtag ggcatgaagc |
| 12361 | tcatagaaaa ttcacagaga gaaaattgga ccaagattat gattccactc catcaagagc |
| 12421 | caaaaattaa tcatataatt aattctaaaa tatttcaaga aaagctcatt tgacattaaa |
| 12481 | aactattttt aatgtgatgc aaaacatatc tgataatcca cactatgtta gaagttcttc |
| 12541 | tgtgaacaaa ggacattcta tagaactccc atgtggcaat catcatggca gcatgggtac |
| 12601 | agataaataa atgtaaaggg tatctgctct aagtgagctc caaatgtaat gaattgatga |
| 12661 | ttagtaaatc tctcagtaaa aatgtatttt gaaggtagtg tccatactag ttctaggcag |
| 12721 | ttttatatca actggactat aatatttaaa agtcagtaac actctatatg ggacaaattc |
| 12781 | tggattaatt gcacattgaa gacttttttt tttaaattga agaagatgaa tctaaaatat |
| 12841 | ttctacagta tttagataag gcatgttgtt tttatttgat actgttatat tgttgttgtc |
| 12901 | ctccagaagc ccagagactt ttatagctgg tatgtcagta aagcaacaca ccaatactcc |
| 12961 | aataaaataa attgatagca gatgctttcc ttctcatttt tagagtctga atatatagcc |
| 13021 | atattggccc tcggtaaaaa cagaattgtt aaaatatctg aaatttaatc caatgctcac |
| 13081 | atttaatcac tgttggtgct gtcctgttaa tttctctgag gtaatggtag ctatgacaac |
| 13141 | aaggggtaaa aaatctaaca tatgcagctt tcaacttatc tctcctttta cattacatta |
| 13201 | tttatagtgt cgtactttaa agtatatgga attttcacaa ggttcatttt ccaacagggg |
| 13261 | gtgactatcc tgaggttttc taggcagttt catatgatta tctttcaaaa taatttggag |
| 13321 | tactagagtg tacatcttgc aggtcaaagg cacaaatatt ttaaatggaa attttgaaat |
| 13381 | aaaacagttg ctctctactg atgaattttt attatagaat tccttacatt ttgttgctgt |
| 13441 | tctgctttat aacaggtgaa tttggagagg tgtgcagtgg tcgcttaaaa cttccttcaa |
| 13501 | aaaaagagat ttcagtggcc attaagaccc tgaaagttgg ctacacagaa aagcagagga |
| 43561 | gagacttcct gggagaagca agcattatgg gacagtttga ccaccccaat atcattcgac |
| 13621 | tggaaggagt tgttaccaaa agtaagtaaa gtagtcataa gacctgtgtt tccgtatgtt |
| 13681 | gagcaaaggt tgtttaaacc caaccccaac catttaagaa ttttggcttt ttttcataag |
| 13741 | tatctcagtt ttaccaaaaa gaaaagttta ggaatagcat gcctttcttt gacagtagag |
| 13801 | cagaccttta aataatattc tattgacagt actgttttag acctcaaaaa attttaaaaa |
| 13861 | aatactagaa gccacatatt agaccattta aaggtacgct gattagcaga cagttaagag |
| 13921 | aagaattgtg gggaattctc ggctatgtaa gtagcaaaga aaaacaatga ctcaagcttg |
| 13981 | acaggaactc ccagctagct gccttttctt ttattgttca tcagttttat ttaaaagtta |
| 14041 | caaatcttta acctcctccc accccctgca tccctgcctt taggaaaatt ttgggagtct |
| 14101 | aggaaatgct tctgagctgt acagattgga atcacaatag acccaaggcc gtttcttggt |
| 14161 | tctctgtgga ttttaaatag ttctgatcta gaaacccagg tggattctta ctgctctgtg |
| 14221 | gaagctctgg ctcacagccc atgatagaga tgtgaactta tttccatgat ggtttctcca |
| 14281 | gcaggtctca aaagggatcc tgtgaggcgg atctgtgatt tttggctcca atccctatat |
| 14341 | aagtctcttg agaatactct gggacatttt tcctcaagat tttggagctc aggatgaaga |
| 14401 | ataagtgaaa ttggcagcaa actttttaga gtgaaagaca acatatggca gggaatttgg |
| 14461 | catcctttag gggataattc attggcacca catttacata ctctatcgtt accaacagac |
| 14521 | atatttttca taattatatt tctctttacc attttataga gcattttcca aatggcacta |
| 14581 | atacttattc tcactttagc atgactaaaa aggcatttcc ttcctcatga gagcatggtt |
| 14641 | taattgaaat agactttgat ttccttttga gttttcattt ataaaaagtg gaattaagaa |
| 14701 | tttaacaatg tggaatagtt caacatacat tgtaagtcca tattaattct tataaggctt |
| 14761 | tttatttgtt tgcctatgta ctctacaaaa ccaggcctta ttaatagaaa tattttctga |
| 14821 | taaaaaacat ttttaaattt taagttaata attaataaaa atgggcataa ttagtaagga |
| 14881 | ttgggaaagt gtatgaatcc tttgttcttt ctgagcaagg ctatactgtt ttatgtctgt |
| 14941 | acagttacag tatcatgtct cattgggatt ttagtgtcat ttagccattt aatgttctcc |
| 15001 | attacatctg catcaagtgg tggttgcctt ttagctcttg agcctcctgg gaagagaaaa |
| 15061 | ctcccttgta cttatgaagc cattcttata actctcaatt tttacaagtt tttcattatg |
| 15121 | aagtatctgt tatgtgccca cattatacta tgacattatt tcattcttac cactaccctg |
| 15181 | tgaattagac atgataattt ctctcttaca tttgaacaaa caataactgt aaagggtaat |
| 15241 | tagcttgtat aactagaaaa tgctagaggt agcactggga acttgttctg ttcatttcac |
| 15301 | cacataccca actctgaacc caaacaggtt tctacttgtc ttttcctgta ttctacttat |
| 15361 | tcttttatat cgttacttta aaaatcttct ctaacatagt gagacattac tcaatcttca |
| 15421 | aaaatttctg gaactattaa taatccttaa tatctaaaac aaaatataaa ctctcaaaga |
| 15481 | gggcaaaaac ccatgttatt tgatgacaag tgactaaaat aaatcctgga tcataacaga |
| 15541 | tacttaattt accaatgttg aaaaattaaa aaaaatattg atgaaggaag gaaggaagaa |
| 15601 | agaaaagaag gaaggaaggg aggacacaac aatatctttt aacaataggt ctttctttag |
| 15661 | tatttgattc attaaattga atacactttt caaaggagtg catttaaata actacttcta |
| 15721 | tcacccagag agtgatgaaa aaaaatgatt actttttagc ttgaatagaa tcataatcta |
| 15781 | acttcttcaa catgtgtaat taagaaaaaa atgagctaag agtcactatc acacaaatat |
| 15841 | taggttctaa tttaagctta ctaataaact cactctatta ttttgtcagt catgagttga |
| 15961 | aaatgcattt actggatgat aagcaacatt aaactataaa tgttgtggca aatttccttt |
| 16021 | agatataaaa ctgaaacaat taaatataga ttatggtaga ctgacaaatc tatgaaacaa |
| 16081 | tctctaagac aaaaaatgta gaaaaatgca ctaaaatgac ttttaaaaat tgcaggtatt |
| 16141 | atatattaaa catcttacta aaaccgactc tgtaacattc taatatggat tacaactgcc |
| 16201 | ttattcttag ggtgagagtt ctaaatttga tatatctccc tggtcaaata agcatgtaag |
| 16261 | aagctgccaa atctgactat ataaatccca gtcatctcat atattgtaaa tataatagag |
| 16321 | tagtagccct agaattagat tttgaactcc ttggtggcat atttataagc tcttagtctg |
| 16381 | gcacccggta acatctgaga ggaaatagaa actccttaca ataaagttga tatgttgtat |
| 16441 | ttaacacaca cacacacaca cacacacaca cacacacaca cacagagcga ttgtgtttcc |
| 16501 | aaaagaagct atttattcta ctgctattat tgtctttgtt tcctgagatt tctaaaacaa |
| 16561 | tccacctgca gagatcagaa agcacatata tacatgcaca catgcacaca cacacacaca |
| 16621 | actagagtga tgcagagctt ataatttaat gaacaaaata atattttcaa aagcaggcat |
| 16681 | atcagtatct agattaataa tcaatgatct gagttcaagt tctgaaacaa ccttactact |
| 16741 | ctgagaactc ctacaaatca gacaattcac acataattca tttcttcgct tgtaacaaaa |
| 16801 | ttaaacagta ctactttact atttttagat actaaagatc aaatgagatt acataagaat |
| 16861 | atattaagtg gcaaagaact gtgcaaatct aaattactcc tctggttgtt tgagatttta |
| 16921 | ccaatcaatt tgggtgtata gaaaaaaatt cttgaaaata attctttttt tgctcaaaga |
| 16981 | taaatctgtt tattattgtt attcgattgt gtgctttgaa acactaggtc aaatacaatg |
| 17041 | tatcattttc aaatcatttg aagaaaactt ttcttaaagc tttttattca gtcattgtat |
| 17101 | tttcatttag ccacaaacaa ttttaatgtt tttgagcctg atggtcttaa acaggagaag |
| 17161 | gtgagagagt ctctgatctt caaagtagca gaaagcgggg ggcacaggct ctgctcacaa |
| 17221 | caaggtacta acatgtgtta tcaattttag ctgaagttca aaaggataga acttgttctt |
| 17281 | agcaaatgtt tgatatttct gccataacac cattagtcaa ataatgttca gactccaaga |
| 17341 | acaccttcca ttggccactt ttcacctagc gaaatagaag gaattatatt ggatgagatt |
| 17401 | aatgtatttt gtcttgcatt attttcagga aattttaaaa aattaccatg taacttaagt |
| 17461 | gtaactttgc attattgagg aatgtcatgg gcagaacgtt aagatgattt tcataattaa |
| 17521 | aacttatctg tagtggcatg agtatccact ttcactaaaa gcttagccat tggcttccag |
| 17581 | ttgcctgttt ccctgcatgt acggtaagga cacatttctg gaataagagg tcatcattta |
| 17641 | gtaacaatgc caaattctag ttattttctg tttgtttctc tctaaaaata ttctaaattt |
| 17701 | gtcttaagtg tactacactt catgtcatct taaaagacaa tacatacgtt gtacttacta |
| 17761 | tactagtaat ttacagagat agtttgttag gataacaaat tttggaaact ctaatgaccc |
| 17821 | aagttttatc atggattcat ggctgcaaat tgttaaaatg agttcagaac atgtctgtct |
| 17881 | aagtaaccac atcaacaaaa ttaaaaaatc actgctcgat atattagaaa aatataagac |
| 17941 | agggaatgtt atttttacat atttaaacag cttttgttac ttcttcatca ctgtccactt |
| 18001 | atgtcccttg gatgaatagg ccattttgac ttatagaaaa gtgtggtatt gtgtattttt |
| 18061 | ctcatttata tgtataggat ggttttctga tatttttgaa aaattaagat caacttacaa |
| 18121 | aatttgttag agcacgattt gtgtgtgccc aaagacacca tttctagagc actggtttac |
| 18181 | atatacagta aatctcctag agaaagaaga aatatgatga agcactagtt tagtagctgt |
| 18241 | ctgaataact gtggtcttac acaacctttg cagaaaggga aaaaaagtat cacctctaaa |
| 18301 | tgccgaggct agtaatatct tgataccaaa ctatacagct attatcagaa agtataataa |
| 18361 | cattattgta atgcataaac aaaaatttaa taacactaga ataattgtga agccatatct |
| 18421 | gaaagtgtat caaaaasgat actacaccat ttcccaagtg tagtttattc caagtatgaa |
| 18481 | aagttggttt tgtatttaac aattctaaaa actcataaca ttaataggta ataagaaagt |
| 18541 | tctatcttaa taaatgcaaa aaataaacta ttttaatttt aattttttat tagcacatca |
| 18601 | tacatagaaa acatgtattc ctgataaagg tatgtaaaca gcccccaaac aaacacaaac |
| 18661 | aaacccacaa atgtcacatt taatagtaaa actttaaaag catttctttt cgagaRaaaa |
| 18721 | caaaaagcct gttgttatta ttatttcaat tcaatattgt acaggaaatc ttaaaccttt |
| 18781 | ttttttgact tttacaaata aatcagctcc tttatttgca ttattttgag cactctgata |
| 18841 | atgacagtct tcactgatat ttttgaaatc ctgttttttg ttaaataagc ttttccaatt |
| 18901 | aggcaagcaa ataaataaaa gaaagcttgg aaaggagaaa aaaacaaaac tttttatgaa |
| 18961 | atcaaacaac atattaagag tattatatac attcattcca cttactgtat gccttacatt |
| 19021 | attgtgctta ctgcatgact cagctctaac tagcataatg gacagtaaat tagttgttat |
| 19081 | ctttgaagat gttaaatttc gtttcaagtt aagcataaat aaatataaat atttttcaag |
| 19141 | cagctgaaag taaagcattc tatgtgttag gattgataga gtagggttgg ttaaggaagt |
| 19201 | cttggagaaa gtgtttctca tcctgatttt gaagaaagaa attatctcta gccagtctga |
| 19261 | aattatagac ctaatttgat caattgtaac tggcacagtg gtaggtgttg gatttagaac |
| 19321 | tacagacaaa tgggcttggt ctttgctctc attaactcat agacgtaata ggggtcattt |
| 19381 | ccacacaata atgtcttgat taaaatggaa tttggaagac gttgccctgt aattttataa |
| 19441 | tgacttaata agaaacccac aaaaaaaaat accctaactc agctcaatat ttcatttcaa |
| 19501 | tatttcagtt caatattcat tacaatattt ttatcagtta ggtatgatat gtgaaaatct |
| 19561 | atgatacatt ttttattctc atcaaagatg attcaaatct tctttcctgg aaataggcaa |
| 19621 | attttgtcaa tttttttcaa ctttccatct cccgtttttt ttttttttcc ttccatcctg |
| 19681 | acactgatta tatagattgc tagcggctaa ggtcaaaggt tttgatttta taatataccc |
| 19741 | tcattagagt gtgtttaatc cttttgaaaa taagacatag agtgttagac ccaatgactg |
| 19801 | ttgttaatag ggagttaata gcatttccat ttgaaattat taaatgtaaa tatttaataa |
| 19861 | tgtttcaata tctgataaac agtttgtttg aatgaaaata ttttaatctg aaaaacaaag |
| 19921 | tttgttagaa aatttcaaag aaaatatctt tatgactgac tatatatgta tatatatatg |
| 19981 | cgtatgtatg tgtatatatt tcacattatt ctactctata agaaatgtga ttgagaaggt |
| 20041 | tgtgttaaaa tagctggtgc tgaatctgga attcttagac attttgaatc atatttcttc |
| 20101 | tgtcatcaag cggcgattca atgactagta atagctccga gccactgggg atagttggga |
| 20161 | aaaggaactg attataacag actgatttgc ttccctttac tttacaattg tgaaagatca |
| 20221 | tcattttatc taaactcata aatcaacttg agaacaaaag caaaactcca ctgcaaattt |
| 20281 | tgttttgctc atttttgatg gtttcatttc agcaagtcat agaacagtac tttcattgag |
| 20341 | tttagtatgt atacccagat gtgttttggg gtttctgaga gccgcaaatg atacaagctt |
| 20401 | tgtctggagg aagacgatca tgtcatttgt aaatcaaaat acacctggaa gagcatgcaa |
| 20461 | acttgagtac aaatacatgg cataatgcgc agatgagctg gcattttata aataatttcc |
| 20521 | caccacattt aatgcatgtt ggaactagtt ctcgccacta cagaagtctc tttttcttta |
| 20561 | accctattta ccataatcaa aatcacaagg gaaaccctaa gtcaagatac tgtttcccaa |
| 20641 | ggtagaatat ttgtaattct gtttactctt tgtatatttg atttgcagaa tctttgaacg |
| 20701 | taatgcaaag cacattttca gacttgccca tcttaactga tgtgtttaca aatacctgaa |
| 20761 | ttctagagtt aaatgcacgc tgatgtgatt tctgaatggt catggggctt tgacttaatt |
| 20821 | gactggtttg atttttcagc tacttttatt tcccttccca aacatcccac tagacatgct |
| 20881 | aatgagaaat ggtcataagg ctgggagcca gaaggaaagg agtgataagt aaaggtctga |
| 20941 | gtagtataaa aatgcagtaa ctggccaatg atattgagag ttggtggtgt gaggcaaaat |
| 21001 | atctgtgact gcatttgaag tgtgcattga attttggtaa catatcattc ctcccagcac |
| 21061 | tgtatgaagg aggaaagaca gaaactgatg gtgatttata gaaagtacac atatgcagga |
| 21121 | ttttattcat tcattcattt attcattcat ttcatttcag gccaggcatc tagaatcaaa |
| 21181 | cagtctctag ggaactgata ctaattcagg agagatctaa gtttccaagg aaatggtgaa |
| 21241 | tcttcccttt ctttttgaat attgtttaat caaagtttag ttggaatttt gataaagcaa |
| 21301 | attctggggg ctcaaggttt tgactgaagc aatttttcag atattactac attaactaga |
| 21361 | tagatgttac atggtgcctt aaagacataa atcagtgtct taacggaaaa tagatcaatg |
| 21421 | tatcaagtgg ttgtaacatg tataaaagtt atctcaaaaa ctatagaatt taaatagatt |
| 21481 | tttccttaat aaataagaca taattcctgg aatatattca aattactgaa tgtaaaagta |
| 21541 | taaccttcat gatgctaact cataatttac attttgtaca aatcaaagag ttttttacac |
| 21601 | atgaaaggaa aaaatcaaga aaaggaagtt attttctagc atcagaatgg caaactaacc |
| 21661 | tcatgataaa taagacacat aggaggaaaa gttgtgttat acagttatgt gtacattaat |
| 21721 | gtttgtttaa catctgttat ttgcttatga gttttcagca gtactcggtt aatactcaac |
| 21781 | tcacgtcttc tggacttgga aaaggagtga ttttccttcc tgtaaaatgc tggacttata |
| 21841 | actaaaatag tataagaata ataaagtaag ggaattacag tgtggtaata tgtataggtt |
| 21901 | ttgaaattag agacagtcgt gtttaaaatt ttcttgactg ctatctaatt gtattacctc |
| 21961 | aacaagttgc ttaaacccac aatgttttct gggtaggaat cagtaaaatg ggaataatgc |
| 22021 | atattgctaa agcataagta agctgtgagg attaagtgat ataacaaatg tcaagtacct |
| 22081 | aatgcaatgt tacataattc taatcatgaa aaaaaaatta ccagtatttc ttttatatca |
| 22141 | aagcatcctt tttttggtat ttttcttttt aaaatgttag ttttacataa tcttcttaat |
| 22201 | tgttaccata atgtatatag aattatattt tcagctttgt ataatattac gctgtaacaa |
| 22261 | ttgtctaaag tgcttaagtc ttaaggtcat cattttaatt taaaatcatc catagcttta |
| 22321 | acatgcaata actgactaaa tcattatctg tagtggaaca tttgttatat tcatagctct |
| 22381 | gtgtatccac tatacttagc acctttgtgt ctgatgcttt ttcatatgtt acatttttcc |
| 22441 | ctcattatca gtttccgaaa gtgacagcat agtgtgtaca ggttgcaact tttagcttta |
| 22501 | taacttgtta gatgtatgat gacaagcaag ttaattagcc tctgtgtctc agttttctca |
| 22561 | tgtgtaaaat ggaaataata atacttaata tgttaatata taaattaatt acaccagcat |
| 22621 | catgctcaca ataagtacta tttaaataca ttaaaatgct gggttaaagg tttcatggta |
| 22681 | ttttaggcta catatttgca aactggttat tacaagttac tgccaatagc aattttaatt |
| 22741 | tcagttttat tttccnttta aacatagcat tcatttcata atttttaagt gttaaacaaa |
| 22801 | ttttgtaaat aattataact attattctga attgttttgt agaactcgtg tagaatccat |
| 22861 | aaaggcagaa attttgatct acttgctccc cactaagcgg cacaaaagag aacacctagc |
| 22921 | acagtgccct acacgtatat atgtgtgtgt gaacatatag cacatataaa tacccataga |
| 22981 | aagaataaat aaatgttaaa ttcctaacat tatcttgggc ttggaggcag aaatatttgc |
| 23041 | tgttgaagat aagtgagaat ttaattaaag agaggaaagg ggcatatttt ttgtttactt |
| 23101 | gaaaggcctg tcaaaccaat ttagatgcta ttggtcatgg gcctttgttc tggaaaccaa |
| 23161 | tcttctccaa tttttgtggt ttttaaagtt ttattttcta atgaaaccta gggcttttta |
| 23221 | aaatttttaa aaattagaag acaggcaaag ttcttacatc tctataatcc tcaggtaaat |
| 23281 | ccaactatat tatatatgtt cattgtataa tacttgaact gtactgatta ttatttatta |
| 23341 | tttactgtat atctaggtaa gccagttatg attgtcacag aatacatgga gaatggttcc |
| 23401 | ttggatagtt tcctacgtgt aagtaagatg cacacacata catatatatg aataaattgc |
| 23461 | tgaaaacatt agagacaccc tccaatattg tgccaagcaa ttcagtaatc taagtttaaa |
| 23521 | gtaaaactga aatcttctga ggctaaatag acagagaagg gctgtaaaat tgcatctttt |
| 23581 | tttgcagcat tcaaatgtta atggtttata tttttaaaac aaatttgtga gttcttcttc |
| 23641 | aaaagactct ttttatactg ccaagattca cactcgttaa ataaaaataa aaaagaatcc |
| 23701 | taaagcaagt aataaaaccc actgatgtaa gacagaaagt ctctttttta agtaatctca |
| 23761 | gtctgatata attatttaat cacagtctga tataagacag accatctact ttccaaacag |
| 23821 | tgctgtcaga aaataagcat gtgctataat gctaaagtat atatagtttt aatttagata |
| 23881 | tatcattttg ctgttagata tgtacattaa ttttttagat tgagaagctt tatacgttta |
| 23941 | tctatcatct atctatctat ctatctatct atatatatat ctatatatat atattttttg |
| 24001 | agatggagtt ttactcttgt tgcccaggca atggtgcaat ggtgcaatgg tgcaatggtg |
| 24061 | caatggtgca atggtgcaat cttggctcac tgcaatcccc gcctgctggg ttcaagtgat |
| 24121 | tttcctgcct cagccttcca aatagcaggg attacaggcg cccaccatca tgcctggcta |
| 24181 | gtttttgtat ttttagtaga ggtggtgttt caccatgttg gccaggctgg tttagaaccc |
| 24241 | ctgacctcag atgatccgcc ctcctcagcc tcccaaattg ataggattac aggcatgagc |
| 24301 | caccgcacat ggccatattt tgacatacta tatttcacca taagaaagtg aatatattaa |
| 24361 | atttacatct atttcaacaa atgttatgtt caacaatttc atgaagttca agacaatacc |
| 24421 | aattaccagt cattgcaggc gataagaaaa gacataggcc ctgtcctcaa ggtgcttttg |
| 24481 | ttgctatata aattgagatg ggcagatgac agaaagatag atagataaat agataaagaa |
| 24541 | aatatgtagt atcattcata atcatggagg aaaattttaa attttctaag actgagaaag |
| 24601 | gatatgcttt atattaaaat ccattagcca aataaaaatt ttaatagcaa ttattttcat |
| 24661 | aatcgttatt tcaaaaataa acatttccaa aatcaggtga gttaaaatat tatgataaat |
| 24721 | attctttcaa aattatcgtt tgtaattaaa catggaattt tatataaata cattatctat |
| 24781 | acattttaaa ccttatgttt ctattttaat ccaagattaa attagattgg aagctggatt |
| 24841 | atgtgattta gtttcttatt gctttctatt ttgtgactac aattcagaag tgggtagaaa |
| 24901 | aacaaatatc caataaatat ttttgactaa ttatgaacta attatttatg ataatgttta |
| 24961 | aaaatgtagc atccacctta tttgaaacaa ttcataaaat atcaggaaac aaagaagggg |
| 25021 | aaatgaaaat tcaatcaaac aaataaatat caacctaaac tagccacaag attcatttca |
| 25081 | cttttttgtt cttaacctta atgagtctga gcaggagtta gtttttttgt cacataagga |
| 25141 | ccaggaaagt ccttgctttt aaactgaagt caaagaacgt taagatactt cctgttacaa |
| 25201 | agtatttgat aacatagcca gtgtgttatt ttgtttcagc cttgtatcca tttgccacat |
| 25261 | atctttgtct tgagtgctta ggactaaagt gtgtatagtc agtcttgtta caaaattcaa |
| 25321 | tatgtgagat tttaaccaat aaagatatat ctttaagaaa taggaacgta tcttaattgt |
| 25381 | acatttgaaa tgcttcccag aaacacgatg cccagtttac tgtcattcag ctagtgggga |
| 25441 | tgcttcgagg gatagcatct ggcatgaagt acctgtcaga catgggctat gttcaccgag |
| 25501 | acctcgctgc tcggaacatc ttgatcaaca gtaacttggt gtgtaaggtt tctgatttcg |
| 25561 | gactttcgcg tgtcctggag gatgacccag aagctgctta tacaacaaga gtgagtaact |
| 25621 | tagattttct ccttttttat cattgttttc catcttgtat catgttgatt tgtaaataag |
| 25681 | tagaaatcat gacccaaaac gtgttgtcaa ttatgctttc cacaatagaa aacatatctt |
| 25741 | aaaattaaaa tattattatt tattctgggt aaatagatgg tcactgttta acatttaatg |
| 25801 | attttaactc tgaaattcta tacctcagtt taatgttccc caccaaaagc agcaagattc |
| 25861 | tcacattcct caaatcttga tatttttaaa tgtacccatc ttttcaccat ggttgagtca |
| 25921 | ccctgagcta gcagattgga taaattctaa ggaccttatc ccagtacata tcattttaaa |
| 25981 | gcactttcaa atcaattttt taataagtaa tacctattta tactgtaaaa atgtcaaaac |
| 26041 | agacctataa agaaaagttg tccataattg tgccatccac agacaacatt tataagattg |
| 26101 | tgagtcaatt cttacaaatt tttcttcata ctcatatgta caaacttgtt catgaatata |
| 26161 | ttatttaaat acaattattc ataatacaat tgtatgccat ttctctcctt accaatacac |
| 26221 | ttcttgaata tatttcattg tcaataaata atctttaaat ttttacatgt agtaggttgc |
| 26281 | tgtattctat attgttgtat cttttttaaa gcaaacccta attagtgagc aaattattat |
| 26341 | tatatttaat ttttccatat tttataccaa tgtatgtgat ctgcctgcat tcataattgt |
| 26401 | tatgagcctc atgattttct aatgatacat tcctaaaaat gaatgttttg tgtcaaagat |
| 26461 | tacgtgaata ttttaaaagc acactttact acactcttga taaaactagg tattttcatt |
| 26521 | aattttacta ttatcagtta ggataaattg aatgtatatg ttttaatttg catccattta |
| 26581 | attcaaaatg catttgaatt ttttgtgagc ttttctgttg ttgttttgtt ttgtgtttgt |
| 26641 | tttttgtttt ttaggcaggg tctcactctg tcacccaggc tggagtgcag tggcacaatt |
| 26701 | acgactcatt gtagcagcct caaactcctg gcctcaagtg attctcccgg gctcaagtga |
| 26761 | tcctccctac tcagcctgat gagtagctgg gattacaggc atggaccacc agccccagct |
| 26221 | aatttttaag tttttcttta aagaaaatta tttccatggt agaaatttag aaaatataga |
| 26881 | taaaaaacaa gtaaatgtct aataatcatt actattaata ctacacaaac ataattatta |
| 26941 | gcattttaaa tttagccttt cgattttttt aaataaattt tcacaaataa ataactcctc |
| 27001 | ttttggcata aagttacaaa aatgggatta tactttatat actgatatat aaaatgattt |
| 27061 | caacataccg ataatttagg cactttactt atatataaat caatgccatt ttgttttctg |
| 27121 | ccctgtttta acttaaaaat ataaaataca taagtttcca gatgattaaa tatttttaca |
| 27181 | atattcaata atgtctaata ctctattcat atacataatt taattaaata aagcactatt |
| 27241 | aattgggcag aattacaata ttttcatatt atgagtgata ctttgatgtc atgatatcta |
| 27301 | ttttgtgtct gtccatgatt acctccttgg gatatttcta tagaattaaa attacaggat |
| 27361 | caaaacgtat acaagtatgt gagaattttt ggcgtatata ttgcccaatt gtctttgctg |
| 27421 | tagtttgtta tcaattttct ctcctaatgg cagtatcttt ctccatattc tcatcagata |
| 27481 | agagaatagg tggttccaaa gatggtagtc ttctgctgtt tcttattatc taattttaaa |
| 27541 | agtttattcc cgattaaaaa aaaaagctca tcctatttgc aaatcactcg agaactccaa |
| 27601 | taatcaaaat tgtcacatgg catgcatatc atgctataat aaagaaccta atatgccaga |
| 27661 | tagcatttca tccacaagta aagccgcaat aatagaacca ctgaataata atgacacaat |
| 27721 | gctgttggga tatttctaga tgcattgata taggaggtag gactctccca atggctttta |
| 27781 | ataataaacc attttgtttt tccaattttc aaatatactc tgtagtgcat ttactttgtt |
| 27841 | catgaatcta aatttttttt taatctgagt attttttaaa aagtacttag acacttaaaa |
| 27901 | tgtgtcctac ataaaaatgt tgttcttaac aaacacaatt caaatggata agagcaagac |
| 27961 | aaattaagac aaatttctta tcttgtagct agttgaatta aatttacaag tgactacaat |
| 28021 | tatatagata tgaacatgat gaaattgcca aacatgggat caatacctca acagtgcttt |
| 28081 | ttagttctcc ttctctgatc accagaaaaa ttagtcagcc aatgtatctg caacaaagca |
| 28141 | tcattttaat aactgagcta ccaattcagt agacactata tgcagcaagt gatacttaac |
| 28201 | agtcttaggg aatgtatttc attctactca cttctactct ttttctaagg ctatccctct |
| 28261 | taacctgtca tgacataagt acttctaaga gtccatgaaa agtatcatgc ttacttatat |
| 28321 | tgtccgtgtt acaacaatca tattatgcca gaaaaatact tttaggttaa ataaaaacta |
| 26381 | tttgataaaa aaattatttg aaatgttaaa tttctggtta ttcagtcagg ttctaatttc |
| 28441 | tatgccatat taaattattt atttatttat ttatttatta atttttgaga cagagcctca |
| 28501 | cgttgtctcc catgctggag tgcagtggcg cagtgtcggt tcactgcaac ctctgcctcc |
| 28561 | cagattcaag caactctcat gcctcagcct cacaagtagt ttggataaca gtggcttgcc |
| 28621 | actacgcctg gctaattttt gtatttttag tacagacggg gttactccat gttggccagt |
| 28681 | ctagtctcaa actgctgatc tcaggtgatc cacctgcctc agcctctgaa agtgctggga |
| 28741 | ttacaggcgt gagccactgt taccagcccc atattaaatt attatgtgag agcatctctg |
| 28801 | tccccataag taggcttgat tgccaggata ctcaaaagaa tagtttctct ggcaggaatt |
| 28861 | ggaaacattt gtccaacttt agatgattca tactctggag aacacagggg gttaactcta |
| 28921 | aatgtttcct ttacactgat tgttgttttg tcagggcctc tttagtgtta gaagaaaagt |
| 28981 | gtcgtatgga aattgctctt taacatttct gaaataaaag tgaaacaggc tctttgcatt |
| 29041 | tctgttccag tcactgaaga ttcttaaaaa cacactgaac aaataggctc attaaccaca |
| 29101 | aataaatagc tgtttcgagt tcttcagggt tcatctttgt atttccttgg aactccctca |
| 29161 | aaatatccat gggagtcctg gctttcttcc gagaagctgt aatttgcatg tgcgtttcta |
| 29221 | gagtaattta aacatcattg atttaatctc tcataatagg ccttattgag tacaataagt |
| 29281 | ttatagttta ctctaaatat gaggtatggt tatgaagtaa taagaagact tcaatttttt |
| 29341 | aaaaaaacaa atataccaaa cagctaaaYg aggcagcatt ctaagttttc cttttggaaa |
| 29401 | taaatatata cttactacag gaatgctgat attgcacaaa atgttttggg aatttctctt |
| 29461 | ttggaattat atttaaagct aaattataac tgatgcatta atcaaacatt tttaagcagg |
| 29521 | tagtattttg ggatataggc ttgactaaga taaaatctgg tctcgataag cagagtgggg |
| 29581 | tggctcacgc ctataatctt gacacttcga gaggccaagg caggaggaca tcttaagctc |
| 29641 | aggagtttga gaccagcctg ggcaacatag caagacttca tctctgttaa aaaatatata |
| 29701 | tatatataaa tatatattta tatatataaa taatatataa tatatataca cttttatata |
| 29761 | taaatgctat atctatataa atactatatc tatataaagt atatatatac tatatagtat |
| 29821 | atataatttt atatataaat acatctatat atatataaat aaattatata tgttatatat |
| 29881 | aaatactata tatatgtgtg tatatgtatt aaaataatat aggacaggtg cggtggctca |
| 29941 | tgcctgtaat cccagcactt tgagaggcta aagcaggcag atcacctgag gtcagaagct |
| 30001 | caagaccagc ctggccaaca tggtgaaacc ccgtgtctac taaaaataca aaaattagct |
| 30061 | gggtgtggtg gtgtgcgcct gtaatcccag ctacttggga ggctgaggca taagaactgc |
| 30121 | ttgaacctgg gaggcagagg ttgcagcgaa cggagattgt gacattgcac tctagcctgg |
| 30181 | gggacagagt gagaatccat ctcaataata ataataataa ttaattaata aaaataaaaa |
| 30241 | catatattag gcatggagac gctcacctgt ggtcccaact actcaggagg ctgaggtgga |
| 30301 | aagatcactt gagtccagga ggttgaagct gcagtgagct aaagcctggg gatcgagcag |
| 30361 | gacccttttt caaaaaataa ataaataata ataaatttaa aaggaagaag aaaagaaaaa |
| 30421 | gaaaaaatac tattacattt ttgtgtatat attgtctgtt tgtcaaccca aagcaatata |
| 30481 | cccagcttag acatctgtct tgtttatttg atttagtgtg ctctgattat tgttatttca |
| 30541 | gaaaaacaag ttcatcattc aaaacaattt gctgaggcat tccttttgga taagagatcc |
| 30601 | tggttaatct gcattaaaaa gtcagtcaca ttaataaagt ttaacgcagt tcatctagtg |
| 30661 | tctgaagttt ctacaatttg gagattaaca tttggtgcct caatgcaatg acccttccct |
| 30721 | ggttgctcct ctgaaagtta ctgcctgtag gtagagcgta gttgcactga agagtcatga |
| 30781 | aggacatttg aatcaatgtc agtggaaaag aatactgaac atagaatgtc tgtcgctctt |
| 30841 | gttttaaaac atctctgtga gtgacaggca gaatagagga atgtatagaa attatataat |
| 30901 | ctaattatgt atttaaaact tcttaaactt tgaagagtat ttgaggagtt gaggaaacac |
| 30961 | ctaagctcaa aacttaattt atcagacagt caaagatatt ttctcacact gtgttctata |
| 31021 | ctgtcttagg tgtatcacaa gctttccttc cttatgttct cgacagcatg ccggatatga |
| 31081 | aagggtcagc taagatagta ctatacattt ttatgtttat tttctctttt acataaggat |
| 31141 | attgtgtaag gactaagatt tttttcccca atactcactt gttgttattt cttctcattt |
| 31201 | cttactgctg tgttacgcag aaattgcaaa tgttgatatt ttccattata caatgttatt |
| 31261 | atgcatcctg taaaactcca ctgtactctc atgggatgtt cagaatgaat aaggctatga |
| 31321 | agtttcagta ttattatgaa aatagtttct atcttgtgga cccctaaaat gctcttgggg |
| 31381 | acccacatac atgctcggat gacagattga aagccactgt tatagatact ttactaggaa |
| 31441 | aaaagtccct taataactaa atgaatattt ttgccgttat tcactgatat aaatctaaat |
| 31501 | tatggcatag ttttatgtga tataatatat tttaaagaag tagctttgaa agcagaactg |
| 31561 | cagctgcata ccaaagtttg caaatcatct tgaaagtccc tggatttcgt ttgggatgga |
| 31621 | tatgattaga ccatttagct ccacaaattt aaatcatcaa agaagttttg tgtctgaaaa |
| 31681 | aaaaaacaga aaaattaatt cctttcctct ggtttctcat catgtgactt tgaaagtata |
| 31741 | attataaaat gtttgttata tttttgccct ggtctactaa tttacttcat tctaacagaa |
| 31801 | tcatgacact attgtagaac agtttgccac tgaatgattt ttcaattttt gcttctatga |
| 31861 | aattttatct ttaactggtt agtatttttg tatatatgca ttcatgcatc tatatagatg |
| 31921 | aatatttcta tattcatatg ttgaaataca tggaatccaa aattccaaaa tggtagtgtt |
| 31981 | taattttctg ttttatacag gatcaagtta ctgaaagcac agacttttat cttatttaga |
| 32041 | tgctgggtaa gccatcactt caattcttcc tatgtcctta agttcatttt gggagcataa |
| 32101 | ttaccacata aactgagttg gaaagtttga gaaaacaaat tgaattgtcc ttggctatat |
| 32161 | tctccattat tcgatttttt tcttttcatt tctattctga agttgcctac ttagtaagta |
| 32221 | ctaactagat tttgttacaa cattttattt tacataataa tttatttggc tttttagatc |
| 32281 | tgtgactctg ccatattgat gccagatata ttctatacaa ctttgttttg tttgatttca |
| 32341 | tttttgtttt tttgttttgt tttgttttgt tttttttgag atggagtctc actctgtcgc |
| 32401 | ccaggctgga gtgcagtggt gggatctcgg ctcactgcaa gctctgcctc ccgggttcac |
| 32461 | gccattctcc tgcctcagcc tccggagtag ctgggaccac aggggcccat cactacaccc |
| 32521 | ggctaatttt tttttgtatt tttagtagag acggggtttc accatgttag ccaggatggt |
| 32581 | ctcgatctcc tgacctcgtg atccgccagc ctcggcctcc caaagtgctg ggattacagg |
| 32641 | tgtgagccac cgtgcctggc ctgtttgatt tcattttaag gataaggcaa aaaaagaaaa |
| 32701 | gtggctaagc aagtggttaa aagctaagag gttaaatatt tcctttcagg taactccaat |
| 32761 | ctaagaacat aaataacaga aaacaggagc ctttgttctg taacattttc aagaaccaaa |
| 32821 | aaaaccatgc ttatcaaaat tggtataata caaaagacac atattaattt gataagtaat |
| 32881 | atatgtcgca tgtcatttat acctaaatga aaaaggtaat aacaaaatat ctaatttgtg |
| 32941 | tccttcatat agtaacagta ttttaaactg tgaaccaaaa aaagtcaact acgaatttat |
| 33001 | tgcctcccct tatgatcaat atgaatatgc ctgcatgtag catcagaaaa tacagccact |
| 33061 | tattcaataa ctagaaaact ctcaaaggtt cagtgatttt aataattaat gttgaatcga |
| 33121 | cacttaatga aaagtctagt actttctacc catctatttc agagagatga gtagcatgaa |
| 33181 | catttaaaca tgtaaaaaac aactatccag gtttatgtgg tgaaaaatta caactataat |
| 33241 | tgtgaagtgg aacacaggag cacacaagta aaggcagaaa agtaaaatga ggaatgcagg |
| 33301 | cagtctgtag agaatgcaat tttatatagg atcgtcagag aaattggtaa aatgacacta |
| 33361 | gagcagaaaa tgggtttgaa tgaaggagca aatactgagg ctaaagaaac attaattttg |
| 33421 | ctttcactaa ttttcaattg aatagaataa ttttaataat tgcactgatt ccaagaggcc |
| 33481 | caccttgcag gaagacaatg taagtcatta gattaattat ttagctcaga ctaaatgtca |
| 33541 | agataatagt aactagtcct ctttttctat tggtgatgca tgcctttagt cccagctact |
| 33601 | caggaggctg acgtgggagg gtcgcttgag cccaggaggt cagggctgca gtgagccttg |
| 33661 | attatgccat tgcactttag gctggggcaa cagagtgaga ttttatctca aaacaattat |
| 33721 | tagaatgaat ttagtattat ttctatcatt tcaattgcaa attgtcagta tcccctgcta |
| 33781 | cgtttctact aatttaccat cgcttcttca aaaaattgtg ttcagagtgc tacaatttca |
| 33841 | gactttgaaa tgaatcacta taacttttaa aaattagaga ttttaaaaac tggagattga |
| 33901 | atatatataa aaaatacaaa atttgacatt taaaacctca ttgaactttt aaaaaagcaa |
| 33961 | gactcatatt aaggcacaca cacacacaca cacacacaca cacacatata tatatgtatg |
| 34021 | tatgtatata gacttactcc ccgatgtttg cattattttt gtagaagata gatatcatac |
| 34021 | ataagattac ttagttttta cactgtttca tgagagtccg aaagtaacat aagcattatg |
| 34141 | agtttgtttt ttatttcctg gttagccatt ataataattt ggaatatgga gattttcctg |
| 34201 | tatttcaagt aatgcttaat catttcacat gatgttgaaa gatctcaaaa gttcgaagaa |
| 34261 | tgaggttttg ctacctaaag gatcatttat ctctgatctc ttgtggcaat caacatttgc |
| 34321 | tgaaactatt ctctcagtgg cgctgagatt tgaactgctg gggataagac tagatgcttc |
| 34381 | ctttgggagc cttcttggat cacccttcac cttgttgtct gcagtgaatg aattttagtc |
| 34441 | cttgacaata catagatgct aaatctttta gccaaataat ttatctctat agttccactt |
| 34501 | ttaacctgag aagctaatat ttccctccag gttaaatact caattacttg ggtttactca |
| 34561 | gataaactct ccaaagtcct ttgtcactct aaatatgacc tgacaaagct caaaaacagg |
| 34621 | aaatgtgaat tataaccagc aaacctttcc taaattggca ggcagagatt tgtacatata |
| 34681 | attacagaaa catttggcag ctatgtgata ttatgcctca atacccagaa atttcagcaa |
| 34741 | ttacactcct tctctttaga aatcagaact tgaagccccg taacccatac cgcatgagct |
| 34801 | gacttcagtt aagctcctga aaaattcagg cttttagcca catgtctcat tgaactgtct |
| 34861 | aaattcatcg agcttaggga actgaattgc agaatctttc aagaacaggg ttaccgtgtg |
| 34921 | attcctcatc taactgctgt atctaggcaa agtggacaag gttttagact tcaactaat |
| 34981 | cctggagtgg agtttagcag ttatgtagtg cctttttcca gagaatcact agcatcttta |
| 35041 | cctgtagcac tttattcatc tcctttacct tttctgtaaa actctataga cgtaaatcga |
| 35101 | ggtgacaccc atgtggatat gcaatttatt ccaaagttag tctttcttta agagtccaaa |
| 35161 | ataattaaca ttgaaaatgt cagcatcctt gatactcatg taggtttaag atagaaagtt |
| 35221 | caaagagcta tggtcataag tcagcaaagc tgttaagctg ttagataaaa ctgaatgtaa |
| 35281 | agtcaaattt tatatagtcc atgaccctgt ttttgaatga caagacagct gtgcagagag |
| 35341 | gttaagacac ctggctaaaa ttaaacagga aggtcaaaac tgaggccaga aaccaagact |
| 35401 | cctgattctt attaaacttc agaatccaaa tggagcaaat gactaaactt accctgtacc |
| 35461 | tccagttatt tagctataca tagatgagaa gtcagcactt ggaggaactg gctgaaggtt |
| 35521 | tggcactgga gatgtaagga gcaaacatta gcttctgatt ctgttaagtt tgaattggaa |
| 35581 | gtctttctga aattgaggtt ttagatacac tccatatgct atgattctgg gcttgctcca |
| 35641 | agcaactgga agcttttctg aaatacataa atatatcttg ggagcttaca ttaggatata |
| 35701 | gagaccagta aaagtaaatg cttcatttta aatgtttaaa tatggttaaa aaacacatct |
| 35761 | tcataaataa ttgttatagt gactagctga gcttttataa tattctaaat gcctagtgtg |
| 35821 | gcacatccct ctaaagcatc tacattttaa aaatgtcctt cacatatata agctaggaaa |
| 35281 | gcaatttcta aaccagaaaa aaataacact catgcaatat gagcttgaat ttgttaaggt |
| 35941 | gggaggtgag aagggaagag gaatttttgt ggtcacaatc aagaacagaa atatgcatac |
| 36001 | gctctgtgat gaattacttg taggaaactt agactcctat atattcaatt tattttagtt |
| 36061 | tgatagtctc agaactacag atcctcagct taaatgcagc aagtcttatt gtaaagtgtg |
| 36121 | actcagttaa aatttattgc cccagcttct tcttttaacc atatttcaga accaaattgg |
| 36181 | gccctcaagg actcttattc gtgtctctct atgccaataa tgtttacgtt tttgtagctg |
| 36241 | tagacagtgt ttgctgttgg agcaagcact tgtatcttac taaaatgctt ggttacttat |
| 36301 | acctccattt aataaaaaaa ttgatgctta atttataaag ttaatttaag ttttactagc |
| 36361 | aaacttaaat taacaggaag ttatcctaaa aaagaaaaag aaaaggaagg aagataggaa |
| 36421 | aaaagaagaa aggagaaaat aaaataaaac ataaaaaaat tccagaaggg atattattac |
| 36481 | atcatcaaaa cccaagtatt aggagattgg ctctggtgaa cttgacccct gtttttctat |
| 36541 | tagtaaaggt atagcctgtg acaggggtat aaaatatccc tagaaagttg atttcactta |
| 36601 | gtcaatataa aatcccacat aatttgtaga cctcatgtgg attactttcc cttgatgatt |
| 36661 | gtacaacatt taaagcagta gttcgcaaat ggggatgatt ttgttcccca gaagatactt |
| 36721 | ggtattgtct agaggcattt ttggctgtca caactgggga gatgctaatg taatctagat |
| 36781 | gacagcggca gaagtgctgc tagatatcct acaacgcaca ggacagccgt cacagcaaag |
| 36841 | aatgatctcg ggtgaaaggt caacagtgcc cttctagaga aaccttgcct acaaggttcc |
| 36901 | catcatgtgc aaagaatggg agtggggatc aaatgtttct ctcaggtact ttctattttt |
| 36961 | tcctgtccat attggactgc aagatgcatg ggacaatata gactacacca tttgtgaaaa |
| 37021 | aatgtgcaac agactagcca tctgttatca ggcctcctag aatgtgaaac ttcaagctat |
| 37081 | cacaaacata atgcaacaaa tcataagtct tgtgtgcctg ttaaaatttc attttcattt |
| 37141 | ggtttcaatt aaacacttaa attacttacc accctccacc ccaggctcta aatttcaaac |
| 37201 | atagccactt tagttgtgat gttctatttt cattttatta agacatcatg gagtaaaatc |
| 37261 | atacatagat gtatatctct agaagataaa tatatcagat attttagtca gtatataagt |
| 37321 | cttacccttg tgtaattttt ttaatgtttt aacattttac ttagcctcat tctacaaagg |
| 37381 | ggtaacgtcc atgtgtgtag actaaaatta ttttttaatt aattgtattt ttaggttggt |
| 37441 | gaagtagcta caacattaat attgaagcag ttgatgtaga atgtgtattg tttatggagt |
| 37501 | caaatgaaaa tctttatagc attctacgaa ctctaaaaca cataatccat acaacatcct |
| 37561 | gtgacatatt tttgtaattc tacagatact ctaaaaaagt tgatgctcag tgaatcttat |
| 37621 | actaactatc gttgtggagt tcttaaaatg tattaatgta acaacactat taaatagtag |
| 37681 | cataagaaaa aagaggacat tagaaagcag tatgatttaa gaaaataaac tattatgttt |
| 37741 | atattggctc attcacattc acatatatta tgaaaaacta gatttaactc attatcaact |
| 37801 | gaaaatgaac tgcaccttta aaatgcacaa aattatcaga aatctttaga aaaatctact |
| 37861 | ttcccaggtc cacaaaataa atcattattc tcacttgaca gaacaacgtg cttttttgca |
| 37921 | agcaacaaag ttttgtaaac agcagaggta gaaaataaga catactttaa actgggggaa |
| 37981 | tacagtggtg aagggggcca ggtggttatt cctctggaca tcccagatta ggcaggataa |
| 38041 | taaagcagta gaaagatcac agaggatgga atgaccaact cttgctggaa gactcctaaa |
| 38101 | tgttcaccca aagagtttac atttgaccaa gatgttcaga taagaacttt acccagttac |
| 38161 | agaaaaaagg aaaaaaagcc tcccaaaaag aaagaccatg tatttgggaa gatagcatgc |
| 38221 | tcacaggggg aattcaatta agatcagtga gcatctgttg aatgcctgca ctgcacattt |
| 38281 | gccaagataa taccaagata atactacaga gagcaagaaa actgctctct gtagtgaatg |
| 38341 | tgtattgttt atggagaaca tagtttcatg tgagttaata ctccttatct tcagctttat |
| 38401 | aatctattca gtttttcata agcttatgat ttgaaataat ggttcgggtt tactattttt |
| 38461 | atttgttgca attgatatga caggcactac tgctactact tttactagtg tagaacataa |
| 38521 | agagtttgaa actacaatga agaatttaaa gagtttatga gaatgtatgt atctatcttt |
| 38581 | tcttaaattc cataataatt ttgctcgttc gaaatttcac ctgtagtttg attttttttt |
| 38641 | ttacttcaaa ttaaatgtgt acaatgttgg tttattattc ttttattttt aaagccagag |
| 38701 | caatttgata ttatttgctt gccatagaat aagataattt ttaaaaattt ctttttatct |
| 38761 | gatttagtat taattgctaa atttgaattg tactttgtag ttaacagtga gctttccctt |
| 38821 | gtagcatctt ttaataaatt tgttatattg acagtaaatg taacaattaa agggaattga |
| 38881 | aaggaaggaa caggcataac atttattgaa tgctctatta aggagcagac actttttaaa |
| 38941 | tggctttata tatattataa attcatgaca aatgacaaaa ttttctaaat aataatatat |
| 39001 | aattctcaat catctttatt ctcttcctgt ttccctcaat gatggatggc atcatttaca |
| 39061 | atatggttag atagaaaaga gataagagtc ttgaatctgt gattatttca ttattggagc |
| 39121 | taaactaatc Mgtctaataa gctacctatt agcagataat ggaaattctc taatgaaaag |
| 39181 | gctaaatgtt tcatgaatat tgaatacatt ctgggctaaa cagctaactc ttggccaggt |
| 39241 | gcgatggctc acacctgtgt aacaccagca ctttggtagg tggaggtggg aaaatgactt |
| 39301 | gaggccaaga gttagagact agcctgacca acatggcaaa accttgtctc tatcaaaaat |
| 39361 | ccaaatatta gccaggcttg gtggcgcaca cctctaatcc cagctactaa ggaggctgag |
| 39421 | gcacaagaat tgcttgagcc tgagaggggg aggttgcagt gagccgagaa cgcactgcac |
| 39481 | caccgcattc cagacctaac tgtctatgga cttcaacact aaaattgggc aaagcaaatc |
| 39541 | acttattcac atcttcttct aaagaaagag tttaaattag ccccagctct tcttttagac |
| 39601 | tgagttgttt ggatatcata tataaaaata taaattaaga gtttttcatt aaaaataaaa |
| 39661 | ataaatatgt ttacttctaa gtatataagc tcaattaact gtgacctggg acagaatctg |
| 39721 | gccagcgagg aaaagaactg gtacaccaaa agttgatcaa tagtagtatc tggataaatt |
| 39781 | acggaaacat ttaaagcact gcaccctggt ttcattgttg aactgattga atgccactta |
| 39841 | attttcgagt gtttgtaata acatgtccaa aataaaatga aaataaatga gattgcccca |
| 39901 | cttccttatg ctttcccaca agggcagcaa ctgaaccctt gatatagtta agagacaaaa |
| 39961 | aggaaaagtg taaatcaaaa acctgaatga cacaagtgaa aagaccatac ctacatttcc |
| 40021 | aaagctagca aaacttgtag caagaggcct ttgccatttc acaggtatct actgcacaat |
| 40081 | gtggccaaga tacttttcca aggtaccaca gtgagctaag agccagaata ttttgttaaa |
| 40141 | aattagtacg aaggtaaaga tagtataaag gtcacctgat cagcaagtat gttacattga |
| 40201 | aaggtttaaa gttgtcattc taacaataat atgtgtgtgt gtgtgtgcag gctacaacaa |
| 40261 | ttacaaaaca caaccagaag agcagaattg aaaatttaag tttttcttat taaaataaat |
| 40321 | tactatacat ttttccaaac atgcatccca tttgtttaaa ttttaagcat tttaataaga |
| 40381 | tttacacagg ttacacaaat agaccacatg taattgggaa atattctgga aataatttgc |
| 40441 | ttgaatctgt aaccatgtat ttcattagag aacatactgt ataagtccca ggcaagttaa |
| 40501 | gagattcagg tgagtgaagc tttccttgaa gatatgatca tgcgaccaca gcagatacct |
| 40561 | ctccaacatt attatgtctg tatttacaac agagcaaaag aggattctcc ttccaagctt |
| 40621 | aagcagaaat atggctctgt tcttcctaaa aatgatcacc tttcagtata tcattatttt |
| 40681 | aagaaattct attaatctca atgctcttct ctctggaaaa tggtgaagaa acacttaaaa |
| 40741 | tgaaacaact cttctgtgtt tctttttaac cctgattgaa ttaaccaaat aaatacactt |
| 40801 | ctctttcatg gctgtcatca acctattgtt tttttcttat gactctattt taatttcaac |
| 40861 | tagagaccat ggtaaggcaa caaatatctg ggcctaatat tttctaaata ggtacaaagt |
| 40921 | tttcagcata gaaagggctt ttttttttct tttttctttt ttttagtgcc atttaagtac |
| 40981 | acttcacaat ttcagtatta ctgaaagtat actgacatga tcctttcagt ataagtatac |
| 41041 | ttatatacct tcagtaatac tgaaagtata cttatacgct ttcagtaatt tggttctgga |
| 41101 | aaaagagaag tagcaaaggc agttagaaaa atgacaagtg tctataacag acctaagata |
| 41161 | tttaattgag atttttaaga tactgtacct cccttgtcct atgaatacag ctctgtttga |
| 41221 | cttttgttaa ttctctgagt tcttaagaag gagaaagtag gcccaatgat tccatttggc |
| 41281 | atcctgagta gattctgttc aatgccacat gtctaagtcc acttttgtcg atgtcaaggg |
| 41341 | gtagtatttg aaattttgaa ttataaaaac ataaacttga tctaggcata tgtagatgag |
| 41401 | tgctttggga gttttagtgc acgttgcaca ctgtagttta tctccctaac ttccgaggct |
| 41461 | atcttttctc tggctccggt ctctccaatt tcatttgttt tacttcatca gtagaaactg |
| 41521 | tcaactattc ttcacctggt agcttcttag gaaggttgcg ctcactgtgt gtctttgcag |
| 41581 | gcttattctt cctgctgatg gtgtttttcc atttcctcaa ttctctgcac tgtttcttgc |
| 41641 | attcttcatg actgtgtaag ctctaccttt tcaaagagct tctggcagta ctctccctga |
| 41701 | acagctcatt tgtgccaaaa caccaaggag aagtgctcag ttttactgag ttagttccaa |
| 41761 | tcttataact ctcctagcca ggataaagtt tcccaagtgc atctgtgatt aaataaaaat |
| 41821 | ttgacatttt agcttcctct tccattatat ttcttcacac aggaaactgt tcttctagaa |
| 41861 | catacagttt tcccctctgt gctttgattt atgccatttg ctcagaatat gagagtattg |
| 41941 | agctaacatt tattgagtcc ttactatgtc ctaggaagaa gactaaatat tttactgaaa |
| 42001 | tttgtcgttt agtcccacat aaatactctg aaccaggact attacattct caataacaga |
| 42061 | tgaggacagt cctgcacaac aaggcttaga aacttgtccc ctaaattgct aatccatttg |
| 42121 | tgtaaagcac tatattgccc ccttgagcat cacttcaaat ccagtggcat gctatgaggt |
| 42181 | ccgatagaaa tcggtcacat tttgacgaac acggcttaag attttgagga tgtgagttct |
| 42241 | ttctttgaaa tatccatccc ttttttctta tatcaaatat tgtacttatc tttcaagtga |
| 42301 | cttcacttga aattgaggtc acttaattca taagtgccct cctcagtctc tctagtgaga |
| 42361 | atcttggctc cacgctccac attacggtgg ctttcactac tgtctttcta ctcagggtaa |
| 42421 | gttccatgga ccagcatcac atggagattg ttggaaatgc agacactggc tacacactca |
| 42481 | acctcctgaa tcaaaatttt cattttactg ggagtaagct gtatatgatt tatatgcatt |
| 42541 | ataaagtttg aaaagcaatg tgctagatac ctatttcgtt ctgtatctag tttttcagag |
| 42601 | taactcatta ttactcctgt ttgcctgaca cttacgaaac aaaagagcca gtatgttatt |
| 42661 | tatcagtttc acccaaatta actagcataa tacttcctat atggttgaca atcagtacat |
| 42721 | ctttattaaa tgtatttatt gaactaagag caaattccat agacattaca cttttatatc |
| 42781 | tcccataact tataaaagca atattatttt cctctaaatt ataagttctg ggaatgtaat |
| 42241 | tattatgtct atctaattta tatggtgctt gcctactatc cagaagacac ttcaatgact |
| 42901 | aatgatgaag cacacagtaa attaatcaag atgattattg tgttgaagag agttttttaa |
| 42961 | aatataattt tattttgagt ttatttaggt aacaaacacc tgtaagtact tatgtgccag |
| 43021 | ggagtggtct aagttcttta caaatactga cttattaaca ttctgaactt cgattgcttg |
| 43081 | gtcaagcata aatctaggtt aaatcaataa aagttgattt gtgatacctc ttttaattct |
| 43141 | aatacatgtc atgtcacatc tgcttcaggt tgttttgttg cagatgatta attaacctgc |
| 43201 | catagttgat ataaaatgtc aatatttaag caattttctt ttttatatat aatttcaaaa |
| 43261 | tccttccaaa ttatttggaa gtaaaatttg ctgtaaatgg tatgctactt ctgcttggta |
| 43321 | ttgaaataga ataacatcaa aatgtttcac agaaatgcat attccatttc agaacagaaa |
| 43381 | tactatgaga aattctgtcc ggtttcagat tatgttatgt atattatatg ttctatgcat |
| 43441 | tgctgattta tgtagacatg attttatatt caaagggagg gaagatccca atcaggtgga |
| 43501 | catcaccaga agctatagcc taccgcaaRt tcacgtcagc cagcgatgta tggagttatg |
| 43561 | ggattgctct ctgggaggtg atgtcttatg gagagagacc atactgggag atgtccaatc |
| 43621 | aggatgtaag tatttgtggt ctatgagtta tgagttcaga tgaaaagatc aagctgtgca |
| 43681 | aggaagtgga acataatgta actgggtggc tcctggtttR taacactgaa ataaaatatt |
| 43741 | tagcagcaat gaaactgttt ccaagtcttg caaggatatt atattaattg aattgttctt |
| 43801 | acagatttac atatttccct caagcaaggg tttagaaata agacaaaaga aacatttgta |
| 43861 | gtgaaaacgt aatgaaaaaa gtgaccaata agataacctc tagattctaa tgcacattga |
| 43921 | agtttgaaag tcactgcttt acaccattga aaagtactaa tgaactaata gttacttaat |
| 43981 | tttacagcac ttaggatttt acaaaaaaaa ttactataat ttggaaggca ccatttgaac |
| 44041 | tgcagtataa ctaaaacctg tatagtaatt ttgtccatat tcattcatat atgttttcca |
| 44101 | tagagaacta tataaccaaa taataatttc tttataacac caaatttgct ctatcattta |
| 44161 | acatttcttt ttattttaac ccaaaaagga tctttacata ctctttccta aatttatgtt |
| 44221 | atttcaatct tcctttatat aatattttag gcatacataa cgtcttattt atttgatcag |
| 44281 | ttgttatcct tttaggcaac taaaaatgag aagttttccc acttaccttt aaaataagca |
| 44341 | tatttgtgag ccccgcccat gaaaacgtaa actaagtgac acttcctgaa aacttcctgg |
| 44401 | ttcctgaaaa ctttgcttct cacacaggta attaaagctg tagatgaggg ctatcgactg |
| 44461 | caacccccca tggactgccc agctgccttg tatcagctga tgctggactg ctggcagaaa |
| 44521 | gacaggaaca acagacccaa gtttgagcag attgttagta ttctggacaa gcttatccgg |
| 44581 | aatcccggca gcctgaagat catcaccagt gcagccgcaa ggtgacacat tcaatttgtt |
| 44641 | atctggcatt cactctgaaa tttgtgtttg ctatctccaa gatgttaatt tttttagccc |
| 44701 | acccccaaaa tgcattattt gaagcttgta ttccaccata ttagagagat gtatttcccc |
| 44761 | tttctttttt aatctttaga actaattctc gtcctcctta atagtaatga tgaaattctg |
| 44821 | agatattttt taacatctat cttgactcta cattcaggct tgtttaagtc tttgggaaat |
| 44881 | aggaatactg gaggggtgcg gtggctcgtg cctgtaatcc aagcactttg gtggatcact |
| 44941 | tgaggtcagg agttcgagac cagcctggcc aacatagtga aatgccgttt ctactaaaaa |
| 45001 | tacaaaaatt agcctggtgt ggtggcaggc acctgtaatc ctagctactc gggaggctga |
| 45061 | ggcaggagaa tctgtgaacc tgggaggcag aggttgcaac gagctgatat catgccactg |
| 45121 | cactccagcc tggtcaacag agcgagactc tctctctaaa taaataaata tactgaacac |
| 45181 | atacttcttt tcacccttca tgaataaaga agtcctgatt catctttcat tcaaaataaa |
| 45241 | gaaaaagaaa ttatccaaaa tattgcactg gctagcaaaa tgcaaatttt ccccagttaa |
| 45301 | ttaaacaaat atttgtctag tctatattgc gtatcaatta ctttgctaga caccattgaa |
| 45361 | gataaaacta agagtaagat aaagccagtg tccctggaca acaacacaaa tgttaatact |
| 45421 | ctgcagctgt aatgcaatcc tggatgcaca aagtctattt aattttatgc atattctttt |
| 45481 | aggttaagaa tcactgagtt ctctcagtgg ttagaacaat gtaaattaaa gtgtatctac |
| 45541 | aaagtacagt ggaaaaaaga aggaagaaga gatgaatttt gtcaggggsc atataagaag |
| 45601 | aaaataaata ttaatatatt tggtgcctct cagagtaatt aaaggcaaag atatcttgtt |
| 45661 | taataatttt gattctcatt gtaagcataa gaaaaaaagg ttcttttaca ttttgataat |
| 45721 | gcattctctt acaggctttt aatttttctc tctgatttat cacatgagtt agtacatgtt |
| 45781 | ttatactttt gaaacattcc tcttgacaac tatagagaga agcatttcag acctatttgg |
| 45841 | gacctctagc tatgtgccta ctcaatggct tggacttgtg agttcaatag tatattgatt |
| 45901 | ggtacaatgg agttttatca gtaacgatga cttgtgggtc attctgcctt ctttgcataa |
| 45961 | ccagttgctt ctcaatatcg cttctcaact gtgttagttt tgggtctcaa taatagtatc |
| 46021 | ccgccctgtt gtttccattg ctacaaagct tagtgactaa agctggcgtt caatgcatgg |
| 46081 | gaaattcctt gcattttgtc ttaagttatc tgtttgaaga tcaagcaggg cgtgtgtgtg |
| 46141 | tgtgtgtgtg tgtgtgtttg tgtgtgtgtg tgttggtgag tgaatatgta ggtacattac |
| 46201 | aagtttgatt aaccatgcct tagtatcata tttttgtgaa aaaaatcaat catatttaaa |
| 46261 | acaatatttt aagatgtcta aaatcaatgc ccctgaaatc cacagggaat aaaatagcaa |
| 46321 | aattttaaca tataaacgtt ttttatccat tcttctgtca gtggatacct atgttgattt |
| 46381 | catatcttgg ctattgtgaa taatgctgca atgaacatgg gaatatagat atctctttga |
| 46441 | aatactgact tcatttcctt tggctatata cccagagtgg gattgctgga tcatatggtg |
| 46501 | attctatttt tagttttttg tggaacctcc atacattttt ccatatggct gttccaattt |
| 46561 | acattccctt acatgccaat gttccctttc tccacatctt cactaacagt tgttatcttt |
| 46621 | tgactttttg ataatagcca tcctaacaag tgtgaggttc tagctcattg tggttttgat |
| 46681 | tttcatttct ctaatgacta gcgatgttga gtacctttac ataaacctgt tggccatttg |
| 46741 | tcttcctcag aaaaaaatgt ctattcaggt cctttgccca tttttaatag gggtattatt |
| 46801 | atttttgcta tcaaattcta tgagttcctt atatattttg gatattagcc acctatgaga |
| 46261 | tatattgact gtaaatattt tctcttcttc tgtggattgc ctttcatttt cttgttttct |
| 46921 | ttactgtgca gaaaattttt aatttgatat aatccctcct gtttaatttg gcttttgttc |
| 46981 | ctatgatttt ggtgtcatct ctacaaaatt gttaccaaga ccagtgtcat agagctttct |
| 47041 | tcctgtattt cctgctagaa gctttacagt attaggtctt cagtttatcc attttgtgtt |
| 47101 | gattatatta aatgaacatt acctgttttt tacataatag tttaataagt taagtaggag |
| 47161 | aatcataaac aagatcttaa tatcagtata taagaatcat agattattta ttgataataa |
| 47221 | cacttagtag gaatcactat gattgaattt agtaataagt ccttaaataa aactaaccat |
| 47281 | aacttattcc ataaggataa atatctatcc caaacttcac agagactgta tagactctta |
| 47341 | gaaattcacc tgacagttta acaactaaca ggataacaaa cctgtccttt cctgggccat |
| 47401 | atggtcctca ttctgtaccc tgaaggcatt tattttgatc tttacagcat agatagtaat |
| 47461 | cacagaaaaa aaaagacttt acctatcata aaaacattca aaccattttc aaagctaact |
| 47521 | catgtaatgt aatgtaatgt aggggtatat aataccagtt tcacagagaa aagtagtctt |
| 47581 | actatcagat cataatatgt gaggctatgt cctagcatcc aaattttaac atactttcat |
| 47641 | aattcacttt tccactctta ttgaaatata tcaaatactt tttcttatat atttttcttt |
| 47701 | aatacatgaa ctaatttgat gtattttgtc ataaaacata attttagaaa aaaatcaata |
| 47761 | ttttctatga aaagacaacc atctcaatgt acagaatata aacatttggg caattatatt |
| 47821 | tatattttta cttattacca ttaaaatgca cgatgggtta gattttgtca taatacattt |
| 47881 | atttttaaca acctcacatt actatatgca actatgtcaa gaagtttttt tctctttaga |
| 47941 | aaatttgctt tcctactttt gacaatgatt atttcatcct tttcataact gattaaaatg |
| 48001 | acctccttct ttaattttta gtgatgtcct tgccaaatgc tttattgaga aaataagcca |
| 48061 | ttagatggga acttcccaca gcaaaacaga aatgtagtgg cacctgcacc tacccatatc |
| 42121 | gtccttcttt cctctaatta taaaaggcag tgtccctcta tttgttagag gctaatcctc |
| 48181 | tactcttcct ctctccctgt ctccctctct ctcttctgca tatccaggct ctttttttct |
| 48241 | gtaggatcat tgccacaaac agatgttcta ttatattctt tctcattata ttattcttat |
| 48301 | caccagggga acatattaca catacaaata tccaggtcta tcccaagaat ctccaggaaa |
| 48361 | agagaatgga gtttggatgt tttataaaaa tgccaggtga tttttttctc ctctggaaaa |
| 48421 | tttaggtgat attgcttgtc aatattaccc ctgctggccc cacatctctg ccaactactt |
| 42481 | ccctacttct ataacactct tcacagccaa acttctcaaa taatggactc acagacataa |
| 48541 | agtcaatatt ctgaaaattt atcaattttc tccccctagc ctgatccttc cactgcctaa |
| 48601 | ttaattgcat acaccagcag ctacccattc acctaaccag gaattagagc aactcttggt |
| 48661 | acctctttct tcccctctac actgtgatca tctggatcaa tgagatctat ctctatccct |
| 48721 | tttcactgaa aacactctcc tcatggctgt ttcgtcttct gacctggacc actgcaatag |
| 48781 | gctcctaact tgtcccctgc tttgattgct ttcaaactcc aaacattttt tttgcacagc |
| 48841 | ctccagaatc ttcttatcaa aacatatatg atatagtttc agtcacctgt ttataatcaa |
| 48901 | tattgaattc acgttacact tMtaaaaaaa tctccttttg gttgtttaaa aagccctgct |
| 48961 | gactgggtgg agtgactcaa aactgcaata ccagctattc aggagactgg ggctggagga |
| 49021 | taacttgtgg ccaggagttg gagaccagtt tgggtgttat agcaagaccc ccatccctac |
| 49081 | aaattttttt ttaaatgggc caggcatggt ggtgtttgcc agtggtccta gctactcagg |
| 49141 | aggctgtagc agaaggattg cttaagtcca ggagtttgag gttacagtga gcaatggtaa |
| 49201 | caacactgca ctcagcctgg gtgacagagc aagacactgt ctcttgaaaa aaattataaa |
| 49261 | ttaattaaaa aagaaaattt tgaaaagtct tgcttctaca atgttattat cttattcaaa |
| 49321 | taaattttat tcgaaaacta tttttatata atacaatgag atgactatag tcaataatac |
| 49381 | cttaattgtg tattttaaaa taaatggtgt tttctgccac tcttgtaaac aacagacaag |
| 49441 | tacagaccag aggaaggaaa taagtaaatg gccgtgatgg gcgataaagg aatagctgcg |
| 49501 | aaagagtctg aggagctgat gtttgagacc tgcaggaagg gaatgagcca gctgtgtaac |
| 49561 | ctcctcctca ccaaaaagaa tattcttggc acaggattaa aaaatgcaaa agtcttgaga |
| 49621 | tgagaaaggg tttggagagt tctaggaagt ggcagagaat aatatgactg aagggagtct |
| 49681 | gtcatgaacc taaggaggta aacagaagcc agagcacttg aggctacaca ggccatggtg |
| 49741 | aggactttga aatatttttc aaatgcatga ggaattcatt aggcatctga accagagaag |
| 49801 | taagatgatt tgatttatat gactgctttg tagagggagg agcaaaagta tgaggaggtg |
| 49861 | ggggtcattt taacctttcg acttgtgcca ccttcatcta aacaatactc taacctcagc |
| 49921 | ggctacttct agttccttca gaaaacaaaa gcatttcaga agattgtagc acatgctgtt |
| 49981 | tgccttgtct cagattctcc tttgcacctt tcttccttaa ggaagcaact tacacatcat |
| 50041 | gtatgtgttg actcattctg tggcagcacc agtgaaataa agtttaacat cattattctt |
| 50101 | ttacagagca ccacattctc gttcttcatc actattttaa ttctgttttc tgtgWttgtg |
| 50161 | tctgtgtatt ctttttaact atccattttc tcaataaact ctaagccctg tgagagcaca |
| 50221 | caccatgctt ctctcgtatt accagtgcag attgataatg ggtgctctgt aaatgtctcc |
| 50281 | taaataccta ataaatatgt gctgacatca ttagtaaatt tacatttcaa aagatgcaat |
| 50341 | ttatagaaaa aattccagtg tttttctgct ctctgaacag atttttttaa tagacattga |
| 50401 | tcatatggta aaagcctaga cctgttctcc taagcaaaat tctgcatgtg tacctttctg |
| 50461 | tcaactcaac tccagaaata cactcctaaa aatttaatag gagacttcaa aactgaattc |
| 50521 | gtggctggta caaagcattc aagtttctga atggtgtagt tattcagatc agtgcataga |
| 50581 | actgtgttta tacacgtacc ctgtagtgtc ttttaagaaa aacaacatca ttctgataga |
| 50641 | aatgatgttt cttcagtctt tctggattta gtacatttta tggtcaaatt ttattttttt |
| 50701 | cttggtggct aagtaccttt tgaaactaat tgtttgaatt taaaaacgtt gttgcatata |
| 50761 | ccgcaataga agtcttttta taatgcatca tttcactttt cagccttcat tagagttgat |
| 50821 | gatttatagc agtaccattc tcaaagaatc ttccgcttag agtgtctcca tgaatatcgt |
| 50881 | cactggaagt ggggaaggtg gaaaagctga tggtcagggt gggccagatc aaacaattac |
| 50941 | ttagatgctg caaagaatca taataaggga aggacagtta cattaaactt tgtgttatgc |
| 51001 | ttgttcccta gtaattgatt gctttttcta aaagtagagt aaatcagagg agatgtgagg |
| 51061 | gttttaagtt ttgatagtac atcagtacaa ggctacagat gtttattcaa caataaccgt |
| 51121 | gtgtgagaca ttatgttagg tcctgtgatg attatatagg taccagatgt agcttccata |
| 51281 | ctcaacagct tacgtttaaa tgtggaagaa aaaagaatat ttagaacact ataatacccc |
| 51241 | tcagaacatg taattattag agcaaaaata aaaggagttc gggaatcccg aggggaggtg |
| 51301 | agtttacttt tatatttgga gttttctggt atttgtatgc aaaagggact tttggatagt |
| 51361 | ggatgaaatt gaaatttcta caggtaggta acaggaaaac aggctggggg aacactacag |
| 51421 | aaaagggaag acatgcaaat aggtaggata cttccaaatc acatYgcata atacggtttt |
| 51481 | gctagactat aggataaaaa aaggaaatta tggggaaaaa gcctgataga tcagggatcc |
| 51541 | tcagcccccg tgccatggac tgctKccagg ccatggcctg ttacgaacca ggtggcaaag |
| 51601 | cagaaggcaa acggcgggct ggtgagcatt accgcctgag ctctacttac tgcagatcag |
| 51661 | caggggcatt agattatcat aggagagcaa accctgttgt gaaccgcaca cacgaggagt |
| 51721 | ctaggttatg cgctcctcat gataatctaa tgcctgatga tctgaggcag aacagtttca |
| 51781 | tactgaaacc atcccccacc acaccaccat tcgtggaaaa attgtcttcc atgaaactgg |
| 51841 | cccctggtgc caaacaggtt ggggaccgct gcgatagatg attcgtcagg tgatggaaga |
| 51901 | ctaaatgtca tttctagaca tttgggcttt tatccataag gaaaggggag acactgaagt |
| 51961 | tactgaagtg atatgttgca ctgtgtttag ggaatagttc tttggcagct acttctaaaa |
| 52021 | agggctggag atgaagggat tcatacgaga gaattaagct ttcaatagtc taagaaaagg |
| 52081 | atctgctatg ctagagcagg gaaagtagac agtgtgtgaa ttcaatatgt tttattaaag |
| 52141 | tgagttctat aggacatagt gactgagatg tgcagagtga cagagaaagt atcagagaat |
| 52201 | acttccaggt tctgaatgtg gagcctttga gatcagcctt ctcattgaca gtggtaggaa |
| 52261 | gaacagaagg aagaacaggt ctgccatgca gataagcatt cttggcaaca gtcaagaaac |
| 52321 | taatttaaat gtttgaatgc ctcctgtttc cattgtatgt aacaaggtgt agtggcagcc |
| 52381 | aatccagatt ttaggcacct tgtaaggggg ctgtggagcc aatcagtgac aggaaatggc |
| 52441 | aatacaagaa ggaaaacaaa tacgaaacat tcactaatat ctcagaatat taactcacta |
| 52501 | cgttgactgc caccaagaga gaaagaaaat tccctttatt ttgagctttg gagagcagtg |
| 52561 | ccagttatat cacagaagaa tgtgaaatgg taggtagaaa cgaggaacaa tcagggtgat |
| 52621 | aggatgaggt ttagactact gcagaaaacc acagtgcaag gcatttccaa aaagtagatt |
| 52681 | caaacggtat taacaaaagc tgaaaaaggg aaagaaaaac aaaaactgag ggaatagact |
| 52741 | tccagttaaa taatagaagg atttataatc cattgggtat atacccagta atgggattgc |
| 52801 | tgggtcaaat ggtatttctg gttctatgtc tttgtggaat cgccacactc tcttctataa |
| 52861 | tggttgaact aatttacacg cccaccaaca gtgtaaaagc attcctattt ctctgcatct |
| 52921 | tcgctggcat ctgttgtttc tagacttttt aatgatcgcc attctaactg gcgtgcacac |
| 52981 | gtatgtttgt tgcaggacta tttacaatag caaaaacttg gaaccaaccc aaatgcccat |
| 53041 | caatgataga ctgaataaag aaaacgtagc acatatacac catggaatac tatgcagcca |
| 53101 | taaaaagaat gagttcatgt cctttgcagg gacgtggatg aagctggaga ccatcattct |
| 53161 | cagcaaacta acacaggaac agaaaaccaa acactgcatg ttctcactca taagtgggag |
| 53221 | ttgaacaatg agaacgcatg gactcaggga gaggaacatc acacaccagg gcctattgga |
| 53281 | gggtgaaggg aaagggaaag gatagcattc tgacaaatac ctaatgcatg tggagcttaa |
| 53341 | aacctagatg atgtgttgat aggtgcagca aaccaccaag tcacatgtat acctatgtaa |
| 53401 | caaacctgca ggttcaacac atgtacccca gaacttaagg taaaatttta aaaaagatag |
| 53461 | aaggaacatt gaggtattaa gaaaactatt ctgaattttg gaataatata tattacattg |
| 53521 | tgagagacct aattataaaa ttggataatg aaaaggcaag gcaaaacaag aagatgtgca |
| 53581 | taaaagaatg aaaacaaaag aagctctcct ctcttttttt ataagaatca aagaaatgtt |
| 53641 | tcttctttct tctctttctc ttcctctttt tcttactctt tctttctttc tttctctttc |
| 53701 | tttcttccct tctttctctc cttccttctc tccttccttc tctccttcct tccttccttc |
| 53761 | cttccttcct tccttcgttc cttcctttct tcctttcttt ctttcttgac agtgtttcac |
| 53821 | tcttgtcgcc cgggctggca tgcaatggtg ccatcctccg cctcctgggt tcaggcgatt |
| 53881 | ctcctgcctc agcctcctga gtatctggaa ttataggcac tccccaccat gcctgactaa |
| 53941 | tgtttgtatt cttagtggag atggggtttc accgtgttga ccaggctggt ctcgaactcc |
| 54001 | tgacctcagg tgatccactt gccttggcct ttcaaattgc tgagaattca ggcttaagcc |
| 54061 | accacatcca gcccaaaggg atgtttttta aagattgttc ttgatagaaa atgccaccat |
| 54121 | gaaaataaat agaaaatagt cttaaaataa aaatattctt taacttccca acacattgtt |
| 54181 | ttgtacatat aagagaaata cacatttatt ttttaaaaac atattttgaa agattataat |
| 54241 | ataacttcta gatttaaata aatttattca cttattttat aactgaagct aaattaaaaa |
| 54301 | ttgactaata acagaaaaat gagaatgtgt ttctattcaa gtagtaagat acacttttta |
| 54361 | tttttgaaga tatgagattt ttattattga caatattaat tttgaagaga tgtatgtgtg |
| 54421 | tgcatatgtg tgtaaaatga aatagagaaa taatttcttc ttaagttcac agaatttcca |
| 54481 | ctagccaggt attgaattct tctatactgt tgtaaattga taaaattcaa attcttgaca |
| 54541 | caaaaaaatt agataataca ttcaaaatat ttagaacgag gttttaaatg gaaggcaaca |
| 54601 | caattcatga cacagactta agcaatatat ttaataatat ttagaacaag gttctgcaga |
| 54661 | cccaattata tttagttaat ttaattgaaa tgtttttaaa tatattgttt cattgcattg |
| 54721 | atcactaact ttgaagggat ttatttttat taacttgcag catagaaagg agtaaaaata |
| 54781 | aacaatactc atattttccc cagttaatga aaataaacaa gtattagtaa aaagcttctg |
| 54841 | atctctaaga gtacataata gaattctttt tgagaataac tgctgttttg acaaatacca |
| 54901 | tatccttcaa gacaaatcga acaaccaaaa gacaatctgc ctttatttct caaaatgact |
| 54961 | ttgacctcaa attctgaaac tgacagggtg actttgctca gtgaataaat atgatacaga |
| 55021 | accttttaga gggaatcgaa tatgacgaat acccaagtga tatctctgtc tctctttttt |
| 55081 | ttttttttta ttatactcta agttttaggg tacatgtgca cattgtgcag gttagttaca |
| 55141 | tatgtataca tgtgccatgc tggtgcgctg cacccactaa tgtgtcatct agcattaggt |
| 55201 | atatatccca atgctatccc tcccccctcc cccgacccca ccacagtccc Cagagtgtga |
| 55261 | tattcccctt cctgtgtcca tgtgatctca ttgttcaatt cccacctatg agtgagaata |
| 55321 | tgcggtgttt ggttttttat tcttgcgata gtttactgag aatgatggtt tccaatttca |
| 55381 | tccatgtccc tacaaaggat atgaactcat cattttttat ggctgcatag tattccatgg |
| 55441 | tgtatatgtg ccacattttc ttaatccagt ctatcattgt tggacatttg ggttggttcc |
| 55501 | aagtctttgc tattgagaat agtgccgcaa taaacatacg tgtgcatgtg tctttatagc |
| 55561 | agcatgattt atactcattt gggtatatac ccagtaatgg gatggctggg tcaaatggta |
| 55621 | tttctagttc tagatccctg aggaatcgcc acactgactt ccacaatggt tgaactagtt |
| 55681 | tacagtccca ccaacagtgt aaaagtgttc ctatttctcc gcatcctctc cagcacctgt |
| 55741 | tgtttcctga ctttttaatg attgccattc taactggtgt gagatgatat ctcatagtgg |
| 55801 | ttttgatttg catttctctg atggccagtg atgatgagca tttcttcatg tgttttttgg |
| 55861 | ctgcataaat gtcttctttt gagaagtgtc tgttcatgtc cttcacccac tttttgatgg |
| 55921 | ggttgtttgt ttttttcttg taaatttgtt tgagttcatt gtagattctg gatattagac |
| 55981 | ctttgtcaga tgagtaggtt gcaaaaattt tctcccatgt tgtaggttgc ctgttcactc |
| 56041 | tgatggtagt ttcttttgct gtgcagaagc tctttagttt aattagatcc catttgtcaa |
| 56101 | ttttgtcttt tgttgccatt gcttttggtg ttttggacat gaagtccttg cccacgccta |
| 56161 | tgtcctgaat ggtaatgcct aggttttctt ctagggtttt tatggtttta ggtttaacgt |
| 56221 | ttaaatcttt aatccatctt gaattgattt ttgtataagg tgtaaggaag ggatccagtt |
| 56281 | tcagctttct acatatggct agccagtttt cccagcacca tttattaaat aaggaatcct |
| 56341 | ttccccattg cttgtttttc tcaggtttgt caaagatcag atagttgtag atatgcggca |
| 56401 | ttatttctga gggctctgtt ctgttccatt gatctatatc tctgttttgg taccagtacc |
| 56461 | atgctgtttt ggttactgta gccttggagt atagtttgaa gtcaggtagt gtgatgcctc |
| 56521 | cagctttgtt cttttggctt aggattgact tggcaatgcg ggctcttttt tggttccats |
| 56581 | tgaactttaa agtagttttt tccaattctg tgaagaaagt cattggtagc ttgatgggga |
| 56641 | tggcattgaa tctgtaaatt accttgggca gtatggccat tttcacgata ttgattcttc |
| 56701 | ctacccatga gcatggaatg ttcttccatt tgtttgtctc ctcttttatt tccttgagca |
| 56761 | gtggtttgta gttctccttg aagaggtcct tcacatccct tgtaagttgg attcctaggt |
| 56821 | attttattct ctttgaagca attgtgaatg ggagttcacc catgatttgg ctctctgttt |
| 56881 | gtctgttgtt ggtgtataag aatgcttgtg atttttgtac attgattttg tatcctgaga |
| 56941 | ctttgctgaa gttgcttatc agcttaagga gattttgggc tgagacgatg gggttttcta |
| 57001 | gataaacaat catgtcgtct gcaaacaggg acaatttgac ttcctctttt cctaattgaa |
| 57061 | taccctttat ttccttctcc tgcctgattg ccctggccag aacttccaac actatgttga |
| 57121 | ataggagcgg tgagagaggg catccctgtc ttgtgccggt tttcaaaggg aatgcttcca |
| 57181 | gtttttgccc attcagtatg atattggctg tgggtttgtc atagatagct cttattattt |
| 57241 | tgaaatacgt cccatcaata cctaatttat tgagagtttt tagcatgaag ggttgttgaa |
| 57301 | ttttgtcaaa ggctttttct gcatctattg agataatcat gtggtttttg tctttggctc |
| 57361 | tatttatatg ctggattaca tttattgatt tgcgtatatt gaaccagcct tgcatcccag |
| 57421 | ggatgaagcc cacttgatca tggtggataa gctttttgat gtgctgctgg attcggtttg |
| 57481 | ccagtatttt attgaggatt tttgcatcaa tgttcatcaa ggatattggt ctaaaattct |
| 57541 | cttttttggt tgtgtctctg cccggctttg gtatcagaat gatgctggcc tcataaaatg |
| 57601 | agttagggag gattccctct ttttctattg attggaatag tttcagaagg aatggtacca |
| 57661 | gttcctcctt gtacctctgg tagaattcgg ctgtgaatcc atctggtcct ggactctttt |
| 57721 | tggttggtaa actattgatt attgccacaa tttcagagcc tgttattggt cgattcagag |
| 57781 | attcaacttc ttcctggttt agtcttggga gagtgtatgt gtcgaggaat gtatccattt |
| 57841 | cttctagatt ttctagttta tttgcgtaga ggtgtttgta gtattctctg atggtagttt |
| 57901 | gtatttctgt gggataggtg gtgatatccc ctttatcatt ttttattgtg tctatttgat |
| 57961 | tcttctctct ttttttcttt attagtcttg ctagcggtct atcaattttg ttgatccttt |
| 58021 | caaaaaacca gctcctggat tcattgattt tttgaagggt tttttgtgtc tctatttact |
| 58081 | tcagttctgc tctgatttta gttatttctt gccttctgct agcttttgaa tgtgtttgct |
| 58141 | cttgcttttc tagttctttt aattgtgatg ttagggtgtc aattttggat ctttcctgct |
| 58201 | ttctcttgta ggcatttagt gctataaatt tccctctaca cactgctttg aatgtgtccc |
| 58261 | agagattctg gtatgtggtg tctttgttct cgttggtttc aaagaacatc tttatttctg |
| 58321 | ccttcatttc gttatgtacc cagtagtcat tcaggagcag gttgttcagt ttccatgtag |
| 58381 | ttgagaggct ttgagtgaga ttcttaatcc tgagttctag tttgattgca ctgtggtctg |
| 58441 | agagatagtt tgttataatt tctgttcttt tacatttgct gaggagagct ttacttccaa |
| 58501 | ctatgtggtc aattttggaa taggtgtggt gtggtgctga aaaaaatgta tattctgttg |
| 58561 | atttggggtg gagagttctg tagatgtcta ttaggtctgc ttggtgcaga gctgagttca |
| 58621 | attcctgggt atccttgttg actttctgtc tcgttgatct gtctaatgtt gacagtgggg |
| 58681 | tgttaaagtc taccattatt aatgtgtggg agtctaagtc tctttgtagg tcactgagga |
| 58741 | cttgctttat gaatctgggt gctcctgtat tgggtgcata aatatttagg atagttagct |
| 58801 | cctcttgttg aattgatccc tttaccatta tgtaatggcc ttctttgtct cttttgatct |
| 58861 | ttgttggttt aaagtctgtt ttatcagaga ctaggattgc aacccctgcc tttttttgtt |
| 58921 | ttccattggc ttggtagatc ttcctccatc cttttatttt gagcctatgt gtgtctctgc |
| 58881 | acgtgagatg ggtttcctga atacagcaca ctgatgggtc ttgactcttt atccaacttg |
| 59041 | ccagtctgtg tcttttaatt gcagaattta gtccatttat atttaaagtt aatattgtta |
| 59101 | tgtgtgaatt tgatcctgtc attatgatgt tagctggtga ttttgctcat tagttgatgc |
| 59161 | agtttcttcc tagtctcgat ggtctttaca ttttggcatg attttgcagc ggctggtacc |
| 59221 | ggttgttcct ttccatgttt agagcttcct tcaggagctc ttttagggca ggcctggtgg |
| 59281 | tgacaaaatc tctcaacatt tgcttgtcta taaagtattt tatttctcct tcacttatga |
| 59341 | agcttagttt ggctggatat gaaattctgg gttgaaaatt cttttcttta agaatgttga |
| 59401 | atattggccc ccactctctt ctggcttgta gggtttctgc cgagagatcc gctgttagtc |
| 59461 | tgatgggctt tcctttgagg gtaacccgac ctttctctct ggctgccctt aacatttttt |
| 59521 | ccttcatttc aactttggtg aatctgacaa ttatgtgtct tggagttgct cttctcgagg |
| 59581 | agtatctttg tggcgttctc tgtatttcct gaatctgaac gttggcctgc cttgctagat |
| 59641 | tggggaagtt ctcctggata atatcctgca gagtgttttc caacttggtt ccattctcca |
| 59701 | catcactttc aggtacacca atcagacgta gatttggtct tttcacatag tcccatattt |
| 59761 | cttggaggct ttgctcattt ctttttattc ttttttctct aaacttccct tctcgcttca |
| 59821 | tttcattcat ttcatcttcc attgctgata ccctttcttc cagttgatcg cataggctac |
| 59881 | tgaggcttct gcattcttca cgtagttctc gagccttggt tttcagctcc atcagctcct |
| 59941 | ttaagcactt atctgtattg gttattctag ttatacattc ttctaaattt ttttcaaagt |
| 60001 | tttcaacttc tttgcctttg gtttgaatgt cctcccgtag ctcagagtaa tttgatcgtc |
| 60061 | tgaagccttc ttctctcagc tcgtcaaaat cattctccat ccagctttgt tctgttgctg |
| 60121 | gtgaggaact gcgttccttt ggaggaggag aggcgctcag cgttttagag tttccagttt |
| 60181 | ttctgttctg ttttttcccc atctttgtgg ttttatctac ttttggtctt tgatgatggt |
| 60241 | gatgtacaga tgggttttcg gtgtagatgt cctttctggt tgttagtttt ccttctaaca |
| 60301 | gacaggaccc tcagctgcag gtctgttgga ataccctgcc gtgtgaggtg tcagtgtgcc |
| 60361 | cctgctgggg ggtgcctccc agttaggctg ctcgggggtc aggagtcagg gacccacttg |
| 60421 | aggaggcagt ctgcctgttc tcagatctcc agctgcgtgc tgggagaacc actgctctct |
| 60481 | tcaaagctgt cagacaggga cacttaagtc tgcagaggtt actgctgtct ttttgtttgt |
| 60541 | ctgtgccctg cccccagagg tggagcctac agaggaagga aggcctcctt gagctgtggt |
| 60601 | gggctccacc cagttcgagc ttcctggctg ctttgtttac ctaagcaagc ctgggcaatg |
| 60661 | gcgggcgccc ctcccccagc ctcgttgccg ccttgcagtt tgatctcaga ctgctgtgct |
| 60721 | agcaatcagc gagactccgt gggcgtagga ccctccgagc caggtgtggg atatagtctt |
| 60781 | gtggtgcgcc gtttcttaag ccggtctgaa aagcgcaata ttcgggtggg agtgacccga |
| 60841 | ttttccaggt gcgtccgtca cccctttctt tgactcggaa agggaactcc ctgacccctt |
| 60901 | gcgcttccca ggtgaggcaa tgcctcgccc tgcttcggct cgcgcacggt gcgcacacac |
| 60961 | actggcctgc gcccactgtc tggcactccc tagtgagatg aacccggtac ctcagatgga |
| 61021 | aatgcagaaa tcaccgtctt atgcgtcgct cacgctggga gctgtagacc ggagctgttc |
| 61081 | ctattcggcc atcttggctc ctccctcctg tctctctttt ttaatcttaa aatcaaaaca |
| 61141 | gctaatttta ccacacggag gaattaaaac aggtgtaacg gctataactt caaagatttt |
| 61201 | tgtccttaag gcctagagat aattaaaata aacacaatga ctttatttct ccttaatgtg |
| 61261 | tcatttatgt actaggttcc tgttgggaac tgtatatatt caaaattatt tttagttact |
| 61321 | gatgaaaaat taaattcatt ttatcgtatc tggattttta attttagtga taaagatcaa |
| 61381 | ggtcatcggc aatcaacatt aaaataaatt ggcaaatata agattttccc cctgaaatac |
| 61441 | attggggaaa catttgaaat tttaaaaaac agttataaaa tgtacggaaa aaatatatta |
| 61501 | ctattctcaa acaataataa attcagatac aattattagt attttggctt taaaaagttg |
| 61561 | ctttttagat ttaatgtatt tcctgaaact attcacaaca aataaaaaat aattataatg |
| 61621 | ttacagtatg catctccaat gttattacct ccttgtagac ataataggca tatacttatt |
| 61681 | cattcaagaa atgtgtatta gttattattt ttgatatttg aggagtaaat ctctttgtaa |
| 61741 | tcaaaaggag tctattaagt gagaaatgga taactagaga ttcagaattc taaggtagca |
| 61801 | ttgaaataag attattacca gaagatattc ctgggattgg cagttgttta ttgagctttt |
| 61861 | tccccagtat ctggtgctaa tttttatgca cctatttaca gctaaaatag gaattgatgg |
| 61921 | gaagaaaata tatatgtacc tctctaatgc aacagtataa aacacatgaa agaataattc |
| 61981 | acaaatctta atagaaagag agagaaagaa agggaagaaa gttatttttc acttcccctg |
| 62041 | agcaaatatg taatttccat gatttctttt taatgactgc ggtctgtagc cagtaggtac |
| 62101 | tgatttatga aaagctgtaa aagatcaggt ggagctctgt aggacatcgt ttacagctgc |
| 62161 | atggattgaa acctatctaa tttgtctagt aaaagtgtga gctcacggtg cacatcaccc |
| 62221 | agcctataaa tttccccttc agaccaatgt ctggggctgg gtctgaaaaa acgccaatgc |
| 62281 | ctgcagctgc tcaaaggccc agattccctt ggtgcgcatg aagagacatc acaggctctt |
| 62341 | cacagtgaga gacaaattcc tatccattga taaactctcc agatgtcctc tctgaggagg |
| 62401 | aaaggaacca atttttattt gaaatcccca gagaaatcct ggaagtggaa aaatgatact |
| 62461 | ttgcagggct agttattctg aggtcattgg cattaaaatt aggtcatttt tttttgtctt |
| 62521 | ggatgcactt tgatccaagt cataaacaga caaactcagt tggggtcact aatccttaat |
| 62581 | ccacacgtcc tgttctattt caatttgttt atactaattt tctctaaact cttgttgagt |
| 62641 | ctggctaaag tgttgagtgt aaaagtacaa actattdtga actgagaaga aagaattctg |
| 62701 | cattttgcta ttaggcacac ttcataaata ttctgaataa atgtcctggg gtttgattct |
| 62761 | tcatacccac aaacccataa ataaaaaagt attactatta atgggactaa aacttgacat |
| 62821 | aagattctgt gctccagcta ctgtagacag agaaactgtg gcgtgtgcgc gcgcatgcct |
| 62881 | gtagatagtt taatgtcttg tctcattcta ggtttcacat gatttttctt ctaaaatctt |
| 62941 | ctcacacctg atttaatgct attcttatca attttttagt ggttcaatga cttatccaat |
| 63001 | gagtctaatg taatgtctaa tgcttgctag tgacaaagac tatttcactc atgataacta |
| 63061 | tgctgaaaca acccatgaat attgtgtaca ttttggtgta tatctgtaat tactcaagtt |
| 63121 | cttaggaaaa gaataaaaat acaaaatttt caattataaa aataagctgt ttttcttcct |
| 63181 | aagatgagta atttaaaaaa tcataaagtc gtttgcatat atgtttcaga agaagataac |
| 63241 | ttatgaaatg ttatattgat attttatagt tttcctgata ttgctaaagg aaaaaattaa |
| 63301 | tatttatata atatggccca atcctttttt agataatctc tgagagttag taaaactaaa |
| 63361 | ctatatcaga gaaatagaaa ttataaactc cataattaag attttcaatt tataatttga |
| 63421 | acttgtgaag taRaaaggat tgaggtaatt gagtttagtc tagtggacag aagtttgaag |
| 63481 | aatccttccc tttcaacttc cctccactac acaaatgacc atcacagaga ggggactatt |
| 63541 | aatctatatt ttccacagag tatgWaaata gagtcagaac caatgttgca attttttttt |
| 63601 | cctgaaactc agggttaaaa ctgcactaaa atgtgaccat ttttatcaaa tacgtttcat |
| 63661 | gcatcgctgt aaaaatccag atagtgaatt cacaaacttc tcatatgttc caagtaatag |
| 63721 | catgtaggtg tagggcacga tctaaaatta tcctggctca cgaatcaact ttacacttga |
| 63781 | gcaggtcttt tgtacataag agagtaagtt gaagaatatc accagaagtg gtataaacct |
| 63841 | ctcaattaaa agacctattt aagtgtgcgc agagctggca cagatttgct tcatagattt |
| 63901 | tctcatttag aacaggagct tttaccacat atcctagatg gtaatcaatg attgctactc |
| 63961 | atatgtaaaa cattatacag ttttatttca tgtatattgt tcattttgct atttcatgct |
| 64021 | atttctaaag atattgaaat tagcccctta gagacctaaa tttatgtcta aaataaggat |
| 64081 | aaatgcatgt cgatatatta tttgttttgg tgtcttctca ccacaaattc taaactttga |
| 64141 | atttaaacac ttcaaatgac agacttcact tagactaatc tttcatttca agtatataag |
| 64201 | aaaaatatga agttctagtt ttaatactcc taattttgga gatttttatt gaaagattct |
| 64261 | gaagaactaa aacggcaaaa aatcaaaaca taccctctta attattaaat aaagattaaa |
| 64321 | ttatatataa agttttaaca ttaatactat attttattac aataatgaaa aaaatcacag |
| 64381 | acagcagaat agcacttctg ttttcaagca tttgcagtgg aatgattgtg aagttgtaaa |
| 64441 | agttactgac tattctaatg aagcccctaa taatggctac ttttcagggt tgttaYacta |
| 64501 | ttatgtacat aatattatat atgactaaat ttctagttac ttaaaatggt ggttaacaag |
| 64561 | atcattgcat tcccagcatt atatataaac tgatatttac tctgctattt tctttttgaa |
| 64621 | gttttcagaa gtttaaccat ttcaccattt acataagaat aatgaacagc taacacatag |
| 64681 | tgcttattat gagccagcac tgttctaagt gcttcccaca tatgctcatt taatcttcat |
| 64741 | ttgtgtgaaa ttgatatcct caccatcatc cccattttac agatgaggaa acagacaaga |
| 64801 | aattcaccaa ggtcacacaa atagtatgtg gtatacctgg gattcaaccc aggccttacg |
| 64861 | gttccagtat ctgtgcacta aacttcaggg catataaaca tcgaatatgc cacatgatgg |
| 64921 | aatcataaat catttaagaa aaaacagggg aaatgcaata tgaacaaagt tttacctaaa |
| 64981 | tggcacagtg taccagtcaa gaatgtggac tgtggtatag aataatgtgg gtctaaaggc |
| 65041 | cagctctgcc tcttttgcct gaggaaactt agcttcgtaa cttaatgtga tttagtttca |
| 65101 | gcttctttat ctatctcaga ggatagcatc agtgcctaac aggtactgta aggattaagg |
| 65161 | tccttcatgt ctgtgtaata ttcttagcat gatgcctgcc acatcgagtg cacatgaacg |
| 65221 | tttgatataa tatgaaaaca agggccttaa aaacttattt ctgtcattgt tattattcat |
| 65281 | ccaaaactaa aggcttttat aactaccttg tgcccaagct cccttcctta ggagaaaaga |
| 65341 | agagagaagt caatatattg ttcaccragt gactggctcc agtcttataa gtcaaaggac |
| 65401 | aattaaagag gccactaaga gctgaagaaa aaaaaaaaca caccaaagac atttcaccaa |
| 65461 | cagtctgctt cttttgcctc ttcagggagg caaacattgt ctcattcatt tcctttatgc |
| 65521 | ctcttctgtt ccattgattt accaggtctt gcaggtggtt atagatagat gtttaggcct |
| 65581 | gtatttatat aaaaaaatcc tagaaggaag tcctttttct ttctctcttt ctttttgaga |
| 65641 | cggagtttcg ctcctgttgc ccaggctgga gtgcaatggc atgaactcgg ctcaccgcaa |
| 65701 | ccttcgcctc ccaggttcaa gcgattctcc tgcttcagcg tcccgagtag ctgggattac |
| 65761 | aggcatgtgc caccatggct aatttggtat ttttagtaga gatggagttt ctccatgttg |
| 65821 | gtgaggttgg tctcgaactc ctgacctcag gtgacccgcc ggcctcaacc tcacaaagtg |
| 65881 | ctgggattac aggagtgagc cactgcgccc ggcccttttt cttaaagctg attgtattgt |
| 65941 | gcaatataaa tcacaattac aatccctctc tgagtctctg tgtgtgtgtg tgtatgtgtg |
| 66001 | tgtgtatata tataaaatat tatgtatata tcatgtgtat ataaacacat tacatatata |
| 66061 | atgtatatat catgaataat gtgttgcata tatatatcat gaacttcatt tggtgtcaag |
| 66121 | tatgcaagaa tgttttttct tatcagtcat ctactttttt ctattatttt ttatttttaa |
| 66181 | tgacatgcag cattatttaa ctctgtattg agtttatgtc ctgctgtgag tgtagcctag |
| 66241 | attgttcaag ccattcttta gacagcaacc tgtgcaatgt caaacaaaca aacaaacatc |
| 66301 | cagtgggaaa tacttaaaat attgaaactg aatccatcat caaagcagac agcaaagaat |
| 66361 | tcagtgtgtc ctgcctactg taaccgtatc ctaaattact accatttttg ctagtgatta |
| 66421 | aattcaatga taataaaata tgtatggata tatttatgtc tttcactgga aaattttcag |
| 66481 | tgatttaaaa gcatttgtgg atggagaagg ggagaactta gacaagatga ttacacggcg |
| 66541 | ttcctccaag ctgccagctt gaacagatga agctttcatg gcagataaat tccacaaatg |
| 66601 | aaaggaacac catatgaagt ctggaagttc ctgccgatgt tagtgtcaaa ttttgacaca |
| 66661 | cagcaactct gctgactcct gatctgtctc cctttggtgt tttttttctt gcaggccatc |
| 66721 | aaaccttctt ctggaccaaa gcaatgtgga tatcactacc ttccRcacaa caggtgactg |
| 66781 | gcttaatggt gtcYggacag cacactgcaa ggaaatcttc acgggYgtgg agtacagttc |
| 66841 | ttgtgacaca atagccaaga tttccacaga gtaagaaaaa aaaattcatt aagaagaatg |
| 66901 | aaggattctt ttgaactttc ttggcttgac atgaaagatt tgtaacatct tggcttgaca |
| 66961 | tgttacaaat attagtggta gatctgaggt actgactacc gcctggaact gctaatcagg |
| 67021 | gccctgggtc tccttgtggc ccacagcttc tggattgata catattgatg tgtcttcctc |
| 67081 | tctcaagata gggcccagga tcggtgtctt tagatatgac cttcagatca accaacagat |
| 67141 | tcacacccat aaccacactg cagactcaag cccctaattt aaaacataag cagacataga |
| 67201 | ttgatgatta ttatatttct gcattttgtg atttttctgt tatttttact aaagcttaac |
| 67261 | atctgtgaat caggaaaatt gagagccaag tagataatgt ggtatacaag gaaacatatt |
| 67321 | aaagtctaaa actggttttc atagttgcta aataaacata acaacatgta ttcatcattg |
| 67381 | tctaccctgt aaaggatatc atgctagaaa atgaagttat aattaggttt aagatagaat |
| 67441 | ttttgggttt tcctcaaaag gcttattgcc agatgagaaa gaaaaaatat gcgtcataat |
| 67501 | attaatatta attgatagct ttatgtgtta aaaaccaata ataatcaagt ataatatgct |
| 67561 | gtattttgtg gacaatcatc tttgggtaac aggaatcttt tgatatgaga aacacatgga |
| 67621 | ctcaaggaaa ggtggtatat aaagaatcaa gtgaatccag tgaatagtca aaaatttggt |
| 67681 | tcttattcac aaagtagagt gtttgggaat tgataaatta agagttggaa gtcacattct |
| 67741 | tcatctcact gtgattgaga accaatttac ttcacttcct aatgtgacta cataattttt |
| 67801 | tctttttctg cacagtgaca ttttctggat tcccatgcgc atagctaaaa actggctttc |
| 67861 | taaactcttg gtcatatagc cttatagtgc cattcaactg accagggtcc aactccctta |
| 67921 | atctaagtta ccaggagaga aagtaggact ggctcagtcc cacttgtaca ttggcacctc |
| 67981 | caaagtcata tgtttactag tatctagtca tttatcttca aaagaagtag aatgaggtag |
| 68041 | tatgaacttg gctctagggg ccttttcctg aagataagat ggagaactgt cccaaagaaa |
| 68101 | gaagtgtata ctggatagat acaccaaaag ttgtccagca tgttgtcgct tgcatggtct |
| 68161 | attgggaaac tgttcatctt tgtgtcagtc tttctttgat ctttgttaaa ttaaataaac |
| 68221 | cctttagtcc cccaagttta agtattatca tctcccacta atatttaaat atttaaatat |
| 68281 | tgttgaatat ataaagactt tcattgccac cctgtcaatc attcaacatc actgtgaaaa |
| 68341 | ataacagatt agatgccaga aaagaacatg ccatgtaatt aatagatata agtgcccaac |
| 68401 | aaacaaataa ataagtttat aatttatggt gtaacattac aatcagaaag acataaagcc |
| 68461 | aaagaaggac ttacaggaac agtttacgat tattgaagaa gaggacttgt tctaaaaaaa |
| 6B521 | taaacttacc ctagtcaatt taacatgcac cagcattctt gttcaaagtg gaatgtggtg |
| 68581 | tttattgaat agacttctga cccaatcgac agtacacttg ggtttctgtg gtacttattt |
| 68641 | tgatcagata cagtctcagg atatagtcat gtgtttaaat atttttataa attcttattt |
| 68701 | tagtaatata aacagttagg gttatgatac acaaaacacc tgctgtagag cacagaagca |
| 68761 | tagcgaaaag caaacacaaa gttgaacatg tccacatcac tgccagattt tcaaaggcaa |
| 68821 | agttatgcaa atgtttactc tcaataaaat atcaacactc aaaactttta gatattgaaa |
| 68881 | cactatattg aaatgtatga ggcagaacac tgttgataag attaactgct agtttgcaat |
| 68941 | tcaaatattg tcagccatta taagctatga atgtgataag aaagctattt gctttaaaaa |
| 69001 | agtgaatgac agcatttgtc atactttcca agttagcgac attacttatg tcagcaatac |
| 69061 | catgttggac gtcaaactct ttttggctga ttggaatatt tttacttaat tgttattatt |
| 69121 | atttgaacaa cctgtgaata tacattcaag atgaaaactg acttttcctg aaagacagct |
| 69281 | ttttcaggtt tgtcctgcac actgagtcag gtggtatgga tatgtccata ttgcactccc |
| 69241 | cacaatattc cttcttctgt cctttctcct caattcaggg acaatatagg attaacacct |
| 69301 | ttgccctaac actccttctt gaaaggagct gcttttttcc ttgagtcttc atagtgtcat |
| 69361 | gcacctcatg ctcctaattt atattacaaa aagagaattt attaaaagtc tttaacatac |
| 69421 | taataatttg ggtaaatata atgaggcaac attttcagca taaatgctgt aaaatattga |
| 69421 | gctagatatt ttagctttct ttcgggttcc tggcttattg tttgaatttg attgctttca |
| 69541 | gtctgtattt tctttgtgcc atccagactc aattcagaaa tgatttgctc gaccaaataa |
| 69601 | ctgtggagat cctcctagaa tagaaaaaga tacctttatt cctattcagg ctgtcaaagt |
| 69661 | ataggaaaag atcaataaaa tttcacattc aaatgcatat taatttattt tcgttagttt |
| 69721 | agagatttca ttgacatgca agctactcaa gttatctcat ttttgaacaa agcccagaac |
| 69781 | acatgcaatt ttgtttaata aatacatatt tcaagacatt atttcataac ctaagatgga |
| 69841 | tttaggaagg ttaaagtgag aaaggaagag agaaagggaa aggttagagt ggctgtgact |
| 69901 | gtggaactag aaagagaatt aagtgtgtgg aatgaatatt aagcagattc attgccacat |
| 69961 | gtcattatcc tgcacccttg gatgccattt tcaaagtgtc atcacctatc cctttgcctt |
| 70021 | tgaagtacca gataacactt taacctctgg tctcagtctg gagctgctct ttagtcttga |
| 70081 | acttttccgc cactcttctg aacaggacag tcaaaatgaa atgagcacac agagtgttat |
| 70141 | ggagacagca gcaactgggg gttccaaatt attttggaca tgcagagtag tggacagaag |
| 70201 | atattcgggc aagaatagag agactagagg gtgtttaaaa gccctagcta aggaataacc |
| 70261 | caaatggata attactttaa ttctcttttc aaaagtccaa tataatcatt tgcattaagt |
| 70321 | aaaaaaattg tatataaaac atttatatgt agtatattta tataaatata taaaaatata |
| 70381 | tattatatat tatatataat atataatgtt tatatattgt agataacata tataaacatg |
| 70441 | tatatatata taaacatgta tatatataag catattatat atatatatgt ttgcttatat |
| 70501 | tgggttcaca gatacagaat gtaaaacagt tttttgcaga acatttcttg aggaatacac |
| 70561 | tcaggaggtg cagctatgaa gaagtgatga ggaatgtctc aaatcRgtct gtgaactcag |
| 70621 | catcacattt ttttttccag ccacttgggg atgagtgctt cagttcagaa ggaagatcag |
| 70681 | ggccaaacat catagcaagc agcactcagt aactatgctt aaatttctca aagagctcag |
| 70741 | gcaattactg cattggaata tataaatatt atatatatat atttactcat ataatataaa |
| 70801 | tattatatat atttactttc catgactatt attattttta tttatttatt tatttattta |
| 70861 | tttatttaat ttttttgagg cggattctcg ctctgtcgcc caggctggag tgcagtggcg |
| 70921 | cgatctcggc tcactgcaag ctccgcctcc tgggttcacg ccattctcct gcctcagcct |
| 70981 | ccggagtagc tgggagtaca ggcgcccgcc accacgcccc gctaattttt ttgtattttt |
| 71041 | aatagagacg gggtatcacc gcgttagtca ggatggtctt gctctcctga cctcgtgatt |
| 71101 | cacccgcctc ggcctcccaa agtgctggga ttacattcca tgactattat tttaggaagg |
| 71161 | ggaaagggaa gaSacaaagg acaaaaaatc atctagtcat aaaacatagg atatggtatt |
| 71221 | ggaagaagat gaaagaaaaa agaaagagtg agaatctatt gatttgcata cccatacttg |
| 71281 | tatctgtcct gttttctggc tctagactca aaaactgact ttgctgtcgt ttggggataa |
| 71341 | gtggagaaat gtttattctt tgttttaaag gtagattcat ggtatgttta tcaaaatcct |
| 71401 | gtaacgcaca ttctcaaagg cagggatcaa cagaagtagc atacattact ccaagtgaag |
| 71461 | gcttccatca tccgcagtac actaagcgtg gcaagcctca cattcccata catccccact |
| 71521 | agccttcctg agaaagagta tgatcagata gggcctaagg caaggcacaa gctctccaga |
| 71581 | gaggatttat cagccctttg acagtcagca gggagggaac ctgaaggtca tcaggaacct |
| 71641 | tctcagccca gatggtagga attcagtaga gtctcggtta atttaatgcc ctaatgctta |
| 71701 | aataacaact acatttattc tcaaatggtt acctattgtt ttcttgaaca cctttattgc |
| 71761 | ctaggaaccc atttcttaag aaatcgatta atccatttct gtgtagttga attcccaatt |
| 71821 | ttttctttct ttatgctcag tataaaggaa tcccctctgt gtgtgtctgt gtgtctgtgt |
| 71881 | ctgtgtgtgg tgtgttcaca cgtgttcttt tttccttgcc attctcaatt ttcctggctc |
| 71941 | tccttgacaa atgatacttt acgccctggt catctttacc cagaaaaata tattttgtat |
| 72001 | gtttctttct ttagcaaatt tgtattgagc ccactaactg gcagccattg tttaggttgc |
| 72061 | tgttggtaaa cagagtacga gaaaaaaaaa agaatcccag ttcacatgga atttaccttc |
| 72121 | ttgtggagta agatagggaa taaagaaaat aaatcagaaa gaaagtcaat atgtcagatg |
| 72181 | gtaataaatg ctttggaaga aaatgaaaca agaaaagaaa acagaaagtg cagatacaag |
| 72241 | tacagaaaag ttttaaaata ttaaatagga gggttgggga agatttcatt agagggggat |
| 72301 | gtttgagcat ctctcaatcc tgaaagacag gaatatatga actaattact tgaaacaaga |
| 72361 | atatataatt aaccatttgc acattagctg tcttttttac ctaaatgaaa ttccttatgt |
| 72421 | ttttataatc tttattttaa tgcctacttt ctgactttaa attattatat atttaataaa |
| 72481 | agagataggt ctttgtcatt gtttttctac tttgaaagaa ctgtgtactt taatgagatt |
| 72541 | attccagtta tctatggctg cacagcagcc tccccgaaaa cttaaaggct aaaacagcaa |
| 72601 | gaggtttatt atacatcata gtgttgtggg ttataaatga gggcagagct caggtgggtg |
| 72661 | attctttgtg ctctgaggtt gatgttgaca gaagcttaca gctgccaaat ggactgatct |
| 72721 | ggtgtggagg gctggagatg ctttcatttc gacgtctgcc ttggtacagt tagctgtaca |
| 72781 | gctggtctca cctgggattg taggccaagg tgcttacata tggcctctcc tgtgaaatag |
| 72841 | tatcaggtag tcagactttt tatatggcag ctctagagcc cccagagagg atatctcaag |
| 72901 | caataagtag aggctggcag tttcctaaga cctgaatgaa gaaagtgata caatgtcaat |
| 72961 | tctggcgtac tctgttagct gaagcagtcc ccagattcaa gggaaagaga taaaacccca |
| 73021 | caaagaatgt gtgatcattg atattagttt cctaaggctg ccacaagaag ttaccacaaa |
| 73081 | tttattggct taaacaacag aagttttttc ttagaattct ggtggcagaa gtctgaaatc |
| 73141 | aagatgttgg cagtaccata attgctacag tctctagggg tgaattcttc ctcgtctctt |
| 73201 | ctagtttctg gtggatcctg gtattctctg gattgtggca acataactcc aatctctgac |
| 73261 | tccatttgta catcgccttc ttctctgtgt atcctattct cttataagga aactgtcact |
| 73321 | ggatttatgt accaccctaa tccagtaaga tattttccca atcggtgtat taattacatc |
| 73381 | tgcagtgacc ctctatccaa ataaaatcgc attttgaggt ttgggattga cagagatttt |
| 73441 | ggggaaactc tattcaaccc actacaccat ctatatagcc ttctacaaga tggtggaacg |
| 73501 | gtccggacat aacacagatg atgcaaatat cagacaatta ggtccacagt actagaatat |
| 73561 | aacatcgtgc aaattgtgct aattgaaaat ctcctgctta tacactatcg aggaaaccga |
| 73621 | ttcttatatt gttttctttt tttacagtga catgaaaaag gttggtgtca ccgtggttgg |
| 73681 | gccacagaag aagatcatca gtagcattaa agctctagaa acgcaatcaa agaatggccc |
| 73741 | agttcccgtg taaagcacgg gacggaagtg cttctggacg gaagtggtgg ctgtggaagg |
| 73801 | cgtagcatca tcctgcagac agacaataat tctggagata ctggtggaag ttccaagtcc |
| 73861 | aataagacac tcaaatatga gtacaaatgc cttaaaatgg aattgaaaaa ctctttattt |
| 73921 | tcccctatca tttattggat gggtgggtgg ggtatttttt tgtaattgct tttttaaata |
| 73981 | ttagttaatg gattaaattt aattcttcag cgtaaaatgg tgaagaacta gcatatagcc |
| 74041 | attgatcata aactgactat cataaaatca aaacaagtga aataacaaaa tggacatggt |
| 74101 | ggctttgttt aggtagagcc acaaaagaaa agacttgtaa tatttttata tacagaggaa |
| 74161 | atctgtaaca ggtattttgt ttcttttaaa gcaagcaaca cagaggaatt tatacctcaa |
| 74221 | actatctggc catatttact accttatcac tgcattattc tcttttatct gtttaaagca |
| 74281 | tatagagatg aagtttgtag ttgttttaag tactacacat ttttaaattg ttagcttcct |
| 74341 | taagtatatc atgtaaagaa atgtcttaat ttttgaaaaa agtacatatt tattttcttt |
| 74401 | tgaattgttt ttattgtttt ctatttatgc cttgatgatt taatatggat ttgttacagc |
| 74461 | caagtgccaa atgctctctc aaattgtcag caatttaact agacacagat aataatgggt |
| 74521 | ttctttcaga ttttttgaac catccactta catatatttt taaaaaatga aatccttttc |
| 74581 | ctgttcatac actaaccaaa tctctcaaat ctgttatccc aatcattgtt gcctctccgt |
| 74641 | ttattataaa ctgtatgctc acaacttagt gtaatatacc agcttgtatg caatggattt |
| 74701 | tcaaccagat aacatacctt tcctgctctg gtgcttagag actatcaact ccctccttta |
| 74761 | gtgaaggagc cgtgttagag cttccgagaa tagctccact ggagagaagt ggaatcctat |
| 74821 | atagaatgct gcactaattg acaacacagc atataggaca atgcatgagt aaaaaaaaaa |
| 74881 | acaattactg gctcactggc tttgaaaagt cacttactat tgttgctgaa acttgctgag |
| 74941 | ctgtttatag agaatgatga taacagaact tttcctctgt atcactggtg tttaggtgaa |
| 75001 | ttaattaaac attgtgatca ttagtaccag gtattattat ctttaagagt cttccacttc |
| 75061 | aatgcacatg gtgcagtttt ggtgtgtaac ttagaaggat tgaacttctt tgaatttact |
| 75121 | ggacataaca ttttcagaat agttggtcat ctagcaaccg cctcaaaatg tgtaagcagg |
| 75181 | agagaaattt ctcatcacag ggatttagac ttactattac ataaaggcta actatgagct |
| 75241 | tgctcattaa ttttgaaaag atgtacctgg tggatatcta gctagtaata tattctgaag |
| 75301 | caacatttta gctctattga tactctttct aatgctgata tgatcttgag tataagaaat |
| 75362 | gcatatgtca ctagaatgga taaaataatg ctgcaaactt aatgttctta tgcaaaatgg |
| 75421 | aacgctaatg aaacacagct tacaatcgca aatcaaaact cacaagtgct catctgttgt |
| 75481 | agatttagtg taataagact tagattgtgc tccttcggat atgattgttt ctcaaatctt |
| 75541 | ggcaatattc cttagtcaaa tcaggctact agaattctgt attggatata taagagcatg |
| 75601 | aaatttttaa aaatacactt gtgattataa aattaatcac aaatttcact tatacctgct |
| 75651 | atcagcagct agaaaacatt ttttttttaa atcaagtatt ttgtgtttgg aatgttagaa |
| 75721 | tgagatctga atgtggtttc aatctaattt tttcccagac tactattttc ttttttaggt |
| 75781 | actattctga gcatactcaa caaaacccat gcatttcata aactaataga agttgaggat |
| 75841 | tgttgaatct atttcactta ttttggctgt ggtttccatc tgaaagtaga ggttgtatac |
| 75901 | accatatact gttcttcatt ttattaatat ttttctcctt gacctctcat aaatttactt |
| 75961 | tacacaattc ttaccctgta catatgtaaa cataagtgta cgattcttaa ccatggagta |
| 76021 | gaggtactag aatgcttacg gccatctctt tgtacaggaa ctgcattgac tttcagtaaa |
| 76081 | cataaagcca caactcctac atgatgttat gtaccatatg atctgttttg tatcttaaat |
| 76141 | ttgatttaca tatattattt atttctggta actcactcag tttatgctgt gctaaatatc |
| 76201 | aatcaagcca tgtataaatg tgatatgatt ggcaatatgt gtttacttta aacttgtctt |
| 76261 | ttcaaaatat tactcagttt atgttgtaca atgtagatgg cctcttacta atgtaaaatg |
| 76321 | atttgtagtg gaaacattta tatttttata ataaacataa tgaaaatatt ttttacagat |
| 76381 | tggaatacag aagtggtctt tgaagttttt taaaaatata taaaactatg tgcttattta |
| 76441 | aacagcaaaa taatgaaatt tacataagtc acaaaaatat gcttctgggc ttttattctc |
| 76501 | cattagtgag gagggattta cattgtataa tccacatgtt tttggtctac atctcattat |
| 76561 | gaataagcca gaaaaataat cagtaaatgt tatttcaaag ttaataaaca caactgtaat |
| 76621 | agRcagttct cttttgattg caaataacag aaaacaaata aattggatta attttctaaa |
| 76681 | aaagaaaatg tttagaataa gtgacagaat tatctgggga taaactccct agctaagaga |
| 76741 | tcagacacag attgaaccag gaattcaaac aatgtcaaca gggttaattc tctcattctt |
| 76801 | tgtcttcttc ccttggccct attttttttc caactgggtt gatttgcaag ctataccttt |
| 76861 | ctacaaggta aaaaatacta ctctctttaa tctaattctg cattgcaact cctatgaaag |
| 76921 | aattactgtc tctctagttc caacagagat catgaggcct ttattggatc acaagcccta |
| 76981 | tcattttggg ctggagaatg aatcactcag attggcaagt actgactcct gtgcttgccc |
| 77041 | ttggagatag tgtgtggggt tatatgaaag tgatgctcag gggttagagg tgtaaaagta |
| 77101 | atttatgtcc aacagaaagt cagaatactg tttatgacaa acgaagaaag gaattctggc |
| 77161 | caggtcaaat aaagcaagta aaaattaaaa aaaaaaaaaa aagtacaact gcacacctga |
| 77221 | tactttgtat acaataggtc ccttgtcatt gcagtccact taaagaaaat gattatatat |
| 77281 | gaaaatcaaa aaaagctaat tcaaaaatat gcaaaataaa ataagtgatc aacattccca |
| 77311 | tagagaagga agtggttagg aagagattaa aaaaacaggt ctgatttgtg tttgccttga |
| 77401 | aggagaaata gcatttggtc aagttgagtg gagtagagaa taccttgtaa gcaggagata |
| 77461 | agttagttaa gttaagggga agagaagagg taaataatat atttattcat tcaataaata |
| 77521 | catagtatgc atgatcatga gccagttgtt ttgctatcat catttacaag ttacctcatt |
| 77581 | aaatagtttg caacagtgct gtgaatggag atgagctact tttatctgag aaaggactcc |
| 77641 | aggctcagaa tgtccaagtc accttcccaa ggtcacccta gtatgtagat agatatttca |
| 77701 | aactggaatc ttcctattct gtattcctcc catcatgtga tagctactat aatctccaca |
| 77761 | atgctctggg gacagtggca atgacaattt gcagaagagg cttgggggag actaggtcta |
| 77821 | atagactatg tgatgttaga aatctatttt ggtattctat gcacaaagat atacatatac |
| 77881 | cccaggatgg tggtaataga gatgtgaaga atggaaagca tacaaaaata tataaaagaa |
| 77941 | agaggattaa caaaatttac cgatttgttc tgagaattct tcgaaagata gaaatcaaag |
| 78001 | caaaacaaaa atttaggtct tgagtgtctg tggaaatgat ggcaccatta gtccaagtag |
| 78061 | agaataagga gaagtcagct taacatcatg ggtttgtcaa atatatagtg taagccagac |
| 78121 | tttaataagt tactaagtat tggggatgag aatctgaagc tttaagaaaa agaattcaag |
| 78181 | ggtaaaaatg tatatggaaa gatgtctttg aaagtatgta aagatctaca ttacaaatat |
| 78241 | ttatgtgttt cctatataga catatatttt tcttggaaga cctcttaggt taatgctagc |
| 78301 | tacatagtta agttctcaaa tattatcaac ttaaaataat aaaaatatgt tttttaacac |
| 78361 | agaatgaKcc aaagtaggtg tttgccaggt ggccttcttt gcaataatta agaatctaca |
| 78421 | ctttctccat cttgtcgagc cttgaaatcc tctacttcca gccaggaaaa tgaaggagag |
| 72481 | gatcatttgg ggggatttta tgaactctgc ttgtaagtgg tacataccac ttcttctctg |
| 78541 | gttccactgg caacaactca accacactgg catacttaac tgcaagagaa cataaagaga |
| 78601 | gttgtctgac agtgtgccta aataaaaatg gaaaatttct ggaatttata tcacaatctc |
| 78661 | tgccacaact ccttttaaca taaataatat gtcagaggca gattgttcta aaaagcttat |
| 78721 | cagagaaaca tcagtttctt gctaactgtg ccaatttctt ttcctctata ggcaaccatt |
| 78781 | tttcattctt ttctcttatt acataaatcc aatacctcta ctaatggcac ttgtcagcta |
| 78841 | tgtgagtaca ctgctcagat ttcctttcaa gagagtttct tgcaagagag cagttagctg |
| 78901 | acaactgtga gctgctatat tttcaaaatc cccagcatat ttttagcagg ccatgctcta |
| 78961 | ccttggttaa tgcctgtcaa tgactgagta tggtggaagt actagYgcca ggccatttct |
| 79021 | tcacaagtgg taaatccctt taacaagcgt attttgtgtt ctaatcatcc tgactgaaRc |
| 79081 | ttgctcaggc ataccctaca gtctgatctc actactactc aattttcctt ccttcactct |
| 79141 | ttccttttat aggtgtcaga actgtatttc tatctgcaag ctccttgtgt tttctcttaa |
| 79201 | accctgttcc aatgatgtta cccaggcatt tctagcaaaa aatatttgcg ggtttaattc |
| 79261 | tttctgtgtc tgattcttga aaatcttaaa cttagatgac tctcaagttg actcacttgg |
| 79321 | aaaactaaaa tgttatcctt cattcttcYc tctRcctttt cttcagtgtc taactgatcc |
| 79381 | cctaacaatg acaccttaga aacgctcact ctcatccctt ttctctactg cccagcagaa |
| 79441 | tctctgttgg atcctcatta cttttctgag ttacagatgg aatttcttaa attgtgcccc |
| 79501 | tgattctagt cttgatatct tcaacctaca aaagtaggtt gaatatgcag ccagactaat |
| 79561 | acggcaaagt taatgttgta gttcctcctc aggaggcttg caaacgcaaa tgcctcaggg |
| 79621 | aactggcagg ttatataaat gagtgaagca agtgaagagt gtggtgaccc ataaaagtgc |
| 79681 | cttttcttcc acgaaaagca aatttctcag gtgtaaggca acaccgatcc agtatgaatt |
| 79741 | cctggttttt ccttttcttt gtttttaagg tttaaggaaa ctaggtttta attttttaag |
| 79801 | tttttgtagg tacatggtaa gtgtatatat taattcttga tttttcaaga gaatccttaa |
| 79861 | tctgagtctt tgtggaaaaa tttctatata tttaaattat aacccactta tttatgaaca |
| 79921 | cacacacaca cacacacaca cacacacaca cactctgaat ttaccctgca gctgccaact |
| 79981 | ttttatttct aaaatacaga ctactcattg gtattggcac atgagaccta tcttgaataa |
| 80041 | aacccggctt ctctccttat tttctggtta aaccactacc tccttacact ctatacaact |
| 80101 | gtactgaccc acttgtagtt ctctgagtat tccactttct taatgcatca tagcttttga |
| 80161 | ctttttKgat cttttgtcta gaatcactta gccaatctct aatcattctt caggtcctct |
| 80221 | tatatcccta aaaactgggt taggtaaccc ttcctcgtgc agcagtagaa ccctgtactt |
| 80281 | aatactgaca cacactatct taaccaaaat tgcctattct ttatgcccta tctttacttc |
| 80341 | tagctgagga caccttgaac tgtgttttgc aaaccattgt tgttgcatca cctattgcaa |
| 80401 | ttttcctggt atataataaa cacaaaattt ttttgagttg aattaatata ttaaaacaat |
| 80461 | gtttgatggc attataaatg gcaatgtatc tgattatttt ttccccgtag gaaaaagtgt |
| 80521 | aaatttcctt cagctcaata aagctagtta atgttctagt tatatatctt ggatcattga |
| 80581 | atatactgat ctctgctaga actggttcat tttcatagaa ttttgtggac ttgtttcatt |
| 80641 | ggtctcaatg ctcccacttt tcttagtgca gttaagttgt ttaagtagac ccttgaacca |
| 80701 | gagcttagct ataaaaattt aacaaggtaa atgccaaagc ttttctgatc ctttctactc |
| 80761 | tctcactatg tacaaataat gtcaacatgc ctactaaaag ctacccttct tatgaggctt |
| 80821 | ctgggatgca cattctgttg acacacaggc tctagatgcc atttgggaaa ataagtggtg |
| 80881 | gagttagata tgccataaaa tgaagagttt ggaggtttgc agaagtcctc accctgaaat |
| 80941 | tctctgggaa aactcagtta catgttgatg aagaatagta ataattgaat cgcattttcc |
| 81001 | aacaaagtgg gctatcagct ttcctctttg aagaaaggta attttccact taacttagaa |
| 81061 | ataaagtggt gctagcaata gtaataatag aagaaaagaa gttgcttttc ttctattatc |
| 81121 | tgcatgttac atatatgtac acacacatat aaactaactt ttctgaataa ttataattga |
| 81181 | tgtacattga acttttactc tgtacaaagc ccttaacata tattatttga tctaattttt |
| 81241 | ccaagaactt aatgaaataa gtatgttgtt atccacttta taggttgcaa cttagaatac |
| 81301 | ctaataatca cacaaggtca tacagctagc aagcagaaga actgaagtca aaatcgctcc |
| 81361 | agatctcttg ttctgaacta ttatgttgta ttagctgtca gtataatatt acaacttgag |
| 81421 | gataaaatct gaaggacaat aataaaaact attccagaat taagatcttg ggtagtctct |
| 81481 | attttccact tttcaatgtg caattgtaat tcagtaattc tgctagtaga aacatagttc |
| 81541 | tgcagaatcc tttgattgtt actaccatgg tagaggaagg aacaggcagt tatccttgat |
| 81601 | ttcatctctg agaactccat ttcaacagca tgagatttgc ttgacaYaca gtgaaccctt |
| 81661 | caagcaaaca tgttcccttg aacattcaac aaataatcta atttggaaac accaaatacc |
| 81721 | acaaggaaaa agtagtactt ttgttctatt ttctgacaga tttccacacc agtgatttat |
| 81781 | ggacgtatgt taagtttagg attcttatat gaagccaaga aaaaaacctt tatatttttg |
| 81841 | aagcctatca tcctcaatga tggaagccaa acccttggaa tgttgctaaa atcaaggatg |
| 81901 | agagaataca gaatgtcatg tactatcaag aacttattga ctttcatatg gttctcattt |
| 81961 | attctcatac atggcaaatt gaaagccttg ggaggaagcc aatacaaatt gttacagata |
| 82021 | acactatcta aatttaactg ccataggtat gcttcatata ataaatatat atttcatatc |
| 82021 | agcaactcag caaggcaaaa attgaaacag actctagtca aaattcattt ggacacaatc |
| 82141 | cttcagagaa gactcaattt gccaatataa ttatttatYt tggtctggag agcccttaat |
| 82201 | actcaatttt atgtttctga ctctagagat tttatacaca cacacatata tatacacata |
| 82261 | catatataca cataaaatac tctatggaag ttagaacatt gaaatcataa caatatctta |
| 82321 | gggtaataaa attaagtttg aatacatgaa agtttagtac ctgcctgcag aatcaaatag |
| 82381 | attaatttgt tgatattttc aaatttttaa agacattttt gcaacagaat tttttcttgg |
| 82441 | tttaatgggt tcagtttgag gcatcatttg agcattaaag gctgtggcat gacattaatt |
| 82501 | tttagcctag tgtgatggtt catttatcaa gtgcttaaca gagtatctag tatacaatag |
| 82561 | gctagtaggc atacaattag tctttgccaa atgaatatta aatgtattgt tggaggacaa |
| 82621 | atgaacaata ttatctattt caacaattgt agcagagatt ctgtgcaaca tgcagttgct |
| 82681 | ttccatttag attaatgttt ctctgaaaca gaaagttttg tagaggaaag tcttatctaa |
| 82741 | cacatagaca gattcagggc ccagcacata gaataaaccc ataattgttg aataatacac |
| 82801 | atttataatg taaaatcact atgggtaaca aaggaatcct attatacact ttcctactct |
| 82861 | ggataaagag aaatatatat atttacatac atatatacac atacttaatt attaaatgga |
| 82921 | ttaattatat acatatataa atatgaatat aatatatata aatatgaaca caatatataa |
| 82981 | atacaaatat gaatacatat atacatatat ttatatatac ttatgaatac atatatacat |
| 83041 | gtttatatat acttataaat acatatatac atatatttat atatgtatat aaacaaattt |
| 83101 | atttatatat atacacatat atttatttat tatctggctt actaatactt aaagaattaa |
| 83161 | taaatgccaa agccagggat atgactaaaa aactaagata tctttccttt taagatagct |
| 83221 | atgattgcat aacatgagac actgtggttg cataatatca ttcagtgact taaaacaaca |
| 83281 | aaagtcactt atttcatgtt ttttgcaggt caggaattta tacatgggaa agcttgtctc |
| 83341 | tgctccatgt tgcctaaact tcagatggaa gactcaaact ctggcattgg attcttctga |
| 83401 | atatttattt gttcctctat ctggttattg gtgttggcta ttatcgggga ttcagctaag |
| 83461 | ctcacacgtg ccttcacaat gtggcggttg gttccaagta cagacatctg aagggagggg |
| 83521 | aggagaaaga ggaaggatgt gagattttac cctacttaca agccaacaat ttggcctgcc |
| 83581 | acattttcat ggatattgKc agttgtcatg aggtttctgg gtcagagaca aagaatagtt |
| 83641 | tggtactcac agcagtggca gtatccagtg tcaccatttt catacacgaa ttccccaagg |
| 83701 | cctaatttct acaaagttct gcaaagagag ccaagtcttg cctgcatatg caatgagttt |
| 83761 | tgttgaagaa aaggaaactc atgtttaagg aactacaatc ttttataatt ggctgcaagc |
| 83821 | aaaactgcct aacaatttct tcagagagaa atgtctttat catatcggag agttaacaaa |
| 83881 | tctaccattt gctgcaaagg ggagtactat gtctttattc caaggttatt taccatacaa |
| 83941 | atatctttga aaagataatg cggaaaagag ggcagttagt gcctctgctt gaaagatatg |
| 84001 | cagaaacatg aaagacccat agagaatctt ttcctccgaa agactccttt caatctttgt |
| 84061 | ggttaagaac aatgaataaa tttaatgtaa actcattaaa agatttaacc tcaaaaaatt |
| 84121 | ttaatatcca catttaagat ttctttagga gagaagaaat caactccctt accctcatcc |
| 84181 | tcctttggtt aggaatgact acacagcata gaaaacttgg gactgttagg cattaaacaa |
| 84241 | tcaaaagatt cttctcaaat taagataata gcttacatga atattttatg aaaaatagcc |
| 84301 | acatttgata ggcttttcat taaacctata tataatttgg gtagaattca cacctttaca |
| 84361 | atgttgagtt tttcaatcca cacatacggt atgcctcttt gtttatctac atcttctctg |
| 84421 | atgtctttca ttagcatttt gtaattttta gcataaagat cttatacaag tattttttta |
| 84481 | atttgtactt aagtacttca attcccttgg cataattata actgatatat atatatgtgt |
| 84541 | atatagatat acatatatat tatttattta tttatttatt tatttattta tttatttatt |
| 84601 | tttgagaact ctgtcaccca gactggagtg cagtgacaca atctcggctc accgctacct |
| 84661 | ctgcctacca ggctcaagct attctgctgc ctcggtctcc tgagtagctg ggactacagg |
| 84721 | agtgcaccac ttctgtccag ctaatttttg gatttttagt agagacagag tttcaccatg |
| 84781 | ttggccaggc tggtctggaa ctcctggcct taaataatct accagcctca gcctgacaaa |
| 84241 | gtgctgggat tacaggagtg agccacttct cctggcccta atattatatt tttaatttcg |
| 84901 | gtttctacct gttaattatt gaaacatgga aatgtgatta attctcttgg gtttggttat |
| 84961 | atattctact atctagttgc attcatacat tagttctaag tttttatttt gtagatgctt |
| 85021 | tgggattttt ctatgtagac aaccatgcca tccccaaact gtgaaatttt tacttttttc |
| 85081 | ccttccactt gtatgcattt ttatttcttt ttctttcttt attactctgg ctagaatttc |
| 85141 | tcctactatg ctaaataaag ttgtgagcag acatctttcc attgttccca gtcttaggaa |
| 85201 | gaaagctatc agtctttcac tactaagttt gatgttacct gaaatttttt gtagatattc |
| 85261 | cttatcaagt tgaagatatt cccctctatt tcaatttttc tgagagtttt ttttcatgaa |
| 85321 | aaagtgttga aatttttcag attttttttc tgcctcaatt agaatgatga tttttttttt |
| 85381 | ctttttgatt tggtgatcat actgattgat ttttgaatat ggaatttgtc ttgcatacct |
| 85441 | ggggcaaaat atgcattgtt catggtgtgt aatcatttaa tacattactg aattcaattt |
| 85501 | cctatgattt tgtagagcaa tttgtgttga actttctgac acatagatag gtgtagtttt |
| 85561 | tgcccatctt ttacttcttt aatttttctt tattttcttt gttgggcttt cgtatcagga |
| 85521 | taatactggc ctcataaaat aagttggaga acattctttt tttattttct gaaagagatt |
| 85681 | gtataaaatt gtgctaattc ttttgtaagt gttagagaat tctctagtga aattacctag |
| 85741 | gcctggagat ttctctctca agctatttta aattataaac tctatttaat aactacacaa |
| 85801 | ctatttagtt tatttcaagt tggttcattt tggtaatttg tagcttttga ggaatttctc |
| 85861 | aattttaaaa gaattgtcaa atgtgtgagt ataaaaatag tttgtattat tcccttgttg |
| 85921 | ttctaatcat tatgaaataa taccacttgt ttcattacca atgtttatga taattgtcat |
| 85981 | gactattttt tggattttta acacttttat taactttttg ggataaaatt tttgttttgt |
| 86041 | tggttttctc tattgttttt ctgtttgatt cttgtttgtg tgtgctgttt ataatttctt |
| 86101 | tcatttgctt gctttgggtt attattttct ctttttttct agtctcttaa ggagcttaga |
| 86161 | ttgtttattt gaagcatttt ctcttttcaa aaataatcat atagtattat aatttttctt |
| 86221 | ttattgctat tttagctgca tctcattaat attgatgttt gttttaattt tctttcaatt |
| 86281 | ctctgtatat tttatttgac ttcctttttt atccatggat tatttaaaag tgtgtttttt |
| 86341 | aattttcaaa tgtttagaga ttttcctctt gtctatcatt gatttcaaat ttgattctat |
| 86401 | taaaattaaa tagtatattc tgcatgatrt cgattatttt aaatctactg aggttagttt |
| 86461 | agtgagccag catatggtct atcctggtga atgtttggtg gacaattaaa tagactgcat |
| 86521 | attgtgctgt tattgagtgg aatgttttat caatgtaagt tgaatcttat tggttgacag |
| 86581 | tgtttttcca ttctcctcta tctttgctct ctttttatct aatgcttcta tttcctgcta |
| 86641 | acagtgggaa tttgaagttc tcaactacag gtatagattg gcccatttct tctttcagct |
| 86701 | ccatccaatt ttgctttgtg tattgaaaag ctctgttttt tttttttttt tttttttttt |
| 86761 | ttgcacatac acatttagga cacgatgtct cttgttagat taatcctgtc attactatgt |
| 86821 | aatacctttc tttgtcccta ctaattttct ttgctcttaa gtccacttta tgtgttatgc |
| 86881 | aattatatat atattacata tatttatatg taatatatat cattatatat tgtattgcct |
| 86941 | cagagctaaa gcaattttac ctcctaaggt aaaatagagt atgcaaacaa atattggcca |
| 87001 | aataccttac ttttaaaaat taatgtttgc ataacatatt ttcatccatc attttattct |
| 87061 | taacacatct gtgtcattat ttttgaagtg agcttttgta gattcaatgt agtggggtca |
| 87121 | tcgtttttat ccattctgtc aacctctgtc ttttaattga tgtatttatt ccatttattt |
| 87181 | gtcaagtaat tatattatgg cttaagtctg ctaatttact gtttgttttc tgtttcaaat |
| 87241 | tatgcccttt ctcttttctt gcttttctgt gggttccttg acctagtttt attttagaat |
| 87301 | tacatttttg tttataaaat ttttgaataa ttttgtataa ttttcttaat atgtgctcaa |
| 87361 | ggtatgacaa taaacacttg caacttcaaa aagacatgca aactcttaaa atgtttatgt |
| 87421 | gaacaaatat tatcttcttt acctgtggca aatgataact gggctagaga tttttataag |
| 87481 | ttcttccagt tctaacaatc tgtattatgt gatatttact gttataaatc tattaaaatt |
| 87541 | tgaaaatgac agtgctgtat ataacaaaat aaaagcctat gactctatcc atgctactta |
| 87601 | gcagggcttt ttgaactggt ttgtccagag tttaaagagt tatttcgaaa ctattcagga |
| 87661 | ccctcttgct tgcttgtggt aattcagaat tattcagtaY ataagaggag ttttaagcac |
| 87721 | ttgtgttctt cgtgctctta aaaataaatg caccctaatt tgcttcttgt attttttata |
| 87781 | attttacata gtaataaaga cttcaatagt tttaagattt gtaaaagtca ctattgtaat |
| 87841 | ataagctgac acctgtgatt ggtttatttg gttgtagatc actgaacaac tgagaaaaga |
| 87901 | aaaacaaaag cacagagtta gcaaagtttc tattatcacc aatataggcc aaaaattcca |
| 87961 | cgcatttatt cagtggcaac aagtaatgta ggttttttgt tgtttgtttg ctttttctga |
| 88021 | gatatctact ctgtgccagg catgatttga ggcctcagct atgttggtga aataggcagg |
| 88081 | taaagactgt agggaagatg gaattaaagt tactgttgaa gcccgaaaaa attctagttc |
| 88141 | tttttgatcc ttccaagagc tcagatgcta agatgatttg ttgttcccac aatccatgca |
| 88201 | tcattaaata aacccataat cactaagtaa actatatatt ttctgttctt tacattctga |
| 88261 | aagcttctaa gggacaccct tagccatgat actgtaaggt cttgaaaatt tggctaaaat |
| 88321 | catttctatt tgtatacctc tggatgtttt aaacactaca gggcttttaa aaagtgacat |
| 88381 | tttgaataga taagaaaata aacattggta tcagcaaata ttagatatta ctgtgagttc |
| 88441 | ttgctgttat gcttgttaaa aagtatcatg atggaaagaa atcaggattt agaatcaaac |
| 88501 | aaatctgtgt tcaagaccct tagctgtttt tgttagttga attcaatgct ggctgaactc |
| 88561 | taaagactca gttttttcac catgtaaaca ataatcatta tataaagcta atagagctgg |
| 88621 | aaagataaag tattatgggt aaactgcttt ccttagttta tctacttaag tagtgctatt |
| 88681 | tcctttcttt atttacttac cttgatccaa gtgaaagcct agttttagga tttactttta |
| 88741 | ccaaaattgt actctgaacc aaatgagctc aaaagttMaa ctttgaaaag tcaaaatcat |
| 88801 | agaaaatttt tacattgaaa ctgctcaata attgttttca aaatgcacag caggacttct |
| 88861 | cttatttacc atggtgaata aaatgacttt agtttttgac atttgaagta tataataata |
| 88921 | aaagttatag aagttagaaa gcttccttca gggaaatgga atatttttac ttcctatgaa |
| 88981 | agtgatattt tggtctcaat ttaagtaaca caaaacttag ttttcatttc ctggttttaa |
| 89041 | atgataagct gtatattcct agtttacagt aaaccacata tttttagaga gttagaaacg |
| 89101 | ttgcttcaaa gtggtagata ttaagtgtca taaatgtcca ggctgtaagt atggtttcct |
| 89161 | gYtgtgtaag acataagtta ttaggtttag tttgaaatga cacaaaatgt tcccgacttc |
| 89221 | taacaattaa aagaagaaat gtgaggctcc gagacaatct cggcaaattt gtattctgat |
| 89281 | taaaataatt atttgatcct atacagttca gtgcatggaa aaatgttgtc tagttactca |
| 89341 | attcaatgtg gttgttagag aagaaaatat gtctcttatg tggggtttga gcttcatgta |
| 89401 | gctttgtgat tgtatttgtt tactcacggt ggaaatctcc ttattaaatt gtgggcaaga |
| 89461 | ggcaacccat ggtggaaatc tccgtattaa attgtgggca agaggcactt ctattggttt |
| 99521 | ttctaacagt aactacttac ttcaggaagg attaggttaa aatactaaat gtagatctct |
| 89581 | agagttgatc ttgtaacaga actagaacac attgacttga tatttagaca tgtgcagaag |
| 89641 | gcaaaccatt tcataaaggg ctggatataa tttcttcttt cagttctaag tcaaaattta |
| 89701 | ataatttttt tctattttta ataaatatat gggcatatgc atctaatagt gaggtttggt |
| 89761 | aaagactctt cttcacttta gttttgcatg tgtgggcaac tgacctttat gttgttacaa |
| 89821 | aatactactt aaaaataaaa agctcaattc tgtacagaaa tgagccattg ttcgttgtta |
| 89881 | ggctctcatt agctgttata gtatcaacac tattgtcaac atgatgtgga tcttgaattg |
| 83941 | ttttcccgtc tccactgcta actgtgtgtt cctcttgttc ttcgggccag ttgaaccata |
| 90001 | catcttgtgt cctaaaacca tatggagcat gtttttgtat gaactctcat gattaaggac |
| 90061 | atctgtctca gcctctacgt ttgccttcag gggatctttg catcatttaa tggtacagtt |
| 90121 | aacagcatgt tttgcatcat gaagttggcc gtacagtttt tctgcggatt ctactgtttt |
| 90181 | cattccttaa aaaaacacat ggacgagtct atattttgtc tttctgtctc ccattctaac |
| 90241 | tttcttgctt ttcttcctcc ataattctat ttcttttttt tttctattaa tatttactgg |
| 90301 | ccatcacctt gtgtttagca ggtcttataa tattccaaaa ttgcacattt tcaaaactgt |
| 90361 | aaagattgac cacaatttct aaatgtaatt cacttttcct ttcaattgct tgttttgagt |
| 90421 | gtaatttgga taccctaaag ttcatgtgtt acttttctat ttattttgag taatatgatt |
| 90481 | cagattcaac tcctttagat attactgaaa atattcttaa gtgaattgat acaaaatatc |
| 90541 | cacattccta ataagttatc aaaccaaaat gttgctaact ctatactgaa attctactaa |
| 90601 | ttcttctgac tgtacagatt gattcgtcag tatatattag caagtattta attaaaatct |
| 90661 | attttaataa ctttaaatgg atatagtagc actctaaata ggaaatgcag tcattgaaac |
| 90721 | tctcaaacac tagggaatag atataaaata tgtgcattta atcaattata tgttgttgat |
| 90781 | ttttacttta taatctatga cttcctttgt tgatgtcaat attgactttc tttaaaaatc |
| 90841 | tctaaattat gaaaaagtct aagttattgt aatctacatt atagactttt tctagtatgt |
| 90901 | tttactcata ggtaaattgt tactagtccc attaattgca aaatgcagtg gtcatgtgtt |
| 90961 | tccaaataat ttcaaattgt agaaataaat acagacagtc tccatgaaag catattgatt |
| 91021 | caagaattct atttattgat atttatttac tcttcctaac atttatattt actattcata |
| 91081 | gtgcataatg tttgttcagc agaaatatca tcaaaaaact attgatgtgc cagttaccat |
| 91141 | gatgcaccga agatacagaa acgaacaaga tgtccccact ctggtgaagg agtctatttg |
| 91201 | ttgttggtat tgacagtgtg agaggcagat gtagaaacaa gattgacaga ctttgtattt |
| 91261 | aaaggtgtaa gtttcataaa tggRtacata gaaaaatgaa cacgtttaaa ttaatgatgt |
| 91321 | aaacatgtta atattcccaa atacaaatat aagtctgtac agtgacttgc ttttttaaaa |
| 91381 | agcattgaag tggaatttgc ataccatgaa attcacacat tttaagtgta taRtatgttg |
| 91441 | atttttgaca aattttaaaa tcttgctagc atcaacacag tctagtttta aaacccgttc |
| 91501 | ctcactccag aatgttccct tgtgcccttt tgcagtccct cactccacat tccagcccca |
| 91561 | aacaatcact gatctgcttt ttatctctgt agattttcct tttctagata tttcatgaaa |
| 91621 | atgcaataat aaataatgta taaatcatta aagatttata gcaaatgtag ttttgttacc |
| 91681 | cttttttcat gtttctcaaa ttgtttgtac aaggaaaatt atttcagttg gtattttgaa |
| 91741 | atgtgggaga ggcctttgac taagaacttt aggagtaaca cccctaactc tcattatctt |
| 91801 | cattttacag atatgaagac tgtgctttat aaaagatcag acatttggct aatgacactg |
| 91861 | ctactaagtg acaacagaga tttaaaacta gttctgtcta attctaaatc cactgataca |
| 91921 | gtttctcaga aagttgaaaa acttttaaca ttataaactc attgatagtg atggatattt |
| 91981 | gttcttttgt tgtttagtga ttacctcaag atctacaaca atgatcagca atacaatggg |
| 92041 | agttttagtt gtaatcagct gtttcatatt ttaataatta ttaaacactc atgtaccaaa |
| 92101 | tacaatgcta gctgccagaa attacaaaag gaataaggtt tgatataaag taccacatag |
| 92161 | gccaggcgca gtggctcaca tgtgtaacac caacactttg ggaggccgag gtgtgtggac |
| 92221 | cacctgaggt caggagttcg agacctgcct ggctaacatg gcgaaacctt gtctctacta |
| 92281 | aaaatgcact tagctgggtg tggtggcaga cacctataat tccagctact tgggaggctg |
| 92341 | aagcaggaga attgctcgaa ccctggaggc ggaggttaca gtgagccgag attgtgccat |
| 92401 | tgtactccag cctgggagaa agagcaagac tctgtctcag aacaaaaggt accatgtagt |
| 92461 | tttcacttgt tctaaaaatc tcctctcatt gtgtaactca agtataaaaa tccatattac |
| 92521 | atctgaaaaa ggagttattc ttctgaactc tatctaacct ccttaaagcc aagtatatgc |
| 92581 | cactttgcta tgtcagttga tttgacttat atttggtaca cagattgcat tttttttttt |
| 92641 | gtaggacaga agaactccct gtcaagctag tttaactgta ttacatctac caatattcaa |
| 92701 | ttatttaatt ttttcaaaag tgaatgcaat cataatacat ccaaatacaa cctgaaactg |
| 92761 | ttgggaagta atgcttagga caaaatttca atctggttta ctggaaaatt ttagcaataa |
| 92821 | gtaaaaatct taatgcgttt ccatatgcat ggtttcacac actttttatc aagtactaga |
| 92881 | aaaacactga atgctaatgc cattgaattt tgctgaatta gtgtactata taaccccttt |
| 92941 | catattaaag attttaccca cttaacctca ccagaagtag tgttttcaca gtgcatgatt |
| 93001 | atgacaaaga tagaatgata gggagagggg aaagtgccat caggttcagg gtcaacagaa |
| 93061 | aataaaactc gttctctttt tgtaatcagt tataattccc atttcaatca aaaaaattat |
| 93121 | tgtaaagtat tttcttgaaa tttgcttaaa tttttcattt ttaacaagaa acgttattta |
| 93181 | taaaacacat tacttgtgtg catataggtg ttgggtctga gaaagtatac attattaatt |
| 93241 | tgatgtgtta ttaaaagatt gctgtcaaaa atctgtttag ttaattgatg ctgtcaattg |
| 93301 | ctcgcattaa tacctaatac tcataatatt tgtacctaaa tttttcttat aaaattgttt |
| 93361 | ctcattggtt tttattcttt ttatttctcc tattttttaa aattgaagtt tgtagtcagg |
| 93421 | tcatgtgacc aaatcaagtc catcattcaa acacgtatca ggaaattgga aatttaaaaa |
| 93482 | tgacttatgc atggatatga tgccagaggc agagacaagg gggtgggggc tgtttctaaa |
| 93541 | taccttccaa taacaataaa aaaacggaag ttatttgtga tcttagaaga tggctaagga |
| 93601 | aaaactgaaa cctgaaactR aggaaacaaa agtttagaca agataatccc aacatcagag |
| 93661 | aaagcatttg tcattgtcag aactccaaag agcaagaaag ctttaaWcca tttcatggac |
| 93721 | cctaggactc atggtctagt aattcttatg tcaccaaacc atattaggat ctgtatgaaa |
| 93781 | ttacagtgtg tcaccactct acatctgcaa tcttaatgtt tccgtccttt gattgtatga |
| 93841 | caaggaaact cttgaaccag agcactgcac ttcacaggag tctgaaagaa ccagctgctc |
| 93901 | ataattttgg agttttatag ggctttttag tctcagaatc cctgtctgac ctatgtccat |
| 93961 | atccagaatt catttttttt catttccaga attattcaga aaaaactctt gctgtctttt |
| 94021 | aacgtcttaa aacatttttc aaaaaatata tagagagaat tagaaagcca atcatagact |
| 94081 | taccctcttt ctccgttgct tcttcagttg tgtaatagtc atgaaatctg gaatgaccat |
| 94141 | tttaaattat attcactgag tttgaaacaa aacgttttta gatggttttg ttcccttaaa |
| 94201 | aatgaatatt attgtctagc attaacattc acccaaattg atagcatatt gaattatgtg |
| 94261 | tgtagattct taaacgtttt gttgtgaaac atcaaaagaa atcactgact ttttatttaa |
| 94321 | cataaataaa ttaagggaag tttagaaatc taattgttgg aaaaataaac ttctaaacat |
| 94381 | aactcttcaa aagtctacgt gctcacattt tctattcccg aacaaaaact tttgcaaagc |
| 94441 | tctctgaaag ccttgtaaaa ggccaatgaa tttcatttag cattaatttc cctactcacc |
| 94501 | acatagcaaa tatatgacac aaYttatagt tctgttaaaa aaaagccaga actgttacct |
| 94561 | gagcaactgc ttataaaaga gcgaggcagc ttacgtttta tctatttttt gtgtgctttc |
| 94621 | tactaagttt tacaatctaa gtgtgttaga aataaaatca aattcattag gaatctctat |
| 94681 | gcataaaagg aaattaaata tcatactcaa tgacttgtgc cacctgtggc aaatagtttt |
| 94741 | acatgccaat |

Following is a first EPHA3 complementary DNA sequence (cDNA;

SEQ ID NO: 2)

ggacagcacactgcaaggaaatcttcacgggtgtggagtacagttcttgt
gacacaatagccaagatttccacagatgacatgaaaaaggttggtgtcac
cgtggttgggccacagaagaagatcatcagtagcattaaagctctagaaa
cgcaatcaaagaatggcccagttcccgtgtaaagcacgggacggaagtgc
ttctggacggaagtggtggctgtggaaggcgtagcatcatcctgcagaca
gacaataattctggagatactggtggaagttccaagtccaataagacact
caaatatgagtacaaatgccttaaaatggaattgaaaaactctttatttt
cccctatcatttattggatgggtgggtggggtatttttttgtaattgctt
ttttaaatattagttaatggattaaatttaattcttcagcgtaaaatggt
gaagaactagcatatagccattgtacataaactgactatcataaaatcaa
aacaagtgaaataacaaaatggacatggtggctttgtttaggtagagcca
caaaagaaaagacttgtaatatttttatatacagaggaaatctgtaacag
gtattttgtttcttttaaagcaagcaacacagaggaatttatacctcaaa
ctatctggccatatttactaccttatcactgcattattctcttttatctg
tttaaagcatatagagatgaagtttgtagttgttttaagtactacacatt
tttaaattgttagcttccttaagtatatcatgtaaagaaatgtcttaatt
tttgaaaaaagtacatatttattttcttttgaattgtttttattgttttc
tatttatgccttgatgatttaatatggatttgttacagccaagtgccaaa
tgctctctcaaattgtcagcaatttaactagacacagataataatgggtt
tctttcagattttttgaaccatccacttacatatatttttaaaaaaatga
aatccttttcctgttcatacactaaccaaatctctcaaatctgttatccc
aatcattgttgcctctccgtttattataaactgtatgctcacaacttagt
gtaatataccagcttgtatgcaatggattttcaaccagataacatacctt
tcctgctctggtgcttagagactatcaactccctcctttagtgaaggagc
cgtgttagagcttccgagaatagctccactggagagaagtggaatcctat
atagaatgctgagtaaaaaaaaaaacaattactggctcactggctttgaa
aagtcacttactattgttgctgaaacttgctgagctgtttatagagaatg
atgataacagaacttttcctctgtatcactggtgtttaggtgaattaatt
aaacattgtgatcattagtaccaggtattattatctttaagagtcttcca
cttcaatgcacatggtgcagttttggtgtgtaacttagaaggattgaact
tctttgaatttactggacataacattttcagaatagttggtcatctagca
accgcctcaaaatgtgtaagcaggagagaaatttctcatcacagggattt
agacttactattacataaaggctaactatgagcttgctcattaattttga
aaagatgtacctggtggatatctagctagtaatatattctgaagcaacat
tttagctctattgatactctttctaatgctgatatgatcttgagtataag
aaatgcatatgtcactagaatggataaaataatgctgcaaacttaatgtt
cttatgcaaaatggaacgctaatgaaacacagcttacaatcgcaaatcaa
aactcacaagtgctcatctgttgtagatttagtgtaataagacttagatt
gtgctccttcggatatgattgtttctcaaatcttggcaatattccttagt
caaatcaggctactagaattctgtattggatatataagagcatgaaattt
ttaaaaatacacttgtgattataaaattaatcacaaatttcacttatacc
tgctatcagcagctagaaaacattttttttttaaatcaagtattttgtgt
ttggaatgttagaatgagatctgaatgtggtttcaatctaattttttccc
agactactattttcttttttaggtactattctgagcatactcaacaaaac
ccatgcatttcataaactaatagaagttgaggattgttgaatctatttca
cttattttggctgtggtttccatctgaaagtagaggttgtatacaccata
tactgttcttcattttattaatatttttctccttgacctctcataaattt
actttacacaattcttaccctgtacatatgtaaacataagtgtacgattc
ttaaccatggagtagaggtactagaatgcttacggccatctctttgtaca
ggaactgcattgactttcagtaaacataaagccacaactcctacatgatg
ttatgtaccatatgatctgttttgtatcttaaatttgatttacatatatt
atttatttctggtaactcactcagtttatgctgtgctaaatatcaatcaa
gccatgtataaatgtgatatgattggcaatatgtgtttactttaaacttg
tcttttcaaaatattactcagtttatgttgtacaatgtagatggcctctt
actaatgtaaaatgatttgtagtggaaacatttatatttttataataaac
ataatgaaaatattttttacagattggaaaaaaaaaaaaaaaaaaa.

Following is a second EPHA3 cDNA sequence

(SEQ ID NO: 3)

cttctccagcaatcagagcgctccccctcacatcagtggcatgcttcatg
gagatatgctcctctcactgccctctgcaccagcaacatggattgtcagc
tctccatcctcctccttctcagctgctctgttctcgacagcttcggggaa
ctgattccgcagccttccaatgaagtcaatctactggattcaaaaacaat
tcaaggggagctgggctggatctcttatccatcacatgggtgggaagaga
tcagtggtgtggatgaacattacacacccatcaggacttaccaggtgtgc
aatgtcatggaccacagtcaaaacaattggctgagaacaaactgggtccc
caggaactcagctcagaagatttatgtggagctcaagttcactctacgag
actgcaatagcattccattggttttaggaacttgcaaggagacattcaac
ctgtactacatggagtctgatgatgatcatggggtgaaatttcgagagca
tcagtttacaaagattgacaccattgcagctgatgaaagtttcactcaaa
tggatcttggggaccgtattctgaagctcaacactgagattagagaagta
ggtcctgtcaacaagaagggattttatttggcatttcaagatgttggtgc
ttgtgttgccttggtgtctgtgagagtatacttcaaaaagtgcccattta
cagtgaagaatctggctatgtttccagacacggtacccatggactcccag
tccctggtggaggttagagggtcttgtgtcaacaattctaaggaggaaga
tcctccaaggatgtactgcagtacagaaggcgaatggcttgtacccattg
gcaagtgttcctgcaatgctggctatgaagaaagaggttttatgtgccaa
gcttgtcgaccaggtttctacaaggcattggatggtaatatgaagtgtgc
taagtgcccgcctcacagttctactcaggaagatggttcaatgaactgca
ggtgtgagaataattacttccgggcagacaaagaccctccatccatggct
tgtacccgacctccatcttcaccaagaaatgttatctctaatataaacga
gacctcagttatcctggactggagttggcccctggacacaggaggccgga
aagatgttaccttcaacatcatatgtaaaaaatgtgggtggaatataaaa
cagtgtgagccatgcagcccaaatgtccgcttcctccctcgacagtttgg
actcaccaacaccacggtgacagtgacagaccttctggcacatactaact
acacctttgagattgatgccgttaatggggtgtcagagctgagctcccca
ccaagacagtttgctgcggtcagcatcacaactaatcaggctgctccatc
accttcctgacattaagaaagatcggaacctccagaaatagcatctcttt
gtcctggcaagaacctgaacatcctaatgggatcatattggactacgagg
tcaaatactatgaaaagcaggaacaagaaacaagttataccattctgagg
gcaagaggcacaaatgttaccatcagtagcctcaagcctgacactatata
cgtattccaaatccgagcccgaacagccgctggatatgggacgaacagcc
gcaagtttgagtttgaaactagtccagactgtatgtaattatttcaatgc
agtctagaggagggggcagggatcttgcaaaagatgtctgatcgtttatt
ctcactgtttctaagttttaaacaaatgtgatacatttaaggtatattgc
ttgggacattgcaatttgcagagccctgtgtctgtatacagtatttgtgt
ttgtgtgggtgtacattttgtgtttctttttttcttgtatgcaaatcaaa
catattctaatgcctgaaatgcttctgttttttttttttagccataaatt
gcttttgaggaacattatttaatatagtaacacacttccagtgtctgtca
tttcagatattccaggttcattgcgtgattcaatgaaccacaaaaaagaa
acttgctgatccatgagaatcttaattttgttttaatccttaacacattc
aatagcatatcacagagagaataaggattttctaaaatgtgttttatcac
ttcattcacattcagaagtaatttgaatagcctgttcctttaaccccaaa
tttggctaaaattggcctaaaactggcaaacatttttccagtaacttttc
tttttttcaaatgaattttcttcatacttaaaaaagccctttgctaaaat
ataattttcaaaaaggtaaaattatgtctatggcactaatataaaatgag
tagaagttaatgattttacttaactcatttttttctttctttcttttttt
ttttttttttttgagacggagtcttgctctgtcacccaggctggagtaca
gcagagcgatctcggctcactgcaagctcctgcaagctccgcctcctggc
ttcacgccattctccccctcagcctcccgagtagctgggactacag.

Following is a first EPHA3 amino acid sequence

(SEQ ID NO: 4)

MDCQLSILLLLSCSVLDSFGELIPQPSNEVNLLDSKTIQGELGWISYPS
HQWEEISGVDEHYTPIRTYQVCNVMDHSQNNWLRTNWVPRNSAQKIYVE
LKFTLRDCNSIPLVLGTCKETFNLYYMESDDDHGVKFREHQFTKIDTIA
ADESFTQMDLGDRILKLNTEIREVGPVNKKGFYLAFQDVGACVALVSVR
VYFKKCPFTVKNLAMFPDTVPMDSQSLVEVRGSCVNNSKEEDPPRMYCS
TEGEWLVPIGKCSCNAGYEERGFMCQACRPGFYKALDGNMKCAKCPPHS
STQESGSMNCRCENNYFRADKDPPSMACTRPPSSPRNVISNINETSVIL
DWSWPLDTGGRKDVTFNIICKKCGWNIKQCEPCSPNVRFLPRQFGLTNT
TVTVTDLLAHTNYTFEIDAVNGVSELSSPPRQFAAVSITTNQAAPSPVL
TIKKDRTSRNSISLSWQEPEHPMGIILDYEVKYYEKQEQETSYTILRAR
GTNVTISSLKPDTIYVFQIRARTAAGYGTNSRKFEFETSPDSFSISGES
SQVVMIAISAAVAIILLTVVIYVLIGRFCGYKSKHGADEKRLHFGNGHL
KLPGLRTYVDPHTYEDPTQAVHEFAKELDATNISIDKVVGAGEFGEVCS
GRLKLPSKKEISVAIKTLKVGYTEKQRRDFLGEASIMGQFDHPNIIRLE
GVVTKSKPVMIVTEYMENGSLDSFLRKHDAQFTVIQLVGMLRGIASGMK
YLSDMGYVHRDLAARNILINSNLVCKVSDFGLSRVLEDDPEAAYTTRGG
KIPIRWTSPEAIAYRKFTSASDVWSYGIVLWEVMSYGERPYWEMSNQDV
IKAVDEGYRLPPPMDCPAALYQLMLDCWQKDRNNRPKFEQIVSILDKLI
RNPGSLKIITSAAARPSNLLLDQSNVDITTFRTTGDWLNGVWTAHCKEI
FTGVEYSSCDTIAKISTDDMKKVGVTVVGPQKKIISSIKALETQSKNGP
VPV.

Following is a second EPHA3 amino acid sequence

(SEQ ID NO: 5)

MDCQLSILLLLSCSVLDSFGELIPQPSNEVNLLDSKTIQQELGWISYPS
HGWEEISGVDEHYTPIRTYQVCNVMDHSQNNWLRTNWVPRNSAQKIYVE
LKFTTLRDCNSIPLVLGTCKETFNLYYMESDDDHGVKFREHQFTIDTIA
ADESFTQMDLGDRILKLNTEIREVGPVNKKGFYLAFQDVGACVVALVSV
RVYFKKVPFTVKNLAMFPDTVPMDSQSLVEVRGSCVNNSKEEDPPRMYC
STEGEWLVPIGKCSCNAGYEERGFMCQACRPGFYKALDGNMKCAKCPPH
SSTQEDGSMNCRCENNYFRADKDPPSMACTRPPSSPRNVISNINETSVI
LDWSWPLDTGGRKDVTFNIICKKCGWNIKQCEPCSPNVRFLPRQFGLTN
TTVTVTDLLAHTNYTFEIDAVNGVSELSSPPRQFAAVSITTNQAAPSPV
LTIKKDRTSRNSISLSWQEPEHPNGIILDYEVKYYEKQEQETSYTILRA
RGTNVTISSLKPDTIYVFQIRARTAAGYGTNSRKFEFETSPDCMYYFNA
V.

Following is an Ephrin-A5 cDNA sequence

(SEQ ID NO: 6)

gcttctctccatcttgtgattcctttttcctcctgaaccctccagtgggg
gtgcgagtttgtctttatcaccccccatcccaccgccttcttttcttctc
gctctcctacccctccccagcttggtgggcgcctctttcctttctcgccc
cctttcatttttatttattcatatttatttggcgcccgctctctctctgt
ccctttgcctgcctccctccctccggatccccgctctctccccggagtgg
cgcgtcgggggctccgccgctggccaggcgtgatgttgcacgtggagatg
ttgacgctggtgtttctggtgctctggatgtgtgtgttcagccaggaccc
gggctccaaggccgtcgccgaccgctacgctgtctactggaacagcagca
accccagattccagaggggtgactaccatattgatgtctgtatcaatgac
tacctggatgttttctgccctcactatgaggactccgtcccagaagataa
gactgagcgctatgtcctctacatggtgaactttgatggctacagtgcct
gcgaccacacttccaaagggttcaagagatgggaatgtaaccggcctcac
tctccaaatggaccgctgaagttctctgaaaaattccagctcttcactcc
cttttctctaggatttgaattcaggccaggccgagaatatttctacatct
cctctgcaatcccagataatggaagaaggtcctgtctaaagctcaaagtc
tttgtgagaccaacaaatagctgtatgaaaactataggtgttcatgatcg
tgttttcgatgttaacgacaaagtagaaaattcattagaaccagcagatg
acaccgtacatgagtcagccgagccatcccgcggcgagaacgcggcacaa
acaccaaggatacccagccgccttttggcaatcctactgttcctcctggc
gatgcttttgacattatagcacagtctcctcccatcacttgtcacagaaa
acatcagggtcttggaacaccagagatccacctaactgctcatcctaaga
agggacttgttattgggttttggcagatgtcagatttttgttttctttct
ttcagcctgaattctaagcaacaacttcaggttgggggcctaaacttgtt
cctgcctccctcaccccaccccgccccacccccagccctggcccttggct
tctctcacccctcccaaattaaatggactccagatgaaaatgccaaattg
tcatagtgacaccagtggttcgtcagctcctgtgcattctcctctaagaa
ctcacctccgttagaccactgtgtcagcgggctatggacaaggaagaata
gtggcagatgcagccagcgctggctagggctgggagggttttgctctcct
atgcaatatttatgccttctcattcagaactgtaagatgatcgcgcaggg
catcatgtcaccatgtcaggtccggaggggaggtattaagaatagatacg
atattacaccatttcctataggagtatgtaaatgaacaggcttctaaaag
gttgagacactggttttttttttt.

Following is an Ephrin-A5 amino acid sequence

(SEQ ID NO: 7)

MLHVEMLTLVFLVLWMCVFSQDPGSKAVADRYAVYWNSSNPRFQRGDYHI
DVCINDYLDVFCPHYEDSVPEDKTERYVLYMVNFDGYSACDHTSKGFKRW
ECNRPHSPNGPLKFSEKFQLFTPFSLGFEFRPGREYFYISSAIPDNGRRS
CLKLKVFVRPTNSCMKTIGVHDRVFDVNDKVENSLEPADDTVHESAEPSR
GENAAQTPRIPSRLLAILLFLLAMLLTL.

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. Also, citation of the above publications or documents is not intended as an admission that and of the foregoing is pertinent prior art, nor does it constitute any admission as to the contents or date of these publications or documents. Each patent, patent application and other publication and document referenced is incorporated herein by reference in its entirety, including drawings, tables and cited documents.

1. A method for identifying a subject at risk of type II diabetes, which comprises detecting the presence or absence of one or more polymorphic variations associated with type II diabetes in a nucleic acid sample from a subject, wherein the one or more polymorphic variations are detected in a nucleotide sequence in SEQ ID NO: 1 or a substantially identical sequence thereof, or a fragment of the foregoing;
whereby the presence of the polymorphic variation is indicative of the subject being at risk of type II diabetes.

2. The method of claim 1, which further comprises obtaining the nucleic acid sample from the subject.

3. The method of claim 1, wherein the one or more polymorphic variations are detected at one or more positions selected from the group consisting of rs1512183, rs1512185, rs1028013, rs987748, rs2881488, rs1157607, rs1157608, rs1912965, rs1912966, rs1054750, rs1499780, rs2117138, rs2346840, rs2048518, rs2048519, rs2048521, rs3762718, rs2196083, rs972030, rs1036286, rs1036285, rs1512188, rs1512189, rs1567657, rs1567658, rs1028012, position 66765 of SEQ ID NO: 1, and position 66794 of SEQ ID NO: 1.

4. The method of claim 1, wherein the one or more polymorphic variations are detected at one or more positions selected from the group consisting of rs 1512185, rs987748, rs1512183, rs1157607, rs1912966, rs1499780, rs2048519, rs972030, rs1567657 and rs1028012.

5. The method of claim 1, wherein the polymorphic variation is detected at position 66794 of SEQ ID NO: 1.

6. The method of claim 1, wherein a polymorphic variation is detected at position between positions 18716 to 94523 of SEQ ID NO: 1.

7. The method of claim 1, wherein the one or more polymorphic variations are detected at one or more positions in linkage disequilibrium with one or more positions in claim 3.

8. The method of claim 1, wherein detecting the presence or absence of the one or more polymorphic variations comprises:
hybridizing an oligonucleotide to the nucleic acid sample, wherein the oligonucleotide is complementary to a nucleotide sequence in the nucleic acid and hybridizes to a region adjacent to the polymorphic variation;
extending the oligonucleotide in the presence of one or more nucleotides, yielding extension products; and
detecting the presence or absence of a polymorphic variation in the extension products.

9. The method of claim 1, wherein the subject is a human.

10. A method for identifying a polymorphic variation associated with type II diabetes proximal to an incident polymorphic variation associated with type II diabetes, which comprises:
identifying a polymorphic variation proximal to the incident polymorphic variation associated with type II diabetes, wherein the polymorphic variation is detected in a nucleotide sequence in SEQ ID NO: 1 or a substantially identical sequence thereof, or a fragment of the foregoing;
determining the presence or absence of an association of the proximal polymorphic variant with type II diabetes.

11. The method of claim 10, wherein the incident polymorphic variation is at one or more positions in claim 3.

12. The method of claim 10, wherein the proximal polymorphic variation is within a region between about 5 kb 5′ of the incident polymorphic variation and about 5 kb 3′ of the incident polymorphic variation.

13. The method of claim 10, which further comprises determining whether the proximal polymorphic variation is in linkage disequilibrium with the incident polymorphic variation.

14. The method of claim 10, which further comprises identifying a second polymorphic variation proximal to the identified proximal polymorphic variation associated with type II diabetes and determining if the second proximal polymorphic variation is associated with type II diabetes.

15. The method of claim 14, wherein the second proximal polymorphic variant is within a region between about 5 kb 5′ of the incident polymorphic variation and about 5 kb 3′ of the proximal polymorphic variation associated with type II diabetes.

16. An isolated nucleic acid which comprises a cytosine at a position corresponding to position of 66794 in SEQ ID NO: 1.

17. An oligonucleotide comprising a nucleotide sequence complementary to a portion of the nucleic acid of claim 16, wherein the 3′ end of the oligonucleotide is adjacent to position 66794 in SEQ ID NO: 1.

18. A microarray comprising an isolated nucleic acid of claim 16 linked to a solid support.

19. An isolated polypeptide which comprises an amino acid sequence identical to or substantially identical to the amino acid sequence of SEQ ID NO: 4, or a fragment thereof, wherein the polypeptide or fragment thereof comprises a histidine corresponding to position 914 in SEQ ID NO: 4, an arginine corresponding to position 924 in SEQ ID NO: 4, or a histidine corresponding to position 914 in SEQ ID NO: 4 and an arginine corresponding to position 924 in SEQ ID NO: 4.

20-42. (canceled)

43. A method for treating type II diabetes, which comprises administering to a subject in need thereof a molecule that specifically interacts with an EPHA3 polypeptide, whereby the molecule is administered in an amount effective to treat the type II diabetes.

44. The method of claim 43, wherein the molecule is an antibody that specifically binds to EPHA3.

45. The method of claim 43, wherein the molecule is an antibody that inhibits an interaction between EPHA3 and an EPHA3 binding partner, ligand or signal partner.

46. The method of claim 45, wherein the antibody inhibits binding between EPHA3 and Ephrin-A5.

47. The method of claim 43, wherein the molecule modulates one or more levels or activities of cellular molecules selected from the group consisting of glucose uptake by cells; glucose transport molecule activity or levels in cells; triacylglycerol content in cells; resistin levels or activities in cells; levels or activities of PPARγ, PI3 kinase, Akt and C/EBPα in cells; levels of activities of Ephrin-A2 and Ephrin-A5; levels or activities of ADAM10; circulating levels of glucose; cell or tissue sensitivity to insulin; progression from impaired glucose tolerance to insulin resistance; glucose uptake in skeletal muscle cells; glucose uptake in adipose cells; glucose uptake in neuronal cells; glucose uptake in red blood cells; glucose uptake in the brain; and postprandial increase in plasma glucose following a meal.

48. A composition comprising a cell from a subject having type II diabetes or at risk of type II diabetes and an antibody that specifically binds to a protein, polypeptide or peptide encoded by a nucleotide sequence identical to or 90% or more identical to a nucleotide sequence in SEQ ID NO: 1-3.

49. The composition of claim 48, wherein the antibody specifically binds to an epitope comprising an arginine at position 924 in an EPHA3 polypeptide (SEQ ID NO: 4).

50. The composition of claim 48, wherein the antibody inhibits the interaction between an EPHA3 polypeptide and a natural binding partner or ligand.

51. The composition of claim 50, wherein the natural binding partner or ligand is Ephrin-A5.

52. A composition comprising a cell from a subject having type II diabetes or at risk of type II diabetes and a RNA, DNA, PNA or ribozyme molecule comprising a nucleotide sequence identical to or 90% or more identical to a portion of a nucleotide sequence in SEQ ID NO: 1-3, or a complementary sequence of the foregoing.

53. The composition of claim 52, wherein the RNA molecule is a short inhibitory RNA molecule.

54. The method of claim 53, wherein the RNA molecule includes a strand comprising a nucleotide sequence selected from the group consisting of GCGGATGGTAACTTCT (SEQ ID NO: 135), GCTCAAGTTCACTCTACGA (SEQ ID NO: 136), CTCTACGAGACTGCAATAG (SEQ ID NO: 137) and AATTTCGAGCATCAGTT (SEQ ID NO: 138).

55. A method of genotyping a nucleic acid which comprises determining the nucleotide corresponding to position 66794 of SEQ ID NO: 1 in the nucleic acid.

56-59. (canceled)


SEQUENCE LISTING (143 sequences)

SEQ ID NO: 1 - DNA, 94750 bp, Homo sapiens
tgtacttaatcagcatttaccgcaagaacaacatatgcaaataagagcataaaagaggctgttccaggaattgtgtgtattttaaaatatctagagattaggatgcttatggagtttgtaggagatgtggctggggatttaaacatgaaacagattctaaatggcttttacacgctctgctatgaaatttgcatcttatcctgaaagttataaagagccaaaatkttcctatgcaaaacttttatgcataggaatgacatggtcaaattcatggtttagaaggaatattctgaaagataaaatgtgtggaaagcatacaaatagagaccaaattaggaatttctaattataattcagaagataaatgaagacttaaactaggatatttaagagtgtttggtgattaactggaggcagaatgattaggagatggagaggagaaggttattttatatttatggtctagattcttgctcctcaaaatgttgtggtctggaatcagcagaatcagcactgcctgtgtgttcataagaaatatacaatctcattccccactccggaatgactgaatctgaaactgcttttccagagattcttgagtggtttttgtgaacattaaagtttgagaagcattggtctgggtcaagagtcagtaaacttttttctgtaataggccagataataaatattttaggctttgtgcacatcattctctattgctgctactcaacttgcccattctagcataaaatcagtcatagacaatatacaaattaatgaatgattgtggctatattccaataaaattgtatttatagacagcaaaatttgaatttcctagatttagaattttcacatgtcatgaaatctcattcttttgactttttacaactgttaaaagaaatgtgaaaccattctaagctcgtgagctgtacaaaaacaggggacgggcaagatttggctcctgggcttttttgccaaccatttgtctaggtgaataaatgggccatttataaaggtaggaaatagagggacagtttgttggtggtatgtgttgaggtccatgtattatatcacttttcaaatgaatatggatattggagttaaaacaagcttaattagaaagctttttataattgtatgctgagaaatcttcctctcttctctaagatcctcccataacttctggttcattcaacgagttttttgccattgaggacagagaaacccagtacagcatattttggacaggagacatttaaataatgtttgattgatgtcattgaaacaaagtatgtacccatattaattaggtagaaatttcaagcaaaattatgggaaatgtctatttttttaaacattaagtaaagaatactttcaacattgtatcagacattaagccatacttcaggtaaaagtagaacacatattaattaattaatgaatcaatcaactgttccatgtagatgttagtcatgattataatacctgttcttattttttctcttcaacctcacagctttctccatctctggtgaaagtagccaagtggtcatgatcgccatttcagcggcagtagcaattattctcctcactgttgtcatctatgttttgattgggaggtgagttcacagtctgtttcactattcactttcwttgttgctttgtttgctgccactgattgctgttaaaatgtgaagagtgtgctcaataaaatattttaacaaataagaatgtctccacttgtagtttaggtcctctctttctcctccttttctttgtttctccatccttagttttgattttctgctctacattaaattcctgtccccactcttttaacatacacccctcctcttctctcttctgacactactcctagtttttactttctttctctaattatctcctcactcttcaaataaccccagatttccttgacactttctcacccagaacaggggtaacactgcacagcactaatcattacccatagagactttacccttcattccttctaacagaagttgttaaaaaatactataaacacattctaaagccattataaaaactgaactggagggtctgcacagtgggaagcaactctgtgtatctcctcattaggcagctttccagggttcccagagagaggggctttcttcagaggattttgaaaatatgaagttattaaactttcactaaagcatttaattgtttgaggaaaatgttttatttttatgctttgcacattctgagcataggtaactattttaaatatattcctcttatgtgttcgctttccttgatttacctccaggttctgtggctataagtcaaaacatggggcagatgaaaaaagacttcattttggcaatgggcattgtaagtttctaaacttggctttttgttttgcttcaccgttttagctttagcagttattgatttacaattggttaacttcctcctgacaaagagacttcaaaagtagttttatggaaaaactgagtacattaaatttattttagaaaaagagggaaattcatggttctgcattaaataatttttacatgtgtacatttcagcatacttaagctaaaataaagggtaatctgtggtttacattcaaataacgcacatgctaggtagtagaaactagtctgctctgcatggctgaaatgcaaagcgtatcttcaatttccttattactagtcctttgctaaactctaaataggagacttttctctttcctaactgccaagtaaatataaacaacctctgtttatttgaagtgccttatcccattttatcaaccaacctagaaagagcctttaaaataaactgtttattttgcagttacatgatatccaactagtagtaaagccagaactttctctccacattttaatttacccttttaattttttttaaatacacatttcagaaacaaagtgtttacaactaacaaaaagactttttttctttttctggtgaaacgaatcattccaggtgttttgcgttccaaactatggctcagcttagtgtggcaattactaatcagtagcttaaatagcatgttctcatggaaattagtacaaagactgtactcatgcttatgtgctccaaaataatctcaatataactaatgtagtcttgtaagcattaatttttttagtattcactattgccttctaaaattatttgcaaattgacatgatactttgtatcagccgttggattatcatctaaaaatgatagctttagttgtaactcaagtaaaaaacttgactacctgttgctaacctgcaggaagcatacagtagcttgaaaataatgcctttcaaaatataaattgatatgagaagaaagcagtataggagtttgaagaagcattattttgttcatccaattgcagcgtctataaaataattcacacaaaaaagccaaagtactacattcctaaaatcaacatcacatcagtccagattaattaggtgaccaactcaaaagtctaaaaagaatgcctgaacaagttacaggatccttgtctttctttttctcaatgggcccacaaaacactagtgttacaatttttatgcataaattagagcagtggcattcttgtggattaagtcaccctcaatttgacttggacaattgtaaatatcatctttagagcaagaccatgctgatttgattttttttttatgggaagcctacatcttacgaattttgagctttggtaaatttctgtgtgccaattattaaacaggttcctctatcgtcccagtctgttttgctattgtttttatggtggatttttgttgaaactttaatttttttcaaatgcacaatatccttatgacaaacaaaagagaaatgcatttcatcctggctgcaaaggcataaagtaacttatatgcatgtcataaactttctgcatcttaatactacagctggtttatttcagaccttttatttggtattactttaaaaagctctagctacgaactgttccctacagccttgttacaaatcctataatattgcttcaaaaggaaagaaatgtgtttcacctcctaagatcctgtccccttgccctttctggctgggcctttttacttctttgctatccttagggcaactcatgctggtaaaatggaaatgtaaattattgtgtttgcacagaatgagctgtaaagtagtgcagcttaacacgtgtgcatgtgtttattacctggttttcatgacagagcccagttactctgtatgttgcccttaattattggtgtgaagggattatgtgcctcgcagcctgaatcacctgattcctggaatctctggggtcagcagttaaagatcagcagccagtgcttctacagcgaacagaccattttacttgggggtgtatatgtttatgtttgtttttttcccccaaatgtcttatggatggaaaagaaattcaccttattttaattaatagaaattaataggattcattttatttgggaatggttatttaattttcaaattatttagtaatagtagtattgcatataggtatccatcatacatatattctctacaagctgactggtacttaagggtaaatctaaatcacacaatttcaagtctgtttaaggccaatgcttatttaaaatatgtgagacattgttcctaacaactcattttataatctttgtaaattctgatggcaggaaatctcccccacgttaatttccttaacttcagcaaagtaaaggagttataattatgtacaaaacagttccattttaccttatgcttaaagtatcattttatttccttgaaaaagattgctagatttctttaccttactgttactaagtaaaggcaaataaaaactggaagatcacacattatagggttgatctgatatgagttttcatccttaaaatgtaactaaattgactaaatgaaaagactccaacaagattttatttcctaacatattttagtaatggttacttacttttacaatttttcttactccacatctagaaaagtatgatagcttttccttaatcttaaagaagtttcttcttttaaaggtaaagggttttagtactggtaatgttagaagttattttaatgtaacaataaagcacaacagattactgtattaaagggtgactctcccatctgtatttataggaaggattcatgcttatatggaagaagcatataagctgatctgtgagctcaaaagtaatggacatttctaagtctaaattctaccattaggaaacatcaattatattaataattgtaaataaatgttcttaacaataaaaacgctcattagggcaatgagctctatcaatacattgagtgagaactatgcaacagtttctcatgctaggcatttgaatagtatagctttttttttttctcaragccctagcttgtcttttttgccaagatattcaagatgaaaaactctaaaagtgaagttctcacttcaacagctactccactcagagtttgtccaaagcaagggacagaacttgtatttccccatttctgtggatattgacttctagatagtggtgtcctttcaaattatccacacctcgcctcaagctggatagcagctgctcttttgttgtagctgtmtcagcaccaggcagaagtaggagtcgtgttagcttttcttctgcattccctatttgccctctcaccaccatccctgctttctattatgctgtgtcaaagtcatgcaaataatagctgaggaaaatagagtttttacccaacctctctctcagccaccatttcttttacaacagtttattctcacacttacgtttttttactcaaaatctctatgaaagcttcacaagaacataaatcgtaatgcattataaccatgggtttcatgaagttgtcttgtagttcaaagaattgatcatttgttaacacatactgtgaaatcctagacttgttggcctaatgttcattgtactgaggcttcccagaaaaactgcgggcatctgcagtctccacaactatttaatacaaactacaaaactaactcaaagaaacgattggttcacgcagggtgttcaattgcaactttcccataattctcccattatgaagtaatattggaaacagtaatatctgatttcatttgttataatttaatgtaatgaatgaaaatatatttcactattaagtatccatattctgtgctaaaacacactattaagtatccatattctgtgctatgaatgttatcaaaattaaaagcaaaatcagtagtcttagcagatataccataggagttacattgcaatttcctttmattttagatcatatttatatagtttagtagatcttagaatcacttttttaaaactcagggtttttgtttagagaaagttaatttattgaaaattgcaaattgtctccactgttatttttccaaattataggaatcttattaatattaaatcaaattaaaatgattatcctgtatcagttgtctgtaatattttaactacaggatatgaacatttttaccttcagaataatttaatgaataaatataatatgaaggagatttttatttaataaattacatatatgacttacgtttggaacttagctccttagcgccattattcaactcatagacaatatttattttacttctaattatgttataaagctatacaatatgattatatttaactgatactatattggtgtgtatacatgtatgtattatatatacacaccaatataatatataatacatacatgtatacacatactaacagcttaaagtagatttgtaattacagtgggacattttgattgtgaatgttaatataagcagtacagtgtcagaaggtatgccattatatatcatttaatttatctataattgcagccattatctcaccttataattgtattgaattattttccaatcttgcattgtaatacatatctggttattataacatttgtgtaatcataaatatgcaatagcataatggtgttcactcactgttgctcctggtagtaatgactaggtctaatcagttctgaattatctttcttcttaagatccccttttttgagtatgcattttccttctgatgtttttgcaaagaaagctcctcattgtggttcctttatataattcatcatagagaacatgttttcttaaatttttcaacacagtccaatgtcactgccagtttctgtgatactacagaataaactgaaggttgtgcaccttatggggtagggatttttttaaaccaataatgccataaaatttgatctataattgtttgtacaaatctagctacaattgcgcctttctttctttcctcaaacagtaaaacttccaggtctcaggacttatgttgacccacatacatatgaagaccctacccaagctgttcatgagtttgccaaggaattggatgccaccaacatatccattgataaagttgttggagcaggtaaccacaatgaccctactgccaacttagtactgtatgtgaatcacgattgctcagtctctgaaacctaagtatattgctaaagaaatgggaattttctgatttcatgatcaaaggcaagtaataaataagtgcaatatttaatggaatgtaatgagttcaaaggtgccagacttaccggctcacagaccatggctgcttttgatttattaaatttctgatgtgacagcagcaacaactcaatcttcccatagctgtgctgatcgtgtgtcacaaaacctatttggctaaataaggataactggataaaagtctatttcaatttagacaacccaggtaatttgaagttttcattttcaattttagaatcatgtttggaattaaaagaacaaacaacacaaagtaatatcatgactcatcttacataaattttatcacaactatcttgggtggatattttaaataatataaggtgctccattttaatgcttgcttgactttgaatagtcttatgtgttaaagctttttttaataaatatacctttgttctaattattaatttaaacaatgataaatttgttaacaatcacctattaagtggttaataacagttgcatatttataagaaaaatttcatatatttgtaaaataacagtattttatatatttacacataaacacatgatatgtgcagtatgcatggaagtaaaactactattcaaaagtaaaaatgaaccatgctcaacaataaaaaattttgtaccatcatctaatccttcaatgtcactccccaaagcaaacataaactagttatgttgatacaggaattctctgtgtaacatcagatcacattttctgcaagtaccgtatttacatatttatgagtgatgtgtctttacaaaaatgtacatgtaatgctttgtttctttttctatacaccaaagtagtactgaaaaatcaagcagtagttagtatgcgaacaggacaatcttgtaaatataatgttaatttactagagcttctcttctaattatcttaatttcttatcctaaaatttacatgtgcaggtaatattaaaatatgaaacttgtgttcatagaaagaaaatggagtagtaacttcttttttacaatctaagacttgagagtaaacatattcgctgtacatctgtgctgatccagtgccaaaacagatatatattgtttactctgtttcatttcttcagtcctgacttctgaacagctccatgatgcagaagggtttagtctggctggttttaatgtttcctcacaagagtgcacttttctccagcacggagcctgcggtagcaaatgcagcacgaggactttgaacactcaactggagaatatgttgagaggaggaaaaggttacttatgctaagccactgtccaaaagccaggcagacattattatgatgttgaaatttaggtacctctccactgataaaatatctttatatagttattattgacagactcagatatacagaacatagtgatttgggaaaaagcacaccactgaaaatagcagctgataaatgagataaggggaggtctatttatactgaatcacttaatccctgatgctcattaaagattagaatagagcctccttcctatttctgtcttcaagtatatatgttaatgattagatatatgcccatgttcagggttttgcagccaatattcacatttgccatgttaagaacaacaaaactgtacttatttgcaaataggtataccttcaaggttagttatacttttcctgaaataattcaatacaatatacacacccaattctagactgccttgatgacctttcagagggtttgtatttaccaagttagaaaatgaaattattattcctaaaagctaacaatttgttaaacattttgttttgtaaaatgtaaaatatgtacacaaataaattaaatagtgtaataataaataaacctgtatgttactcatttctaaaatagcttttataacaatgaacaaatggcaaccttggttggtatatacatttacctacttcctacattcttaatcttaaaaaaaatatgttatttataaaaatttaataattattctaaattgattaccaactggcttccattgttagaaaggcaaacctttcagccaactacataactttcagagtcaattaaatttacagaaagaatatatatatatatatatatatataagctaattctcttaaatttctgtcagaaattttacataaatgaattgatgttatggaaactctcttgtatacattggactatagaaagaattatacttatactataaatgataaaattaagacatatgaattagcttcccacagtagtagattactagaagtgaggaattcatgactacaattcctaattttactcctcaatccaaccattctttcaacgattgaatactaaaaatttctgaagaaagtaaatacttagtaagtccttaatttatcctgtgcattcccatgatatttaattttattatcctttttagataggtattattatctccatttcagcacgtgaataaacaaaagctgagaatttgaatagacttgtttggatcaaatgaaaaaccaaaatttgaatcttatactttccaaatttaaagcccatgctggacactggagatcatagtagtgtcttcttcatagtatttttttttgaggattaaacaagataagttgtgcaggggacttagctgacacagaagcattgttcaatatgtgtttttattatgcttactttatgaaaacagtagaaggagggtaggctttaaagctagtgttggagggagtcattctatttaaatgtcggctttttttaattgtttaaatgaaattagcataagaaattttattttgagcagcttttacattgtgcattttaatcagaatacagtccccattcttataaatatacctatttcaaagagcaaataaaattgccgcacttattagaattaaaaaatcaagatctggcagccatgcttttatctagaaatgacaagatctgaatttttgtttcatatgggtgcaaagttaattgaataagacaatataaaacttggccagatgaaactattgcaaattgcatttgcccagtctttgttaacttttacaatacagttcttttattttttatttttaatctttttctatttttagcagctttattgagatacaatttaccaaccataaagttcagctatttaagtatacaattcagtgatttttagtatattttcagagttgtaataaccataattgctatcccaatgcagttattttaaattattaaaaatataattgaattaatattaagcatttctatgtcttttataatatattggcggctatttctagtgtttatctgatgaaataatcacattactaaatgtggggaattttaaaaactagcattgctgaaacacaattttcatgaatttcgaaggtaaatatatacatgtacatgtttatgtgtccaggtgtcccacttacaacaaagaacactcttaattggctcatttaaaatttgaactcctacttttcatatctctagtatgctatcctttacaaatatatgtccagtaagacatgagccttgttatcgccatcattaccataaccatctcttacattaacatagaatttggcattttataaagcactttcatattcatttgtcagtttgatttaaacagaaactcttaaagggtagttaggcaatttatcctttaggtgggtctgaactcagagagataaatagtgaccaacacttagatgtcacctactgtgtgtctggcactgtttcaagtacttaacatacattagttcctttaatcccacaacagctttatgaggtagatacaataagtgtcccccatagagataggaaaagataggtacagagaggttaagttacttaaaatcacacagctaatatgtggcagagtcagaattggacccgaagcaattcggctgcaggatccatgctcttaaccagagttctgtactatcactcctgtaaactgacaagtgacatgtcacttagaatatgtgatgatgacatctacatttaaaacatgctctattataccatgttgatgtatgataatggcagaactttctggttttcccaagcttagtgtttacacaaagtgaaacaaagtaaatgggagcgctctgataatgttcggataaccctatatgaggaaagaaactcttactcttgtttacaattctgagaataattttatcacaccataacttctctttctcctaatattctcatatctaaatggcatattcttccttcgaattagcaagatcaatgaactttctcaagttctactccctgattctttctgtatcctaaggaacactatagctgtagatatcattatcttctctacacttcctcaaagggaataaactagtcctgtcttgtttaccattatatctatctctgtgtgttttatactgtatgcaatacagttgctattatgaatatttattaaataaatgcatgaatatctgcttttggattttaagtgactttgccaaattaaagcataagtcattgctttgaaaacagggcaagagtggatttctggggatgagtcaagcctggggaaggttaaagcttcccagggagagagacaccatttgtagtaatgtagctggcaaatcaataggacaacctgctacttttgtggttcaaaccacttgcttgaaaacaaaaatagagaagaatcatatcacttcaatctgactgacacgtagggcatgaagctcatagaaaattcacagagagaaaattggaccaagattatgattccactccatcaagagccaaaaattaatcatataattaattctaaaatatttcaagaaaagctcatttgacattaaaaactatttttaatgtgatgcaaaacatatctgataatccacactatgttagaagttcttctgtgaacaaaggacattctatagaactcccatgtggcaatcatcatggcagcatgggtacagataaataaatgtaaagggtatctgctctaagtgagctccaaatgtaatgaattgatgattagtaaatctctcagtaaaaatgtattttgaaggtagtgtccatactagttctaggcagttttatatcaactggactataatatttaaaagtcagtaacactctatatgggacaaattctggattaattgcacattgaagacttttttttttaaattgaagaagatgaatctaaaatatttctacagtatttagataaggcatgttgtttttatttgatactgttatattgttgttgtcctccagaagcccagagacttttatagctggtatgtcagtaaagcaacacaccaatactccaataaaataaattgatagcagatgctttccttctcatttttagagtctgaatatatagccatattggccctcggtaaaaacagaattgttaaaatatctgaaatttaatccaatgctcacatttaatcactgttggtgctgtcctgttaatttctctgaggtaatggtagctatgacaacaaggggtaaaaaatctaacatatgcagctttcaacttatctctccttttacattacattatttatagtgtcgtactttaaagtatatggaattttcacaaggttcattttccaacagggggtgactatcctgaggttttctaggcagtttcatatgattatctttcaaaataatttggagtactagagtgtacatcttgcaggtcaaaggcacaaatattttaaatggaaattttgaaataaaacagttgctctctactgatgaatttttattatagaattccttacattttgttgctgttctgctttataacaggtgaatttggagaggtgtgcagtggtcgcttaaaacttccttcaaaaaaagagatttcagtggccattaagaccctgaaagttggctacacagaaaagcagaggagagacttcctgggagaagcaagcattatgggacagtttgaccaccccaatatcattcgactggaaggagttgttaccaaaagtaagtaaagtagtcataagacctgtgtttccgtatgttgagcaaaggttgtttaaacccaaccccaaccatttaagaattttggctttttttcataagtatctcagttttaccaaaaagaaaagtttaggaatagcatgcctttctttgacagtagagcagacctttaaataatattctattgacagtactgttttagacctcaaaaaattttaaaaaaatactagaagccacatattagaccatttaaaggtacgctgattagcagacagttaagagaagaattgtggggaattctcggctatgtaagtagcaaagaaaaacaatgactcaagcttgacaggaactcccagctagctgccttttcttttattgttcatcagttttatttaaaagttacaaatctttaacctcctcccaccccctgcatccctgcctttaggaaaattttgggagtctaggaaatgcttctgagctgtacagattggaatcacaatagacccaaggccgtttcttggttctctgtggattttaaatagttctgatctagaaacccaggtggattcttactgctctgtggaagctctggctcacagcccatgatagagatgtgaacttatttccatgatggtttctccagcaggtctcaaaagggatcctgtgaggcggatctgtgatttttggctccaatccctatataagtctcttgagaatactctgggacatttttcctcaagattttggagctcaggatgaagaataagtgaaattggcagcaaactttttagagtgaaagacaacatatggcagggaatttggcatcctttaggggataattcattggcaccacatttacatactctatcgttaccaacagacatatttttcataattatatttctctttaccattttatagagcattttccaaatggcactaatacttattctcactttagcatgactaaaaaggcatttccttcctcatgagagcatggtttaattgaaatagactttgatttccttttgagttttcatatataaaaagtggaattaagaatttaacaatgtggaatagttcaacatacattgtaagtccatattaattcttataaggctttttatttgtttgcctatgtactctacaaaaccaggccttattaatagaaatattttctgataaaaaacatttttaaattttaagttaataattaataaaaatgggcataattagtaaggattgggaaagtgtatgaatcctttgttctttctgagcaaggctatactgttttatgtctgtacagttacagtatcatgtctcattgggattttagtgtcatttagccatttaatgttctccattacatctgcatcaagtggtggttgccttttagctcttgagcctcctgggaagagaaaactcccttgtacttatgaagccattcttctaactctcaatttttacaagtttttcattatgaagtatctgttatgtgcccacattatactatgacattatttcattcttaccactaccctgtgaattagacatgataatttctctcttacatttgaacaaacaataactgtaaagggtaattagcttgtataactagaaaatgctagaggtagcactgggaacttgttctgttcatttcaccccatccccaactctgaacccaaacaggtttctacttgtcttttcctgtattctacttattcttttatatcgttactttaaaaatcttctctaacatagtgagacattcctcaatcttcaaaaatttctggaactattaataatccttaatatctaaaacaaaatataaactctcaaagagggcaaaaacccatgttatttgatgacaagtgactaaaataaatcctggatcataatagatacttaatttaccaatgttgaaaaattaaaaaaaatattgatgaaggaaggaaggaagaaagaaaagaaggaaggaagggaggacacaacaatatcttttaacaataggtctttctttagtatttgattcattaaattgaatacacttttcaaaggagtgcatttaaataactacttctatcacccagagagtgatgaaaaaaaatgattactttttagcttgaatagaatcataatctaacttcttcaacatgtgtaattaagaaaaaaatgagctaagagtcactatcacacaaatattaggttctaatttaagcttactaataaactcactctattattttgtcagtcatgagttgatttttttgtttcagtgttatgaattatgaaagctactccttatattttcctgtttcctataaatgcatttactggatgataagcaacattaaactataaatgttgtggcaaatttcctttagatataaaactgaaacaattaaatatagattatggtagactgacaaatctatgaaacaatctctaagacaaaaaatgtagaaaaatgcactaaaatgacttttaaaaattgcaggtattatatattaaacatcttactcaaaccgactctgtaacattctaatatggattacaactgccttattcttagggtgagagttctaaatttgatatatctccctggtcaaataagcatgtaagaagctgccaaatctgactatataaatcccagtcatctcatatattgtaaatataatagagtagtagccctagaattagattttgaactccttggtggcatatttataagctcttagtctggcacccggtaacatctgagaggaaatagaaactccttacaataaagttgatatgttgtatttaacacacacacacacacacacacacacacacacacacacacagagcgattgtgtttccaaaagaagctatttattctactgctattattgtctttgtttcctgagatttctaaaacaatccacctgcagagatcagaaagcacatctatacatgcacacatgcacacacacacacacaactagagtgatgcagagcttataatttaatgaacaaaataatattttcaaaagcaggcatatcagtatctagattaataatcaatgatctgagttcaagttctgaaacaaccttactcctctgagaactcctacaaatcagacaattcacacataattcatttcttcgcttgtaacaaaattaaacagtactactttactatttttagatactaaagatcaaatgagattacataagaatatattaagtggcaaagaactgtgcaaatctaaattactcctctggttgtttgagattttaccaatcaatttgggtgtatagaaaaaaattcttgaaaataattctttttttgctcaaagataaatctgtttattattgttattcgattgtgtgctttgaaacactaggtcaaatacaatgtatcattttcaaatcatttgaagaaaacttttcttaaagctttttattcagtcattgtattttcatttagccacaaacaattttaatgtttttgagcctgatggtcttaaacaggagaaggtgagagagtctctgatcttcaaagtagcagaaagcggggggcacaggctctgctcacaacaaggtactaacatgtgttatcaattttagctgaagttcaaaaggatagaacttgttcttagcaaatgtttgatatttctgccatcacaccattagtcaaataatgttcagactccaagaacaccttccattggccacttttcacctagcgaaatagaaggaattatattggatgagattaatgtattttgtcttgcattattttcaggaaattttaaaaaattaccatgtaacttaagtgtaactttgcattattgaggaatgtcatgggcagaacgttaagatgattttcataattaaaacttatctgtagtggcatgagtatccactttcactaaaagcttagccattggcttccagttgcctgtttccctgcatgtacggtaaggacacatttctggaataagaggtcatcatttagtaacaatgccaaattctagttattttctgtttgtttctctctaaaaatattctaaatttgtcttaagtgtactacacttcatgtcatcttaaaagacaatacatacgttgtacttactatactagtaatttacagagatagtttgttaggataacaaattttggaaactctaatgacccaagttttatcatggattcatggctgcaaattgttaaaatgagttcagaacatgtctgtctaagtaaccacatcaacaaaattaaaaaatcactgctcgatatattagaaaaatataagacagggaatgttatttttacatatttaaacagcttttgttacttcttcatcactgtccacttatgtcccttggatgaataggccattttgacttatagaaaagtgtggtattgtgtatttttctcatttatatgtataggatggttttctgatatttttgaaaaattaagatcaacttacaaaatttgttagagcacgatttgtgtgtgcccaaagacaccatttctagagcactggtttacatatacagtaaatctcctagagaaagaagaaatatgatgaagcactagtttagtagctgtctgaataactgtggtcttacacaacctttgcagaaagggaaaaaaagtatcacctctaaatgccgaggctagtaatatcttgataccaaactatacagctattatcagaaagtataataacattattgtaatgcataaacaaaaatttaataacactagaataattgtgaagccatatctgaaagtgtatcaaaaaagatactacaccatttcccaagtgtagtttattccaagtatgaaaagttggttttgtatttaacaattctaaaaactcataacattaataggtaataagaaagttctatcttaataaatgcaaaaaataaactattttaattttaattttttattagcacatcatacatagaaaatatgtattcctgataaaggtatgtaaacagcccccaaacaaacacaaacaaacccacaaatgtcacatttaatagtaaaactttaaaagcatttcttttcgagaraaaacaaaaagcctgttgttattattatttcaattcaatattgtacaggaaatcttaaacctttttttttgacttttacaaataaatcagctcctttatttgcattattttgagcactctgataatgacagtcttcactgatatttttgaaatcctgttttttgttaaataagcttttccaattaggcaagcaaataaataaaagaaagcttggaaaggagaaaaaaacaaaactttttatgaaatcaaacaacatattaagagtattatatacattcattccacttactgtatgccttacattattgtgcttactgcatgactcagctctaactagcataatggacagtaaattagttgttatctttgaagatgttaaatttcgtttcaagttaagcataaataaatataaatatttttcaagcagctgaaagtaaagcattctatgtgttaggattgatagagtagggttggttaaggaagtcttggagaaagtgtttctcatcctgattttgaagaaagaaattatctctagccagtctgaaattatagacctaatttgatcaattgtaactggcacagtggtaggtgttggatttagaactacagacaaatgggcttggtctttgctctcattaactcatagacgtaataggggtcatttccacacaataatgtcttgattaaaatggaatttggaagacgttgccctgtaattttataatgatttaataagaaacccacaaaaaaaaataccctaactcagctcaatatttcatttcaatatttcagttcaatattcattacaatatttttatcagttaggtatgatatgtgaaaatctatgatacattttttattctcatcaaagatgattcaaatcttctttcctggaaataggcaaattttgtcaatttttttcaactttccatctcccgttttttttttttttccttccatcctgacactgattatatagattgctagcggctaaggtcaaaggttttgattttataatataccctcattagagtgtgtttaatccttttgaaaataagacatagagtgttagacccaatgactgttgttaatagggagttaatagcatttccatttgaaattattaaatgtaaatatttaataatgtttcaatatctgataaacagtttgtttgaatgaaaatattttaatctgaaaaacaaagtttgttagaaaatttcaaagaaaatatctttatgactgactatatatgtatatatatatgcgtatgtatgtgtatatatttcacattattctactctataagaaatgtgattgagaaggttgtgttaaaatagctggtgctgaatctggaattcttagacattttgaatcatatttcttctgtcatcaagcggcgattcaatgactagtaatagctccgagccactggggatagttgggaaaaggaactgattataacagactgatttgcttccctttactttacaattgtgaaagatcatcattttatctaaactcataaatcaacttgagaacaaaagcaaaactccactgcaaattttgttttgctcatttttgatggtttcatttcagcaagtcatagaacagtactttcattgagtttagtatgtatacccagatgtgttttggggtttctgagagccgcaaatgatacaagctttgtctggaggaagacgatcatgtcatttgtaaatcaaaatacacctggaagagcatgcaaacttgagtacaaatacatggcataatgcgcagatgagctggcattttataaataatttcccaccacatttaatgcatgttggaactagttctcgccactacagaagtctctttttctttaaccctatttaccataatcaaaatcacaagggaaaccctaagtcaagatactgtttcccaaggtagaatatttgtaattctgtttactctttgtatatttgatttgcagaatctttgaacgtaatgcaaagcacattttcagacttgcccatcttaactgatgtgtttacaaatacctgaattctagagttaaatgcacgctgatgtgatttctgaatggtcatggggctttgacttaattgactggtttgatttttcagctacttttatttcccttcccaaacatcccactagacatgctaatgagaaatggtcataaggctgggagccagaaggaaaggagtgataagtaaaggtctgagtagtataaaaatgcagtaactggccaatgatattgagagttggtggtgtgaggcaaaatatctgtgactgcatttgaagtgtgcattgaattttggtaacatatcattcctcccagcactgtatgaaggaggaaagacagaaactgatggtgatttatagaaagtacacatatgcaggattttattcattcattcatttattcattcatttcatttcaggccaggcatctagaatcaaacagtctctagggaactgatactaattcaggagagatctaagtttccaaggaaatggtgaatcttccctttctttttgaatattgtttaatcaaagtttagttggaattttgataaagcaaattctgggggctcaaggttttgactgaagcaatttttcagatattactacattaactagatagatgttacatggtgccttaaagacataaatcagtgtcttaacggaaaatagatcaatgtatcaagtggttgtaacatgtataaaagttatctcaaaaactatagaatttaaatagatttttccttaataaataagacataattcctggaatatattcaaattactgaatgtaaaagtataaccttcatgatgctaactcataatttacattttgtacaaatcaaagagttttttacacatgaaaggaaaaaatcaagaaaaggaagttattttctagcatcagaatggcaaactaacctcatgataaataagacacataggaggaaaagttgtgttatacagttatgtgtacattaatgtttgtttaacatctgttatttgcttatgagttttcagcagtactcggttaatactcaactcacgtcttctggacttggaaaaggagtgattttccttcctgtaaaatgctggacttataactaaaatagtataagaataataaagtaagggaattacagtgtggtaatatgtataggttttgaaattagagacagtcgtgtttaaaattttcttgactgctatctaattgtattacctcaacaagttgcttaaacccacaatgttttctgggtaggaatcagtaaaatgggaataatgcatattgctaaagcataagtaagctgtgaggattaagtgatataacaaatgtcaagtacctaatgcaatgttacataattctaatcatgaaaaaaaaattaccagtatttcttttatatcaaagcatcctttttttggtatttttctttttaaaatgttagttttacataatcttcttaattgttaccataatgtatatagaattatattttcagctttgtataatattacgctgtaacaattgtctaaagtgcttaagtcttaaggtcatcattttaatttaaaatcatccatagctttaacatgcaataactgactaaatcattatctgtagtggaacatttgttatattcatagctctgtgtatccactatacttagcacctttgtgtctgatgctttttcatatgttacatttttccctcattatcagtttccgaaagtgacagcatagtgtgtacaggttgcaacttttagctttataacttgttagatgtatgatgaccagcaagttaattagcctctgtgtctcagttttctcatgtgtaaaatggaaataataatacttaatatgttaatatataaattaattacaccagcatcatgctcacaataagtactatttaaatacattaaaatgctgggttaaaggtttcatggtattttaggctacatatttgcaaactggttattacaagttactgccaatagcaattttaatttcagttttattttccatttaaacatagcattcatttcataatttttaagtgttaaacaaattttgtaaataattataactattattctgaattgttttgtagaactcgtgtagaatccataaaggcagaaattttgatctacttgctccccactaagcggcacaaaagagaacacctagcacagtgccctacacgtatatatgtgtgtgtgcacatatagcacatataaatacccatagaaagaataaataaatgttaaattcctaacattatcttgggcttggaggcagaaatatttggtgttgaagataagtgagaatttaattaaagagaggaaaggggcatattttttgtttacttgaaaggcctgtcaaaccaatttagatgctattggtcatgggcctttgttctggaaaccaatcttctccaatttttgtggtttttaaagttttattttctaatgaaacctagggctttttaaaatttttaaaaattagaagacaggcaaagttcttacatctctataatcctcaggtaaatccaactatattatatatgttcattgtataatacttgaactgtactgattattatttattatttactgtatatctaggtaagccagttatgattgtcacagaatacatggagaatggttccttggatagtttcctacgtgtaagtaagatgcacacacatacatatatatgaataaattgctgaaaacattagagacaccctccaatattgtgccaagcaattcagtaatctaagtttaaagtaaaactgaaatcttctgaggctaaatagacagagaagggctgtaaaattgcatctttttttgcagcattcaaatgttaatggtttatatttttaaaacaaatttgtgagttcttcttcaaaagactctttttatactgccaagattcacactcgttaaataaaaataaaaaagaatcctaaagcaagtaataaaacccactgatgtaagacagaaagtctcttttttaagtaatctcagtctgatataattatttaatcacagtctgatataagacagcccatctactttccaaacagtgctgtcagaaaataagcatgtgctataatgctaaagtatatatagttttaatttagatatatcattttgctgttagatatgtacattaattttttagattgagaagctttatacgtttatctatcatctatctatctatctatctatctatctatctatctatctatctatattttttgagatggagttttactcttgttgcccaggcaatggtgcaatggtgcaatggtgcaatggtgcaatggtgcaatggtgcaatcttggctcactgcaatccccgcctgctgggttcaagtgattttcctgcctcagccttccaaatagcagggattacaggcgcccaccatcatgcctggctagtttttgtatttttagtagaggtggtgtttcaccatgttggccaggctggtttagaacccctgacctcagatgatccgccctcctcagcctcccaaattgctaggattacaggcatgagccaccgcacctggccatattttgacatactatatttcaccataagaaagtgaatatattaaatttacatctatttcaacaaatgttatgttcaacaatttcatgaagttcaagacaataccaattaccagtcattgcaggcgataagaaaagacataggccctgtcctcaaggtgcttttgttgctatataaattgagatgggcagatgacagaaagatagatagataaatagataaagaaaatatgtagtatcattcataatcatggaggaaaattttaaattttctaagactgagaaaggatatgctttatattaaaatccattagccaaataaaaattttaatagcaattattttcataatcgttatttcaaaaataaacatttccaaaatcaggtgagttaaaatattatgataaatattctttcaaaattatcgtttgtaattaaacatggaattttatataaatacattatctatacattttaaaccttatgtttctattttaatccaagattaaattagattggaagctggattatgtgatttagtttcttattgctttctattttgtgactacaattcagaagtgggtagaaaaacaaatatccaataaatatttttgactaattatgaactaattatttatgataatgtttaaaaatgtagcatccaccttatttgaaacaattcataaaatatcaggaaacaaagaaggggaaatgaaaattcaatcaaacaaataaatatcaacctaaactagccacaagattcatttcacttttttgttcttaaccttaatgagtctgagcaggagttagtttttttgtcacataaggaccaggaaagtccttgcttttaaactgaagtcaaagaacgttaagatacttcctgttacaaagtatttgataacatagccagtgtgttattttgtttcagccttgtatccatttgccacatatctttgtcttgagtgcttaggactaaagtgtgtatagtcagtcttgttacaaaattcaatatgtgagattttaaccaataaagatatatctttaagaaataggaacgtatcttaattgtacatttgaaatgcttcccagaaacacgatgcccagtttactgtcattcagctagtggggatgcttcgagggatagcatctggcatgaagtacctgtcagacatgggctatgttcaccgagacctcgctgctcggaacatcttgatcaacagtaacttggtgtgtaaggtttctgatttcggactttcgcgtgtcctggaggatgacccagaagctgcttatacaacaagagtgagtaacttagattttctccttttttatcattgttttccatcttgtatcatgttgatttgtaaataagtagaaatcatgacccaaaacgtgttgtcaattatgctttccacaatagaaaacatatcttaaaattaaaatattattatttattctgggtaaatagatggtcactgtttaacatttaatgattttaactctgaaattctatacctcagtttaatgttccccaccaaaagcagcaagattctcacattcctcaaatcttgatatttttaaatgtacccatcttttcaccatggttgagtcaccctgagctagcagattggataaattctaaggaccttatcccagtacatatcattttaaagcactttcaaatcaattttttaataagtaatacctatttatactgtaaaaatgtcaaaacagacctataaagaaaagttgtccataattgtgccatccacagacaacatttataagattgtgagtcaattcttacaaatttttcttcatactcatatgtacaaacttgttcatgaatatattatttaaatacaattattcataatacaattgtatgccatttctctccttaccaatacacttcttgaatatatttcattgtcaataaataatctttaaatttttacatgtagtaggttgctgtattctatattgttgtatcttttttaaagcaaaccctaattagtgagcaaattattattatatttaatttttccatattttataccaatgtatgtgatctgcctgcattcataattgttatgagcctcatgattttctaatgatacattcctaaaaatgaatgttttgtgtcaaagattacgtgaatattttaaaagcacactttactacactcttgataaaactaggtattttcattaattttactattatcagttaggataaattgaatgtatatgttttaatttgcatccatttaattcaaaatgcatttgaattttttgtgagcttttctgttgttgttttgttttgtgtttgttttttgttttttaggcagggtctcactctgtcacccaggctggagtgcagtggcacaattacgactcattgtagcagcctcaaactcctggcctcaagtgattctcccgggctcaagtgatcctccctcctcagcctgctgagtagctgggattacaggcatggaccaccagccccagctaatttttaagtttttctttaaagaaaattatttccatggtagaaatttagaaaatatagataaaaaacaagtaaatgtctaataatcattactattaatactacacaaacataattattagcattttaaatttagcctttcgatttttttaaataaattttcacaaataaataactcctcttttggcataaagttacaaaaatgggattatactttatatactgatatataaaatgatttcaacataccgataatttaggcactttacttatatataaatcaatgccattttgttttctgccctgttttaacttaaaaatataaaatacataagtttccagatgattaaatatttttacaatattcaataatgtctaatactctattcatatacataatttaattaaataaagcactattaattgggcagaattacaatattttcatattatgagtgatactttgatgtcatgatatctattttgtgtctgtccatgattacctccttgggatatttctatagaattaacattacaggatcaaaacgtatacaagtatgtgagaatttttggcgtatatattgcccaattgtctttgctgtagtttgttatcaattttctctcctaatggcagtatctttctccatattctcatcagctaagagaataggtggttccaaagatggtagtcttctgctgtttcttattatctaattttaaaagtttattcccgattaaaaaaaaaagctcatcctatttgcaaatcactcgagaactccaataatcaaaattgtcacatggcatgcatatcatgctataataaagaacctaatatgccagatagcatttcatccacaagtaaagccgcaataatagaaccactgaataataatgacacaatgctgttgggatatttctagatgcattgatataggaggtaggactctcccaatggcttttaataataaacccttttgtttttccaattttcaaatatactctgtagtgcatttactttgttcatgaatctaaatttttttttaatctgagtattttttaaaaagtccttagacacttaaaatgtgtcctacataaaaatgttgttcttaacaaacacaattcaaatggataagagcatgacaaattaagacaaatttcttatcttgtagctagttgaattaaatttacaagtgactacaattatatagctctgaacatgatgaaattgccaaacatgggatcaatacctcaacagtgctttttagttctccttctctgatcaccagaaaaattagtcagccaatgtatctgcaacaaagcatcattttaataactgagctaccaattcagtagacactatatgcagcaagtgatacttaacagtcttagggaatgtatttcattctactcacttctactctttttctaaggctatccctcttaacctgtcatgacataagtacttctaagagtccatgaaaagtatcatgcttacttatattgtccgtgttacaacaatcatattatgccagaaaaatacttttaggttaaataaaaactatttgataaaaaaattatttgaaatgttaaatttctggttattcagtcaggttctaatttctatgccatattaaattatttatttatttatttatttattaatttttgagacagagcctcacgttgtctcccatgctggagtgcagtggcgcagtgtcggttcactgcaacctctgcctcccagattcaagcaactctcatgcctcagcctcacaagtagtttggataacagtggcttgccaccacgcctggctaatttttgtatttttagtacagacggggttactccatgttggccagtctagtctcaaactgctgatctcaggtgatccacctgcctcagcctctgaaagtgctgggattacaggcgtgagccactgttaccagccccatattaaattattatgtgagagcatctctgtccccataagtaggcttgattgccaggatactcaaaagaatagtttctctggcaggaattggaaacatttgtccaactttagatgattcatactctggagaacacagggggttaactctaaatgtttcctttacactgattgttgttttgtcagggcctctttagtgttagaagaaaagtgtcgtatggaaattgctctttaacatttctgaaataaaagtgaaacaggctctttgcatttctgttccagtcactgaagattcttaaaaacacactgaacaaataggctcattcaccaccaataaatagctgtttcgagttcttcagggttcatctttgtatttccttggaactccctcaaaatatccatgggagtcctggctttcttccgagaagctgtaatttgcatgtgcgtttctagagtaatttaaacatcattgatttaatctctcataataggccttattgagtacaataagtttatagtttactctaaatatgaggtatggttatgaagtaataagaagacttcaattttttaaaaaaacaaatataccaaacagctaaaygaggcagcattctaagttttccttttggaaataaatatatacttactccaggaatgctgatattgcacaaaatgttttgggaatttctcttttggaattatatttaaagctaaattataactgatgcattaatcaaacatttttaagcaggtagtattttgggatataggcttgactaagataaaatctggtctcgataagcagagtggggtggctcacgcctataatcttgacacttcgagaggccaaggcaggaggacctcttaagctcaggagtttgagaccagcctgggcaacatagcaagacttcatctctgttaaaaaatatatatatatataaatatatatttatatatataaataatatataatatatatacacttttatatataaatgctatatctatataaatactatatctatataaagtatatatatactatatagtatatataattttatatataaatacatctatatatatataaataaattatatatgttatatataaatactatatatatgtgtgtatatgtattaaaataatataggacaggtgcggtggctcatgcctgtaatcccagcactttgagaggctaaagcaggcagatcacctgaggtcagaagctcaagaccagcctggccaacatggtgaaaccccgtgtctactaaaaatacaaaaattagctgggtgtggtggtgtgcgcctgtaatcccagctacttgggaggctgaggcataagaactgcttgaacctgggaggcagaggttgcagcgaacggagattgtgccattgcactctagcctgggggacagagtgagaatccatctcaataataataataataattaattaataaaaataaaaacatatattaggcatggagacgctcacctgtggtcccaactactcaggaggctgaggtggaaagatcacttgagtccaggaggttgaagctgcagtgagctaaagcctggggatggagcaggaccctttttcaaaaaataaataaataataataaatttaaaaggaagaagaaaagaaaaagaaaaaatactattacatttttgtgtatatattgtctgtttgtcaacccaaagcaatatacccagcttagacatctgtcttgtttatttgatttagtgtgctctgattattgttatttcagaaaaacaagttcatcattcaaaacaatttgctgaggcattccttttggataagagatcctggttaatctgcattaaaaagtcagtcacattaataaagtttaacgcagttcatctagtgtctgaagtttctacaatttggagattaacatttggtgcctcaatgcaatgaccctttcctggttgctcctctgaaagttactgcctgtaggtagagcgtagttgcactgaagagtcatgaaggacatttgaatcaatgtcagtggaaaagaatactgaacatagaatgtctgtcgctcttgttttaaaacatctctgtgagtgacaggcagaatagaggaatgtatagaaattatataatctaattatgtatttaaaacttcttaaactttgaagagtatttgaggagttgaggaaacacctaagctcaaaacttaatttatcagacagtcaaagatattttctcacactgtgttctatactgtcttaggtgtatcacaagctttccttccttatgttctcgacagcatgccggatatgaaagggtcagctaagatagtactatacatttttatgtttattttctcttttacataaggatattgtgtaaggaatcagatttttttccccaatactcacttgttgttatttcttctcatttcttactgctgtgttacgcagaaattgcaaatgttgatattttccattatacaatgttattatgcatcctgtaaaactccactgtactctcatgggatgttcagaatgaataaggctatgaagtttcagtattattatgaaaatagtttctatcttgtggacccctaaaatgctcttggggacccacatacatgctcggatgacagattgaaagccactgttatagatactttactaggaaaaaagtcccttaataactaaatgaatatttttgccgttattcactgatataaatctaaattatggcatagttttatgtgatataatatattttaaagaagtagctttgaaagcagaactgcagctgcataccaaagtttgcaaatcatcttgaaagtccctggatttcgtttgggatggatatgattagaccatttagctccacaaatttaaatcatcaaagaagttttgtgtctgaaaaaaaaaacagaaaaattaattcctttcctctggtttctcatcatgtgactttgaaagtataattataacatgtttgttatatttttgccctggtctactaatttacttcattctaacagaatcatgacactattgtagaacagtttgccactgaatgatttttcaatttttgcttctatgaaattttatctttaactggttagtatttttgtatatatgcattcatgcatctatatagatgaatatttctatattcatatgttgaaatacatggaatccaaaattccaaaatggtagtgtttaattttctgttttatacaggatcaagttactgaaagcacagacttttatcttatttagatgctgggtaagccatcacttcaattcttcctatgtccttaagttcattttgggagcataattaccacataaactgagttggaaagtttgagaaaacaaattgaattgtccttggctatattctccattattcgatttttttcttttcatttctattctgaagttgcctacttagtaagtactaactagattttgttacaacattttattttacataataatttatttggctttttagatctgtgactctgccatattgatgccagatatattctatacaactttgttttgtttgatttcatttttgtttttttgttttgttttgttttgttttttttgagatggagtctcactctgtcgcccaggctggagtgcagtggtgggatctcggctcactgcaagctctgcctcccgggttcacgccattctcctgcctcagcctccggagtagctgggaccacaggggcccatcactacacccggctaatttttttttgtatttttagtagagacggggtttcaccatgttagccaggatggtctcgatctcctgacctcgtgatccgccagcctcggcctcccaaagtgctgggattacaggtgtgagccaccgtgcctggcctgtttgatttcattttaaggataaggcaaaaaaagaaaagtggctaagcaagtggttaaaagctaagaggttaaatatttcctttcaggtaactccaatctaagaacataaataacagaaaacaggagcctttgttctgtaacattttcaagaaccaaaaaaaccatgcttatcaaaattggtataatacaaaagacacatattaatttgataagtaatatatgtcgcatgtcatttatacctaaatgaaaaaggtaataacaaaatatctaatttgtgtccttcatatagtaacagtattttaaactgtgaaccaaaaaaagtcaactacgaatttattgcctccccttatgatcaatatgaatatgcctgcatgtagcatcagaaaatacagccacttattcaataactagaaaactctcaaaggttcagtgattttaataattaatgttgaatcgacacttaatgaaaagtctagtactttctacccatctatttcagagagatgagtagcatgaacatttaaacatgtaaaaaacaactatccaggtttatgtggtgaaaaattacaactataattgtgaagtggaacacaggagcacacaagtaaaggcagaaaagtaaaatgaggaatgcaggcagtctgtagagaatgcaattttatataggatcgtcagagaaattggtaaaatgacactagagcagaaaatgggtttgaatgaaggagcaaatactgaggctaaagaaacattaattttgctttcactaattttcaattgaatagaataattttaataattgcactgattccaagaggcccaccttgcaggaagacaatgtaagtcattagattaattatttagctcagactaaatgtcaagctaatagtaactagtcctctttttctattggtgatgcatgcctttagtcccagctactcaggaggctgacgtgggagggtcgcttgagcccaggaggtcagggctgcagtgagccttgattatgccattgcactttaggctggggcaacagagtgagattttatctcaaaacaattattagaatgaatttagtattatttctatcatttcaattgcaaattgtcagtatcccctgctacgtttctactaatttaccatcgcttcttcaaaaaattgtgttcagagtgctacaatttcagactttgaaatgaatcactataacttttaaaaattagagattttaaaaactggagattgaatatatataaaaaatacaaaatttgacatttaaaacctcattgaacttttaaaaaagccagactcatattaaggcacacacacacacacacacacacacacacacatatatatatgtatgtatgtatatagacttactccccgatgtttgcattatttttgtagaagatagatatcatacatcagattacttagtttttacactgtttcatgagagtccgaaagtaacataagcattatgagtttgttttttatttcctggttagccattataataatttggaatatggagattttcctgtatttcaagtaatgcttaatcatttcacatgatgttgaaagatctcaaaagttcgaagaatgaggttttgctacctaaaggatcatttatctctgatctcttgtggcaatcaacatttgctgaaactattctctcagtggcgctgagatttgaactgctggggataagactagatgcttcctttgggagccttcttggatcacccttcaccttgttgtctgcagtgaatgaattttagtccttgacaatacatagatgctaaatcttttagccaaataatttatctctatagttccacttttaacctgagaagctaatatttccctccaggttaaatactcaattacttgggtttactcagataaactctccaaagtcctttgtcactctaaatatgacctgacaaagctcaaaaacaggaaatgtgaattataaccagcaaacctttcctaaattggcaggcagagatttgtacatataattacagaaacatttggcagctatgtgatattatgcctcaatacccagaaatttcagcaattacactccttctctttagaaatcagaacttgaagccccgtaacccataccgcatgagctgacttcagttaagctcctgaaaaattcaggcttttagccacatgtctcattgaactgtctaaattcatcgagcttagggaactgaattgcagaatctttcaagaacagggttaccgtgtgattcctcatctaactgctgtatctaggcaaagtggacaaggttttagactctcaactaatcctggagtggagtttagcagttatgtagtgcctttttccagagaatcactagcatctttacctgtagcactttattcatctcctttaccttttctgtaaaactctatagacgtaaatcgaggtgacacccatgtggatatgcaatttattccaaagttagtctttctttaagagtccaaaataattaacattgaaaatgtcagcatccttgatactcatgtaggtttaagatagaaagttcaaagagctatggtcataagtcagcaaagctgttaagctgttagataaaactgaatgtaaagtcaaattttatatagtccatgaccctgtttttgaatgacaagacagctgtgcagagaggttaagacacctggctaaaattaaacaggaaggtcaaaactgaggccagaaaccaagactcctgattcttattaaacttcagaatccaaatggagcaaatgactaaacttaccctgtacctccagttatttagctatacatagatgagaagtcagcacttggaggaactggctgaaggtttggcactggagatgtaaggagcaaacattagcttctgattctgttaagtttgaattggaagtctttctgaaattgaggttttagatacactccatatgctatgattctgggcttgctccaagcaactggaagcttttctgaaatacataaatatatcttgggagcttacattaggatatagagaccagtaaaagtaaatgcttcattttaaatgtttaaatatggttaaaaaacacatcttcataaataattgttatagtgactagctgagcttttataatattctaaatgcctagtgtggcacatccctctaaagcatctacattttaaaaatgtccttcacatatataagctaggaaagcaatttctaaaccagaaaaaaataacactcatgcaatatgagcttgaatttgttaaggtgggaggtgagaagggaagaggaatttttgtggtcacaatcaagaacagaaatatgcatacgctctgtgatgaattacttgtaggaaacttagactcctatatattcaatttattttagtttgatagtctcagaactacagatcctcagcttaaatgcagcaagtcttattgtaaagtgtgactcagttaaaatttattgccccagcttcttcttttaaccatatttcagaaccaaattgggccctcaaggactcttattcgtgtctctctatgccaataatgtttacgtttttgtagctgtagacagtgtttgctgttggagcaagcacttgtatcttactaaaatgcttggttacttatacctccatttaataaaaaaattgatgcttaatttataaagttaatttaagttttactagcaaacttaaattaacaggaagttatcctaaaaaagaaaaagaaaaggaaggaagataggaaaaaagaagaaaggagaaaataaaataaaacataaaaaaattccagaagggatattattacatcatcaaaacccaagtattaggagattggctctggtgaacttgacccctgtttttctattagtaaaggtatagcctgtgacaggggtataaaatatccctagaaagttgatttcacttagtcaatataaaatcccacataatttgtagacctcatgtggattactttcccttgatgattgtacaacatttaaagcagtagttcgcaaatggggatgattttgttccccagaagatacttggtattgtctagaggcatttttggctgtcacaactggggagatgctaatgtaatctagatgacagcggcagaagtgctgctagatatcctacaacgcacaggacagccgtcacagcaaagaatgatctcgggtgaaaggtcaacagtgcccttctagagaaaccttgcctacaaggttcccatcatgtgcaaagaatgggagtggggatcaaatgtttctctcaggtactttctattttttcctgtccatattggactgcaagatgcatgggacaatatagactacaccatttgtgaaaaaatgtgcaacagactagccatctgttatcaggcctcctagaatgtgaaacttcaagctatcacaaacataatgcaacaaatcataagtcttgtgtgcctgttaaaatttcattttcatttggtttcaattaaacacttaaattacttaccaccctccaccccaggctctaaatttcaaacatagccactttagttgtgatgttctattttcattttattaagacatcatggagtaaaatcatacatagatgtatatctctagaagataaatatatcagatattttagtcagtatataagtcttacccttgtgtaatttttttaatgttttaacattttacttagcctcattctacaaaggggtaacgtccatgtgtgtagactaaaattattttttaattaattgtatttttaggttggtgaagtagctacaacattaatattgaagcagttgatgtagaatgtgtattgtttatggagtcaaatgaaaatctttatagcattctacgaactctaaaacacataatccatacaacatcctgtgacatatttttgtaattctacagatactctaaaaaagttgatgctcagtgaatcttatactaactatcgttgtggagttcttaaaatgtattaatgtaacaacactattaaatagtagcataagaaaaaagaggacattagaaagcagtatgatttaagaaaataaactattatgtttatattggctcattcacattcacatatattatgaaaaactagatttaactcattatcaactgaaaatgaactgcacctttaaaatgcacaaaattatcagaaatctttagaaaaatctactttcccaggtccacaaaataaatcattattctcacttgacagaacaacgtgcttttttgcaagcaacaaagttttgtaaacagcagaggtagaaaataagacatactttaaactgggggaatacagtggtgaagggggccaggtggttattcctctggacatcccagattaggcaggataataaagcagtagaaagatcacagaggatggaatgaccaactcttgctggaagactcctaaatgttcacccaaagagtttacatttgaccaagatgttcagataagaactttacccagttacagaaaaaaggaaaaaaagcctcccaaaaagaaagaccatgtatttgggaagatagcatgctcacagggggaattcaattaagatcagtgagcatctgttgaatgcctgcactgcacatttgccaagataataccaagataatactacagagagcaagaaaactgctctctgtagtgaatgtgtattgtttatggagaacatagtttcatgtgagttaatactccttatcttcagctttataatctattcagtttttcataagcttatgatttgaaataatggttcgggtttactatttttatttgttgcaattgatatgacaggcactactgctactacttttactagtgtagaacataaagagtttgaaactacaatgaagaatttaaagagtttatgagaatgtatgtatctatcttttcttaaattccataataattttgctcgtttgaaatttcacctgtagtttgatttttttttttacttcaaattaaatgtgtacaatgttggtttattattcttttatttttaaagccagagcaatttgatattatttgcttgccatagaataagataatttttaaaaatttctttttatctgatttagtattaattgctaaatttgaattgtactttgtagttaacagtgagctttcccttgtagcatcttttaataaatttgttatattgacagtaaatgtaacaattaaagggaattgaaaggaaggaacaggcataacatttattgaatgctctattaaggagcagacactttttaaatggctttatatatattataaattcatgacaaatgacaaaattttctaaataataatatataattctcaatcatctttattctcttcctgtttccctcaatgatggatggcatcatttacaatatggttagatagaaaagagataagagtcttgaatctgtgattatttcattattggagctaaactaatcmgtctaataagctacctattagcagataatggaaattctctaatgaaaaggctaaatgtttcatgaatattgaatacattctgggctaaacagctaactcttggccaggtgcgatggctcacacctgtgtaacaccagcactttggtaggtggaggtgggaaaatgacttgaggccaagagttagagactagcctgaccaacatggcaaaaccttgtctctatcaaaaatccaaatattagccaggcttggtggcgcacacctctaatcccagctactaaggaggctgaggcacaagaattgcttgagcctgagagggggaggttgcagtgagccgagaacgcactgcaccaccgcattccagacctaactgtctatggacttcaacactaaaattgggcaaagcaaatcacttattcacatcttcttctaaagaaagagtttaaattagccccagctcttcttttagactgagttgtttggatatcatatataaaaatataaattaagagtttttcattaaaaataaaaataaatatgtttacttctaagtatataagctcaattaactgtgacctgggacagaatctggccagcgaggaaaagaactggtacaccaaaagttgatcaatagtagtatctggataaattacggaaacatttaaagcactgcaccctggtttcattgttgaactgattgaatgccacttaatttttgagtgtttgtaataacatgtccaaaataaaatgaaaataaatgagattgccccacttccttatgctttcccacaagggcagcaactgaacccttgatatagttaagagacaaaaaggaaaagtgtaaatcaaaaacctgaatgacacaagtgaaaagaccatacctacatttccaaagctagcaaaacttgtagcaagaggcctttgccatttcacaggtatctactgcacaatgtggccaagatacttttccaaggtaccacagtgagctaagagccagaatattttgttaaaaattagtacgaaggtaaagatagtataaaggtcacctgatcagcaagtatgttacattgaaaggtttaaagttgtcattctaacaataatatgtgtgtgtgtgtgtgcaggctacaacaattacaaaacacaaccagaagagcagaattgaaaatttaagtttttcttattaaaataaattactatacatttttccaaacatgcatcccatttgtttaaattttaagcattttaataagatttacacaggttacacaaatagaccacatgtaattgggaaatattctggaaataatttgcttgaatctgtaaccatgtatttcattagagaacatactgtataagtcccaggcaagttaagagattcaggtgagtgaagctttccttgaagatatgatcatgcgaccacagcagatacctctccaacattattatgtctgtatttacaacagagcaaaagaggattctccttccaagcttaagcagaaatatggctctgttcttcctaaaaatgatcacctttcagtatatcattattttaagaaattctattaatctcaatgctcttctctctggaaaatggtgaagaaacacttaaaatgaaacaactcttctgtgtttctttttaaccctgattgaattaaccaaataaatacacttctctttcatggctgtcatcaacctattgtttttttcttatgactctattttaatttcaactagagaccatggtaaggcaacaaatatctgggcctaatattttctaaataggtacaaagttttcagcatagaaagggcttttttttttcttttttcttttttttagtgccatttaagtacacttcacaatttcagtattactgaaagtatactgacatgatcctttcagtataagtatacttatataccttcagtaatactgaaagtatacttatacgctttcagtaatttggttctggaaaaagagaagtagcaaaggcagttagaaaaatgacaagtgtctataacagacctaagatatttaattgagatttttaagatactgtacctcccttgtcctatgaatacagctctgtttgacttttgttaattctctgagttcttaagaaggagaaagtaggcccaatgattccatttggcatcctgagtagattctgttcaatgccacatgtctaagtccacttttgtcgatgtcaaggggtagtatttgaaattttgaattataaaaacataaacttgatctaggcatatgtagatgagtgctttgggagttttagtgcacgttgcacactgtagtttatctccctaacttccgaggctatcttttctctggctccggtctctccaatttcatttgttttacttcatcagtagaaactgtcaactattcttcacctggtagcttcttaggaaggttgcgctcactgtgtgtctttgcaggcttattcttcctgctgatggtgtttttccatttcctcaattctctgcactgtttcttgcattcttcatgactgtgtaagctctaccttttcaaagagcttctggcagtactctccctgaacagctcatttgtgccaaaacaccaaggagaagtgctcagttttactgagttagttccaatcttatcactctcctagccaggataaagtttcccaagtgcatctgtgattaaataaaaatttgacattttagcttcctcttccattatatttcttcacacaggaaactgttcttctagaacatacagttttctcctctgtgctttgatttatgccatttgctcagaatatgagagtattgagctaacatttattgagtccttactatgtcctaggaagaagactaaatattttactgaaatttgtcgtttagtcccacataaatactctgaaccaggactattacattctcaataacagatgaggacagtcctgcacaacaaggcttagaaacttgtcccctaaattgctaatccatttgtgtaaagcactatattgcccccttgagcatcacttcaaatccagtggcatgctatgaggtccgatagaaatcggtcacattttgacgaacacggcttaagattttgaggatgtgagttctttctttgaaatatccatcccttttttcttatatcaaatattgtacttatctttcaagtgacttcacttgaaattgaggtcacttaattcataagtgccctcctcagtctctctagtgagaatcttggctccacgttccacattacggtggctttcactactgtctttctactcagggtaagttccatggaccagcatcacatggagattgttggaaatgcagacactggctacacactcaacctcctgaatcaaaattttcattttactgggagtaagctgtatatgatttatatgcattataaagtttgaaaagcaatgtgctagatacctatttcgttctgtatctagtttttcagagtaactcattattactcctgtttgcctgacacttacgaaacaaaagagccagtatgttatttatcagtttcacccaaattaactagcataatacttcctatatggttgacaatcagtacatctttattaaatgtatttattgaactaagagcaaattccatagacattacacttttatatctcccataacttataaaagcaatattattttcctctaaattataagttctgggaatgtaattattatgtctatctaatttatatggtgcttgcctactatccagaagacacttcaatgactaatgatgaagcacacagtaaattaatcaagatgattattgtgttgaagagagttttttaaaatataattttattttgagtttatttaggtaacaaacacctgtaagtacttatgtgccagggagtggtctaagttctttacaaatactgacttattcacattctgaacttcgattgcttggtcaagcataaatctaggttaaatcaataaaagttgatttgtgatacctcttttaattctaatacatgtcatgtcacatctgcttcaggttgttttgttgcagatgattaattaacctgccatagttgatataaaatgtcaatatttaagcaattttcttttttatatataatttcaaaatccttccaaattatttggaagtaaaatttgctgtaaatggtatgctacttctgcttggtattgaaatagaataacatcaaaatgtttcacagaaatgcatattccatttcagaacagaaatactatgagaaattctgtccggtttcagattatgttatgtatattatatgttctatgcattgctgatttatgtagacatgattttatattcaaagggagggaagatcccaatcaggtggacatcaccagaagctatagcctaccgcaarttcacgtcagccagcgatgtatggagttatgggattgttctctgggaggtgatgtcttatggagagagaccatactgggagatgtccaatcaggatgtaagtatttgtggtctatgagttatgagttcagatgaaaagatcaagctgtgcaaggaagtggaacataatgtaactgggtggctcctggtttrtaacactgaaataaaatatttagcagcaatgaaactgtttccaagtcttgcaaggatattatattaattgaattgttcttacagatttacatatttccctcaagcaagggtttagaaataagacaaaagaaacatttgtagtgaaaacgtaatgaaaaaagtgaccaataagataacctctagattctaatgcacattgaagtttgaaagtcactgctttacaccattgaaaagtactaatgaactaatagttacttaattttacagcacttaggattttacaaaaaaaattactataatttggaaggcaccatttgaactgcagtataactaaaacctgtatagtaattttgtccatattcattcatatatgttttccatagagaactatataaccaaataataatttctttataacaccaaatttgctctatcatttaacatttctttttattttaacccaaaaaggatctttacatactctttcctaaatttatgttatttcaatcttcctttatataatattttaggcatacataacgtcttatttatttgatcagttgttatccttttaggcaactaaaaatgagaagttttcccacttacctttaaaataagcatatttgtgagccccgcccatgaaaacgtaaactaagtgacacttcctgaaaacttcctggttcctgaaaactttgcttctcacacaggtaattaaagctgtagatgagggctatcgactgccaccccccatggactgcccagctgccttgtatcagctgatgctggactgctggcagaaagacaggaacaacagacccaagtttgagcagattgttagtattctggacaagcttatccggaatcccggcagcctgaagatcatcaccagtgcagccgcaaggtgacacattcaatttgttatctggcattcactctgaaatttgtgtttgctatctccaagatgttaatttttttagcccacccccaaaatgcattatttgaagcttgtattccaccatattagagagatgtatttcccctttcttttttaatctttagaactaattctcgtcctccttaatagtaatgatgaaattctgagatattttttaacatctatcttgactctacattcaggcttgtttaagtctttgggaaataggaatactggaggggtgcggtggctcgtgcctgtaatccaagcactttggtggatcacttgaggtcaggagttcgagaccagcctggccaacatagtgaaatgccgtttctactaaaaatacaaaaattagcctggtgtggtggcaggcacctgtaatcctagctactcgggaggctgaggcaggagaatctgtgaacctgggaggcagaggttgcaacgagctgatatcatgccactgcactccagcctggtcaacagagcgagactctctctctaaataaataaatatactgaacacatacttcttttcacccttcatgaataaagaagtcctgattcatctttcattcaaaataaagaaaaagaaattatccaaaatattgcactggctagcaaaatgcaaattttccccagttaattaaacaaatatttgtctagtctatattgcgtatcaattactttgctagacaccattgaagataaaactaagagtaagataaagccagtgtccctggacaacaacacaaatgttaatactctgcagctgtaatgcaatcctggatgcacaaagtctatttaattttatgcatattcttttaggttaagaatcactgagttctctcagtggttagaacaatgtaaattaaagtgtatctacaaagtacagtggaaaaaagaaggaagaagagatgaattttgtcaggggscatataagaagaaaataaatattaatatatttggtgcctctcagagtaattaaaggcaaagatatcttgtttaataattttgattctcattgtaagcataagaaaaaaaggttcttttacattttgataatgcattctcttacaggcttttaatttttctctctgatttatcacatgagttagtacatgttttatacttttgaaacattcctcttgacaactatagagagaagcatttcagacctatttgggacctctagctatgtgcctactcaatggcttggacttgtgagttcaatagtatcttgattggtacaatggagttttatcagtaacgatgacttgtgggtcattctgccttctttgcataaccagttgcttctcaatatcgcttctcaactgtgttagttttgggtctcaataatagtatcccgccctgttgtttccattgctacaaagcttagtgactaaagctggcgttcaatgcatgggaaattccttgcattttgtcttaagttatctgtttgaagatcaagcagggcgtgtgtgtgtgtgtgtgtgtgtgtgtttgtgtgtgtgtgtgttggtgagtgaatatgtaggtacattacaagtttgattaaccatgccttagtatcatatttttgtgaaaaaaatcaatcatatttaaaacaatattttaagatgtctaaaatcaatgcccctgaaatccacagggaataaaatagcaaaattttaacatataaacgttttttatccattcttctgtcagtggatacctatgttgatttcatatcttggctattgtgaataatgctgcaatgaacatgggaatatagatatctctttgaaatactgacttcatttcctttggctatatacccagagtgggattgctggatcatatggtgattctatttttagttttttgtggaacctccatacatttttccatatggctgttccaatttacattcccttacatgccaatgttccctttctccacatcttcactaacagttgttatcttttgactttttgataatagccatcctaacaagtgtgaggttctagctcattgtggttttgattttcatttctctaatgactagcgatgttgagtacctttacataaacctgttggccatttgtcttcctcagaaaaaaatgtctattcaggtcctttgcccatttttaataggggtattattatttttgctatcaaattctatgagttccttatatattttggatattagccacctatgagatatattgactgtaaatattttctcttcttctgtggattgcctttcattttcttgttttctttactgtgcagaaaatttttaatttgatataatccctcctgtttaatttggcttttgttcctatgattttggtgtcatctctacaaaattgttaccaagaccagtgtcatagagctttcttcctgtatttcctgctagaagctttacagtattaggtcttcagtttatccattttgtgttgattatattaaatgcacattacctgttttttacataatagtttaataagttaagtaggagaatcataaacaagatcttaatatcagtatataagaatcatagattatttattgataataacacttagtaggaatcactatgattgaatttagtaataagtccttaaataaaactacccataacttattccataaggataaatatctatcccaaacttcacagagactgtatagactcttagaaattcacctgacagtttaacaactaacaggataacaaacctgtcctttcctgggccatatggtcctcattctgtaccctgaaggcatttattttgatctttacagcatagatagtaatcacagaaaaaaaaagactttacctatcataaaaacattcaaaccattttcaaagctaactcatgtaatgtaatgtaatgtaggggtatctaataccagtttcacagagaaaagtagtcttactatcagatcataatatgtgaggctatgtcctagcatccaaattttaacatactttcataattcacttttccactcttattgaaatatatcaaatactttttcttatatatttttctttaatacatgaactaatttgatgtattttgtcataaaacataattttagaaaaaaatcaatattttctatgaaaagacaaccatctcaatgtacagaatataaacatttgggcaattatatttatatttttacttattaccattaaaatgcacgatgggttagattttgtcataatacatttatttttaacaacctcacattactatatgcaactatgtcaagaagtttttttctctttagaaaatttgctttcctacttttgacaatgattatttcatccttttcataactgattaaaatgacctccttctttaatttttagtgatgtccttgccaaatgctttattgagaaaataagccattagatgggaacttcccacagcaaaacagaaatgtagtggcacctgcacctacccatatcgtccttctttcctctaattataaaaggcagtgtccctctatttgtcagaggctaatcctctactcttcctctctccctgtctccctctctctcttctgcatatccaggctctttttttctgtaggatcattgccacaaacagatgttctattatattctttctcattatcttattcttatcaccaggggaacctattacacatacaaatatccaggtctatcccaagaatctccaggaaaagagaatggagtttggatgttttataaaaatgccaggtgatttttttctcctctggaaaatttaggtgatattgcttgtcaatattacccctgctggccccacatctctgccaactacttccctacttctctaacactcttcacagccaaacttctcaaataatggactcacagacataaagtcaatattctgaaaatttatcaattttctccccctagcctgatccttccactgcctaattaattgcatacaccagcagctacccattcacctaaccaggaattagagcaactcttggtacctctttcttcccctctacactgtgatcatctggatcaatgagatctatctctatcccttttcactgaaaacactctcctcatggctgtttcgtcttctgacctggaccactgcaataggctcctaacttgtcccctgctttgattgctttcaaactccaaacattttttttgcacagcctccagaatcttcttatcaaaacatatatgatatagtttcagtcacctgtttataatcaatattgaattcacgttacacttmtaaaaaaatctccttttggttgtttaaaaagccctgctgactgggtggagtgactcaaaactgcaataccagctattcaggagactggggctggaggataacttgtggccaggagttggagaccagtttgggtgttatagcaagacccccatccctacaaatttttttttaaatgggccaggcatggtggtgtttgccagtggtcctagctactcaggaggctgtagcagaaggattgcttaagtccaggagtttgaggttacagtgagcaatggtaacaacactgcactcagcctgggtgacagagcaagacactgtctcttgaaaaaaattataaattaattaaaaaagaaaattttgaaaagtcttgcttctccaatgttattatcttattcaaataaattttattcgaaaactatttttatataatacaatgagatgactatagtcaataataccttaattgtgtattttaaaataaatggtgttttctgccactcttgtaaacaacagacaagtacagaccagaggaaggaaataagtaaatggccgtgatgggcgataaaggaatagctgcgaaagagtctgaggagctgatgtttgagacctgcaggaagggaatgagccagctgtgtaacctcctcctcaccaaaaagaatattcttggcacaggattaaaaaatgcaaaagtcttgagatgagaaagggtttggagagttctaggaagtggcagagaataatatgactgaagggagtctgtcatgaacctaaggaggtaaacagaagccagagcacttgaggctacacaggccatggtgaggactttgaaatatttttcaaatgcatgaggaattcattaggcatctgaaccagagaagtaagatgatttgatttatatgactgctttgtagagggaggagcaaaagtatgaggaggtgggggtcattttaacctttcgacttgtgccaccttcatctaaacaatactctaacctcagtggctacttctagttccttcagaaaacaaaagcatttcagaagattgtagcacatgctgtttgccttgtctcagattctcctttgcacctttcttccttaaggaagcaacttacacatcatgtatgtgttgactcattctgtggcagcaccagtgaaataaagtttaacatcattattcttttacagagcaccatattctcgttcttcatcactattttaattctgttttctgtgwttgtgtctgtgtattctttttaactatccattttctcaataaactctaagccctgtgagagcacacaccatgcttctctcgtattaccagtgcagattgataatgggtgctctgtaaatgtctcctaaatacctaataaatatgtgctgacatcattagtaaatttacatttcaaaagatgcaatttatagaaaaaattccagtgtttttctgctctctgaacagatttttttaatagacattgatcatatggtaaaagcctagacctgttctcctaagcaaaattctgcatgtgtacctttctgtcaactcaactccagaaatacactcctaaaaatttaataggagacttcaaaactgaattcgtggctggtacaaagcattcaagtttctgaatggtgtagttattcagatcagtgcatagaactgtgtttatacacgtaccctgtagtgtcttttaagaaaaacaacatcattctgatagaaatgatgtttcttcagtctttctggatttagtacattttatggtcaaattttatttttttcttggtggctaagtaccttttgaaactaattgtttgaatttaaaaacgttgttgcatataccgcaatagaagtctttttataatgcatcatttcacttttcagccttcattagagttgatgatttatagcagtaccattctcaaagaatcttccgcttagagtgtctccatgaatatcgtcactggaagtggggaaggtggaaaagctgatggtcagggtgggccagatcaaacaattacttagatgctgcaaagaatcataataagggaaggacagttacattaaactttgtgttatgcttgttccctagtaattgattgctttttctaaaagtagagtaaatcagaggagatgtgagggttttaagttttgatagtacatcagtacaaggctacagatgtttattcaacaataaccgtgtgtgagacattatgttaggtcctgtgatgattatataggtaccagatgtagcttccatcctcaacagcttacgtttaaatgtggaagaaaaaagaatatttagaacactataatacccctcagaacatgtaattattagagcaaaaataaaaggagttcgggaatcccgaggggaggtgagtttacttttatatttggagttttctggtatttgtatgcaaaagggacttttggatagtggatgaaattgaaatttctacaggtaggtaacaggaaaacaggctgggggaacactacagaaaagggaagacatgcaaataggtaggatacttccaaatcacatygcataatacggttttgctagactataggataaaaaaaggaaattatggggaaaaagcctgatagatcagggatcctcagcccccgtgccatggactgctkccaggccatggcctgttacgaaccaggtggcaaagcagaaggcaaacggcgggctggtgagcattaccgcctgagctctacttactgcagatcagcaggggcattagattatcataggagagcaaaccctgttgtgaaccgcacacacgaggagtctaggttatgcgctcctcatgataatctaatgcctgatgatctgaggcagaacagtttcatactgaaaccatcccccaccacacccccattcgtggaaaaattgtcttccatgaaactggcccctggtgccaaacaggttggggaccgctgcgatagatgattcgtcaggtgatggaagactaaatgtcatttctagacatttgggcttttatccataaggaaaggggagacactgaagttactgaagtgatatgttgcactgtgtttagggaatagttctttggcagctacttctaaaaagggctggagatgaagggattcatacgagagaattaagctttcaatagtctaagaaaaggatctgctatgctagagcagggaaagtagacagtgtgtgaattcaatatgttttattaaagtgagttctataggacatagtgactgagatgtgcagagtgacagagaaagtatcagagaatacttccaggttctgaatgtggagcctttgagatcagccttctcattgacagtggtaggaagaacagaaggaagaacaggtctgccatgcagataagcattcttggcaacagtcaagaaactaatttaaatgtttgaatgcctcctgtttccattgtatgtaacaaggtgtagtggcagccaatccagattttaggcaccttgtaagggggctgtggagccaatcagtgacaggaaatggcaatacaagaaggaaaacaaatacgaaacattcactaatatctcagaatattaactcactacgttgactgccaccaagagagaaagaaaattccctttattttgagctttggagagcagtgccagttatatcacagaagaatgtgaaatggtaggtagaaacgaggaacaatcagggtgataggatgaggtttagactactgcagaaaaccacagtgcaaggcatttccaaaaagtagattcaaacggtattaacaaaagctgaaaaagggaaagaaaaacaaaaactgagggaatagacttccagttaaataatagaaggatttataatccattgggtatatacccagtaatgggattgctgggtcaaatggtatttctggttctatgtctttgtggaatcgccacactctcttctataatggttgaactaatttacacgcccaccaacagtgtaaaagcattcctatttctctgcatcttcgctggcatctgttgtttctagactttttaatgatcgccattctaactggcgtgcacacgtatgtttgttgcaggactatttacaatagcaaaaacttggaaccaacccaaatgcccatcaatgatagactgaataaagaaaacgtagcacatatacaccatggaatactatgcagccataaaaagaatgagttcatgtcctttgcagggacgtggatgaagctggagaccatcattctcagcaaactaacacaggaacagaaaaccaaacactgcatgttctcactcataagtgggagttgaacaatgagaacgcatggactcagggagaggaacatcacacaccagggcctattggagggtgaagggaaagggaaaggatagcattctgacaaatacctaatgcatgtggagcttaaaacctagatgatgtgttgataggtgcagcaaaccaccaagtcacatgtatacctatgtaacaaacctgcaggttcaacacatgtaccccagaacttaaggtaaaattttaaaaaagatagaaggaacattgaggtattaagaaaactattctgaattttggaataatatatattacattgtgagagacctaattataaaattggataatgaaaaggcaaggcaaaacaagaagatgtgcataaaagaatgaaaacaaaagaagctctcctctctttttttataagaatcaaagaaatgtttcttctttcttctctttctcttcctctttttcttactctttctttctttctttctctttctttcttcccttctttctctccttccttctctccttccttctctccttccttccttccttccttccttccttccttcgttccttcctttcttcctttctttctttcttgacagtgtttcactcttgtcgcccgggctggcatgcaatggtgccatcctccgcctcctgggttcaggcgattctcctgcctcagcctcctgagtatctggaattataggcactccccaccatgcctgactaatgtttgtattcttagtggagatggggtttcaccgtgttgaccaggctggtctcgaactcctgacctcaggtgatccacttgccttggcctttcaaattgctgagaattcaggcttaagccaccacatccagcccaaagggatgttttttaaagattgttcttgatagaaaatgccaccatgaaaataaatagaaaatagtcttaaaataaaaatattctttaacttcccaacacattgttttgtacatataagagaaatacacatttattttttaaaaacatattttgaaagattataatataacttctagatttaaataaatttattcacttattttataactgaagctaaattaaaaattgactaataacagaaaaatgagaatgtgtttctattcaagtagtaagataccctttttatttttgaagatatgagatttttattattgacaatattaattttgaagagatgtatgtgtgtgcatatgtgtgtaaaatgaaatagagaaataatttcttcttaagttcacagaatttccactagccaggtattgaattcttctatactgttgtaaattgataaaattcaaattcttgacacaaaaaaattagataatacattcaaaatatttagaacgaggttttaaatggaaggcaacacaattcatgacacagacttaagcaatatatttaataatatttagaacaaggttctgcagacccaattatatttagttaatttaattgaaatgtttttaaatatattgtttcattgcattgatcactaactttgaagggatttatttttattaacttgcagcatagaaaggagtaaaaataaacaatactcatattttccccagttaatgaaaataaacaagtattagtaaaaagcttctgatctctaagagtacataatagaattctttttgagaataactgctgttttgacaaataccatatccttcaagacaaatcgaacaaccaaaagacaatctgcctttatttctcaaaatgactttgacctcaaattctgaaactgacagggtgactttgctcagtgaataaatatgatacagaaccttttagagggaatcgaatatgccgaatacccaagtgatctctctgtctctctttttttttttttttattatactctaagttttagggtacatgtgcacattgtgcaggttagttacatatgtatacatgtgccatgctggtgcgctgcacccactaatgtgtcatctagcattaggtatatctcccaatgctatccctcccccctcccccgaccccaccacagtccccagagtgtgatattccccttcctgtgtccatgtgatctcattgttcaattcccacctatgagtgagaatatgcggtgtttggttttttattcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtctttgctattgagaatagtgccgcaataaacatacgtgtgcatgtgtctttatagcagcatgatttatactcatttgggtatatacccagtaatgggatggctgggtcaaatggtatttctagttctagatccctgaggaatcgccacactgacttccacaatggttgaactagtttacagtcccaccaacagtgtaaaagtgttcctatttctccgcatcctctccagcacctgttgtttcctgactttttaatgattgccattctaactggtgtgagatgatatctcatagtggttttgatttgcatttctctgatggccagtgatgatgagcatttcttcatgtgttttttggctgcataaatgtcttcttttgagaagtgtctgttcatgtccttcacccactttttgatggggttgtttgtttttttcttgtaaatttgtttgagttcattgtagattctggatattagccctttgtcagatgagtaggttgcaaaaattttctcccatgttgtaggttgcctgttcactctgatggtagtttcttttgctgtgcagaagctctttagtttaattagatcccatttgtcaattttgtcttttgttgccattgcttttggtgttttggacatgaagtccttgcccacgcctatgtcctgaatggtaatgcctaggttttcttctagggtttttatggttttaggtttaacgtttaaatctttaatccatcttgaattgatttttgtataaggtgtaaggaagggatccagtttcagctttctacatatggctagccagttttcccagcaccatttattaaataaggaatcctttccccattgcttgtttttctcaggtttgtcaaagatcagatagttgtagatatgcggcattatttctgagggctctgttctgttccattgatctatatctctgttttggtaccagtaccatgctgttttggttactgtagccttggagtatagtttgaagtcaggtagtgtgatgcctccagctttgttcttttggcttaggattgacttggcaatgcgggctcttttttggttccatatgaactttaaagtagttttttccaattctgtgaagaaagtcattggtagcttgatggggatggcattgaatctgtaaattaccttgggcagtatggccattttcacgatattgattcttcctacccatgagcatggaatgttcttccatttgtttgtctcctcttttatttccttgagcagtggtttgtagttctccttgaagaggtccttcacatcccttgtaagttggattcctaggtattttattctctttgaagcaattgtgaatgggagttcacccatgatttggctctctgtttgtctgttgttggtgtataagaatgcttgtgatttttgtacattgattttgtatcctgagactttgctgaagttgcttatcagcttaaggagattttgggctgagacgatggggttttctagataaacaatcatgtcgtctgcaaacagggacaatttgacttcctcttttcctaattgaataccctttatttccttctcctgcctgattgccctggccagaacttccaacactatgttgaataggagcggtgagagagggcatccctgtcttgtgccggttttcaaagggaatgcttccagtttttgcccattcagtatgatattggctgtgggtttgtcatagatagctcttattattttgaaatacgtcccatcaatacctaatttattgagagtttttagcatgaagggttgttgaattttgtcaaaggctttttctgcatctattgagataatcatgtggtttttgtctttggctctatttatatgctggattacatttattgatttgcgtatattgaaccagccttgcatcccagggatgaagcccacttgatcatggtggataagctttttgatgtgctgctggattcggtttgccagtattttattgaggatttttgcatcaatgttcatcaaggatattggtctaaaattctcttttttggttgtgtctctgcccggctttggtatcagaatgatgctggcctcataaaatgagttagggaggattccctctttttctattgattggaatagtttcagaaggaatggtaccagttcctccttgtacctctggtagaattcggctgtgaatccatctggtcctggactctttttggttggtaaactattgattattgccacaatttcagagcctgttattggtcgattcagagattcaacttcttcctggtttagtcttgggagagtgtatgtgtcgaggaatgtatccatttcttctagattttctagtttatttgcgtagaggtgtttgtagtattctctgatggtagtttgtatttctgtgggatcggtggtgatatcccctttatcattttttattgtgtctatttgattcttctctctttttttctttattagtcttgctagcggtctatcaattttgttgatcctttcaaaaaaccagctcctggattcattgattttttgaagggttttttgtgtctctatttccttcagttctgctctgattttagttatttcttgccttctgctagcttttgaatgtgtttgctcttgcttttctagttcttttaattgtgatgttagggtgtcaattttggatctttcctgctttctcttgtaggcatttagtgctataaatttccctctacacactgctttgaatgtgtcccagagattctggtatgtggtgtctttgttctcgttggtttcaaagaacatctttatttctgccttcatttcgttatgtacccagtagtcattcaggagcaggttgttcagtttccatgtagttgagcggctttgagtgagattcttaatcctgagttctagtttgattgcactgtggtctgagagatagtttgttataatttctgttcttttacatttgctgaggagagctttacttccaactatgtggtcaattttggaataggtgtggtgtggtgctgaaaaaaatgtatattctgttgatttggggtggagagttctgtagatgtctattaggtctgcttggtgcagagctgagttcaattcctgggtatccttgttgactttctgtctcgttgatctgtctaatgttgacagtggggtgttaaagtctcccattattaatgtgtgggagtctaagtctctttgtaggtcactgaggacttgctttatgaatctgggtgctcctgtattgggtgcataaatatttaggatagttagctcctcttgttgaattgatccctttaccattatgtaatggccttctttgtctcttttgatctttgttggtttaaagtctgttttatcagagactaggattgcaacccctgcctttttttgttttccattggcttggtagatcttcctccatccttttattttgagcctatgtgtgtctctgcacgtgagatgggtttcctgaatacagcacactgatgggtcttgactctttatccaacttgccagtctgtgtcttttaattgcagaatttagtccatttatatttaaagttaatattgttatgtgtgaatttgatcctgtcattatgatgttagctggtgattttgctcattagttgatgcagtttcttcctagtctcgatggtctttacattttggcatgattttgcagcggctggtaccggttgttcctttccatgtttagcgcttccttcaggagctcttttagggcaggcctggtggtgacaaaatctctcaacatttgcttgtctataaagtattttatttctccttcacttatgaagcttagtttggctggatatgaaattctgggttgaaaattcttttctttaagaatgttgaatattggcccccactctcttctggcttgtagggtttctgccgagagatccgctgttagtctgatgggctttcctttgagggtaacccgacctttctctctggctgcccttaacattttttccttcatttcaactttggtgaatctgacaattatgtgtcttggagttgctcttctcgaggagtatctttgtggcgttctctgtatttcctgaatctgaacgttggcctgccttgctagattggggaagttctcctggataatatcctgcagagtgttttccaacttggttccattctccacatcactttcaggtacaccaatcagacgtagatttggtcttttcacatagtcccatatttcttggaggctttgctcatttctttttattcttttttctctaaacttcccttctcgcttcatttcattcatttcatcttccattgctgataccctttcttccagttgatcgcatcggctcctgaggcttctgcattcttcacgtagttctcgagccttggttttcagctccatcagctcctttaagcacttctctgtattggttattctagttatacattcttctaaatttttttcaaagttttcaacttctttgcctttggtttgaatgtcctcccgtagctcagagtaatttgatcgtctgaagccttcttctctcagctcgtcaaaatcattctccatccagctttgttctgttgctggtgaggaactgcgttcctttggaggaggagaggcgctctgcgttttagagtttccagtttttctgttctgttttttccccatctttgtggttttatctacttttggtctttgatgatggtgatgtacagatgggttttcggtgtagatgtcctttctggttgttagttttccttctaacagacaggaccctcagctgcaggtctgttggaataccctgccgtgtgaggtgtcagtgtgcccctgctggggggtgcctcccagttaggctgctcgggggtcaggagtcagggacccacttgaggaggcagtctgcctgttctcagatctccagctgcgtgctgggagaaccactgctctcttcaaagctgtcagacagggacacttaagtctgcagaggttactgctgtctttttgtttgtctgtgccctgcccccagaggtggagcctacagaggcaggcaggcctccttgagctgtggtgggctccacccagttcgagcttcctggctgctttgtttacctaagcaagcctgggcaatggcgggcgcccctcccccagcctcgttgccgccttgcagtttgatctcagactgctgtgctagcaatcagcgagactccgtgggcgtaggaccctccgagccaggtgtgggatatagtcttgtggtgcgccgtttcttaagccggtctgaaaagcgcaatattcgggtgggagtgacccgattttccaggtgcgtccgtcacccctttctttgactcggaaagggaactccctgaccccttgcgcttcccaggtgaggcaatgcctcgccctgcttcggctcgcgcacggtgcgcacacacactggcctgcgcccactgtctggcactccctagtgagatgaacccggtacctcagatggaaatgcagaaatcaccgtcttatgcgtcgctcacgctgggagctgtagaccggagctgttcctattcggccatcttggctcctccctcctgtctctcttttttaatcttaaaatcaaaacagctaattttaccacacggaggaattaaaccaggtgtaacggctataacttcaaagatttttgtccttaaggcctagagataattaaaataaacacaatgactttatttctccttaatgtgtcatttatgtactaggttcctgttgggaactgtatatattcaaaattatttttagttactgatgaaaaattaaattcattttatcgtatctggatttttaattttagtgataaagatcaaggtcatcggcaatcaacattaaaataaattggcaaatataagattttccccctgaaatacattggggaaacatttgaaattttaaaaaacagttataaaatgtacggaaaaaatatattactattctcaaacaataataaattcagatacaattattagtattttggctttaaaaagttgctttttagatttaatgtatttcctgaaactattcacaacaaataaaaaataattataatgttacagtatgcatctccaatgttattacctccttgtagacataataggcatatacttattcattcaagaaatgtgtattagttattatttttgatatttgaggagtaaatctctttgtaatcaaaaggagtctattaagtgagaaatggataactagagattcagaattctaaggtagcattgaaataagattattacccgaagatattcctgggattggcagttgtttattgagctttttccccagtatctggtgctaatttttatgcacctatttacagctaaaataggaattgatgggaagaaaatatatatgtacctctctaatgcaacagtataaaacccatgaaagaataattcacaaatcttaatagaaagagagagaaagaaagggaagaaagttatttttcacttcccctgagcaaatatgtaatttccatgatttctttttaatgactgcggtctgtagccagtaggtactgatttatgaaaagctgtaaaagatcaggtggagctctgtaggacatcgtttacagctgcatggattgaaacctatctaatttgtctagtaaaagtgtgagctcacggtgcacatcacccagcctataaatttccccttcagaccaatgtctggggctgggtctgaaaaaacgccaatgcctgcagctgctcaaaggcccagattcccttggtgcgcatgaagagacatcacaggctcttcacagtgagagacaaattcctatccattgataaactctccagatgtcctctctgaggaggaaaggaaccaatttttatttgaaatccccagagaaatcctggaagtggaaaaatgatactttgcagggctagttattctgaggtcattggcattaaaattaggtcatttttttttgtcttggatgcactttgatccaagtcataaacagacaaactcagttggggtcactaatccttaatccacacgtcctgttctatttcaatttgtttatactaattttctctaaactcttgttgagtctggctaaagtgttgagtgtaaaagtacaaactattctgaactgagaagaaagaattctgcattttgctattaggcacacttcataaatattctgaataaatgtcctggggtttgattcttcatacccacaaacccataaataaaaaagtattactattcatgggactaaaacttgacataagattctgtgctccagctactgtagacagagaaactgtggcgtgtgcgcgcgcatgcctgtagatagtttaatgtcttgtctcattctaggtttcacatgatttttcttctaaaatcttctcacacctgatttaatgctattcttatcaattttttagtggttcaatgacttatccaatgagtctaatgtaatgtctaatgcttgctagtgacaaagactatttcactcatgataactatgctgaaacaacccatgaatattgtgtacattttggtgtatatctgtaattactcaagttcttaggaaaagaataaaaatacaaaattttcaattataaaaataagctgtttttcttcctaagatgagtaatttaaaaaatcataaagtcgtttgcatatatgtttcagaagaagataacttatgaaatgttatattgatattttatagttttcctgatattgctaaaggaaaaaattaatatttatataatatggcccaatccttttttagataatctctgagagttagtaaaactaaactatatcagagaaatagaaattataaactccataattaagattttcaatttataatttgaacttgtgaagtaraaaggattgaggtaattgagtttagtctagtggacagaagtttgaagaatccttccctttcaacttccctccactacacaaatgaccatcacagagaggggactattaatctatattttccacagagtatgwaaatagagtcagaaccaatgttgcaatttttttttcctgaaactcagggttaaaactgcactaaaatgtgaccatttttatcaaatacgtttcatgcatcgctgtaaaaatccagatagtgaattcacaaacttctcatatgttccaagtaatagcatgtaggtgtagggcacgatctaaaattatcctggctcacgaatcaactttacacttgagcaggtcttttgtacataagagagtaagttgaagaatatcaccagaagtggtataaacctctcaattaaaagacctatttaagtgtgcgcagagctggcacagatttgcttcatagattttctcatttagaacaggagcttttaccacatatcctagatggtaatcaatgattgctactcatatgtaaaacattatacagttttatttcatgtatattgttcattttgctatttcatgctatttctaaagatattgaaattagccccttagagacctaaatttatgtctaaaataaggataaatgcatgtcgatatattatttgttttggtgtcttctcaccacaaattctaaactttgaatttaaacacttcaaatgacagacttcacttagactaatctttcatttcaagtatataagaaaaatatgaagttctagttttaatactcctaattttggagatttttattgaaagattctgaagaactaaaacggcaaaaaatcaaaatataccctcttaattattaaataaagattaaattatatataaagttttaacattaatactatattttattacaataatgaaaaaaatcacagacagcagaatagcacttctgttttcaagcatttgcagtggaatgattgtgaagttgtaaaagttactgactattctaatgaagcccctaataatggctacttttcagggttgttayactattatgtacataatattatatatgactaaatttctagttacttaaaatggtggttaacaagatcattgcattcccagcattatatataaactgatatttactctgctattttctttttgaagttttcagaagtttaaccatttcaccatttacataagaataatgaacagctaacacatagtgcttattatgagccagcactgttctaagtgcttcccacatatgctcatttaatcttcatttgtgtgaaattgatatcctcaccatcatccccattttacagatgaggaaacagacaagaaattcaccaaggtcacacaaatagtatgtggtatacctgggattcaacccaggccttacggttccagtatctgtgcactaaacttcagggcatataaacatcgaatatgccacatgatggaatcataaatcatttaagaaaaaacaggggaaatgcaatatgaacaaagttttacctaaatggcacagtgtaccagtcaagaatgtggactgtggtatagaataatgtgggtctaaaggccagctctgcctcttttgcctgaggaaacttagcttcgtaacttaatgtgatttagtttcagcttctttatctatctcagaggatagcatcagtgcctaacaggtactgtaaggattaaggtccttcatgtctgtgtaatattcttagcatgatgcctgccacattgagtgcacatgaacgtttgatataatatgaaaacaagggccttaaaaacttatttctgtcattgttattattcatccaaaactaaaggcttttataactaccttgtgcccaagctcccttccttaggagaaaagaagagagaagtcaatatattgttcacctagtgactggctccagtcttataagtcaaaggacaattaaagaggccactaagagctgaagaaaaaaaaaaacacaccaaagacatttcaccaacagtctgcttcttttgcctcttcagggaggcaaacattgtctcattcatttcctttatgcctcttctgttccattgatttaccaggtcttgcaggtggttatagatagatgtttaggcctgtatttatataaaaaaatcctagaaggaagtcctttttctttctctctttctttttgagacggagtttcgctcctgttgcccaggctggagtgcaatggcatgaactcggctcaccgcaaccttcgcctcccaggttcaagcgattctcctgcttcagcgtcccgagtagctgggattacaggcatgtgccaccatggctaatttggtatttttagtagagatggagtttctccatgttggtgaggttggtctcgaactcctgacctcaggtgacccgccggcctcaacctcccaaagtgctgggattacaggagtgagccactgcgcccggccctttttcttaaagctgattgtattgtgcaatataaatcacaattacaatccctctctgagtctctgtgtgtgtgtgtgtatgtgtgtgtgtatatatataaaatattatgtatatatcatgtgtatataaacacattacatatataatgtatatatcatgaataatgtgttgcatatatatatcatgaacttcatttggtgtcaagtatgcaagaatgttttttcttatcagtcatctacttttttctattattttttatttttaatgacatgcagcattatttaactctgtattgagtttatgtcctgctgtgagtgtagcctagattgttcaagccattctttagacagcaacctgtgcaatgtcaaacaaacaaacaaacatccagtgggaaatacttaaaatattgaaactgaatccatcatcaaagcagacagcaaagaattcagtgtgtcctgcctactgtaaccgtatcctaaattactaccatttttgctagtgattaaattcaatgataataaaatatgtatggatatatttatgtctttcactggaaaattttcagtgatttaaaagcatttgtggatggagaaggggagaacttagacaagatgattacacggcgttcctccaagctgccagcttgaacagatgaagctttcatggcagataaattccacaaatgaaaggaacaccatatgaagtctggaagttcctgccgatgttagtgtcaaattttgacacacagcaactctgctgactcctgatctgtctccctttggtgttttttttcttgcaggccatcaaaccttcttctggaccaaagcaatgtggatatcactaccttccrcacaacaggtgactggcttaatggtgtcyggacagcacactgcaaggaaatcttcacgggygtggagtacagttcttgtgacacaatagccaagatttccacagagtaagaaaaaaaaattcattaagaagaatgaaggattcttttgaactttcttggcttgacatgaaagatttgtaacatcttggcttgacatgttacaaatattagtggtagatctgaggtactgactaccgcctggaactgctaatcagggccctgggtctccttgtggcccacagcttctggattgatacatattgatgtgtcttcctctctcaagatagggcccaggatcggtgtctttagatatgaccttcagatcaaccaacagattcacacccataaccacactgcagactcaagcccctaatttaaaacataagcagacatagattgatgattattatatttctgcattttgtgatttttctgttatttttactaaagcttaacatctgtgaatcaggaaaattgagagccaagtagataatgtggtatacaaggaaacatattaaagtctaaaactggttttcatagttgctaaataaacataacaacatgtattcatcattgtctaccctgtaaaggatatcatgctagaaaatgaagttataattaggtttaagatagaatttttgggttttcctcaaaaggcttattgccagatgagaaagaaaaaatatgcgtcataatattaatattaattgatagctttatgtgttaaaaaccaataataatcaagtataatatgctgtattttgtggacaatcatctttgggtaacaggaatcttttgatatgagaaacacatggactcaaggaaaggtggtatataaagaatcaagtgaatccagtgaatagtcaaaaatttggttcttattcacaaagtagagtgtttgggaattgataaattaagagttggaagtcacattcttcatctcactgtgattgagaaccaatttacttcacttcctaatgtgactacataattttttctttttctgcacagtgacattttctggattcccatgcgcatagctaaaaactggctttctaaactcttggtcatatagccttatagtgccattcaactgaccagggtccaactcccttaatctaagttaccaggagagaaagtaggactggctcagtcccacttgtacattggcacctccaaagtcatatgtttactagtatctagtcatttatcttcaaaagaagtagaatgaggtagtatgaacttggctctaggggccttttcctgaagataagatggagaactgtcccaaagaaagaagtgtatactggatagatacaccaaaagttgtccagcatgttgtcgcttgcatggtctattgggaaactgttcatctttgtgtcagtctttctttgatctttgttaaattaaataaaccctttagtcccccaagtttaagtattatcatctcccactaatatttaaatatttaaatattgttgaatatataaagactttcattgccaccctgtcaatcattcaacatcactgtgaaaaataacagattagatgccagaaaagaacatgccatgtaattaatagatataagtgcccaacaaacaaataaataagtttataatttatggtgtaacattacaatcagaaagacataaagccaaagaaggacttacaggaacagtttacgattattgaagaagaggacttgttctaaaaaaataaacttaccctagtcaatttaacatgcaccagcattcttgttcaaagtggaatgtggtgtttattgaatagacttctgacccaatcgacagtacacttgggtttctgtggtacttattttgatcagatacagtctcaggatatagtcatgtgtttaaatatttttataaattcttattttagtaatataaacagttagggttatgatacacaaaacacctgctgtagagcacagaagcatagcgaaaagcaaacacaaagttgaacatgtccacatcactgccagattttcaaaggcaaagttatgcaaatgtttactctcaataaaatatcaacactcaaaacttttagatattgaaacactatattgaaatgtatgaggcagaacactgttgataagattaactgctagtttgcaattcaaatattgtcagccattataagctatgaatgtgataagaaagctatttgctttaaaaaagtgaatgacagcatttgtcatactttccaagttagcgacattacttatgtcagcaataccatgttggacgtcaaactctttttggctgattggaatatttttacttaattgttattattatttgaacaacctgtgaatatacattcaagatgaaaactgacttttcctgaaagacagctttttcaggtttgtcctgcacactgagtcaggtggtatggatatgtccatattgcactccccacaatattccttcttctgtcctttctcctcaattcagggacaatataggattaacacctttgccctaacactccttcttgaaaggagctgcttttttccttgagtcttcatagtgtcatgcacctcatgctcctaatttatattacaaaaagagaatttattaaaagtctttaacatactaataatttgggtaaatataatgaggcaacattttcagcataaatgctgtaaaatattgagctagatattttagctttctttcgggttcctggcttattgtttgaatttgattgctttcagtctgtattttctttgtgccatccagactcaattcagaaatgatttgctcgaccaaataactgtggagatcctcctagaatagaaaaagatacctttattcctattcaggctgtcaaagtataggaaaagatcaataaaatttcacattcaaatgcatattaatttattttcgttagtttagagatttcattgacatgcaagctactcaagttatctcatttttgaacaaagcccagaacacatgcaattttgtttaataaatacatatttcaagacattatttcataacctaagatggatttaggaaggttaaagtgagaaaggaagagagaaagggaaaggttagagtggctgtgactgtggaactagaaagagaattaagtgtgtggaatgaatattaagcagattcattgccacatgtcattatcctgcacccttggatgccattttcaaagtgtcatcacctatccctttgcctttgaagtaccagataacactttaacctctggtctcagtctggagctgctctttagtcttgaacttttccgccactcttctgaacaggacagtcaaaatgaaatgagcacacagagtgttatggagacagcagcaactgggggttccaaattattttggacatgcagagtagtggacagaagatattcgggcaagaatagagagactagagggtgtttaaaagccctagctaaggaataacccaaatggataattactttaattctcttttcaaaagtccaatataatcatttgcattaagtaaaaaaattgtatataaaacatttatatgtagtatatttatataaatatataaaaatatatattatatattatatataatatataatgtttatatattgtagataacatatataaacatgtatatatatataaacatgtatatatataagcatattatatatatatatgtttgcttatattgggttcacagatacagaatgtaaaacagttttttgcagaacatttcttgaggaatacactcaggaggtgcagctatgaagaagtgatgaggaatgtctcaaatcrgtctgtgaactcagcatcacatttttttttccagccacttggggatgagtgcttcagttcagaaggaagatcagggccaaacatcatagcaagcagcactcagtaactatgcttaaatttctcaaagagctcaggcaattactgcattggaatatataaatattatatatatatatttactcatataatataaatattatatatatttactttccatgactattattatttttatttatttatttatttatttatttatttaatttttttgaggcggattctcgctctgtcgcccaggctggagtgcagtggcgcgatctcggctcactgcaagctccgcctcctgggttcacgccattctcctgcctcagcctccggagtagctgggagtacaggcgcccgccaccacgccccgctaatttttttgtatttttaatagagacggggtatcaccgcgttagtcaggatggtcttgctctcctgacctcgtgattcacccgcctcggcctcccaaagtgctgggattacattccatgactattattttaggaaggggaaagggaagasacaaaggacaaaaaatcatctagtcataaaacataggatatggtattggaagaagatgaaagaaaaaagaaagagtgagaatctattgatttgcatacccatacttgtatctgtcctgttttctggctctagactcaaaaactgactttgctgtcgtttggggataagtggagaaatgtttattctttgttttaaaggtagattcatggtatgtttatcaaaatcctgtaacgcacattctcaaaggcagggatcaacagaagtagcatacattactccaagtgaaggcttccatcatccgcagtacactaagcgtggcaagcctcacattcccatacatccccactagccttcctgagaaagagtatgatcagatagggcctaaggcaaggcacaagctctccagagaggatttatcagccctttgacagtcagcagggagggaacctgaaggtcatcaggaaccttctcagcccagatggtaggaattcagtagagtctcggttaatttaatgccctaatgcttaaataacaactacatttcttctcaaatggttacctattgttttcttgaacacctttattgcctaggaacccatttcttaagaaatcgattaatccatttctgtgtagttgaattcccaattttttctttctttatgctcagtataaaggaatcccctctgtgtgtgtgtgtgtgtctgtgtctgtgtgtggtgtgttcacacgtgttcttttttccttgccattctcaattttcctggctctccttgacaaatgatactttacgccctggtcatctttacccagaaaaatatattttgtatgtttctttctttagcaaatttgtattgagcccactaactggcagccattgtttaggttgctgttggtaaacagagtacgagaaaaaaaaaagaatcccagttcacatggaatttaccttcttgtggagtaagatagggaataaagaaaataaatcagaaagaaagtcaatatgtcagatggtaataaatgctttggaagaaaatgaaacaagaaaagaaaacagaaagtgcagatacaagtacagaaaagttttaaaatattaaataggagggttggggaagatttcattagagggggatgtttgagcatctctcaatcctgaaagacaggaatatatgaactaattacttgaaacaagaatatataattaaccatttgcacattagctgtcttttttacctaaatgaaattccttatgtttttataatctttattttaatgcctactttctgactttaaattattatatatttaataaaagagataggtctttgtcattgtttttctactttgaaagaactgtgtactttaatgagattattccagttatctatggctgcacagcagcctccccgaaaacttaaaggctaaaacagcaagaggtttattatacatcatagtgttgtgggttataaatgagggcagagctcaggtgggtgattctttgtgctctgaggttgatgttgacagaagcttacagctgccaaatggactgatctggtgtggagggctggagatgctttcatttcgacgtctgccttggtacagttagctgtacagctggtctcacctgggattgtaggccaaggtgcttacatatggcctctcctgtgaaatagtatcaggtagtcagactttttatatggcagctctagagcccccagagaggatatctcaagcaataagtagaggctggcagtttcctaagacctgaatgaagaaagtgatacaatgtcaattctggcgtactctgttagctgaagcagtccccagattcaagggaaagagataaaaccccacaaagaatgtgtgatcattgatattagtttcctaaggctgccacaagaagttaccacaaatttattggcttaaacaacagaagttttttcttagaattctggtggcagaagtctgaaatcaagatgttggcagtaccataattgctccagtctctaggggtgaattcttcctcgtctcttctagtttctggtggatcctggtattctctggattgtggcaacataactccaatctctgactccatttgtacatcgccttcttctctgtgtatcctattctcttataaggaaactgtcactggatttatgtaccaccctaatccagtaagatattttcccaatcggtgtattaattacatctgcagtgaccctctatccaaataaaatcgcattttgaggtttgggattgacagagattttggggaaactctattcaacccactacaccatctatatagccttctacaagatggtggaacggtccggacataacacagatgatgcaaatatcagacaattaggtccacagtactagaatataacatcgtgcaaattgtgctaattgaaaatctcctgcttatacactatcgaggaaaccgattcttatattgttttctttttttacagtgacatgaaaaaggttggtgtcaccgtggttgggccacagaagaagatcatcagtagcattaaagctctagaaacgcaatcaaagaatggcccagttcccgtgtaaagcacgggacggaagtgcttctggacggaagtggtggctgtggaaggcgtagcatcatcctgcagacagacaataattctggagatactggtggaagttccaagtccaataagacactcaaatatgagtacaaatgccttaaaatggaattgaaaaactctttattttcccctatcatttattggatgggtgggtggggtatttttttgtaattgcttttttaaatattagttaatggattaaatttaattcttcagcgtaaaatggtgaagaactagcatatagccattgatcataaactgactatcataaaatcaaaacaagtgaaataacaaaatggacatggtggctttgtttaggtagagccacaaaagaaaagacttgtaatatttttatatacagaggaaatctgtaacaggtattttgtttcttttaaagcaagcaacacagaggaatttatacctcaaactatctggccatatttactaccttatcactgcattattctcttttatctgtttaaagcatatagagatgaagtttgtagttgttttaagtactacacatttttaaattgttagcttccttaagtatatcatgtaaagaaatgtcttaatttttgaaaaaagtacatatttattttcttttgaattgtttttattgttttctatttatgccttgatgatttaatatggatttgttacagccaagtgccaaatgctctctcaaattgtcagcaatttaactagacacagataataatgggtttctttcagattttttgaaccatccacttacatatatttttaaaaaatgaaatccttttcctgttcatacactaaccaaatctctcaaatctgttatcccaatcattgttgcctctccgtttattataaactgtatgctcacaacttagtgtaatataccagcttgtatgcaatggattttcaaccagataacatacctttcctgctctggtgcttagagactatcaactccctcctttagtgaaggagccgtgttagagcttccgagaatagctccactggagagaagtggaatcctatatagaatgctgcactaattgacaacacagcctataggccaatgcatgagtaaaaaaaaaaacaattactggctcactggctttgaaaagtcacttactattgttgctgaaacttgctgagctgtttatagagaatgatgataacagaacttttcctctgtatcactggtgtttaggtgaattaattaaacattgtgatcattagtaccaggtattattatctttaagagtcttccacttcaatgcacatggtgcagttttggtgtgtaacttagaaggattgaacttctttgaatttactggacataacattttcagaatagttggtcatctagcaaccgcctcaaaatgtgtaagcaggagagaaatttctcatcacagggatttagacttactattacataaaggctaactatgagcttgctcattaattttgaaaagatgtacctggtggatatctagctagtaatatattctgaagcaacattttagctctattgatactctttctaatgctgatatgatcttgagtataagaaatgcatatgtcactagaatggataaaataatgctgcaaacttaatgttcttatgcaaaatggaacgctaatgaaacacagcttacaatcgcaaatcaaaactcacaagtgctcatctgttgtagatttagtgtaataagacttagattgtgctccttcggatatgattgtttctcaaatcttggcaatattccttagtcaaatcaggctactagaattctgtattggatatataagagcatgaaatttttaaaaatacacttgtgattataaaattaatcacaaatttcacttatacctgctatcagcagctagaaaacattttttttttaaatcaagtattttgtgtttggaatgttagaatgagatctgaatgtggtttcaatctaattttttcccagactactattttcttttttaggtactattctgagcatactcaacaaaacccatgcatttcataaactaatagaagttgaggattgttgaatctatttcacttattttggctgtggtttccatctgaaagtagaggttgtatacaccatatactgttcttcattttattaatatttttctccttgacctctcataaatttactttacacaattcttaccctgtacatatgtaaacataagtgtacgattcttaaccatggagtagaggtactagaatgcttacggccatctctttgtacaggaactgcattgactttcagtaaacataaagccacaactcctacatgatgttatgtaccatatgatctgttttgtatcttaaatttgatttacatatattatttatttctggtaactcactcagtttatgctgtgctaaatatcaatcaagccatgtataaatgtgatatgattggcaatatgtgtttactttaaacttgtcttttcaaaatattactcagtttatgttgtacaatgtagatggcctcttactaatgtaaaatgatttgtagtggaaacatttatatttttataataaacataatgaaaatattttttacagattggaatacagaagtggtctttgaagttttttaaaaatatataaaactatgtgcttatttaaacagcaaaataatgaaatttacataagtcacaaaaatatgcttctgggcttttattctccattagtgaggagggatttacattgtataatccacatgtttttggtctacatctcattatgaataagccagaaaaataatcagtaaatgttatttcaaagttaataaacacaactgtaatagrcagttctcttttgattgcaaataacagaaaacaaataaattggattaattttctaaaaaagaaaatgtttagaataagtgacagaattatctggggataaactccctagctaagagatcagacacagattgaaccaggaattcaaacaatgtcaacagggttaattctctcattctttgtcttcttcccttggccctattttttttccaactgggttgatttgcaagctatacctttctacaaggtaaaaaatactactctctttaatctaattctgcattgcaactcctatgaaagaattactgtctctctagttccaacagagatcatgaggcctttcttggatcacaagccctatcattttgggctggagaatgaatcactcagattggcaagtactgactcctgtgcttgcccttggagatagtgtgtggggttatatgaaagtgatgctcaggggttagaggtgtaaaagtaatttatgtccaacagaaagtcagaatactgtttatgacaaacgaagaaaggaattctggccaggtcaaataaagcaagtaaaaattaaaaaaaaaaaaaaaagtacaactgcacacctgatactttgtatacaataggtcccttgtcattgcagtccacttaaagaaaatgattatatatgaaaatcaaaaaaagctaattcaaaaatatgcaaaataaaataagtgatcaacattcccatagagaaggaagtggttaggaagagattaaaaaaacaggtctgatttgtgtttgccttgaaggagaaatagcatttggtcaagttgagtggagtagagaataccttgtaagcaggagataagttagttaagttaaggggaagagaagaggtaaataatatatttattcattcaataaatacatagtatgcatgatcatgagccagttgttttgctatcatcatttacaagttacctcattaaatcgtttgcaacagtgctgtgaatggagatgagctacttttatctgagaaaggactccaggctcagaatgtccaagtcaccttcccaaggtcaccctagtatgtagatagatatttcaaactggaatcttcctattctgtattcctcccatcatgtgatagctactataatctccacaatgctctggggacagtggcaatgacaatttgcagaagaggcttgggggagactaggtctaatagactatgtgatgttagaaatctattttggtattctatgcacaaagatatacatctaccccaggatggtggtaatagagatgtgaagaatggaaagcatacaaaaatatataaaagaaagaggattaacaaaatttaccgatttgttctgagaattcttcgaaagatagaaatcaaagcaaaacaaaaatttaggtcttgagtgtctgtggaaatgatggcaccattagtccaagtagagaataaggagaagtcagcttaacatcatgggtttgtcaaatatatagtgtaagccagactttaataagttactaagtattggggatgagaatctgaagctttaagaaaaagaattcaagggtaaaaatgtatatggaaagatgtctttgaaagtatgtaaagatctacattacaaatatttatgtgtttcctatatagacatatatttttcttggaagacctcttaggttaatgctagctacatagttaagttctcaaatattatcaacttaaaataataaaaatatgttttttaacacagaatgakccaaagtaggtgtttgccaggtggccttctttgcaataattaagaatctacactttctccatcttgtcgagccttgaaatcctctacttccagccaggaaaatgaaggagaggatcatttggggggattttatgaactctgcttgtaagtggtacataccacttcttctctggttccactggcaacaactcaaccacactggcatacttaactgcaagagaacataaagagagttgtctgacagtgtgcctaaataaaaatggaaaatttctggaatttatatcacaatctctgccacaactccttttaacataaataatatgtcagaggcagattgttctaaaaagcttatcagagaaacatcagtttcttgctaactgtgccaatttcttttcctctataggcaaccatttttcattcttttctcttattacataaatccaatacctctactaatggcacttgtcagctatgtgagtacactgctcagatttcctttcaagagagtttcttgcaagagagcagttagctgacaactgtgagctgctatattttcaaaatccccagcatatttttagcaggccatgctctaccttggttaatgcctgtcaatgactgagtatggtggaagtactagygccaggccatttcttcacaagtggtaaatccctttaacaagcgtattttgtgttctaatcatcctgactgaarcttgctcaggcataccctacagtctgatctccctactactcaattttccttccttcactctttccttttataggtgtcagaactgtatttctatctgcaagctccttgtgttttctcttaaaccctgttccaatgatgttacccaggcatttctagcaaaaaatatttgcgggtttaattctttctgtgtctgattcttgaaaatcttaaacttagatgactctcaagttgactcacttggaaaactaaaatgttatccttcattcttcyctctrccttttcttcagtgtctaactgatcccctaacaatgccaccttagaaacgctcactctcatcccttttctctactgcccagcagaatctctgttggatcctcattacttttctgagttacagatggaatttcttaaattgtgcccctgattctagtcttgatatcttcaacctacaaaagtaggttgaatatgcagccagactaatacggcaaagttaatgttgtagttcctcctcaggaggcttgcaaacgcaaatgcctcagggaactggcaggttatataaatgagtgaagcaagtgaagagtgtggtgacccataaaagtgccttttcttccacgaaaagcaaatttctcaggtgtaaggcaacaccgatccagtatgaattcctggtttttccttttctttgtttttaaggtttaaggaaactaggttttaattttttaagtttttgtaggtacatggtaagtgtatatattaattcttgatttttcaagagaatccttaatctgagtctttgtggaaaaatttctatatatttaaattataacccacttatttatgaacacacacacacacacacacacacacacacacacactctgaatttaccctgcagctgccaactttttatttctaaaatacagactactcattggtattggcacatgagacctatcttgaataaaacccggcttctctccttattttctggttaacccactacctccttacactctatacaactgtactgacccacttgtagttctctgagtattccactttcttaatgcatcatagcttttgactttttkgatcttttgtctagaatcacttagccaatctctaatcattcttcaggtcctcttatatccctaaaaactgggttaggtaacccttcctcgtgcagcagtagaaccctgtacttaatactgacacacactatcttaaccaaaattgcctattctttatgccctatctttacttctagctgaggacaccttgaactgtgttttgcaaaccattgttgttgcatcacctattgcaattttcctggtatataataaacacaaaatttttttgagttgaattaatatattaaaacaatgtttgatggcattataaatggcaatgtatctgattattttttccccgtaggaaaaagtgtaaatttccttcagctcaataaagctagttaatgttctagttatatatcttggatcattgaatatactgatctctgctagaactggttcattttcatagaattttgtggacttgtttcattggtctcaatgctcccacttttcttagtgcagttaagttgtttaagtagacccttgaaccagagcttagctataaaaatttaacaaggtaaatgccaaagcttttctgatcctttctactttctcactatgtacaaataatgtcaacatgcctactaaaagctacccttcttatgaggcttctgggatgcacattctgttgacacacaggctctagatgccatttgggaaaataagtggtggagttagatatgccataaaatgaagagtttggaggtttgcagaagtcctcaccctgaaattctctgggaaaactcagttacatgttgatgaagaatagtaataattgaatcgcattttccaacaaagtgggctatcagctttcctctttgaagaaaggtaattttccacttaacttagaaataaagtggtgctagcaatagtaataatagaagaaaagaagttgcttttcttctattatgtgcatgttacatatatgtacacacacatataaactaacttttctgaataattataattgatgtacattgaacttttactctgtacaaagcccttaacatatattatttgatctaatttttccaagaacttaatgaaataagtatgttgttatccactttataggttgcaacttagaatagctaataatcacacaaggtcatacagctagcaagcagaagaactgaagtcaaaatcgctccagatctcttgttctgaactattatgttgtattagctgtcagtataatattacaacttgaggataaaatctgaaggacaataataaaaactattccagaattaagatcttgggtagtctctattttccacttttcaatgtgcaattgtaattcagtaattctgctagtagaaacatagttctgcagaatcctttgattgttactaccatggtagaggaaggaacaggcagttatccttgatttcatttctgagaactccatttcaacagcatgagatttgcttgacayacagtgaacccttcaagcaaacatgttcccttgaacattcaacaaataatctaatttggaaacaccaaataccacaaggaaaaagtagtacttttgttctattttctgacagatttccacaccagtgatttatggacgtatgttaagtttaggattcttatctgaagccaagaaaaaaacctttatatttttgaagcctatcatcctcaatgatggaagccaaacccttggaatgttgctaaaatcaaggatgagagaatacagaatgtcatgtactatcaagaacttattgactttcatatggttctcatttattctcatacatggcaaattgaaagccttgggaggaagccaatacaaattgttacagataacactatctaaatttaactgccataggtatgcttcatataataaatatatatttcatatcagcaactcagcaaggcaaaaattgaaacagactctagtcaaaattcatttggacacaatccttcagagaagactcaatttgccaatataattatttatyttggtctggagagcccttaatactcaattttatgtttctgactctagagattttatacacacacacatatatatacacatacatatatacacataaaatactctatggaagttagaacattgaaatcataacaatatcttagggtaataaaattaagtttgaatacatgaaagtttagtacctgcctgcagaatcaaatagattaatttgttgatattttcaaatttttaaagacatttttgcaacagaattttttcttggtttaatgggttcagtttgaggcatcatttgagcattaaaggctgtggcatgacattaatttttagcctagtgtgatggttcatttatcaagtgcttaacagagtatctagtatacaataggctagtaggcatacaattagtctttgccaaatgaatattaaatgtattgttggaggacaaatgaacaatattatctatttcaacaattgtagcagagattctgtgcaacatgcagttgctttccatttagattaatgtttctctgaaacagaaagttttgtagaggaaagtcttatctaacacatagacagattcagggcccagcacatagaataaacccataattgttgaataatacacatttataatgtaaaatcactatgggtaacaaaggaatcctattatacactttcctactctggataaagagaaatatatatatttacatacatatatacacatacttaattattaaatggattaattatatacatatataaatatgaatataatatatataaatatgaacacaatatataaatacaaatatgaatacatatatacatatatttatatatacttatgaatacatatatacatgtttatatatacttataaatacatatatacatatatttatatatgtatataaacaaatttatttatatatatacacatatatttatttattatctggcttactaatacttaaagaattaataaatgccaaagccagggatatgactaaaaaactaagatatctttccttttaagatagctatgattgcataacatgagacactgtggttgcataatatcattcagtgacttaaaacaacaaaagtcacttatttcatgttttttgcaggtcaggaatttatacatgggaaagcttgtctctgctccatgttgcctaaacttcagatggaagactcaaactctggcattggattcttctgaatatttatttgttcctctatctggttattggtgttggctattatcggggattcagctaagctcacacgtgccttcacaatgtggcggttggttccaagtacagacatctgaagggaggggaggagaaagaggaaggatgtgagattttaccctacttacaagccaacaatttggcctgccacattttcatggatattgkcagttgtcatgaggtttctgggtcagagacaaagaatagtttggtactcacagcagtggcagtatccagtgtcaccattttcatacacgaattccccaaggcctaatttctacaaagttctgcaaagagagccaagtcttgcctgcatatgcaatgagttttgttgaagaaaaggaaactcatgtttaaggaactacaatcttttataattggctgcaagcaaaactgcctaacaatttcttcagagagaaatgtctttatcatatcggagagttaacaaatctaccatttgctgcaaaggggagtactatgtctttattccaaggttatttaccatacaaatatctttgaaaagataatgcggaaaagagggcagttagtgcctctgcttgaaagatatgcagaaacatgaaagacccatagagaatcttttcctccgaaagactcctttcaatctttgtggttaagaacaatgaataaatttaatgtaaactcattaaaagatttaacctcaaaaaattttaatatccacatttaagatttctttaggagagaagaaatcaactcccttaccctcatcctcctttggttaggaatgactacacagcatagaaaacttgggactgttaggcattaaacaatcaaaagattcttctcaaattaagataatagcttacatgaatattttatgaaaaatagccacatttgataggcttttcattaaacctatatataatttgggtagaattcacacctttacaatgttgagtttttcaatccacacatacggtatgcctctttgtttatctacatcttctctgatgtctttcattagcattttgtaatttttagcataaagatcttatacaagtatttttttaatttgtacttaagtacttcaattcccttggcataattataactgatatatatatatgtgtatatagatatacatatatattatttatttatttatttatttatttatttatttatttatttttgagaactctgtcacccagactggagtgcagtgacacaatctcggctcaccgctacctctgcctaccaggctcaagctattctgctgcctcggtctcctgagtagctgggactacaggagtgcaccacttctgtccagctaatttttggatttttagtagagacagagtttcaccatgttggccaggctggtctggaactcctggccttaaataatctaccagcctcagcctgacaaagtgctgggattacaggagtgagccacttctcctggccctaatattatatttttaatttcggtttctacctgttaattattgaaacatggaaatgtgattaattctcttgggtttggttatatattctactatctagttgcattcatacattagttctaagtttttattttgtagatgctttgggatttttctatgtagacaaccatgccatccccaaactgtgaaatttttacttttttcccttccacttgtatgcatttttatttctttttctttctttattactctggctagaatttctcctactatgctaaataaagttgtgagcagacatctttccattgttcccagtcttaggaagaaagctatcagtctttcactactaagtttgatgttacctgaaattttttgtagatattccttatcaagttgaagatattcccctctatttcaatttttctgagagttttttttcatgaaaaagtgttgaaatttttcagattttttttctgcctcaattagaatgatgattttttttttctttttgatttggtgatcatactgattgatttttgaatatggaatttgtcttgcatacctggggcaaaatatgcattgttcatggtgtgtaatcatttaatacattactgaattcaatttcctatgattttgtagagcaatttgtgttgaactttctgacacatagataggtgtagtttttgcccatcttttacttctttaatttttctttattttctttgttgggctttcgtatcaggataatactggcctcataaaataagttggagaacattctttttttattttctgaaagagattgtataaaattgtgctaattcttttgtaagtgttagagaattctctagtgaaattacctaggcctggagatttctctctcaagctattttaaattataaactctatttaataactacacaactatttagtttatttcaagttggttcattttggtaatttgtagcttttgaggaatttctcaattttaaaagaattgtcaaatgtgtgagtataaaaatagtttgtattattcccttgttgttctaatcattatgaaataataccacttgtttcattaccaatgtttatgataattgtcatgactattttttggatttttaacacttttattaactttttgggataaaatttttgttttgttggttttctctattgtttttctgtttgattcttgtttgtgtgtgctgtttataatttctttcatttgcttgctttgggttattattttctctttttttctagtctcttaaggagcttagattgtttatttgaagcattttctcttttcaaaaataatcatatagtattataatttttcttttattgctattttagctgcatctcattaatattgatgtttgttttaattttctttcaattctctgtatattttatttgacttccttttttatccatggattatttaaaagtgtgttttttaattttcaaatgtttagagattttcctcttgtctatcattgatttcaaatttgattctattaaaattaaatagtatattctgcatgatttcgattattttaaatctactgaggttagtttagtgagccagcatatggtctatcctggtgaatgtttggtggacaattaaatagactgcatattgtgctgttattgagtggaatgttttatcaatgtaagttgaatcttattggttgacagtgtttttccattctcctctatctttgctctctttttatctaatgcttctatttcctgctaacagtgggaatttgaagttctcaactacaggtatagattggcccatttcttctttcagctccatccaattttgctttgtgtattgaaaagctctgtttttttttttttttttttttttttttgcacatacacatttaggacacgatgtctcttgttagattaatcctgtcattactatgtaatacctttctttgtccctactaattttctttgctcttaagtccactttatgtgttatgcaattatatatatattacatatatttatatgtaatatatatcattatatattgtattgcctcagagctaaagcaattttacctcctaaggtaaaatagagtatgcaaacaaatattggccaaataccttacttttaaaaattaatgtttgcataacatattttcatccatcattttattcttaacacatctgtgtcattatttttgaagtgagcttttgtagattcaatgtagtggggtcatcgtttttatccattctgtcaacctctgtcttttaattgatgtatttattccatttatttgtcaagtaattatattatggcttaagtctgctaatttactgtttgttttctgtttcaaattatgccctttctcttttcttgcttttctgtgggttccttgacctagttttattttagaattacatttttgtttataaaatttttgaataattttgtataattttcttaatatgtgctcaaggtatgacaataaacacttgcaacttcaaaaagacatgcaaactcttaaaatgtttatgtgaacaaatattatcttctttacctgtggcaaatgataactgggctagagatttttataagttcttccagttctaacaatctgtattatgtgatatttactgttataaatctattaaaatttgaaaatgacagtgctgtatataacaaaataaaagcctatgactctatccatgctacttagcagggctttttgaactggtttgtccagagtttaaagagttatttcgaaactattcaggaccctcttgcttgcttgtggtaattcagaattattcagtayataagaggagttttaagcacttgtgttcttcgtgctcttaaaaataaatgcaccctaatttgcttcttgtattttttataattttacatagtaataaagacttcaatagttttaagatttgtaaaagtcactattgtaatataagctgacacctgtgattggtttatttggttgtagatcactgaacaactgagaaaagaaaaacaaaagcacagagttagcaaagtttctattatcaccaatataggccaaaaattccacgcatttattcagtggcaacaagtaatgtaggttttttgttgtttgtttgctttttctgagatatctactctgtgccaggcatgatttgaggcctcagctatgttggtgaaataggcaggtaaagactgtagggaagatggaattaaagttactgttgaagcccgaaaaaattctagttctttttgatccttccaagagctcagatgctaagatgatttgttgttcccacaatccatgcatcattaaataaacccataatcactaagtaaactatatattttctgttctttacattctgaaagcttctaagggacacccttagccatgatactgtatggtcttgaaaatttggctaaaatcatttctatttgtatacctctggatgttttaaacactacagggcttttaaaaagtgacattttgaatagataagaaaataaacattggtatcagcaaatattagatattactgtgagttcttgctgttatgcttgttaaaaagtatcatgatggaaagaaatcaggatttagaatcaaacaaatctgtgttcaagacccttagctgtttttgttagttgaattcaatgctggctgaactctaaagactcagttttttcaccatgtaaacaataatcattatataaagctaatagagctggaaagataaagtattatgggtaaactgctttccttagtttatctacttaagtagtgctatttcctttctttatttacttaccttgatccaagtgaaagcctagttttaggatttacttttaccaaaattgtactctgaaccaaatgagctcaaaagttmaactttgaaaagtcaaaatcatagaaaatttttacattgaaactgctcaataattgttttcaaaatgcacagcaggacttctcttatttaccatggtgaataaaatgactttagtttttgacatttgaagtatataataataaaagttatagaagttagaaagcttccttcagggaaatggaatatttttacttcctatgaaagtgatattttggtctcaatttaagtaacacaaaacttagttttcatttcctggttttaaatgataagctgtatattcctagtttacagtaaaccacatatttttagagagttagaaacgttgcttcaaagtggtagatattaagtgtcataaatgtccaggctgtaagtatggtttcctgytgtgtaagacataagttattaggtttagtttgaaatgacacaaaatgttcccgacttctaacaattaaaagaagaaatgtgaggctccgagacaatctcggcaaatttgtattctgattaaaataattatttgatcctatacagttcagtgcatggaaaaatgttgtctagttactcaattcaatgtggttgttagagaagaaaatatgtctcttatgtggggtttgagcttcatgtagctttgtgattgtatttgtttactcacggtggaaatctccttattaaattgtgggcaagaggcaacccatggtggaaatctccgtattaaattgtgggcaagaggcacttctattggtttttctaacagtaactacttacttcaggaaggattaggttaaaatactaaatgtagatctctagagttgatcttgtaacagaactagaacacattgacttgatatttagacatgtgcagaaggcaaaccatttcataaagggctggatataatttcttctttcagttctaagtcaaaatttaataatttttttctatttttaataaatatatgggcatatgcatctaatagtgaggtttggtaaagactcttcttcactttagttttgcatgtgtgggcaactgacctttatgttgttacaaaatactacttaaaaataaaaagctcaattctgtacagaaatgagccattgttcgttgttaggctctcattagctgttatagtatcaacactattgtcaacatgatgtggatcttgaattgttttcccgtctccactgctaactgtgtgttcctcttgttcttcgggccagttgaaccatacatcttgtgtcctaaaaccatatggagcatgtttttgtatgaactctcatgattaaggacatctgtctcagcctctacgtttgccttcaggggatctttgcatcatttaatggtacagttaacagcatgttttgcatcatgaagttggccgtacagtttttctgcggattctactgttttcattccttaaaaaaacacatggacgagtctatattttgtctttctgtctcccattctaactttcttgcttttcttcctccctcattctctttcttttttttttctattaatatttactggccatcaccttgtgtttagcaggtcttataatattccaaaattgcacattttcaaaactgtaaagattgaccacaatttctaaatgtaattcacttttcctttcaattgcttgttttgagtgtaatttggataccctaaagttcatgtgttacttttctatttattttgagtaatatgattcagattcaactcctttagatattactgaaaatattcttaagtgaattgatacaaaatatccacattcctcataagttatcaaaccaaaatgttgctaactctatactgaaattctactaattcttctgactgtacagattgattcgtcagtatatattagcaagtatttaattaaaatctattttaataactttaaatggatatagtagcactctaaataggaaatgcagtcattgaaactctcaaacactagggaatagatataaaatatgtgcatttaatcaattatatgttgttgatttttactttataatctatgacttcctttgttgatgtcaatattgactttctttaaaaatctctaaattatgaaaaagtctaagttattgtaatctacattatagactttttctagtatgttttactcataggtaaattgttactagtcccattaattgcaaaatgcagtggtcatgtgtttccaaataatttcaaattgtagaaataaatacagacagtctccatgaaagcatattgattcaagaattctatttattgatatttatttactcttcctaacatttatatttactcttcatagtgcataatgtttgttcagcagaaatatcatcaaaaaactattgatgtgccagttaccatgatgcaccgaagatacagaaacgaacaagatgtccccactctggtgaaggagtctatttgttgttggtattgacagtgtgagaggcagatgtagaaacaagattgacagactttgtatttaaaggtgtaagtttcataaatggrtacatagaaaaatgaacacgtttaaattaatgatgtaaacatgttaatattcccaaatacaaatataagtctgtacagtgacttgcttttttaaaaagcattgaagtggaatttgcataccatgaaattcacccattttaagtgtatartatgttgatttttgacaaattttaaaatcttgctagcatcaacacagtctagttttaaaacccgttcctcactccagaatgttcccttgtgcccttttgcagtccctcactccacattccagccccaaacaatcactgatctgctttttatctctgtagattttccttttctagatatttcatgaaaatgcaataataaataatgtataaatcattaaagatttatagcaaatgtagttttgttacccttttttcatgtttctcaaattgtttgtacaaggaaaattatttcagttggtattttgaaatgtgggagaggcctttgactaagaactttaggagtaacacccctaactctcattatcttcattttacagatatgaagactgtgctttataaaagatcagacatttggctaatgacactgctactaagtgacaacagagatttaaaactagttctgtctaattctaaatccactgatacagtttctcagaaagttgaaaaacttttaacattataaactcattgatagtgatggatatttgttcttttgttgtttagtgattacctcaagatctacaacaatgatcagcaatacaatgggagttttagttgtaatcagctgtttcatattttaataattattaaacactcatgtaccaaatacaatgctagctgcctgaaattacaaaaggaataaggtttgatataaagtaccacataggccaggcgcagtggctcacatgtgtaacaccaacactttgggaggccgaggtgtgtggaccacctgaggtcaggagttcgagacctgcctggctaacatggcgaaaccttgtctctactaaaaatgcacttagctgggtgtggtggcagacacctataattccagctacttgggaggctgaagcaggagaattgctcgaaccctggaggcggaggttacagtgagccgagattgtgccattgcactccagcctgggagaaagagcaagactctgtctcagaacaaaaggtaccatgtagttttcacttgttctaaaaatctcctctcattgtgtaactcaagtataaaaatccatattacatctgaaaaaggagttattcttctgaactctctctaccctccttaaagccaagtatatgccactttgctatgtcagttgatttgacttatatttggtacacagattgcatttttttttttgtaggacagaagaactccctgtcaagctagtttaactgtattacatctaccaatattcaattatttaattttttcaaaagtgaatgcaatcataatacatccaaatacaacctgaaactgttgggaagtaatgcttaggacaaaatttcaatctggtttactggaaaattttagcaataagtaaaaatcttaatgtgtttccatatgcatggtttcacacactttttatcaagtactagaaaaacactgaatgctaatgccattgaattttgctgaattagtgtactatataactcctttcatattaaagattttacccacttaacctcaccagaagtagtgttttcacagtgcatgattatgacaaagatagaatgatagggagaggggaaagtgccatcaggttcagggtcaacagaaaataaaactcgttctctttttgtaatcagttataattcccatttcaatcaaaaaaattattgtaaagtattttcttgaaatttgcttaaatttttcatttttaacaagaaacgttatttataaaacacattacttgtgtgcatataggtgttgggtctgagaaagtatacattattaatttgatgtgttattaaaagattgctgtcaaaaatctgtttagttaattgatgctgtcaattgctcgcattaatacctaatactcataatatttgtacctaaatttttcttataaaattgtttctcattggtttttattctttttatttctcctattttttaaaattgaagtttgtagtcaggtcatgtgaccacatcaagtccatcattcaaacacgtatcaggaaattggaaatttaaaaatgacttatgcatggatatgatgccagaggcagagacaagggggtgggggctgtttctaaataccttccaataacaataaaaaaacggaagttatttgtgatcttagaagatggctaaggaaaaactgaaacctgaaactraggaaacaaaagtttagacaagataatcccaacatcagagaaagcatttgtcattgtcagaactccaaagagcaagaaagctttaawccatttcatggaccctaggactcatggtctagtaattcttatgtcaccaaaccatcttaggatctgtatgaaattacagtgtgtcaccactctacatctgcaatcttaatgtttccgtcctttgattgtatgacaaggaaactcttgaaccagagcactgcacttcacaggagtctgaaagaaccagctgctcataattttggagttttatagggctttttagtctcagaatccctgtctgacctatgtccatatccagaattcattttttttcatttccagaattcttcagaaaaaactcttgctgtcttttaacgtcttaaaacatttttcaaaaaatatatagagagaattagaaagccactcatagacttaccctctttctccgttgcttcttcagttgtgtaatagtcatgaaatctggaatgaccattttaaattatattcactgagtttgaaacaaaacgtttttagatggttttgttcccttaaaaatgaatattattgtctagcattaacattcacccaaattgatagcatattgaattatgtgtgtagattcttaaacgttttgttgtgaaacatcaaaagaaatcactgactttttatttaacataaataaattaagggaagtttagaaatctaattgttggaaaaataaacttctaaacataactcttcaaaagtctacgtgctcacattttctattcccgaacaaaaacttttgcaaagctctctgaaagccttgtaaaaggccaatgaatttcatttagcattaatttccctactcaccacatagcaaatatatgacacaayttatagttctgttaaaaaaaagccagaactgttacctgagcaactgcttataaaagagcgaggcagcttacgttttatctattttttgtgtgctttctactaagttttacaatctaagtgtgttagaaataaaatcaaattcattaggaatctctatgcataaaaggaaattaaatatcatactcaatgacttgtgccacctgtggcaaatagttttacatgccaat

SEQ ID NO: 2 - DNA, 2831 bp, Homo sapiens
ggacagcacactgcaaggaaatcttcacgggtgtggagtacagttcttgtgacacaatagccaagatttccacagatgacatgaaaaaggttggtgtcaccgtggttgggccacagaagaagatcatcagtagcattaaagctctagaaacgcaatcaaagaatggcccagttcccgtgtaaagcacgggacggaagtgcttctggacggaagtggtggctgtggaaggcgtagcatcatcctgcagacagacaataattctggagatactggtggaagttccaagtccaataagacactcaaatatgagtacaaatgccttaaaatggaattgaaaaactctttattttcccctatcatttattggatgggtgggtggggtatttttttgtaattgcttttttaaatattagttaatggattaaatttaattcttcagcgtaaaatggtgaagaactagcatatagccattgatcataaactgactatcataaaatcaaaacaagtgaaataacaaaatggacatggtggctttgtttaggtagagccacaaaagaaaagacttgtaatatttttatatacagaggaaatctgtaacaggtattttgtttcttttaaagcaagcaacacagaggaatttatacctcaaactatctggccatatttactaccttatcactgcattattctcttttatctgtttaaagcatatagagatgaagtttgtagttgttttaagtactacacatttttaaattgttagcttccttaagtatatcatgtaaagaaatgtcttaatttttgaaaaaagtacatatttattttcttttgaattgtttttattgttttctatttatgccttgatgatttaatatggatttgttacagccaagtgccaaatgctctctcaaattgtcagcaatttaactagacacagataataatgggtttctttcagattttttgaaccatccacttacatatatttttaaaaaatgaaatccttttcctgttcatacactaaccaaatctctcaaatctgttatcccaatcattgttgcctctccgtttattataaactgtatgctcacaacttagtgtaatataccagcttgtatgcaatggattttcaaccagataacatacctttcctgctctggtgcttagagactatcaactccctcctttagtgaaggagccgtgttagagcttccgagaatagctccactggagagaagtggaatcctatatagaatgctgcactaattgacaacacagcctataggccaatgcatgagtaaaaaaaaaaacaattactggctcactggctttgaaaagtcacttactattgttgctgaaacttgctgagctgtttatagagaatgatgataacagaacttttcctctgtatcactggtgtttaggtgaattaattaaacattgtgatcattagtaccaggtattattatctttaagagtcttccacttcaatgcacatggtgcagttttggtgtgtaacttagaaggattgaacttctttgaatttactggacataacattttcagaatagttggtcatctagcaaccgcctcaaaatgtgtaagcaggagagaaatttctcatcacagggatttagacttactattacataaaggctaactatgagcttgctcattaattttgaaaagatgtacctggtggatatctagctagtaatatattctgaagcaacattttagctctattgatactctttctaatgctgatatgatcttgagtataagaaatgcatatgtcactagaatggataaaataatgctgcaaacttaatgttcttatgcaaaatggaacgctaatgaaacacagcttacaatcgcaaatcaaaactcacaagtgctcatctgttgtagatttagtgtaataagacttagattgtgctccttcggatatgattgtttctcaaatcttggcaatattccttagtcaaatcaggctactagaattctgtattggatatataagagcatgaaatttttaaaaatacacttgtgattataaaattaatcacaaatttcacttatacctgctatcagcagctagaaaacattttttttttaaatcaagtattttgtgtttggaatgttagaatgagatctgaatgtggtttcaatctaattttttcccagactactattttcttttttaggtactattctgagcatactcaacaaaacccatgcatttcataaactaatagaagttgaggattgttgaatctatttcacttattttggctgtggtttccatctgaaagtagaggttgtatacaccatatactgttcttcattttattaatatttttctccttgacctctcataaatttactttacacaattcttaccctgtacatatgtaaacataagtgtacgattcttaaccatggagtagaggtactagaatgcttacggccatctctttgtacaggaactgcattgactttcagtaaacataaagccacaactcctacatgatgttatgtaccatatgatctgttttgtatcttaaatttgatttacatatattatttatttctggtaactcactcagtttatgctgtgctaaatatcaatcaagccatgtataaatgtgatatgattggcaatatgtgtttactttaaacttgtcttttcaaaatattactcagtttatgttgtacaatgtagatggcctcttactaatgtaaaatgatttgtagtggaaacatttatatttttataataaacataatgaaaatattttttacagattggaaaaaaaaaaaaaaaaaaa

SEQ ID NO: 3 - DNA, 2546 bp, Homo sapiens
cttctccagcaatcagagcgctccccctcacatcagtggcatgcttcatggagatatgctcctctcactgccctctgcaccagcaacatggattgtcagctctccatcctcctccttctcagctgctctgttctcgacagcttcggggaactgattccgcagccttccaatgaagtcaatctactggattcaaaaacaattcaaggggagctgggctggatctcttatccatcacatgggtgggaagagatcagtggtgtggatgaacattacacacccatcaggacttaccaggtgtgcaatgtcatggaccacagtcaaaacaattggctgagaacaaactgggtccccaggaactcagctcagaagatttatgtggagctcaagttcactctacgagactgcaatagcattccattggttttaggaacttgcaaggagacattcaacctgtactacatggagtctgatgatgatcatggggtgaaatttcgagagcatcagtttacaaagattgacaccattgcagctgatgaaagtttcactcaaatggatcttggggaccgtattctgaagctcaacactgagattagagaagtaggtcctgtcaacaagaagggattttatttggcatttcaagatgttggtgcttgtgttgccttggtgtctgtgagagtatacttcaaaaagtgcccatttacagtgaagaatctggctatgtttccagacacggtacccatggactcccagtccctggtggaggttagagggtcttgtgtcaacaattctaaggaggaagatcctccaaggatgtactgcagtacagaaggcgaatggcttgtacccattggcaagtgttcctgcaatgctggctatgaagaaagaggttttatgtgccaagcttgtcgaccaggtttctacaaggcattggatggtaatatgaagtgtgctaagtgcccgcctcacagttctactcaggaagatggttcaatgaactgcaggtgtgagaataattacttccgggcagacaaagaccctccatccatggcttgtacccgacctccatcttcaccaagaaatgttatctctaatataaacgagacctcagttatcctggactggagttggcccctggacacaggaggccggaaagatgttaccttcaacatcatatgtaaaaaatgtgggtggaatataaaacagtgtgagccatgcagcccaaatgtccgcttcctccctcgacagtttggactcaccaacaccacggtgacagtgacagaccttctggcacatactaactacacctttgagattgatgccgttaatggggtgtcagagctgagctccccaccaagacagtttgctgcggtcagcatcacaactaatcaggctgctccatcacctgtcctgacgattaagaaagatcggacctccagaaatagcatctctttgtcctggcaagaacctgaacatcctaatgggatcatattggactacgaggtcaaatactatgaaaagcaggaacaagaaacaagttataccattctgagggcaagaggcacaaatgttaccatcagtagcctcaagcctgacactatatacgtattccaaatccgagcccgaacagccgctggatatgggacgaacagccgcaagtttgagtttgaaactagtccagactgtatgtattatttcaatgcagtctagaggagggggcagggatcttgcaaaagatgtctgatcgtttattctcactgtttctaagttttaaacaaatgtgatacatttaaggtatattgcttgggacattgcaatttgcagagccctgtgtctgtatacagtatttgtgtttgtgtgggtgtacattttgtgtttctttttttcttgtatgcaaatcaaacatattctaatgcctgaaatgcttctgttttttttttttagccataaattgcttttgaggaacattatttaatatagtaacacacttccagtgtctgtcatttcagatattccaggttcattgcgtgattcaatgaaccacaaaaaagaaacttgctgatccatgagaatcttaattttgttttaatccttaacacattcaatagcatatcacagagagaataaggattttctaaaatgtgttttatcacttcattcacattcagaagtaatttgaatagcctgttcctttaaccccaaatttggctaaaattggcctaaaactggcaaacatttttccagtaacttttctttttttcaaatgaattttcttcatacttaaaaaagccctttgctaaaatataattttcaaaaaggtaaaattatgtctatggcactaatataaaatgagtagaagttaatgattttacttaactcatttttttctttctttctttttttttttttttttttgagacggagtcttgctctgtcacccaggctggagtacagcagagcgatctcggctcactgcaagctcctgcaagctccgcctcctggcttcacgccattctccccctcagcctcccgagtagctgggactacag

SEQ ID NO: 4 - protein, 983 aa, Homo sapiens
MDCQLSILLLLSCSVLDSFGELIPQPSNEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDSFSISGESSQVVMIAISAAVAIILLTVVIYVLIGRFCGYKSKHGADEKRLHFGNGHLKLPGLRTYVDPHTYEDPTQAVHEFAKELDATNISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLIRNPGSLKIITSAAARPSNLLLDQSNVDITTFRTTGDWLNGVWTAHCKEIFTGVEYSSCDTIAKISTDDMKKVGVTVVGPQKKIISSIKALETQSKNGPVPV

SEQ ID NO: 5 - protein, 539 aa, Homo sapiens
MDCQLSILLLLSCSVLDSFGELIPQPSNEVNLLDSKTIQGELGWISYPSHGWEEISGVDEHYTPIRTYQVCNVMDHSQNNWLRTNWVPRNSAQKIYVELKFTLRDCNSIPLVLGTCKETFNLYYMESDDDHGVKFREHQFTKIDTIAADESFTQMDLGDRILKLNTEIREVGPVNKKGFYLAFQDVGACVALVSVRVYFKKCPFTVKNLAMFPDTVPMDSQSLVEVRGSCVNNSKEEDPPRMYCSTEGEWLVPIGKCSCNAGYEERGFMCQACRPGFYKALDGNMKCAKCPPHSSTQEDGSMNCRCENNYFRADKDPPSMACTRPPSSPRNVISNINETSVILDWSWPLDTGGRKDVTFNIICKKCGWNIKQCEPCSPNVRFLPRQFGLTNTTVTVTDLLAHTNYTFEIDAVNGVSELSSPPRQFAAVSITTNQAAPSPVLTIKKDRTSRNSISLSWQEPEHPNGIILDYEVKYYEKQEQETSYTILRARGTNVTISSLKPDTIYVFQIRARTAAGYGTNSRKFEFETSPDCMYYFNAV

SEQ ID NO: 6 - DNA, 1574 bp, Homo sapiens
gcttctctccatcttgtgattcctttttcctcctgaaccctccagtgggggtgcgagtttgtctttatcaccccccatcccaccgccttcttttcttctcgctctcctacccctccccagcttggtgggcgcctctttcctttctcgccccctttcatttttatttattcatatttatttggcgcccgctctctctctgtccctttgcctgcctccctccctccggatccccgctctctccccggagtggcgcgtcgggggctccgccgctggccaggcgtgatgttgcacgtggagatgttgacgctggtgtttctggtgctctggatgtgtgtgttcagccaggacccgggctccaaggccgtcgccgaccgctacgctgtctactggaacagcagcaaccccagattccagaggggtgactaccatattgatgtctgtatcaatgactacctggatgttttctgccctcactatgaggactccgtcccagaagataagactgagcgctatgtcctctacatggtgaactttgatggctacagtgcctgcgaccacacttccaaagggttcaagagatgggaatgtaaccggcctcactctccaaatggaccgctgaagttctctgaaaaattccagctcttcactcccttttctctaggatttgaattcaggccaggccgagaatatttctacatctcctctgcaatcccagataatggaagaaggtcctgtctaaagctcaaagtctttgtgagaccaacaaatagctgtatgaaaactataggtgttcatgatcgtgttttcgatgttaacgacaaagtagaaaattcattagaaccagcagatgacaccgtacatgagtcagccgagccatcccgcggcgagaacgcggcacaaacaccaaggatacccagccgccttttggcaatcctactgttcctcctggcgatgcttttgacattatagcacagtctcctcccatcacttgtcacagaaaacatcagggtcttggaacaccagagatccacctaactgctcatcctaagaagggacttgttattgggttttggcagatgtcagatttttgttttctttctttcagcctgaattctaagcaacaacttcaggttgggggcctaaacttgttcctgcctccctcaccccaccccgccccacccccagccctggcccttggcttctctcacccctcccaaattaaatggactccagatgaaaatgccaaattgtcatagtgacaccagtggttcgtcagctcctgtgcattctcctctaagaactcacctccgttagcgcactgtgtcagcgggctatggacaaggaagaatagtggcagatgcagccagcgctggctagggctgggagggttttgctctcctatgcaatatttatgccttctcattcagaactgtaagatgatcgcgcagggcatcatgtcaccatgtcaggtccggaggggaggtattaagaatagatacgatattacaccatttcctataggagtatgtaaatgaacaggcttctaaaaggttgagacactggttttttttttt

SEQ ID NO: 7 - protein, 228 aa, Homo sapiens
MLHVEMLTLVFLVLWMCVFSQDPGSKAVADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDNGRRSCLKLKVFVRPTNSCMKTIGVHDRVFDVNDKVENSLEPADDTVHESAEPSRGENAAQTPRIPSRLLAILLFLLAMLLTL

SEQ ID NO: 8 - DNA, 3410 bp, Homo sapiens
gaattcgaggatccgggtaccatgggcggcggcaggcctagcagcacgggaaccgtcccccgcgcgcatgcgcgcgcccctgaagcgcctgggggacgggtatgggcgggaggtaggggcgcggctccgcgtgccagttgggtgcccgcgcgtcacgtggtgaggaaggaggcggaggtctgagtttcgagggagggggggagagaagagggaacgagcaagggaaggaaagcggggaaaggaggaaggaaacgaacgagggggagggaggtccctgttttggaggagctaggagcgttgccggcccctgaagtggagcgagagggaggtgcttcgccgtttctcctgccaggggaggtcccggcttcccgtggaggctccggaccaagccccttcagcttctccctccggatcgatgtgctgctgttaacccgtgaggaggcggcggcggcggcagcggcagcggaagatggtgttgctgagagtgttaattctgctcctctcctgggcggcggggatgggaggtcagtatgggaatcctttaaataaatatatcagacattatgaaggattatcttacaatgtggattcattacaccaaaaacaccagcgtgccaaaagagcagtctcacatgaagaccaatttttacgtctagatttccatgcccatggaagacatttcaacctacgaatgaagagggacacttcccttttcagtgatgaatttaaagtagaaacatcaaataaagtacttgattatgatacctctcatatttacactggacatatttatggtgaagaaggaagttttagccatgggtctgttattgatggaagatttgaaggattcatccagactcgtggtggcacattttatgttgagccagcagagagatatattaaagaccgaactctgccatttcactctgtcatttatcatgaagatgatattaactatccccataaatacggtcctcaggggggctgtgcagatcattcagtatttgaaagaatgaggaaataccagatgactggtgtagaggaagtaacacagatacctcaagaagaacatgctgctaatggtccagaacttctgaggaaaaaacgtacaacttcagctgaaaaaaatacttgtcagctttatattcagactgatcatttgttctttaaatattacggaacacgagaagctgtgattgcccagatatccagtcatgttaaagcgattgatacaatttaccagaccacagacttctccggaatccgtaacatcagtttcatggtgaaacgcataagaatcaatacaactgctgatgagaaggaccctacaaatcctttccgtttcccaaatattggtgtggagaagtttctggaattgaattctgagcagaatcatgatgactactgtttggcctatgtcttcacagaccgagattttgatgatggcgtacttggtctggcttgggttggagcaccttcaggaagctctggaggaatatgtgaaaaaagtaaactctattcagatggtaagaagaagtccttaaacactggaattattactgttcagaactatgggtctcatgtacctcccaaagtctctcacattacttttgctcacgaagttggacataactttggatccccacatgattctggaacagagtgcacaccaggagaatctaagaatttgggtcaaaaagaaaatggcaattacatcatgtatgcaagagcaacatctggggacaaacttaacaacaataaattctcactctgtagtattagaaatataagccaagttcttgagaagaagagaaacaactgttttgttgaatctggccaacctatttgtggaaatggaatggtagaacaaggtgaagaatgtgattgtggctatagtgaccagtgtaaagatgaatgctgcttcgatgcaaatcaaccagagggaagaaaatgcaaactgaaacctgggaaacagtgcagtccaagtcaaggtccttgttgtacagcacagtgtgcattcaagtcaaagtctgagaagtgtcgggatgattcagactgtgcaagggaaggaatatgtaatggcttcacagctctctgcccagcatctgaccctaaaccaaacttcacagactgtaataggcatacacaagtgtgcattaatgggcaatgtgcaggttctatctgtgagaaatatggcttagaggagtgtacgtgtgccagttctgatggcaaagatgataaagaattatgccatgtatgctgtatgaagaaaatggacccatcaacttgtgccagtacagggtctgtgcagtggagtaggcacttcagtggtcgaaccatcaccctgcaacctggatccccttgcaacgattttagaggttactgtgatgttttcatgcggtgcagattagtagatgctgatggtcctctagctaggcttaaaaaagcaatttttagtccagagctctatgaaaacattgctgaatggattgtggctcattggtgggcagtattacttatgggaattgctctgatcatgctaatggctggatttattaagatatgcagtgttcatactccaagtagtaatccaaagttgcctcctcctaaaccacttccaggcactttaaagaggaggagacctccacagcccattcagcaaccccagcgtcagcggccccgagagagttatcaaatgggacacatgagacgctaactgcagcttttgccttggttcttcctagtgcctacaatgggaaaacttcactccaaagagaaacctattaagtcatcatctccaaactaaaccctcacaagtaacagttgaagaaaaaatggcaagagatcatatcctcagaccaggtggaattacttaaattttaaagcctgaaaattccaatttgggggtgggaggtggaaaaggaacccaattttcttatgaacagatatttttaacttaatggcacaaagtcttagaatattattatgtgccccgtgttccctgttcttcgttgctgcattttcttcacttgcaggcaaacttggctctcaataaacttttaccacaaattgaaataaatatatttttttcaactgccaatcaaggctaggaggctcgaccacctcaacattggagacatcacttgccaatgtacataccttgttatatgcagacatgtatttcttacgtacactgtacttctgtgtgcaattgtaaacagaaattgcaatatggatgtttctttgtattataaaatttttccgctcttaattaaaaattactgtttaattgacatactcaggataacagagaatggtggtattcagtggtccaggattctgtaatgctttacacaggcagttttgaaatgaaaatcaatttaccccatggtacccggatcctcgaattc

SEQ ID NO: 9 - protein, 748 aa, Homo sapiens
MVLLRVLILLLSWAAGMGGQYGNPLNKYIRHYEGLSYNVDSLHQKHQRAKRAVSHEDQFLRLDFHAHGRHFNLRMKRDTSLFSDEFKVETSNKVLDYDTSHIYTGHIYGEEGSFSHGSVIDGRFEGFIQTRGGTFYVEPAERYIKDRTLPFHSVIYHEDDINYPHKYGPQGGCADHSVFERMRKYQMTGVEEVTQIPQEEHAANGPELLRKKRTTSAEKNTCQLYIQTDHLFFKYYGTREAVIAQISSHVKAIDTIYQTTDFSGIRNISFMVKRIRINTTADEKDPTNPFRFPNIGVEKFLELNSEQNHDDYCLAYVFTDRDFDDGVLGLAWVGAPSGSSGGICEKSKLYSDGKKKSLNTGIITVQNYGSHVPPKVSHITFAHEVGHNFGSPHDSGTECTPGESKNLGQKENGNYIMYARATSGDKLNNNKFSLCSIRNISQVLEKKRNNCFVESGQPICGNGMVEQGEECDCGYSDQCKDECCFDANQPEGRKCKLKPGKQCSPSQGPCCTAQCAFKSKSEKCRDDSDCAREGICNGFTALCPASDPKPNFTDCNRHTQVCINGQCAGSICEKYGLEECTCASSDGKDDKELCHVCCMKKMDPSTCASTGSVQWSRHFSGRTITLQPGSPCNDFRGYCDVFMRCRLVDADGPLARLKKAIFSPELYENIAEWIVAHWWAVLLMGIALIMLMAGFIKICSVHTPSSNPKLPPPKPLPGTLKRRRPPQPIQQPQRQRPRESYQMGHMRR

SEQ ID NO: 10 - DNA, 23 bp, Artificial Sequence
  Description of Artificial Sequence Synthetic nucleotide motif
aannnnnnnnnnnnnnnnnnntt

SEQ ID NO: 11 - DNA, 21 bp, Artificial Sequence
  Description of Artificial Sequence Synthetic primer
cagggcttagagtttattgag

SEQ ID NO: 12 - DNA, 20 bp, Artificial Sequence
  Description of Artificial Sequence Synthetic primer
ctcgttcttcatcactattt

SEQ ID NO: 13 - DNA, 24 bp, Artificial Sequence
  Description of Artificial Sequence Synthetic primer
actattttaattctgttttctgtg

SEQ ID NO: 14 - DNA, 20 bp, Artificial Sequence
  Description of Artificial Sequence Synthetic primer
cttttacacgctctgctatg

SEQ ID NO: 15 - DNA, 20 bp, Artificial Sequence
  Description of Artificial Sequence Synthetic primer
tgaccatgtcattcctatgc

SEQ ID NO: 16 - DNA, 20 bp, Artificial Sequence
  Description of Artificial Sequence Synthetic primer
attgggaggtgagttcacag

SEQ ID NO: 17 - DNA, 20 bp, Artificial Sequence
  Description of Artificial Sequence Synthetic primer
agcaatcagtggcagcaaac

SEQ ID NO: 18 - DNA, 20 bp, Artificial Sequence
  Description of Artificial Sequence Synthetic primer
tcttggcaaaaaagacaagc

SEQ ID NO: 19 - DNA, 20 bp, Artificial Sequence
  Description of Artificial Sequence Synthetic primer
caacagtttctcatgctagg

SEQ ID NO: 20 - DNA, 20 bp, Artificial Sequence
  Description of Artificial Sequence Synthetic primer
ggatagcagctgctcttttg

SEQ ID NO: 21 - DNA, 20 bp, Artificial Sequence
  Description of Artificial Sequence Synthetic primer
agggcaaatagggaatgcag

SEQ ID NO: 22 - DNA, 20 bp, Artificial Sequence
  Description of Artificial Sequence Synthetic primer
taccataggagttacattgc

SEQ ID NO: 23 - DNA, 20 bp, Artificial Sequence
  Description of Artificial Sequence Synthetic primer
ccctgagttttaaaaaagtg

SEQ ID NO: 24 - DNA, 20 bp, Artificial Sequence
  Description of Artificial Sequence Synthetic primer
aacaaacccacaaatgtcac

SEQ ID NO: 25 - DNA, 20 bp, Artificial Sequence
  Description of Artificial Sequence Synthetic primer
aggtttaagatttcctgtac

SEQ ID NO: 26 - DNA, 20 bp, Artificial Sequence
  Description of Artificial Sequence Synthetic primer
atcagcattcctggagtaag

SEQ ID NO: 27 - DNA, 20 bp, Artificial Sequence
  Description of Artificial Sequence Synthetic primer
tatgaggtatggttatgaag

SEQ ID NO: 28 - DNA, 21 bp, Artificial Sequence
  Description of Artificial Sequence Synthetic primer
ccattatctgctaataggtag

SEQ ID NO: 29 - DNA, 21 bp, Artificial Sequence
  Description of Artificial Sequence Synthetic primer
gataagagtcttgaatctgtg

SEQ ID NO: 30 - DNA, 20 bp, Artificial Sequence
  Description of Artificial Sequence Synthetic primer
catcaccagaagctatagcc

SEQ ID NO: 31 - DNA, 20 bp, Artificial Sequence
  Description of Artificial Sequence Synthetic primer
tcacctcccagagaacaatc

SEQ ID NO: 32 - DNA, 20 bp, Artificial Sequence
  Description of Artificial Sequence Synthetic primer
ataatgtaactgggtggctc

SEQ ID NO: 33 - DNA, 20 bp, Artificial Sequence
  Description of Artificial Sequence Synthetic primer
cttgcaagacttggaaacag

SEQ ID NO: 34 - DNA, 20 bp, Artificial Sequence
  Description of Artificial Sequence Synthetic primer
cagtggaaaaaagaaggaag

SEQ ID NO: 35 - DNA, 20 bp, Artificial Sequence
  Description of Artificial Sequence Synthetic primer
taattactctgagaggcacc

SEQ ID NO: 36 - DNA, 20 bp, Artificial Sequence
  Description of Artificial Sequence Synthetic primer
gtcagcagggctttttaaac

SEQ ID NO: 37 - DNA, 20 bp, Artificial Sequence
  Description of Artificial Sequence Synthetic primer
gatatagtttcagtcacctg

SEQ ID NO: 38 - DNA, 20 bp, Artificial Sequence
  Description of Artificial Sequence Synthetic primer
gggaacactacagaaaaggg

SEQ ID NO: 39 - DNA, 20 bp, Artificial Sequence
  Description of Artificial Sequence Synthetic primer
cctatagtctagcaaaaccg

SEQ ID NO: 40 - DNA, 20 bp, Artificial Sequence
  Description of Artificial Sequence Synthetic primer
atagatcagggatcctcagc

SEQ ID NO: 41 - DNA, 20 bp, Artificial Sequence
  Description of Artificial Sequence Synthetic primer
tttgccttctgctttgccac

SEQ ID NO: 42 - DNA, 20 bp, Artificial Sequence
  Description of Artificial Sequence Synthetic primer
ctgtccactagactaaactc

SEQ ID NO: 43 - DNA, 20 bp, Artificial Sequence
  Description of Artificial Sequence Synthetic primer
tctctgagagttagtaaaac

SEQ ID NO: 44 - DNA, 20 bp, Artificial Sequence
  Description of Artificial Sequence Synthetic primer
attgcaacattggttctgac

SEQ ID NO: 45 - DNA, 20 bp, Artificial Sequence
  Description of Artificial Sequence Synthetic primer
cctccactacacaaatgacc

SEQ ID NO: 46 - DNA, 20 bp, Artificial Sequence
  Description of Artificial Sequence Synthetic primer
ataatggctacttttcaggg

SEQ ID NO: 47 - DNA, 21 bp, Artificial Sequence
  Description of Artificial Sequence Synthetic primer
gcaatgatcttgttaaccacc

SEQ ID NO: 48 - DNA, 20 bp, Artificial Sequence
  Description of Artificial Sequence Synthetic primer
cagcacactgcaaggaaatc

SEQ ID NO: 49 - DNA, 20 bp, Artificial Sequence
  Description of Artificial Sequence Synthetic primer
gtcatctgtggaaatcttgg

SEQ ID NO: 50 - DNA, 20 bp, Artificial Sequence
  Description of Artificial Sequence Synthetic primer
tgaactgaagcactcatccc

SEQ ID NO: 51 - DNA, 20 bp, Artificial Sequence
  Description of Artificial Sequence Synthetic primer
ggtgcagctatgaagaagtg

SEQ ID NO: 52 - DNA, 24 bp, Artificial Sequence
  Description of Artificial Sequence Synthetic primer
actattattttaggaaggggaaag

SEQ ID NO: 53 - DNA, 20 bp, Artificial Sequence
  Description of Artificial Sequence Synthetic primer
cttcttccaataccatatcc

SEQ ID NO: 54 - DNA, 20 bp, Artificial Sequence
  Description of Artificial Sequence Synthetic primer
tccccagataattctgtcac

SEQ ID NO: 55 - DNA, 21 bp, Artificial Sequence
  Description of Artificial Sequence Synthetic primer
caaagttaataaacacaactg

SEQ ID NO: 56 - DNA, 21 bp, Artificial Sequence
  Description of Artificial Sequence Synthetic primer
gctagctacatagttaagttc

SEQ ID NO: 57 - DNA, 20 bp, Artificial Sequence
  Description of Artificial Sequence Synthetic primer
attgcaaagaaggccacctg

SEQ ID NO: 58 - DNA, 20 bp, Artificial Sequence
  Description of Artificial Sequence Synthetic primer
gctctaccttggttaatgcc

SEQ ID NO: 59 - DNA, 20 bp, Artificial Sequence
  Description of Artificial Sequence Synthetic primer
gggatttaccacttgtgaag

SEQ ID NO: 60 - DNA, 20 bp, Artificial Sequence
  Description of Artificial Sequence Synthetic primer
gatcagactgtagggtatgc

SEQ ID NO: 61 - DNA, 20 bp, Artificial Sequence
  Description of Artificial Sequence Synthetic primer
cttcacaagtggtaaatccc

SEQ ID NO: 62 - DNA, 20 bp, Artificial Sequence
  Description of Artificial Sequence Synthetic primer
ctaaggtggcattgttaggg

SEQ ID NO: 63 - DNA, 20 bp, Artificial Sequence
  Description of Artificial Sequence Synthetic primer
tctcaagttgactcacttgg

SEQ ID NO: 64 - DNA, 20 bp, Artificial Sequence
  Description of Artificial Sequence Synthetic primer
ctaaggtggcattgttaggg

SEQ ID NO: 65 - DNA, 20 bp, Artificial Sequence
  Description of Artificial Sequence Synthetic primer
tctcaagttgactcacttgg

SEQ ID NO: 66 - DNA, 21 bp, Artificial Sequence
  Description of Artificial Sequence Synthetic primer
gtagttctctgagtattccac

SEQ ID NO: 67 - DNA, 21 bp, Artificial Sequence
  Description of Artificial Sequence Synthetic primer
ggacctgaagaatgattagag

SEQ ID NO: 68 - DNA, 20 bp, Artificial Sequence
  Description of Artificial Sequence Synthetic primer
gaacatgtttgcttgaaggg

SEQ ID NO: 69 - DNA, 20 bp, Artificial Sequence
  Description of Artificial Sequence Synthetic primer
ggaacaggcagttatccttg

SEQ ID NO: 70 - DNA, 20 bp, Artificial Sequence
  Description of Artificial Sequence Synthetic primer
ttgagtattaagggctctcc

SEQ ID NO: 71 - DNA, 20 bp, Artificial Sequence
  Description of Artificial Sequence Synthetic primer
ttggacacaatccttcagag

SEQ ID NO: 72 - DNA, 20 bp, Artificial Sequence
  Description of Artificial Sequence Synthetic primer
ttgtctctgacccagaaacc

SEQ ID NO: 73 - DNA, 20 bp, Artificial Sequence
  Description of Artificial Sequence Synthetic primer
aagccaacaatttggcctgc

SEQ ID NO: 74 - DNA, 20 bp, Artificial Sequence
  Description of Artificial Sequence Synthetic primer
aagagcacgaagaacacaag

SEQ ID NO: 75 - DNA, 20 bp, Artificial Sequence
  Description of Artificial Sequence Synthetic primer
actattcaggaccctcttgc

SEQ ID NO: 76 - DNA, 20 bp, Artificial Sequence
  Description of Artificial Sequence Synthetic primer
aagtcctgctgtgcattttg

SEQ ID NO: 77 - DNA, 20 bp, Artificial Sequence
  Description of Artificial Sequence Synthetic primer
gtactctgaaccaaatgagc

SEQ ID NO: 78 - DNA, 20 bp, Artificial Sequence
  Description of Artificial Sequence Synthetic primer
gaagtcgggaacattttgtg

SEQ ID NO: 79 - DNA, 20 bp, Artificial Sequence
  Description of Artificial Sequence Synthetic primer
agtgtcataaatgtccaggc

SEQ ID NO: 80 - DNA, 20 bp, Artificial Sequence
  Description of Artificial Sequence Synthetic primer
gaggcagatgtagaaacaag

SEQ ID NO: 81 - DNA, 20 bp, Artificial Sequence
  Description of Artificial Sequence Synthetic primer
aagcaagtcactgtacagac

SEQ ID NO: 82 - DNA, 20 bp, Artificial Sequence
  Description of Artificial Sequence Synthetic primer
gcataccatgaaattcaccc

SEQ ID NO: 83 - DNA, 20 bp, Artificial Sequence
  Description of Artificial Sequence Synthetic primer
actgtgttgatgctagcaag

SEQ ID NO: 84 - DNA, 21 bp, Artificial Sequence
  Description of Artificial Sequence Synthetic primer
ggctaaggaaaaactgaaacc

SEQ ID NO: 85 - DNA, 20 bp, Artificial Sequence
  Description of Artificial Sequence Synthetic primer
atgctttctctgatgttggg

SEQ ID NO: 86 - DNA, 20 bp, Artificial Sequence
  Description of Artificial Sequence Synthetic primer
tcagaactccaaagagcaag

SEQ ID NO: 87 - DNA, 20 bp, Artificial Sequence
  Description of Artificial Sequence Synthetic primer
tcctaagatggtttggtgac

SEQ ID NO: 88 - DNA, 20 bp, Artificial Sequence
  Description of Artificial Sequence Synthetic primer
ttccctactcaccacatagc

SEQ ID NO: 89 - DNA, 20 bp, Artificial Sequence
  Description of Artificial Sequence Synthetic primer
tgctcaggtaacagttctgg

SEQ ID NO: 90 - DNA, 22 bp, Artificial Sequence
  Description of Artificial Sequence Synthetic primer
gaaagttataaagagccaaaat

SEQ ID NO: 91 - DNA, 20 bp, Artificial Sequence
  Description of Artificial Sequence Synthetic primer
ctgtttcactattcactttc

SEQ ID NO: 92 - DNA, 20 bp, Artificial Sequence
  Description of Artificial Sequence Synthetic primer
aaaaaagacaagctagggct

SEQ ID NO: 93 - DNA, 19 bp, Artificial Sequence
  Description of Artificial Sequence Synthetic primer
gctcttttgttgtagctgt

SEQ ID NO: 94 - DNA, 20 bp, Artificial Sequence
  Description of Artificial Sequence Synthetic primer
gttacattgcaatttccttt

SEQ ID NO: 95 - DNA, 19 bp, Artificial Sequence
  Description of Artificial Sequence Synthetic primer
aaagcatttcttttcgaga

SEQ ID NO: 96 - DNA, 17 bp, Artificial Sequence
  Description of Artificial Sequence Synthetic primer
acttagaatgctgcctc

SEQ ID NO: 97 - DNA, 23 bp, Artificial Sequence
  Description of Artificial Sequence Synthetic primer
tgctaataggtagcttattagac

SEQ ID NO: 98 - DNA, 20 bp, Artificial Sequence
  Description of Artificial Sequence Synthetic primer
gaagctatagcctaccgcaa

SEQ ID NO: 99 - DNA, 17 bp, Artificial Sequence
  Description of Artificial Sequence Synthetic primer
tgggtggctcctggttt

SEQ ID NO: 100 - DNA, 19 bp, Artificial Sequence
  Description of Artificial Sequence Synthetic primer
agatgaattttgtcagggg

SEQ ID NO: 101 - DNA, 23 bp, Artificial Sequence
  Description of Artificial Sequence Synthetic primer
acaaccaaaaggagattttttta

SEQ ID NO: 102 - DNA, 20 bp, Artificial Sequence
  Description of Artificial Sequence Synthetic primer
aggatacttccaaatcacat

SEQ ID NO: 103 - DNA, 20 bp, Artificial Sequence
  Description of Artificial Sequence Synthetic primer
cccccgtgccatggactgct

SEQ ID NO: 104 - DNA, 22 bp, Artificial Sequence
  Description of Artificial Sequence Synthetic primer
aactcaattacctcaatccttt

SEQ ID NO: 105 - DNA, 20 bp, Artificial Sequence
  Description of Artificial Sequence Synthetic primer
cattggttctgactctattt

SEQ ID NO: 106 - DNA, 19 bp, Artificial Sequence
  Description of Artificial Sequence Synthetic primer
ctacttttcagggttgtta

SEQ ID NO: 107 - DNA, 17 bp, Artificial Sequence
  Description of Artificial Sequence Synthetic primer
aaggaaatcttcacggg

SEQ ID NO: 108 - DNA, 20 bp, Artificial Sequence
  Description of Artificial Sequence Synthetic primer
gtgatgctgagttcacagac

SEQ ID NO: 109 - DNA, 17 bp, Artificial Sequence
  Description of Artificial Sequence Synthetic primer
gaaggggaaagggaaga

SEQ ID NO: 110 - DNA, 20 bp, Artificial Sequence
  Description of Artificial Sequence Synthetic primer
ttgcaatcaaaagagaactg

SEQ ID NO: 111 - DNA, 24 bp, Artificial Sequence
  Description of Artificial Sequence Synthetic primer
aatatgttttttaacacagaatga

SEQ ID NO: 112 - DNA, 21 bp, Artificial Sequence
  Description of Artificial Sequence Synthetic primer
tgagtatggtggaagtactag

SEQ ID NO: 113 - DNA, 18 bp, Artificial Sequence
  Description of Artificial Sequence Synthetic primer
agggtatgcctgagcaag

SEQ ID NO: 114 - DNA, 18 bp, Artificial Sequence
  Description of Artificial Sequence Synthetic primer
actgaagaaaaggcagag

SEQ ID NO: 115 - DNA, 19 bp, Artificial Sequence
  Description of Artificial Sequence Synthetic primer
ttagacactgaagaaaagg

SEQ ID NO: 116 - DNA, 24 bp, Artificial Sequence
  Description of Artificial Sequence Synthetic primer
atgcatcatagcttttgacttttt

SEQ ID NO: 117 - DNA, 17 bp, Artificial Sequence
  Description of Artificial Sequence Synthetic primer
cttgaagggttcactgt

SEQ ID NO: 118 - DNA, 17 bp, Artificial Sequence
  Description of Artificial Sequence Synthetic primer
agggctctccagaccaa

SEQ ID NO: 119 - DNA, 18 bp, Artificial Sequence
  Description of Artificial Sequence Synthetic primer
gaaacctcatgacaactg

SEQ ID NO: 120 - DNA, 20 bp, Artificial Sequence
  Description of Artificial Sequence Synthetic primer
gtgcttaaaactcctcttat

SEQ ID NO: 121 - DNA, 23 bp, Artificial Sequence
  Description of Artificial Sequence Synthetic primer
tatgattttgacttttcaaagtt

SEQ ID NO: 122 - DNA, 23 bp, Artificial Sequence
  Description of Artificial Sequence Synthetic primer
cctaataacttatgtcttacaca

SEQ ID NO: 123 - DNA, 22 bp, Artificial Sequence
  Description of Artificial Sequence Synthetic primer
aaggtgtaagtttcataaatgg

SEQ ID NO: 124 - DNA, 23 bp, Artificial Sequence
  Description of Artificial Sequence Synthetic primer
aattcacccattttaagtgtata

SEQ ID NO: 125 - DNA, 20 bp, Artificial Sequence
  Description of Artificial Sequence Synthetic primer
aaaaactgaaacctgaaact

SEQ ID NO: 126 - DNA, 22 bp, Artificial Sequence
  Description of Artificial Sequence Synthetic primer
ccaaagagcaagaaagctttaa

SEQ ID NO: 127 - DNA, 23 bp, Artificial Sequence
  Description of Artificial Sequence Synthetic primer
cacatagcaaatatatgacacaa

SEQ ID NO: 128 - DNA, 19 bp, Artificial Sequence
  Description of Artificial Sequence Synthetic primer
agttcctgccgatgttagt

SEQ ID NO: 129 - DNA, 20 bp, Artificial Sequence
  Description of Artificial Sequence Synthetic primer
ctgtggaaatcttggctatt

SEQ ID NO: 130 - DNA, 20 bp, Artificial Sequence
  Description of Artificial Sequence Synthetic primer
ccgtgaagatttccttgcag

SEQ ID NO: 131 - DNA, 20 bp, Artificial Sequence
  Description of Artificial Sequence Synthetic primer
gtggatatcactaccttccg

SEQ ID NO: 132 - DNA, 17 bp, Artificial Sequence
  Description of Artificial Sequence Synthetic primer
cttgcagtgtgctgtcc

SEQ ID NO: 133 - DNA, 21 bp, Artificial Sequence
  Description of Artificial Sequence Synthetic primer
gtcgcttcctgatgtcggtca

SEQ ID NO: 134 - DNA, 21 bp, Artificial Sequence
  Description of Artificial Sequence Synthetic primer
ggccagcctggactatatgag

SEQ ID NO: 135 - DNA, 19 bp, Artificial Sequence
  Description of Artificial Sequence Synthetic oligonucleotide
gcggagcatggtaacttct

SEQ ID NO: 136 - DNA, 19 bp, Artificial Sequence
  Description of Artificial Sequence Synthetic oligonucleotide
gctcaagttcactctacga

SEQ ID NO: 137 - DNA, 19 bp, Artificial Sequence
  Description of Artificial Sequence Synthetic oligonucleotide
ctctacgagactgcaatag

SEQ ID NO: 138 - DNA, 19 bp, Artificial Sequence
  Description of Artificial Sequence Synthetic oligonucleotide
aatttcgagagcatcagtt

SEQ ID NO: 139 - RNA, 19 nt, Artificial Sequence
  Description of Artificial Sequence Synthetic oligonucleotide
cguacgcggaauacuucga

SEQ ID NO: 140 - DNA, 30 bp, Artificial Sequence
  Description of Artificial Sequence Synthetic primer
acgttggatgggtgtggagtacagttcttg

SEQ ID NO: 141 - DNA, 30 bp, Artificial Sequence
  Description of Artificial Sequence Synthetic primer
acgttggatgcggtgacaccaacctttttc

SEQ ID NO: 142 - DNA, 18 bp, Artificial Sequence
  Description of Artificial Sequence Synthetic primer
tttttcatgtcatctgtg

SEQ ID NO: 143 - DNA, 73 bp, Artificial Sequence
  Description of Artificial Sequence Synthetic primer
cggtgacaccaacctttttcatgtcatctgtgcaaatcttggctattgtgtcacaagaactgtactccacacc